(19) United States
(12) Patent Application Publication
(10) Pub. No.: US 2012/0301887 A1
(43) Pub. Date: Nov. 29, 2012

(54) Gene Expression Profiling for the Identification, Monitoring, and Treatment of Prostate Cancer

(76) Inventors: Danute M. Bankaitis-Davis, Longmont, CO (US); Lisa Siconolfi, Westminster, CO (US); Kathleen Storm, Longmont, CO (US); Karl Weissmann, Dover, MA (US)

(21) Appl. No.: 13/143,171

(22) PCT Filed: Jan. 6, 2010
(86) PCT No.: PCT/US10/00037

§ 371 Date: Sep. 12, 2011

Related U.S. Application Data
(60) Provisional application No. 61/228,004, filed on Jul. 23, 2009.
(60) Provisional application No. 61/178,342, filed on May 14, 2009.
(60) Provisional application No. 61/170,017, filed on Apr. 16, 2009.
(60) Provisional application No. 61/163,354, filed on Mar. 25, 2009.
(60) Provisional application No. 61/150,666, filed on Feb. 6, 2009.
(60) Provisional application No. 61/142,789, filed on Jan. 6, 2009.

(51) Int. Cl. C12Q 1/68

(52) U.S. Cl. 435/6.12; 435/6.14

(57) ABSTRACT

A method is provided in various embodiments for determining a profile data set for a subject with prostate cancer or a condition related to prostate cancer based on a sample from the subject, wherein the sample provides a source of RNAs. The method includes using amplification for measuring the amount of RNA corresponding to at least 1 constituent from Table 1 and/or Table 8 in conjunction with PSA. The profile data set comprises the measure of each constituent, and amplification is performed under measurement conditions that are substantially repeatable.

### REFERENCE TO RELATED APPLICATIONS

This application claims the benefit of U.S. Provisional Application No. 61/142,789, filed Jan. 6, 2009, U.S. Provisional Application No. 61/150,666, filed Feb. 6, 2009, U.S. Provisional Application No. 61/163,354, filed Mar. 25, 2009, U.S. Provisional Application No. 61/170,017, filed Apr. 16, 2009, U.S. Provisional Application No. 61/178,342, filed May 14, 2009, and U.S. Provisional Application No. 61/228,004, filed Jul. 23, 2009, each of which are hereby incorporated by reference in their entireties.

### FIELD OF THE INVENTION

The present invention relates generally to the identification of biological markers associated with the identification of prostate cancer. More specifically, the present invention relates to the use of gene expression data in the identification, monitoring and treatment of prostate cancer and in the characterization and evaluation of conditions induced by or related to prostate cancer.

### BACKGROUND OF THE INVENTION

Prostate cancer is the most common cancer diagnosed among American men, with more than 234,000 new cases per year. As a man increases in age, his risk of developing prostate cancer increases exponentially. Under the age of 40, 1 in 1000 men will be diagnosed; between ages 40-59, 1 in 38 men will be diagnosed and between the ages of 60-69, 1 in 14 men will be diagnosed. More that 65% of all prostate cancers are diagnosed in men over 65 years of age. Beyond the significant human health concerns related to this dangerous and common form of cancer, its economic burden in the U.S. has been estimated at $8 billion dollars per year, with average annual costs per patient of approximately $12,000.

Prostate cancer is a heterogeneous disease, ranging from asymptomatic to a rapidly fatal metastatic malignancy. Survival of the patient with prostatic carcinoma is related to the extent of the tumor. When the cancer is confined to the prostate gland, median survival in excess of 5 years can be anticipated. Patients with locally advanced cancer are not usually curable, and a substantial fraction will eventually die of their tumor, though median survival may be as long as 5 years. If prostate cancer has spread to distant organs, current therapy will not cure it. Median survival is usually 1 to 3 years, and most such patients will die of prostate cancer. Even in this group of patients, however, indolent clinical courses lasting for many years may be observed. Other factors affecting the prognosis of patients with prostate cancer that may be useful in making therapeutic decisions include histologic grade of the tumor, patient's age, other medical illnesses, and PSA levels.

Early prostate cancer usually causes no symptoms. As a result, early forms of prostate cancer oftentimes go undetected until it has advanced into a more aggressive form of the disease. However, the symptoms that do present are often similar to those of diseases such as benign prostatic hypertrophy. Such symptoms include frequent urination, increased urination at night, difficulty starting and maintaining a steady stream of urine, blood in the urine, and painful urination. Prostate cancer may also cause problems with sexual function, such as difficulty achieving erection or painful ejaculation.

Currently, there is no single diagnostic test capable of differentiating early prostated cancer from benign prostatic hyperplasia, or capable of distinguishing clinically aggressive from clinically benign disease. Since individuals can have prostate cancer for several years and remain asymptomatic while the disease progresses and metastasizes, screenings are essential to detect prostate cancer at the earliest stage possible. Although detection of prostate cancer is routinely achieved with physical examination and/or clinical tests such as serum prostate-specific antigen (PSA) test, this test is not definitive, since PSA levels can also be elevated due to prostate infection, enlargement, race and age effects. For example, a PSA level of 3 or less is considered in the normal range for a male under 60 years old, a level of 4 or less is considered normal for a male between the ages of 60-69, and a level of 5 or less is normal for males over the age of 70. Generally, the higher the level of PSA, the more likely prostate cancer is present. However, a PSA level above the normal range (depending on the age of the patient) could be due to benign prostatic disease. In such instances, a diagnosis would be impossible to confirm without biopsying the prostate and assigning a Gleason score. Additionally, regular screening of asymptomatic men remains controversial since the PSA screening methods currently available are associated with high false-positive rates, resulting in unnecessary biopsies, which can result in significant morbidity.

Additionally, the clinical course of prostate cancer disease can be unpredictable, and the prognostic significance of the current diagnostic measures remains unclear. Furthermore, current tests do not reliably identify patients who are likely to respond to specific therapies—especially for cancer that has spread beyond the prostate gland. Information on any condition of a particular patient and a patient's response to types and dosages of therapeutic or nutritional agents has become an important issue in clinical medicine today not only from the aspect of efficiency of medical practice for the health care industry but for improved outcomes and benefits for the patients. Thus, there is the need for tests which can aid in the diagnosis of prostate cancer disease as well as monitor the progression and treatment of prostate cancer.

### SUMMARY OF THE INVENTION

The invention is in based in part upon the identification of gene expression profiles (Precision Profiles™) associated with prostate cancer. These genes are referred to herein as prostate cancer associated genes or prostate cancer associated constituents. More specifically, the invention is based upon the surprising discovery that detection of as few as one prostate cancer associated gene in a subject derived sample is capable of identifying individuals with or without prostate cancer with at least 55% accuracy, preferably at least 75% accuracy. More particularly, the invention is based upon the surprising discovery that the methods provided by the invention are capable of detecting prostate cancer by assaying blood samples.

The invention provides methods of detecting and/or evaluating the presence or absence (e.g., diagnosing or prognosing) of prostate cancer, based on a sample from the subject, the sample providing a source of RNAs, and determining a quantitative measure of the amount of at least one constituent of any constituent of Table 1 and arriving at a measure of each constituent.

In various aspects, the invention also provides methods for detecting/identifying subjects with or at risk for developing aggressive forms of prostate cancer (i.e. a high Gleason score such as 7 (4+3) or higher). Optionally, the PSA level of the subject may be measured in conjunction with the at least one constituent of Table 1 and/or Table 8 to in order to evaluate the presence, absence, or nature of prostate cancer. In some embodiments, the constituent that is measured is not IL-8. In another particular embodiment, the methods of the present invention are used in conjunction with Gleason score when Gleason score is above 2 but under 10, more preferably above 2 but under 8, more preferably above 2 but under 6, and even more preferably above 2 but under 4.

Also provided are methods of assessing or monitoring the response to therapy in a subject having prostate cancer, based on a sample from the subject, the sample providing a source of RNAs, by determining a quantitative measure of the amount of at least one constituent of Table 1 and/or Table 8, and arriving at a measure of each constituent.

In a further aspect the invention provides methods of monitoring the progression of prostate cancer in a subject, based on a sample from the subject, the sample providing a source of RNAs, by determining a quantitative measure of the amount of at least one constituent of Table 1 and/or Table 8 as a distinct RNA constituent in a sample obtained at a first period of time to produce a first subject data set and determining a quantitative measure of the amount of at least one constituent of Table 1 and/or Table 8 as a distinct RNA constituent in a sample obtained at a second period of time to produce a second subject data set. Optionally, the constituents measured in the first sample are the same constituents measured in the second sample. The first subject data set and the second subject data set are compared allowing the progression of prostate cancer in a subject to be determined. The second subject is taken e.g., one day, one week, one month, two months, three months, 1 year, 2 years, or more after the first subject sample. Optionally the first subject sample is taken prior to the subject receiving treatment, e.g. chemotherapy, radiation therapy, or surgery and the second subject sample is taken after treatment.

In various aspects the invention provides a method for determining a profile data set, i.e., a prostate cancer profile, for characterizing a subject with prostate cancer or conditions related to prostate cancer based on a sample from the subject, the sample providing a source of RNAs, by using amplification for measuring the amount of RNA in a panel of constituents including at least 1 constituent from Table 1 and/or Table 8 and arriving at a measure of each constituent. The profile data set contains the measure of each constituent of the panel.

The methods of the invention further include comparing the quantitative measure of the constituent in the subject derived sample to a reference value or a baseline value, e.g. baseline data set. The reference value is for example an index value. Comparison of the subject measurements to a reference value allows for the presence or absence of prostate cancer to be determined, response to therapy to be monitored, the progression of prostate cancer to be determined, or the nature of the tumor to be assessed, such as an aggressive tumor (e.g., Gleason score of 7 (4+3) or higher) or non-aggressive tumor (e.g., Gleason score of 7 (3+4) or less). For example, a similarity in the subject data set compared to a baseline data set derived from a subject having prostate cancer indicates that presence of prostate cancer or response to therapy that is not efficacious. Whereas a similarity in the subject data set compared to a baseline data set derived from a subject not having prostate cancer indicates the absence of prostate cancer or response to therapy that is efficacious. In various embodiments, the baseline data set is derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure, and the baseline profile data set may be derived from one or more other samples from one or more different subjects.

The baseline data set or reference values may be derived from one or more other samples from the same subject taken under circumstances different from those of the first sample, and the circumstances may be selected from the group consisting of (i) the time at which the first sample is taken (e.g., before, after, or during treatment cancer treatment), (ii) the site from which the first sample is taken, (iii) the biological condition of the subject when the first sample is taken.

The measure of the constituent is increased or decreased in the subject compared to the expression of the constituent in the reference, e.g., normal reference sample or baseline value. The measure is increased or decreased 10%, 25%, 50% compared to the reference level. Alternately, the measure is increased or decreased 1, 2, 5 or more fold compared to the reference level.

In various aspects of the invention the methods are carried out wherein the measurement conditions are substantially repeatable, particularly within a degree of repeatability of better than ten percent, five percent or more particularly within a degree of repeatability of better than three percent, and/or wherein efficiencies of amplification for all constituents are substantially similar, more particularly wherein the efficiency of amplification is within ten percent, more particularly wherein the efficiency of amplification for all constituents is within five percent, and still more particularly wherein the efficiency of amplification for all constituents is within three percent or less.

In addition, the one or more different subjects may have in common with the subject at least one of age group, gender, ethnicity, geographic location, nutritional history, medical condition, clinical indicator, medication, physical activity, body mass, and environmental exposure. A clinical indicator may be used to assess prostate cancer or a condition related to prostate cancer of the one or more different subjects, and may also include interpreting the calibrated profile data set in the context of at least one other clinical indicator, wherein the at least one other clinical indicator includes blood chemistry (e.g., PSA levels), X-ray or other radiological or metabolic imaging technique, molecular markers in the blood, other chemical assays, and physical findings.

At least 1, 2, 3, 4, 5, 6, 7, 8, 9, 10, 15, 20, 30 40, 50 or more constituents are measured. The constituents are selected so as to distinguish a prostate cancer diagnosed subject from a normal, healthy reference subject. The constituents may also be selected so as to distinguish a prostate cancer diagnosed subject from an otherwise healthy subject with benign prostatic hyperplasia (also known as benign prostatic hypertrophy, or “BPH”), which oftentimes includes signs and/or symptoms similar to the signs and symptoms of prostate cancer. In some embodiments, the prostate cancer-diagnosed subject is diagnosed with different stages of cancer.

In particular embodiments the constituents are selected so as to identify, predict and/or discriminate between prostate cancer-diagnosed subjects having an aggressive versus non-aggressive form of prostate cancer. The skilled artisan would recognize that a Gleason score of 7 can be obtained by either a primary grade plus secondary grade of (3+4) or (4+3), the former indicative of less aggressive tumors and the latter with more aggressive tumors. Thus, in a particular embodiment, the constituents are selected so as to identify, predict and/or discriminate between prostate cancer subjects having a Gleason scores of <8 from prostate cancer subjects with a Gleason score of 8-9. In another particular embodiment, the constituents are selected so as to identify, predict and/or discriminate between prostate cancer subjects with a Gleason score of 6-7 (3+4) (i.e., less aggressive form of cancer) from prostate cancer subjects with a Gleason scores of 7 (4+3), 8 or 9 (i.e., more aggressive form of cancer). In yet another particular embodiment the constituents are selected so as to identify, predict and/or discriminate between prostate cancer subjects with a Gleason score of <7 (i.e., less aggressive form of cancer) from those with Gleason scores of 7, 8 or 9 (i.e., more aggressive form of cancer).

Alternatively, the panel of constituents is selected as to permit characterizing the severity of prostate cancer in relation to a normal subject over time so as to track movement toward normal as a result of successful therapy and away from normal in response to cancer recurrence. Thus in some embodiments, the methods of the invention are used to determine efficacy of treatment of a particular subject.

Preferably, the constituents are selected so as to distinguish, e.g., classify between a prostate cancer-diagnosed subject and a normal subject, or between a prostate cancer-diagnosed subject and an otherwise healthy subject with BPH, or between a prostate cancer-diagnosed subject having a high Gleason score (e.g., Gleason score of (7 (4+3) or higher; i.e., more aggressive form of cancer) from those having a low Gleason score (e.g., Gleason score of 7 (3+4), 6 or less; i.e., less aggressive form of cancer), with at least 55%, 60%, 65%, 60%, 75%, 80%, 85%, 90%, 95%, 97%, 98%, 99% or greater accuracy. Accuracy is determined for example by comparing the results of the Gene Precision Profiling™ to standard accepted clinical methods of diagnosing prostate cancer, e.g., PSA test, digital rectal exam, biopsy procedures, and combinations thereof.

The selected constituents (i.e., gene models) can be used iteratively/incrementally. For example, two or more gene models can be used to discriminate first between prostate cancer patients and normal or otherwise healthy subjects with BPH, then to further identify, predict and/or discriminate between prostate cancer patients having high versus low Gleason scores (e.g., Gleason score 7 (4+3) or higher) vs. Gleason score of 7 (3+4), 6 or lower)).

For example without limitation, any of the 3-gene models enumerated in Table 2A, any of the 3-gene models enumerated in Table 3, any of the 2-gene, 4-gene and 6-gene models listed in Table 4, any of the 8-gene models enumerated in Table 17B, can be measured to distinguish a prostate cancer diagnosed subject from a normal, healthy reference subject with at least 55% accuracy, preferably at least 75% accuracy. As a further example, without limitation, any of the 3-gene models enumerated in Table 5A, and any of the 1-gene, 2-gene, 3-gene and 5-gene models listed in Table 6, can be measured to distinguish a prostate cancer-diagnosed subject from a subject with BPH with at least 55% accuracy, preferably at least 75% accuracy.

In one embodiment, at least 1 constituent from Table 1 and/or Table 8 is measured to distinguish a prostate cancer diagnosed subject from a normal, healthy reference subject (or otherwise healthy subject with BPH), wherein the at least 1 constituents is selected from IL18, RP51077B9.4, and S100A6.

In another embodiment, at least 2 constituents from Table 1 and/or Table 8 are measured to distinguish a prostate cancer diagnosed subject from a normal, healthy reference subject (or otherwise healthy subject with BPH), wherein the at least two constituents are selected from the following combinations of constituents: a) ABL1 and BRCA1; b) MAP2K1 and MAPK1; c) BRCA1 and MAP2K1; d) PTPRC and RP51077B9.4; e) CD97 and SP1; CD97 and S100A6; g) IL18 and RP5107B9.4; h) MAP2K1 and S100A6, i) RP51077B9.4 and S100A6; and j) RP51077B9.4 and SP1.

In still another embodiment, at least 3 constituents from Table 1 and/or Table 8 are measured to distinguish a prostate cancer diagnosed subject from a normal, healthy reference subject (or otherwise healthy subject with BPH) with at least 55% accuracy, preferably at least 75% accuracy, wherein the at least 3 constituents are selected from the following combinations of constituents: a) MAP2K1, MYC and S100A6; b) MAP2K1, S100A6 and SMAD3; and c) MAP2K1, S100A6 and TP53.

In yet another embodiment, at least 4 constituents from Table 1 and/or Table 8 are measured to distinguish a prostate cancer diagnosed subject from a normal, healthy reference subject (or otherwise healthy subject with BPH) with at least 55% accuracy, preferably at least 75% accuracy, wherein the at least 4 constituents are selected from the following combinations of constituents: a) CD97, CDK2, RP51077B9.4 and SP1; b) BRCA1, GSK3B, RB1 and TNF.

In a particular embodiment, at least 5 constituents from Table 1 and/or Table 8 are measured to distinguish a prostate cancer diagnosed subject from a normal, healthy reference subject (or otherwise healthy subject with BPH) with at least 55% accuracy, preferably at least 75% accuracy, wherein the at least 5 constituents are selected from the following combinations of constituents: a) S100A6, MYC, MAP2K1, C1QA, and RP51077B9.4; b) MAP2K1, SMAD3, S100A6, CCNE1, and TP53; and c) MAP2K1, TP53, S100A6, CCNE1 and ST14.

In another particular embodiment, at least 6 constituents from Table 1 and/or Table 8 are measured to distinguish a prostate cancer diagnosed subject from a normal, healthy reference subject (or otherwise healthy subject with BPH) with at least 55% accuracy, preferably at least 75% accuracy, wherein the at least 6 constituents are selected from the following combinations of constituents: a) RP51077B9.4, CD97, CDKN2A, SP1, S100A6, and IQGAP1; and b) CD97, GSK3B, PTPRC, RP51077B9.4, SP1 and TNF.

In yet another particular embodiment, at least 8 constituents from Table 1 and/or Table 8 are measured to distinguish a prostate cancer diagnosed subject from a normal, healthy reference subject (or otherwise healthy subject with BPH) with at least 55% accuracy, preferably at least 75% accuracy, wherein the at least 8 constituents are selected from the following combinations of constituents: a) BRCA1, CD97, CDK2, IQGAP1, PTPRC, RP51077B9.4, SP1, and TNF; b) ABL1, BRCA1, CD97, IL18, IQGAP1, RP51077B9.4, SP1, and TNF; c) RP51077B9.4, IQGAP1, ABL1, BRCA1, RB1, TNF, and CD97; d) RP51077B9.4, CD97, CDKN2A, IQGAP1, ABL1, BRCA1 and PTPRC; and d) SP1, CD97, IQGAP1, RP51077B9.4, ABL1, BRCA1, CDKN2A and PTPRC.

In yet further examples, at least one constituent from Table 1 and/or Table 8 is measured to distinguish a prostate cancer diagnosed subject having a high versus low Gleason score. For example, at least one constituent from Table 1 and/or Table 8 is measured to distinguish a′ prostate cancer diagnosed subject having a Gleason score of 8-9 from a prostate cancer diagnosed subject having a Gleason score <8 with at least 55% accuracy, preferably at least 75% accuracy, wherein the at least 1 constituent is selected from the group consisting of C1QA, CCND2, COL6A2, and TIMP1. In another example, without limitation, at least 2 constituents from Table 1 and/or Table 8 are measured to distinguish a prostate cancer diagnosed subject having a Gleason score of 8-9 from a prostate cancer diagnosed subject having a Gleason score <8 with at least 55% accuracy, preferably at least 75% accuracy, wherein the at least 2 constituents are CCND2 and COL6A2. As another example, without limitation, at least 3 constituents from Table 1 and/or Table 8 are measured to distinguish a prostate cancer diagnosed subject having a Gleason score of 8-9 from a prostate cancer diagnosed subject having a Gleason score <8 with at least 55% accuracy, preferably at least 75% accuracy, wherein the at least 3 constituents are CCND2, COL6A2 and CDKN2A.

In a further example, at least 2 constituents are measured to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. For example, any of the 2- or 3-gene models enumerated in Table 7A, Table 9 or Table 10 can measured to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. In one particular embodiment, CD4 and TP53 are measured. As a yet another example, as least three constituents from Table 1 and/or Table 8 are measured to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. In particular embodiments, CASP9, and two constituents selected from the following combination of constituents are measured: PLEK2 and RB1; SIAH2 and VEGF; RB1 and XK; IGF2BP2 and VEGF; NCOA4 and VEGF; VEGF and XK; SRF and XK; and IGF2BP2 and RB1. In other particular embodiments, CASP1, and two constituents selected from the following combination of constituents are measured: CD44 and POV1; EP300 and MTF1; NFKB1 and POV1; and IGF2BP2 and SERPING1. In yet other embodiments, CDKN2A, and two constituents selected from the following combination of constituents are measured: CTSD and VHL; and KAI1 and VHL. In still other embodiments, MTA1, POV1 and RB1 are measured. As a further example, CD44, POV1 and RB1 are measured. In yet another example, G1P3, PLEK2 and VEGF are measured. In still another example, C1QB, CASP1 and KAI1. In yet another example, CD4, TP53 and E2F1 are measured.

As even further examples, at least two constituents from Table 1 and/or Table 8 are measured to distinguish between prostate cancer subjects having a Gleason score of 7 or higher (i.e., more aggressive form of cancer) from those having less a Gleason score of 6 or lower (i.e., less aggressive form of cancer) with at least 55% accuracy, preferably at least 75% accuracy. For example, any of the 2- or 3-gene models enumerated in Table 7A, Table 9 or Table 10 can measured to distinguish between prostate cancer subjects having a Gleason score of 7 or higher from those having less a Gleason score of 6 or lower. For example, CASP9 and SOCS3 are measured. In even further examples, at least three constituents from Table 1 and/or Table 8 are measured to distinguish between prostate cancer subjects having a Gleason score of 7 or higher (i.e., more aggressive form of cancer) from those having less a Gleason score of 6 or lower (i.e., less aggressive form of cancer). For example, ELA2, and two constituents selected from the following combination of constituents are measured: RB1 and SIAH2; RB1 and XK; and PLEK2 and RB1. As another example, CASP1, ELA2 and PLEK2 are measured. As yet another example, ANLN, and two constituents selected from the following combination of constituents are measured: CASP1 and PLEK2; and PLEK2 and RB1.

In yet other examples, any of the 2- or 3-gene models enumerated in Tables 9 or 10 can be measured to distinguish between prostate cancer subjects having a high versus a low Gleason score (e.g., Gleason score 7(4+3) or higher versus Gleason score of 7(3+4) or less, or Gleason score 7 or higher versus Gleason score 6 or less) with at least 55% accuracy, preferably at least 75% accuracy.

In a particular embodiment, the methods of the present invention are used in conjunction with the PSA test when PSA levels are above 2 but under 100, more preferably above 3 but under 50, more preferably above 3 but under 30, more preferably above 3 but under 15, and even more preferably above 3 but under 10. In particular embodiments, the methods of the present invention are used in conjunction with age-adjusted PSA criteria. Use of the methods of the present invention in conjunction with PSA levels provides a better diagnosis and/or prognosis of prostate cancer, over the use of PSA levels alone.

For example without limitation, For example without limitation, any of the 3-gene models enumerated in Table 2A, any of the 3-gene models enumerated in Table 3, any of the 2-gene, 4-gene and 6-gene models listed in Table 4, any of the 8-gene models enumerated in Table 17B, can be measured in conjunction with PSA to distinguish a prostate cancer diagnosed subject from a normal, healthy reference subject with at least 55% accuracy, preferably at least 75% accuracy. As a further example, without limitation, any of the 3-gene models enumerated in Table 5A, and any of the 1-gene, 2-gene, 3-gene and 5-gene models listed in Table 6, can be measured in conjunction with PSA to distinguish a prostate cancer diagnosed subject from a normal, healthy reference subject with at least 55% accuracy, preferably at least 75% accuracy.

In one embodiment, at least 1 constituent from Table 1 and/or Table 8 is measured in conjunction with PSA to distinguish a prostate cancer diagnosed subject from a normal, healthy reference subject (or otherwise healthy subject with BPH), wherein the at least 1 constituents is selected from IL18, RP51077B9.4, and S100A6.

In another embodiment, at least 2 constituents from Table 1 and/or Table 8 are measured in conjunction with PSA to distinguish a prostate cancer diagnosed subject from a normal, healthy reference subject (or otherwise healthy subject with BPH) with at least 55% accuracy, preferably at least 75% accuracy, wherein the at least two constituents are selected from the following combinations of constituents: a) ABL1 and BRCA1; b) MAP2K1 and MAPK1; c) BRCA1 and MAP2K1; d) PTPRC and RP51077B9.4; e) CD97 and SP1; f) CD97 and S100A6; g) IL18 and RP5107B9.4; h) MAP2K1 and S100A6, i) RP51077B9.4 and S100A6; and j) RP51077B9.4 and SP1.

In still another embodiment, at least 3 constituents from Table 1 and/or Table 8 are measured in conjunction with PSA to distinguish a prostate cancer diagnosed subject from a normal, healthy reference subject (or otherwise healthy subject with BPH) with at least 55% accuracy, preferably at least 75% accuracy, wherein the at least 3 constituents are selected from the following combinations of constituents: a) MAP2K1, MYC and S100A6; b) MAP2K1, S100A6 and SMAD3; and c) MAP2K1, S100A6 and TP53.

In yet another embodiment, at least 4 constituents from Table 1 and/or Table 8 are measured in conjunction with PSA to distinguish a prostate cancer diagnosed subject from a normal, healthy reference subject (or otherwise healthy subject with BPH) with at least 55% accuracy, preferably at least 75% accuracy, wherein the at least 4 constituents are selected from the following combinations of constituents: a) CD97, CDK2, RP51077B9.4 and SP1; b) BRCA1, GSK3B, RB1 and TNF.

In a particular embodiment, at least 5 constituents from Table 1 and/or Table 8 are measured in conjunction with PSA to distinguish a prostate cancer diagnosed subject from a normal, healthy reference subject (or otherwise healthy subject with BPH) with at least 55% accuracy, preferably at least 75% accuracy, wherein the at least 5 constituents are selected from the following combinations of constituents: a) S100A6, MYC, MAP2K1, C1QA, and RP51077B9.4; b) MAP2K1, SMAD3, S100A6, CCNE1, and TP53; and c) MAP2K1, TP53, S100A6, CCNE1 and ST14.

In another particular embodiment, at least 6 constituents from Table 1 and/or Table 8 are measured in conjunction with PSA to distinguish a prostate cancer diagnosed subject from a normal, healthy reference subject (or otherwise healthy subject with BPH) with at least 55% accuracy, preferably at least 75% accuracy, wherein the at least 6 constituents are selected from the following combinations of constituents: a) RP51077B9.4, CD97, CDKN2A, SP1, S100A6, and IQGAP1; and b) CD97, GSK3B, PTPRC, RP51077B9.4, SP1 and TNF.

In yet another particular embodiment, at least 8 constituents from Table 1 and/or Table 8 are measured in conjunction with PSA to distinguish a prostate cancer diagnosed subject from a normal, healthy reference subject (or otherwise healthy subject with BPH) with at least 55% accuracy, preferably at least 75% accuracy, wherein the at least 8 constituents are selected from the following combinations of constituents: a) BRCA1, CD97, CDK2, IQGAP1, PTPRC, RP51077B9.4, SP1, and TNF; b) ABL1, BRCA1, CD97, IL18, IQGAP1, RP51077B9.4, SP1, and TNF; c) RP51077B9.4, IQGAP1, ABL1, BRCA1, RB1, TNF, and CD97; d) RP51077B9.4, CD97, CDKN2A, IQGAP1, ABL1, BRCA1 and PTPRC; and d) SP1, CD97, IQGAP1, RP51077B9.4, ABL1, BRCA1, CDKN2A and PTPRC.

In yet further examples, at least one constituent from Table 1 and/or Table 8 is measured in conjunction with PSA to distinguish a prostate cancer diagnosed subject having a high versus low Gleason score. For example at least one constituent from Table 1 and/or Table 8 is measured in conjunction with PSA to distinguish a prostate cancer diagnosed subject having a Gleason score of 8-9 from a prostate cancer diagnosed subject having a Gleason score <8 with at least 55% accuracy, preferably at least 75% accuracy, wherein the at least one constituent is selected from the group consisting of C1QA, CCND2, COL6A2, and TIMP1. In another example, without limitation, at least 2 constituents from Table 1 and/or Table 8 are measured in conjunction with PSA to distinguish a prostate cancer diagnosed subject having a Gleason score of 8-9 from a prostate cancer diagnosed subject having a Gleason score <8 with at least 55% accuracy, preferably at least 75% accuracy, wherein the at least 2 constituents are CCND2 and COL6A2. As another example, without limitation, at least 3 constituents from Table 1 and/or Table 8 are measured in conjunction with PSA to distinguish a prostate cancer diagnosed subject having a Gleason score of 8-9 from a prostate cancer diagnosed subject having a Gleason score <8 with at least 55% accuracy, preferably at least 75% accuracy, wherein the at least 3 constituents are CCND2, COL6A2 and CDKN2A.

In a further example, at least 2 constituents are measured in conjunction with PSA to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. For example, any of the 2- or 3-gene models enumerated in Table 7A, Table 9 or Table 10 can measured in conjunction with PSA to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. In a particular embodiment, CD4 and TP53 are measured in conjunction with PSA. As a yet another example, as least three constituents from Table 1 and/or Table 8 are measured in conjunction with PSA to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. In particular embodiments, CASP9, and two constituents selected from the following combination of constituents are measured in conjunction with PSA: PLEK2 and RB1; SIAH2 and VEGF; RB1 and XK; IGF2BP2 and VEGF; NCOA4 and VEGF; VEGF and XK; SRF and XK; and IGF2BP2 and RB1. In other particular embodiments, CASP1, and two constituents selected from the following combination of constituents are measured in conjunction with PSA: CD44 and POV1; EP300 and MTF1; NFKB1 and POV1; and IGF2BP2 and SERPING1. In yet other particular embodiments, CDKN2A, and two constituents selected from the following combination of constituents are measured in conjunction with PSA: CTSD and VHL; and KAI1 and VHL; In still another embodiment, MTA1, POV1 and RB1 are measured in conjunction with PSA. As a further example, PSA is measured in conjunction with CD44, POV1 and RB1. In yet another example, PSA is measured in conjunction with G1P3, PLEK2 and VEGF. In still another example, PSA is measured in conjunction with C1QB, CASP1 and KAI1. In yet another example, PSA is measured in conjunction with CD4, TP53 and E2F1.

As even further examples, at least two constituents from Table 1 and/or Table 8 are measured in conjunction with PSA to distinguish between prostate cancer subjects having a Gleason score of 7 or higher (i.e., more aggressive form of cancer) from those having less a Gleason score of 6 or lower (i.e., less aggressive form of cancer) with at least 55% accuracy, preferably at least 75% accuracy. For example, PSA is measured in conjunction with CASP9 and SOCS3. In even further examples, at least three constituents from Table 1 and/or Table 8 are measured in conjunction with PSA to distinguish between prostate cancer subjects having a Gleason score of 7 or higher (i.e., more aggressive form of cancer) from those having less a Gleason score of 6 or lower (i.e., less aggressive form of cancer) with at least 55% accuracy, preferably at least 75% accuracy. For example, ELA2, and two constituents selected from the following combination of constituents are measured in conjunction with PSA: RB1 and SIAH2; RB1 and XK; and PLEK2 and RB1. As another example, PSA is measured in conjunction with CASP1, ELA2 and PLEK2. As yet another example, ANLN, and two constituents selected from the following combination of constituents are measured in conjunction with PSA: CASP1 and PLEK2; and PLEK2 and RB1.

In yet other examples, any of the 2- or 3-gene models enumerated in Tables 9 or 10 can be measured in conjunction with PSA to distinguish between prostate cancer subjects having a high versus a low Gleason score (e.g., Gleason score 7(4+3) or higher versus Gleason score of 7(3+4) or less, or Gleason score 7 or higher versus Gleason score 6 or less) with at least 55% accuracy, preferably at least 75% accuracy.

By prostate cancer or conditions related to prostate cancer is meant the malignant growth of abnormal cells in the prostate gland, capable of invading and destroying other prostate cells, and spreading (metastasizing) to other parts of the body, including bones and lymph nodes.

The sample is any sample derived from a subject which contains RNA. For example, the sample is whole blood, a blood fraction (e.g., T-cells, B-cells, monocytes, or natural killer (NK) cells), body fluid, a population of cells or tissue from the subject, a prostate cell, or a rare circulating tumor cell or circulating endothelial cell found in the blood.

Optionally one or more other samples can be taken over an interval of time that is at least one month between the first sample and the one or more other samples, or taken over an interval of time that is at least twelve months between the first sample and the one or more samples, or they may be taken pre-therapy intervention or post-therapy intervention. In such embodiments, the first sample may be derived from blood and the baseline profile data set may be derived from tissue or body fluid of the subject other than blood. Alternatively, the first sample is derived from tissue or bodily fluid of the subject and the baseline profile data set is derived from blood.

Also included in the invention are kits for the detection of prostate cancer in a subject, containing at least one reagent for the detection or quantification of any constituent measured according to the methods of the invention and instructions for using the kit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A is a table showing the sample sizes of untreated localized prostate cancer subjects, healthy, normal subjects (without BPH) and BPH subjects by age and test group (i.e., Training Dataset and Test Dataset); FIG. 1B is a table showing the mean PSA values of untreated localized prostate cancer subjects, healthy, normal subjects (without BPH) and BPH subjects by age and test group (i.e., Training Dataset and Test Dataset); FIG. 1C is a table showing the percent of untreated localized prostate cancer subjects, healthy, normal subjects (without BPH) and BPH subjects amongst different test groups (i.e., Training and Test Datasets) meeting specified age-adjusted PSA criteria.

FIG. 2 is a ROC curve based on PSA screening showing that PSA provides discrimination of prostate cancer patients (CaP) from age-matched normal, healthy subjects (without BPH) with a specificity of 94.7% (healthy normal subjects correctly classified) and a sensitivity of 71.1% (prostate cancer subjects correctly classified).

FIG. 3 is a ROC curve for a 6-gene logit model (RP51077B9.4, CD97, CDKN2A, SP1, S100A6 and IQGAP1) compared to a model based on age-adjusted PSA criteria alone; the area under the curve (AUC) is 0.842 for PSA alone whereas the AUC is 0.946 for the 6-gene model.

FIG. 4 is a ROC curve comparing the 6-gene logit model (RP51077B9.4, CD97, CDKN2A, SP1, S100A6 and IQGAP1) combined with PSA to a model based on PSA alone; the area under the curve (AUC) is 0.842 for PSA alone whereas the AUC is 0.994 for the 6-gene+PSA model.

FIG. 5 is a scatterplot showing that a 6-gene logit model (RP51077B9.4, CD97, CDKN2A, SP1, S100A6 and IQGAP1) combined with PSA discriminates prostate cancer patients (CaP) from age-matched normal, healthy subjects (without BPH). Only 2 of the 76 CaP and 3 of the 76 normal subjects are misclassified by the 6-gene+PSA model, based on a cut-off of 0.5.

FIG. 6 is a discrimination plot showing that a 6-gene logit model (RP51077B9.4, CD97, CDKN2A, SP1, S100A6 and IQGAP1) combined with PSA discriminates prostate cancer patients (CaP) from age-matched normal, healthy subjects (without BPH) with 97.4% sensitivity (CaP subjects correctly classified; 74/76 subjects correctly classified=97.4%) and 96.1% specificity (normal subject correctly classified; 73/76 correctly classified=96.1%)).

FIG. 7 is a discrimination plot of individual subject predicted probability scores based on a 6-gene logit model (RP51077B9.4, CD97, CDKN2A, SP1, S100A6 and IQGAP1) combined with PSA, showing that the 6-gene+PSA model provides good discrimination between prostate cancer (CaP) subjects from age-matched normal subjects.

FIG. 8 is a ROC curve for a logit model based on PSA and age only, showing that PSA and age alone discriminates between prostate cancer (CaP) subjects and BPH subjects with 86.7% specificity (BPH subjects correctly classified) and 88.2% sensitivity (CaP subjects correctly classified).

FIG. 9 is a ROC curve for a 5-gene logit model (S100A6, MYC, MAP2K1, C1QA and RP51077B9.4) combined with PSA and age showing that the 5-gene+PSA+age model discriminates between prostate cancer patients (CaP) and BPH subjects with 96.1% sensitivity (CaP correctly classified) and 93.3% specificity (BPH subjects correctly classified).

FIG. 10 is a ROC curve comparing a 5-gene logit model (S100A6, MYC, MAP2K1, C1QA and RP51077B9.4) combined with PSA and age to a logit model based on PSA and age alone; the area under the curve (AUC)=0.871 for the model based on PSA and age alone, whereas AUC=0.989 for the 5-gene+PSA+age model.

FIG. 11 is a discrimination plot based on the 5-gene logit model (S100A6, MYC, MAP2K1, C1QA and RP51077B9.4) combined with PSA and age showing that the 5-gene+PSA+age model discriminates between prostate cancer patients (CaP) and BPH subjects with a sensitivity of 96.1% (i.e., CaP correctly classified; 73/76 correctly classified=96.1%) and specificity of 93.3% (i.e., BPH correctly classified; 28/30 correctly classified=93.3%).

FIG. 12 is a discrimination plot of individual subject predicted probability scores based on the 5-gene logit model (S100A6, MYC, MAP2K1, C1QA and RP51077B9.4) combined with PSA and age showing that the cut-off can be modulated to alter sensitivity and specificity of the model. A cut-off (probability of CaP) of 0.5 results in misclassification of 3 CaP subjects and 2 BPH subjects; a cut-off of 0.43 results in misclassification of 2 CaP subjects and 2 BPH subjects; and a cut-off of 0.17 results in misclassification of zero CaP subjects and 4 BPH subjects.

FIG. 13 is a bivariate discrimination plot based on a 6-gene logit model (RP51077B9.4, CD97, CDKN2A, SP1, S100A6 and IQGAP1)+PSA (Y-axis) and a 5-gene logit model (S100A6, MYC, MAP2K1, C1QA and RP51077B9.4)+PSA (X-axis) demonstrating that iterative classification based on the two models can yield almost perfect discrimination between untreated, localized prostate cancer subjects can be perfectly distinguished from normal healthy subjects (with and without BPH).

FIG. 14 is a graph showing a comparison of differences in mean delta C_{T} (cycle threshold) values for prostate cancer patients (CaP) versus normal subjects in two different test groups (Training Dataset versus Test Dataset).

FIG. 15 depicts two scatterplots comparing the results obtained by using a 6-gene logit model (RP51077B9.4, CD97, CDKN2A, SP1, S100A6 and IQGAP1) alone (i.e., not used in combination with any other predictors) to discriminate between prostate cancer subjects (CaP) and normal, healthy subjects (without BPH) in two different test groups (Training Dataset versus Test Dataset).

FIG. 16 depicts two ROC curves comparing the results obtained by using a 6-gene logit model (RP51077B9.4, CD97, CDKN2A, SP1, S100A6 and IQGAP1) alone (i.e., not used in combination with any other predictor) to discriminate between prostate cancer subjects (CaP) and normal, healthy subjects (without BPH) in two different test groups (Training Dataset versus Test Dataset).

FIG. 17 depicts two scatterplots comparing the results obtained by using a 6-gene logit model (RP51077B9.4, CD97, CDKN2A, SP1, S100A6 and IQGAP1)+PSA to discriminate between prostate cancer subjects (CaP) and normal, healthy subjects (without BPH) in two different test groups (Training Dataset versus Test Dataset).

FIG. 18 depicts two ROC curves comparing the results obtained by using a 6-gene logit model (RP51077B9.4, CD97, CDKN2A, SP1, S100A6 and IQGAP1)+PSA to discriminate between prostate cancer subjects (CaP) and normal, healthy subjects (without BPH) in two different test groups (Training Dataset versus Test Dataset).

FIG. 19 is a ROC curve comparing the results obtained by using a 6-gene logit model (RP51077B9.4, CD97, CDKN2A, SP1, S100A6 and IQGAP1)+PSA to discriminate between prostate cancer subjects (CaP) and normal, healthy subjects (with and without BPH).

FIGS. 20A and 20B are tables of re-estimated model parameters for the 6-gene logit model (RP51077B9.4, CD97, CDKN2A, SP1, S100A6 and IQGAP1) (with PSA-FIG. 19B; without PSA FIG. 19A) based on the combined results of two different test groups (Training and Test Datasets).

FIG. 21 depicts two scatterplots comparing the combined results from two different test groups (Training Dataset and Test Dataset) of a 6-gene logit model (RP51077B9.4, CD97, CDKN2A, SP1, S100A6 and IQGAP1) used with and without PSA to discriminate between prostate cancer subjects (CaP) and normal, healthy subjects (without BPH), using the re-estimated parameters shown in FIGS. 19A and 19B.

FIG. 22 is a ROC curve comparing the combined results from two different test groups (Training Dataset and Test Dataset) of a 6-gene logit model (RP51077B9.4, CD97, CDKN2A, SP1, S100A6 and IQGAP1) used with and without PSA to discriminate between prostate cancer subjects (CaP) and normal, healthy subjects (without BPH), using the re-estimated parameters shown in FIGS. 19A and 19B.

FIG. 23 a discrimination plot showing that the 2-gene logit model (CCND2 and COL6A2) discriminates prostate cancer patients (CaP) having a Gleason Score of 8-9 from CaP patients having a Gleason Score of less than 8 (Gleason score 8-9, 78.8% correct classification; Gleason score <8, 81.8% correct classification).

FIG. 24 a discrimination plot showing that the 2-gene logit model (CCND2 and COL6A2) plus PSA values discriminates prostate cancer patients (CaP) having a Gleason Score of 8-9 from CaP patients having a Gleason Score of less than 8 (Gleason score 8-9, 100% correct classification; Gleason score <8, 78.8.8% correct classification).

FIG. 25 a discrimination plot showing that the 3-gene logit model (CCND2, COL6A2 and CDKN2A) discriminates prostate cancer patients (CaP) having a Gleason Score of 8-9 from CaP patients having a Gleason Score of less than 8 (Gleason score 8-9, 100% correct classification; Gleason score <8, 81.8% correct classification).

FIG. 26A is a is a bar graph depicting the distribution of scale parameters among 2-gene qualifying models capable of distinguishing between prostate cancer (CaP) subjects with lower versus higher Gleason scores, estimated using ordinal logit methodology based on the 174 genes shown in the Precision Profile™ for Prostate Cancer Detection (Table 1); FIG. 26B is a bar graph depicting the distribution of scale parameters among 3-gene qualifying models capable of distinguishing between prostate cancer (CaP) subjects with lower versus higher Gleason scores, estimated using ordinal logit methodology based on the 174 genes shown in the Precision Profile™ for Prostate Cancer Detection (Table 1).

FIG. 27A is a ROC curve for a 3-gene logit model (C1QB, CASP1, and KAI1)+PSA, capable of discriminating between prostate cancer patients (CaP) having a low Gleason score of 6-7(3+4) and higher Gleason scores (7(4+3), 8, 9) with 92.9% sensitivity (percent GL=7(3+4), 8, 9 correctly classified) and 90% specificity (% GL=6-7(3+4) correctly classified).

FIG. 27B is a scatterplot of for a 3-gene logit model (C1QB, CASP1, and KAI1)+PSA, capable of discriminating between prostate cancer patients (CaP) having a low Gleason score of 6-7(3+4) and higher Gleason scores (7(4+3), 8, 9) with 92.9% sensitivity (percent GL=7(3+4), 8, 9 correctly classified) and 90% specificity (% GL=6-7(3+4) correctly classified).

FIG. 27C is a table which depicts the prediction of Gleason score groups among 74 prostate cancer subjects based on a 3-gene logit model (C1QB, CASP1, and KAI1) combined with PSA.

FIG. 27D is a table which depicts the prediction of Gleason score groups among 74 prostate cancer subjects based on a 3-gene logit model (C1QB, CASP1, and KAI1) combined with age-adjusted PSA criterion.

FIG. 28 is a table which depicts the Validation log-likelihood for individual predictors included in the validation of a 3-gene logit model (C1QB, CASP1, and KAI1)+PSA.

FIG. 29A is a ROC curve for a 3-gene logit model (ELA2, PLEK2 and RB1)+PSA, capable of discriminating between prostate cancer patients (CaP) having a low Gleason score of 6 and higher Gleason scores (7, 8, 9) with 80% sensitivity (percent GL=7, 8, 9 correctly classified) and 84.1% specificity (% GL=6 correctly classified); FIG. 29B is a scatterplot of for a 3-gene logit model (ELA2, PLEK2 and RB1)+PSA, capable of discriminating between prostate cancer patients (CaP) having a low Gleason score of 6 and higher Gleason scores (7, 8, 9) with 80% sensitivity (percent GL=7, 8, 9 correctly classified) and 84.1% specificity (% GL=6 correctly classified).

FIG. 30 depicts a table which lists eighteen 3-gene+PSA models capable of discriminating between prostate cancer subjects having a low Gleason score of 6-7(3+4) from prostate cancer subjects having a higher Gleason score of 7(4+3), 8, or 9 (i.e., Type 1 models).

FIG. 31 depicts a table which lists six 3-gene+PSA models capable of discriminating between prostate cancer subjects having a low Gleason score of 6 from prostate cancer subjects having a higher Gleason score of 7, 8, or 9 (i.e., Type 2 models)

FIG. 32depicts a table which lists pre-specified gene coefficients and fixed cut-off points which will be used to validate the eighteen 3-gene+PSA models shown in FIG. 28.

FIG. 33 depicts a table which lists pre-specified gene coefficients and fixed-cutoff points which will be used to validate the six 3-gene+PSA models shown in FIG. 29.

FIG. 34 is a bivariate discrimination plot based on a 6-gene logit model (RP51077B9.4, CD97, CDKN2A, SP1, S100A6 and IQGAP1)+PSA (X-axis) and a 3-gene model (C1QB, CASP1, KAI1)+PSA (Y-axis) demonstrating that iterative classification based on the two models can yield almost perfect discrimination of prostate cancer patients into high and low Gleason score groups.

FIG. 35 is a bivariate discrimination plot based on a 5-gene logit model S100A6, MYC, MAP2K1, C1QA, RP1077B9.4)+PSA (X-axis) and a 3-gene model (C1QB, CASP1, KAI1)+PSA (Y-axis) demonstrating that iterative classification based on the two models can yield almost perfect discrimination of prostate cancer patients into high and low Gleason score groups.

FIG. 36 is a diagram depicting the assumption of local independence in a latent class modeling system for using gene expression to classify subjects having high versus low Gleason scores.

FIG. 37 is a ROC curve for a latent class model consisting of combined 3-gene (TP53, CD4 and E2F1) and 2-gene (SOCS3 and CASP9) models plus age.

FIG. 38 are tables depicting descriptive Gleason statistics for PSA and age by Gleason Scores

FIG. 39 is a table depicting descriptive Gleason statistics of genes in the Type 2 model, CASP9 and SOCS3.

FIG. 40 is a table depicting descriptive Gleason statistics of genes in the Type 1 model, TP53, CD4 and E2F1.

FIG. 41 is a table depicting descriptive Gleason means and statistics for the genes TP53, CD4, E2F1, CASP9 and SOCS3, as well as PSA and age.

FIG. 42A is a bar graph depicting gene expression response for enriched B cells relative to PBMC's in samples derived from 14 subjects with newly diagnosed, localized prostate cancer (cohort 1 (Cht 1) subjects); FIG. 42B is a bar graph depicting gene expression response for depleted B cells relative to PBMC's in samples derived from 14 subjects with newly diagnosed, localized prostate cancer (cohort 1 (Cht 1) subjects).

FIG. 43 is a bar graph depicting gene expression response for enriched monocytes relative to PBMC's in samples derived from 14 subjects with newly diagnosed, localized prostate cancer (cohort 1 (Cht 1) subjects); FIG. 43B is a bar graph depicting gene expression response for depleted monocytes relative to PBMC's in samples derived from 14 subjects with newly diagnosed, localized prostate cancer (cohort 1 (Cht 1) subjects).

FIG. 44A is a bar graph depicting gene expression response for enriched NK cells relative to PBMC's in samples derived from 14 subjects with newly diagnosed, localized prostate cancer (cohort 1 (Cht 1) subjects); FIG. 44B is a bar graph depicting gene expression response for depleted NK cells relative to PBMC's in samples derived from 14 subjects with newly diagnosed, localized prostate cancer (cohort 1 (Cht 1) subjects).

FIG. 45A is a bar graph depicting gene expression response for enriched T cells relative to PBMC's in samples derived from 14 subjects with newly diagnosed, localized prostate cancer (cohort 1 (Cht 1) subjects); FIG. 45B is a bar graph depicting gene expression response for depleted T cells relative to PBMC's in samples derived from 14 subjects with newly diagnosed, localized prostate cancer (cohort 1 (Cht 1) subjects).

FIGS. 46A and 46B are bar graphs depicting gene expression response for enriched and depleted cell types relative to PBMC's in samples derived from 14 subjects with newly diagnosed, localized prostate cancer (cohort 1 (Cht 1) subjects).

FIG. 47A is a bar graph depicting gene expression response for enriched B cells relative to PBMC's in samples derived from 14 medically defined normal subjects (MDNO); FIG. 47B is a bar graph depicting gene expression response for depleted B cells relative to PBMC's in samples derived from 14 medically defined normal subjects (MDNO).

FIG. 48A is a bar graph depicting gene expression response for enriched monocytes relative to PBMC's in samples derived from 14 medically defined normal subjects (MDNO); FIG. 48B is a bar graph depicting gene expression response for depleted monocytes cells relative to PBMC's in samples derived from 14 medically defined normal subjects (MDNO).

FIG. 49A is a bar graph depicting gene expression response for enriched NK cells relative to PBMC's in samples derived from 14 medically defined normal subjects (MDNO); FIG. 49B is a bar graph depicting gene expression response for depleted NK cells relative to PBMC's in samples derived from 14 medically defined normal subjects (MDNO).

FIG. 50A is a bar graph depicting gene expression response for enriched T cells relative to PBMC's in samples derived from 14 medically defined normal subjects (MDNO); FIG. 50B is a bar graph depicting gene expression response for depleted T cells relative to PBMC's in samples derived from 14 medically defined normal subjects (MDNO).

FIGS. 51A and 51B are bar graphs depicting gene expression response for enriched and depleted cell types relative to PBMC's in samples derived from 14 medically defined normal subjects (MDNO).

FIG. 52A is a bar graph depicting a comparison of gene expression response for enriched B cells derived from medically defined normal subjects (MDNO) vs. subjects newly diagnosed with localized prostate cancer (cohort 1 (Cht 1); FIG. 52B is a bar graph depicting a comparison of gene expression response for depleted B cells derived from medically defined normal subjects (MDNO) vs. subjects newly diagnosed with localized prostate cancer (cohort 1 (Cht 1).

FIG. 53A is a bar graph depicting a comparison of gene expression response for enriched monocytes derived from medically defined normal subjects (MDNO) vs. subjects newly diagnosed with localized prostate cancer (cohort 1 (Cht 1); FIG. 53B is a bar graph depicting a comparison of gene expression response for depleted monocytes derived from medically defined normal subjects (MDNO) vs. subjects newly diagnosed with localized prostate cancer (cohort 1 (Cht 1).

FIG. 54A is a bar graph depicting a comparison of gene expression response for enriched NK cells derived from medically defined normal subjects (MDNO) vs. subjects newly diagnosed with localized prostate cancer (cohort 1 (Cht 1); FIG. 54B is a bar graph depicting a comparison of gene expression response for depleted NK cells derived from medically defined normal subjects (MDNO) vs. subjects newly diagnosed with localized prostate cancer (cohort 1 (Cht 1).

FIG. 55A is a bar graph depicting a comparison of gene expression response for enriched T cells derived from medically defined normal subjects (MDNO) vs. subjects newly diagnosed with localized prostate cancer (cohort 1 (Cht 1); FIG. 55B is a bar graph depicting a comparison of gene expression response for depleted T cells derived from medically defined normal subjects (MDNO) vs. subjects newly diagnosed with localized prostate cancer (cohort 1 (Cht 1).

FIG. 56 is a bar graph depicting gene expression response of prostate cancer cohort 1 (Cht 1) enriched cell types relative to respective enriched medically defined normal (MDNO) cells.

FIG. 57 is a flow chart depicting the steps for validating multi-gene models capable of discriminating between prostate cancer subjects from normal, healthy subjects (referred to as Category 2 models).

FIG. 58A is a ROC curve for a 6-gene+PSA model (CD97, CDKN2A, IQGAP1, RP51077B9.4, SP1, S100A6, plus PSA), capable of discriminating prostate cancer subjects from normal, healthy subjects as compared to age-adjusted PSA alone.

FIG. 58B is a ROC curve for a 6-gene+PSA model (CD97, GSK3B, PTPRC, RP51077B9.4, SP1, TNF, plus PSA), capable of discriminating prostate cancer subjects from normal, healthy subjects as compared to age-adjusted PSA alone.

FIG. 58C is a ROC curve for a 4-gene+PSA model (BRCA1, GSK3B, RB1, TNF plus PSA), capable of discriminating prostate cancer subjects from normal, healthy subjects as compared to age-adjusted PSA alone.

FIG. 58D is a ROC curve for a 4-gene+PSA model (CD97, CDK2, RP51077B9.4, SP1, plus PSA), capable of discriminating prostate cancer subjects from normal, healthy subjects as compared to age-adjusted PSA alone.

FIG. 58E is a ROC curve for a 2-gene+PSA model (CD97, SP1, plus PSA), capable of discriminating prostate cancer subjects from normal, healthy subjects as compared to age-adjusted PSA alone.

FIG. 58F is a ROC curve for a 2-gene+PSA model (PTPRC, RP51077B9.4, plus PSA), capable of discriminating prostate cancer subjects from normal, healthy subjects as compared to age-adjusted PSA alone.

FIG. 58G is a ROC curve for a 2-gene+PSA model (BRCA1, MAP2K1, plus PSA), capable of discriminating prostate cancer subjects from normal, healthy subjects as compared to age-adjusted PSA alone.

FIG. 58G is a ROC curve for a 2-gene+PSA model (MAP2K1, MAPK1, plus PSA), capable of discriminating prostate cancer subjects from normal, healthy subjects as compared to age-adjusted PSA alone.

FIG. 58I is a ROC curve for a 2-gene+PSA model (ABL1, BRCA1, plus PSA), capable of discriminating prostate cancer subjects from normal, healthy subjects as compared to age-adjusted PSA alone.

FIG. 59 is a flow chart depicting the steps for validating multi-gene models capable of discriminating between prostate cancer subjects from subjects presenting with benign prostatic hyperplasia (BPH) (referred to as Category 3 models).

FIG. 60A is a ROC curve for a 5-gene+PSA+Age model (MAP2K1, TP53, S100A6, CCNE1, ST14, plus PSA, plus age), capable of discriminating prostate cancer subjects from subjects presenting with presenting with benign prostatic hyperplasia (BPH), as compared to age-adjusted PSA alone.

FIG. 60B is a ROC curve for a 5-gene+PSA+Age model (MAP2K1, SMAD3, S100A6, CCNE1, TP53, plus PSA, plus age), capable of discriminating prostate cancer subjects from subjects presenting with presenting with benign prostatic hyperplasia (BPH), as compared to age-adjusted PSA alone.

FIG. 60C is a ROC curve for a 5-gene+PSA+Age model (MAP2K1, MYC, S100A6, RP51077B9.4, C1QA, plus PSA, plus age), capable of discriminating prostate cancer subjects from subjects presenting with presenting with benign prostatic hyperplasia (BPH), as compared to age-adjusted PSA alone.

FIG. 60D is a ROC curve for a 3-gene+PSA+Age model (MAP2K1, S100A6, SMAD3, plus PSA, plus age), capable of discriminating prostate cancer subjects from subjects presenting with presenting with benign prostatic hyperplasia (BPH), as compared to age-adjusted PSA alone.

FIG. 60E is a ROC curve for a 3-gene+PSA+Age model (MAP2K1, S100A6, TP53, plus PSA, plus age), capable of discriminating prostate cancer subjects from subjects presenting with presenting with benign prostatic hyperplasia (BPH), as compared to age-adjusted PSA alone.

FIG. 60F a is a ROC curve for a 3-gene+PSA+Age model (MAP2K1, MYC, S100A6, plus PSA, plus age), capable of discriminating prostate cancer subjects from subjects presenting with presenting with benign prostatic hyperplasia (BPH), as compared to age-adjusted PSA alone.

FIG. 60G a is a ROC curve for a 2-gene+PSA+Age model (RP51077B9.4, S100A6, plus PSA, plus age), capable of discriminating prostate cancer subjects from subjects presenting with presenting with benign prostatic hyperplasia (BPH), as compared to age-adjusted PSA alone.

FIG. 60H a is a ROC curve for a 2-gene+PSA+Age model (MAP2K1, S100A6, plus PSA, plus age), capable of discriminating prostate cancer subjects from subjects presenting with presenting with benign prostatic hyperplasia (BPH), as compared to age-adjusted PSA alone.

FIG. 60I a is a ROC curve for a 2-gene+PSA+Age model (IL18, RP51077B9.4, plus PSA, plus age), capable of discriminating prostate cancer subjects from subjects presenting with presenting with benign prostatic hyperplasia (BPH), as compared to age-adjusted PSA alone.

FIG. 60J a is a ROC curve for a 2-gene+PSA+Age model (CD97, S100A6, plus PSA, plus age), capable of discriminating prostate cancer subjects from subjects presenting with presenting with benign prostatic hyperplasia (BPH), as compared to age-adjusted PSA alone.

FIG. 60K a is a ROC curve for a 1-gene+PSA+Age model (S100A6, plus PSA, plus age), capable of discriminating prostate cancer subjects from subjects presenting with presenting with benign prostatic hyperplasia (BPH), as compared to age-adjusted PSA alone.

FIG. 60L a is a ROC curve for a 1-gene+PSA+Age model (RP51077B9.4, plus PSA, plus age), capable of discriminating prostate cancer subjects from subjects presenting with presenting with benign prostatic hyperplasia (BPH), as compared to age-adjusted PSA alone.

FIG. 60M a is a ROC curve for a 1-gene+PSA+Age model (IL18, plus PSA, plus age), capable of discriminating prostate cancer subjects from subjects presenting with presenting with benign prostatic hyperplasia (BPH), as compared to age-adjusted PSA alone.

FIG. 61 is a scatterplot depicting an 8-gene Model (RP51077B9.4, CD97, CDKN2A, SP1, IQGAP1, ABL1, PTPRC, BRCA1) without PSA, capable of discriminating between prostate cancer subjects (CaP) and normal healthy subjects with a sensitivity of 87.7% (i.e., 87.7% of the CaP subjects are correctly predicted by the model (above the arrow indicated line), and a specificity of 87.6% (i.e., 87.6% of the Normal subjects are correctly predicted by the model (below the arrow indicated line).

### DETAILED DESCRIPTION

### Definitions

The following terms shall have the meanings indicated unless the context otherwise requires:

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus. “Amplification” in the context of a quantitative RT-PCR assay is a function of the number of DNA replications that are required to provide a quantitative determination of its concentration.

“Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar.

A “baseline profile data set” is a set of values associated with constituents of a Gene Expression Panel (Precision Profile™) resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

“Benign prostatic hyperplasia” or “benign prostatic hypertrophy” (“BPH”) refers to an increase in the size of the prostate in middle-aged to elderly men characterized by hyperplasia of prostatic stromal and epithelial cells, resulting in the formation of large, discrete nodules in the periurethral region of the prostate which are benign (i.e., not considered to be premalignant lesions).

A “biological condition” of a subject is the condition of the subject in a pertinent realm that is under observation, and such realm may include any aspect of the subject capable of being monitored for change in condition, such as health; disease including cancer; trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Body fluid” of a subject includes blood, urine, spinal fluid, lymph, mucosal secretions, prostatic fluid, semen, haemolymph or any other body fluid known in the art for a subject.

“Calibrated profile data set” is a function of a member of a first profile data set and a corresponding member of a baseline profile data set for a given constituent in a panel.

A “circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.

A “circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells.

A “clinical indicator” is any physiological datum used alone or in conjunction with other data in evaluating the physiological condition of a collection of cells or of an organism. This term includes pre-clinical indicators.

“Clinical parameters” encompasses all non-sample or non-Precision Profiles™ of a subject's health status or other characteristics, such as, without limitation, age (AGE), ethnicity (RACE), gender (SEX), and family history of cancer.

A “composition” includes a chemical compound, a nutraceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

To “derive” a profile data set from a sample includes determining a set of values associated with constituents of a Gene Expression Panel (Precision Profile™) either (i) by direct measurement of such constituents in a biological sample.

“Distinct RNA or protein constituent” in a panel of constituents is a distinct expressed product of a gene, whether RNA or protein. An “expression” product of a gene includes the gene product whether RNA or protein resulting from translation of the messenger RNA.

The term “evaluating” the presence of prostate cancer” encompasses the detection, diagnosis, staging and prognosis of prostate cancer.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, statistical technique, or comparison, that takes one or more continuous or categorical inputs (herein called “parameters”) and calculates an output value, sometimes referred to as an “index” or “index value.” Non-limiting examples of “formulas” include comparisons to reference values or profiles, sums, ratios, and regression operators, such as coefficients or exponents, value transformations and normalizations (including, without limitation, those normalization schemes based on clinical parameters, such as gender, age, or ethnicity), rules and guidelines, statistical classification models, and neural networks trained on historical populations. Of particular use in combining constituents of a Gene Expression Panel (Precision Profile™) are linear and non-linear equations and statistical significance and classification analyses to determine the relationship between levels of constituents of a Gene Expression Panel (Precision Profile™) detected in a subject sample and the subject's risk of prostate cancer. In panel and combination construction, of particular interest are structural and syntactic statistical classification algorithms, and methods of risk index construction, utilizing pattern recognition features, including, without limitation, such established techniques such as cross-correlation, Principal Components Analysis (PCA), factor rotation, Logistic Regression Analysis (LogReg), Kolmogorov Smirnoff tests (KS), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques (CART, LART, LARTree, FlexTree, amongst others), Shrunken Centroids (SC), StepAIC, K-means, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a constituents of a Gene Expression Panel (Precision Profile™) selection technique, such as forward selection, backwards selection, or stepwise selection, complete enumeration of all potential panels of a given size, genetic algorithms, voting and committee methods, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other clinical studies, or cross-validated within the study they were originally trained in, using such techniques as Bootstrap, Leave-One-Out (LOO) and 10-Fold cross-validation (10-Fold CV). At various steps, false discovery rates (FDR) may be estimated by value permutation according to techniques known in the art.

A “Gene Expression Panel” (Precision Profile™) is an experimentally verified set of constituents, each constituent being a distinct expressed product of a gene, whether RNA or protein, wherein constituents of the set are selected so that their measurement provides a measurement of a targeted biological condition.

A “Gene Expression Profile” is a set of values associated with constituents of a Gene Expression Panel (Precision Profile™) resulting from evaluation of a biological sample (or population or set of samples).

A “Gene Expression Profile Inflammation Index” is the value of an index function that provides a mapping from an instance of a Gene Expression Profile into a single-valued measure of inflammatory condition.

A Gene Expression Profile Cancer Index” is the value of an index function that provides a mapping from an instance of a Gene Expression Profile into a single-valued measure of a cancerous condition.

A “Gleason Score” is the value given to prostate cancer based on its microscopic appearance, in accordance with the Gleason Staging System which predicts prostate cancer prognosis and helps guide therapy. A pathologist assigns a grade to the most common/prevalent tumor pattern (i.e., the primary grade) and a second grade to the next most common tumor pattern (i.e., the secondary grade). The primary and secondary grades are added together to get a Gleason Score. The Gleason grade ranges from 1 to 5, with 5 having the worst prognosis. The Gleason Score (i.e., sum of the primary and secondary grades) ranges from 2 to 10, with 10 having the worst prognosis. It is noted that for a Gleason Score 7 having a primary grade of 4 and secondary grade of 3 (4+3) is a more aggressive cancer than a Gleason Score 7 composed of a primary grade of 3 and a secondary grade of 4.

The “health” of a subject includes mental, emotional, physical, spiritual, allopathic, naturopathic and homeopathic condition of the subject.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response initiated or sustained by any number of chemical, physical or biological agents or combination of agents.

“Inflammatory state” is used to indicate the relative biological condition of a subject resulting from inflammation, or characterizing the degree of inflammation.

A “large number” of data sets based on a common panel of genes is a number of data sets sufficiently large to permit a statistically significant conclusion to be drawn with respect to an instance of a data set based on the same panel.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating the Predictive Value of a Diagnostic Test, How to Prevent Misleading or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing the Relationships Among Serum Lipid and Apolipoprotein Concentrations in Identifying Subjects with Coronary Artery Disease,” Clin. Chem., 1992, 38(8): 1425-1428. An alternative approach using likelihood functions, BIC, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

A “normal” subject is a subject who is generally in good health, has not been diagnosed with prostate cancer, is asymptomatic for prostate cancer, and lacks the traditional laboratory risk factors for prostate cancer.

A “normative” condition of a subject to whom a composition is to be administered means the condition of a subject before administration, even if the subject happens to be suffering from a disease.

A “panel” of genes is a set of genes including at least two constituents.

A “population of cells” refers to any group of cells wherein there is an underlying commonality or relationship between the members in the population of cells, including a group of cells taken from an organism or from a culture of cells or from a biopsy, for example.

“Positive predictive value” or “PPV” is calculated by TP/(TP+FP) or the true positive fraction of all positive test results. It is inherently impacted by the prevalence of the disease and pre-test probability of the population intended to be tested.

“Prostate cancer” is the malignant growth of abnormal cells in the prostate gland, capable of invading and destroying other prostate cells, and spreading (metastasizing) to other parts of the body, including bones and lymph nodes. As defined herein, the term “prostate cancer” includes Stage 1, Stage 2, Stage 3, and Stage 4 prostate cancer as determined by the Tumor/Nodes/Metastases (“TNM”) system which takes into account the size of the tumor, the number of involved lymph nodes, and the presence of any other metastases; or Stage A, Stage B, Stage C, and Stage D, as determined by the Jewitt-Whitmore system.

“Prostate Specific Antigen” or “PSA” is a protein produced by the cells of the prostate gland which is present in small quantities in the serum of normal (i.e., healthy) men, and is often elevated in the presence of prostate cancer and in other prostate disorders such as benign prostatic hyperplasia.

“Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of lower risk cohorts, across population divisions (such as tertiles, quartiles, quintiles, or deciles, etc.) or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, and/or the rate of occurrence of the event or conversion from one disease state to another, i.e., from a normal condition to cancer or from cancer remission to cancer, or from primary cancer occurrence to occurrence of a cancer metastasis. Risk evaluation can also comprise prediction of future clinical parameters, traditional laboratory risk factor values, or other indices of cancer results, either in absolute or relative terms in reference to a previously measured population. Such differing use may require different constituents of a Gene Expression Panel (Precision Profile™) combinations and individualized panels, mathematical algorithms, and/or cut-off points, but be subject to the same aforementioned measurements of accuracy and performance for the respective intended use.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art. The sample is whole blood, a blood fraction (e.g., T-cells, B-cells, monocytes or natural killer (NK) cells), urine, spinal fluid, lymph, mucosal secretions, prostatic fluid, semen, haemolymph or any other body fluid known in the art for a subject. The sample is also a tissue sample. The sample is or contains a circulating endothelial cell or a circulating tumor cell.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less and statistically significant at a p-value of 0.10 or less. Such p-values depend significantly on the power of the study performed.

A “set” or “population” of samples or subjects refers to a defined or selected group of samples or subjects wherein there is an underlying commonality or relationship between the members included in the set or population of samples or subjects.

A “Signature Profile” is an experimentally verified subset of a Gene Expression Profile selected to discriminate a biological condition, agent or physiological mechanism of action.

A “Signature Panel” is a subset of a Gene Expression Panel (Precision Profile™), the constituents of which are selected to permit discrimination of a biological condition, agent or physiological mechanism of action.

A “subject” is a cell, tissue, or organism, human or non-human, whether in vivo, ex vivo or in vitro, under observation. As used herein, reference to evaluating the biological condition of a subject based on a sample from the subject, includes using blood or other tissue sample from a human subject to evaluate the human subject's condition; it also includes, for example, using a blood sample itself as the subject to evaluate, for example, the effect of therapy or an agent upon the sample.

A “stimulus” includes (i) a monitored physical interaction with a subject, for example ultraviolet A or B, or light therapy for seasonal affective disorder, or treatment of psoriasis with psoralen or treatment of cancer with embedded radioactive seeds, other radiation exposure, and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

“TN” is true negative, which for a disease state test means classifying a non-disease or normal subject correctly.

“TP” is true positive, which for a disease state test means correctly classifying a disease subject.

The PCT patent application publication number WO 01/25473, published Apr. 12, 2001, entitled “Systems and Methods for Characterizing a Biological Condition or Agent Using Calibrated Gene Expression Profiles,” filed for an invention by inventors herein, and which is herein incorporated by reference, discloses the use of Gene Expression Panels (Precision Profiles™) for the evaluation of (i) biological condition (including with respect to health and disease) and (ii) the effect of one or more agents on biological condition (including with respect to health, toxicity, therapeutic treatment and drug interaction). The PCT patent application publication no. WO 08/121,132, filed Nov. 6, 2007, entitled “Gene Expression Profiling for Identification, Monitoring and Treatment of Prostate Cancer”, filed for an invention by the inventors herein, and which is herein incorporated by reference in its entirety, discloses the use of Gene Expression Panels (Precision Profiles™) for evaluating the presence or likelihood of prostate cancer in a subject, and for monitoring response to therapy in a prostate cancer-diagnosed subject, and for monitoring the progression of prostate cancer in a prostate-cancer-diagnosed subject (i.e., cancer versus a normal, healthy, disease free state).

The present invention provides an additional Gene Expression Panel (Precision Profiles™) for the detection (i.e., evaluation and characterization) of prostate cancer and conditions related to prostate cancer in a subject, and for identifying or predicting aggressive forms of prostate cancer in a prostate cancer-diagnosed subject. The Gene Expression Panel described herein may be employed with respect to samples derived from subjects in order to evaluate the presence or absence of prostate cancer, or the nature of a tumor in a prostate cancer-diagnosed subject, such as an aggressive tumor (e.g., Gleason score of 7 (4+3) or higher) or non-aggressive tumor (e.g., Gleason score of 7 (3+4), 6 or less). In addition, the Gene Expression Panel described herein also provides for the evaluation of the effect of one or more agents for the treatment of prostate cancer and conditions related to prostate cancer. The Gene Expression Panel (Precision Profile™) referred to herein is the Precision Profile™ for Prostate Cancer Detection. The Precision Profile™ for Prostate Cancer Detection includes one or more genes, e.g., constituents, listed in Table 1 and/or Table 8, whose expression is associated with prostate cancer, conditions related to prostate cancer and/or inflammation. Each gene of the Precision Profile™ for Prostate Cancer Detection is referred to herein as a prostate cancer associated gene or a prostate cancer associated constituent.

For example without limitation, any of the 3-gene models enumerated in Table 2A, any of the 3-gene models enumerated in Table 3, any of the 2-gene, 4-gene and 6-gene models listed in Table 4, any of the 8-gene models enumerated in Table 17B, can be measured to distinguish a prostate cancer diagnosed subject from a normal, healthy reference subject with at least 55% accuracy, preferably at least 75% accuracy. As a further example, without limitation, any of the 3-gene models enumerated in Table 5A, and any of the 1-gene, 2-gene, 3-gene and 5-gene models listed in Table 6, can be measured to distinguish a prostate cancer-diagnosed subject from a subject with BPH with at least 55% accuracy, preferably at least 75% accuracy.

In one embodiment, at least 1 constituent from Table 1 and/or Table 8 is measured to distinguish a prostate cancer diagnosed subject from a normal, healthy reference subject (or otherwise healthy subject with BPH), wherein the at least 1 constituents is selected from IL18, RP51077B9.4, and S100A6.

In another embodiment, at least 2 constituents from Table 1 and/or Table 8 are measured to distinguish a prostate cancer diagnosed subject from a normal, healthy reference subject (or otherwise healthy subject with BPH), wherein the at least two constituents are selected from the following combinations of constituents: a) ABL1 and BRCA1; b) MAP2K1 and MAPK1; c) BRCA1 and MAP2K1; d) PTPRC and RP51077B9.4; e) CD97 and SP1; f) CD97 and S100A6; g) IL18 and RP5107B9.4; h) MAP2K1 and S100A6, i) RP51077B9.4 and S100A6; and j) RP51077B9.4 and SP1.

In still another embodiment, at least 3 constituents from Table 1 and/or Table 8 are measured to distinguish a prostate cancer diagnosed subject from a normal, healthy reference subject (or otherwise healthy subject with BPH), wherein the at least 3 constituents are selected from the following combinations of constituents: a) MAP2K1, MYC and S100A6; b) MAP2K1, S100A6 and SMAD3; and c) MAP2K1, S100A6 and TP53.

In yet another embodiment, at least 4 constituents from Table 1 and/or Table 8 are measured to distinguish a prostate cancer diagnosed subject from a normal, healthy reference subject (or otherwise healthy subject with BPH), wherein the at least 4 constituents are selected from the following combinations of constituents: a) CD97, CDK2, RP51077B9.4 and SP1; b) BRCA1, GSK3B, RB1 and TNF.

In a particular embodiment, at least 5 constituents from Table 1 and/or Table 8 are measured to distinguish a prostate cancer diagnosed subject from a normal, healthy reference subject (or otherwise healthy subject with BPH), wherein the at least 5 constituents are selected from the following combinations of constituents: a) S100A6, MYC, MAP2K1, C1QA, and RP51077B9.4; b) MAP2K1, SMAD3, S100A6, CCNE1, and TP53; and c) MAP2K1, TP53, S100A6, CCNE1 and ST14.

In another particular embodiment, at least 6 constituents from Table 1 and/or Table 8 are measured to distinguish a prostate cancer diagnosed subject from a normal, healthy reference subject (or otherwise healthy subject with BPH), wherein the at least 6 constituents are selected from the following combinations of constituents: a) RP51077B9.4, CD97, CDKN2A, SP1, S100A6, and IQGAP1; and b) CD97, GSK3B, PTPRC, RP51077B9.4, SP1 and TNF.

In yet another particular embodiment, at least 8 constituents from Table 1 and/or Table 8 are measured to distinguish a prostate cancer diagnosed subject from a normal, healthy reference subject (or otherwise healthy subject with BPH), wherein the at least 8 constituents are selected from the following combinations of constituents: a) BRCA1, CD97, CDK2, IQGAP1, PTPRC, RP51077B9.4, SP1, and TNF; b) ABL1, BRCA1, CD97, IL18, IQGAP1, RP51077B9.4, SP1, and TNF; c) RP51077B9.4, IQGAP1, ABL1, BRCA1, RB1, TNF, and CD97; d) RP51077B9.4, CD97, CDKN2A, IQGAP1, ABL1, BRCA1 and PTPRC; and d) SP1, CD97, IQGAP1, RP51077B9.4, ABL1, BRCA1, CDKN2A and PTPRC.

In yet further examples, at least one constituent from Table 1 and/or Table 8 is measured to distinguish a prostate cancer diagnosed subject having a high versus low Gleason score. For example, at least one constituent from Table 1 and/or Table 8 is measured to distinguish a prostate cancer diagnosed subject having a Gleason score of 8-9 from a prostate cancer diagnosed subject having a Gleason score <8, wherein the at least 1 constituent is selected from the group consisting of C1QA, CCND2, COL6A2, and TIMP1. In another example, without limitation, at least 2 constituents from Table 1 and/or Table 8 are measured to distinguish a prostate cancer diagnosed subject having a Gleason score of 8-9 from a prostate cancer diagnosed subject having a Gleason score <8, wherein the at least 2 constituents are CCND2 and COL6A2. As another example, without limitation, at least 3 constituents from Table 1 and/or Table 8 are measured to distinguish a prostate cancer diagnosed subject having a Gleason score of 8-9 from a prostate cancer diagnosed subject having a Gleason score <8, wherein the at least 3 constituents are CCND2, COL6A2 and CDKN2A.

In a further example, at least 2 constituents are measured to distinguish between prostate cancer subjects having a Gleason score of 7 (4+3)) or higher (i.e., more aggressive form of cancer) from those having less a Gleason score of 7(3+4) or lower (i.e., less aggressive form of cancer). For example, any of the 2- or 3-gene models enumerated in Table 7A, Table 9 or Table 10 can measured to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. In one particular embodiment, CD4 and TP53 are measured. As a yet another example, as least three constituents from Table 1 and/or Table 8 are measured to distinguish between prostate cancer subjects having a Gleason score of 7 (4+3)) or higher (i.e., more aggressive form of cancer) from those having less a Gleason score of 7(3+4) or lower (i.e., less aggressive form of cancer). In particular embodiments, CASP9, and two constituents selected from the following combination of constituents are measured: PLEK2 and RB1; SIAH2 and VEGF; RB1 and XK; IGF2BP2 and VEGF; NCOA4 and VEGF; VEGF and XK; SRF and XK; and IGF2BP2 and RB1. In other particular embodiments, CASP1, and two constituents selected from the following combination of constituents are measured: CD44 and POV1; EP300 and MTF1; NFKB1 and POV1; and IGF2BP2 and SERPING1. In yet other embodiments, CDKN2A, and two constituents selected from the following combination of constituents are measured: CTSD and VHL; and KAI1 and VHL. In still other embodiments, MTA1, POV1 and RB1 are measured. As a further example, CD44, POV1 and RB1 are measured. In yet another example, G1P3, PLEK2 and VEGF are measured. In still another example, C1QB, CASP1 and KAI1. In yet another example, CD4, TP53 and E2F1 are measured.

As even further examples, at least two constituents from Table 1 and/or Table 8 are measured to distinguish between prostate cancer subjects having a Gleason score of 7 or higher (i.e., more aggressive form of cancer) from those having less a Gleason score of 6 or lower (i.e., less aggressive form of cancer). For example, any of the 2- or 3-gene models enumerated in Table 7A, Table 9 or Table 10 can measured to distinguish between prostate cancer subjects having a Gleason score of 7 or higher from those having less a Gleason score of 6 or lower. For example, CASP9 and SOCS3 are measured. In even further examples, at least three constituents from Table 1 and/or Table 8 are measured to distinguish between prostate cancer subjects having a Gleason score of 7 or higher (i.e., more aggressive form of cancer) from those having less a Gleason score of 6 or lower (i.e., less aggressive form of cancer). For example, ELA2, and two constituents selected from the following combination of constituents are measured: RB1 and SIAH2; RB1 and XK; and PLEK2 and RB1. As another example, CASP1, ELA2 and PLEK2 are measured. As yet another example, ANLN, and two constituents selected from the following combination of constituents are measured: CASP1 and PLEK2; and PLEK2 and RB1.

In yet other examples, any of the 2- or 3-gene models enumerated in Tables 9 or 10 can be measured to distinguish between prostate cancer subjects having a high versus a low Gleason score (e.g., Gleason score 7(4+3) or higher versus Gleason score of 7(3+4) or less, or Gleason score 7 or higher versus Gleason score 6 or less).

In a particular embodiment, the methods of the present invention are used in conjunction with the PSA test when PSA levels are above 2 but under 100, more preferably above 3 but under 50, more preferably above 3 but under 30, more preferably above 3 but under 15, and even more preferably above 3 but under 10. In particular embodiments, the methods of the present invention are used in conjunction with age-adjusted PSA criteria. Use of the methods of the present invention in conjunction with PSA levels provides a better diagnosis and/or prognosis of prostate cancer, over the use of PSA levels alone.

For example without limitation, For example without limitation, any of the 3-gene models enumerated in Table 2A, any of the 3-gene models enumerated in Table 3, any of the 2-gene, 4-gene and 6-gene models listed in Table 4, any of the 8-gene models enumerated in Table 17B, can be measured in conjunction with PSA to distinguish a prostate cancer diagnosed subject from a normal, healthy reference subject with at least 55% accuracy, preferably at least 75% accuracy. As a further example, without limitation, any of the 3-gene models enumerated in Table 5A, and any of the 1-gene, 2-gene, 3-gene and 5-gene models listed in Table 6, can be measured in conjunction with PSA to distinguish a prostate cancer diagnosed subject from a normal, healthy reference subject with at least 55% accuracy, preferably at least 75% accuracy.

In one embodiment, at least 1 constituent from Table 1 and/or Table 8 is measured in conjunction with PSA to distinguish a prostate cancer diagnosed subject from a normal, healthy reference subject (or otherwise healthy subject with BPH), wherein the at least 1 constituents is selected from IL18, RP51077B9.4, and S100A6.

In another embodiment, at least 2 constituents from Table 1 and/or Table 8 are measured in conjunction with PSA to distinguish a prostate cancer diagnosed subject from a normal, healthy reference subject (or otherwise healthy subject with BPH), wherein the at least two constituents are selected from the following combinations of constituents: a) ABL1 and BRCA1; b) MAP2K1 and MAPK1; c) BRCA1 and MAP2K1; d) PTPRC and RP51077B9.4; e) CD97 and SP1; f) CD97 and S100A6; g) IL18 and RP5107B9.4; h) MAP2K1 and S100A6, i) RP51077B9.4 and S100A6; and j) RP51077B9.4 and SP1.

In still another embodiment, at least 3 constituents from Table 1 and/or Table 8 are measured in conjunction with PSA to distinguish a prostate cancer diagnosed subject from a normal, healthy reference subject (or otherwise healthy subject with BPH), wherein the at least 3 constituents are selected from the following combinations of constituents: a) MAP2K1. MYC and S100A6; b) MAP2K1, S100A6 and SMAD3; and c) MAP2K1, S100A6 and TP53.

In yet another embodiment, at least 4 constituents from Table 1 and/or Table 8 are measured in conjunction with PSA to distinguish a prostate cancer diagnosed subject from a normal, healthy reference subject (or otherwise healthy subject with BPH), wherein the at least 4 constituents are selected from the following combinations of constituents: a) CD97, CDK2, RP51077B9.4 and SP1; b) BRCA1, GSK3B, RB1 and TNF.

In a particular embodiment, at least 5 constituents from Table 1 and/or Table 8 are measured in conjunction with PSA to distinguish a prostate cancer diagnosed subject from a normal, healthy reference subject (or otherwise healthy subject with BPH), wherein the at least 5 constituents are selected from the following combinations of constituents: a) S100A6, MYC, MAP2K1, C1QA, and RP51077B9.4; b) MAP2K1, SMAD3, S100A6, CCNE1, and TP53; and c) MAP2K1, TP53, S100A6, CCNE1 and ST14.

In another particular embodiment, at least 6 constituents from Table 1 and/or Table 8 are measured in conjunction with PSA to distinguish a prostate cancer diagnosed subject from a normal, healthy reference subject (or otherwise healthy subject with BPH), wherein the at least 6 constituents are selected from the following combinations of constituents: a) RP51077B9.4, CD97, CDKN2A, SP1, S100A6, and IQGAP1; and b) CD97, GSK3B, PTPRC, RP51077B9.4, SP1 and TNF.

In yet another particular embodiment, at least 8 constituents from Table 1 and/or Table 8 are measured in conjunction with PSA to distinguish a prostate cancer diagnosed subject from a normal, healthy reference subject (or otherwise healthy subject with BPH), wherein the at least 8 constituents are selected from the following combinations of constituents: a) BRCA1, CD97, CDK2, IQGAP1, PTPRC, RP51077B9.4, SP1, and TNF; b) ABL1, BRCA1, CD97, IL18, IQGAP1, RP51077B9.4, SP1, and TNF; c) RP51077B9.4, IQGAP1, ABL1, BRCA1, RB1, TNF, and CD97; d) RP51077B9.4, CD97, CDKN2A, IQGAP1, ABL1, BRCA1 and PTPRC; and d) SP1, CD97, IQGAP1, RP51077B9.4, ABL1, BRCA1, CDKN2A and PTPRC.

In yet further examples, at least one constituent from Table 1 and/or Table 8 is measured in conjunction with PSA to distinguish a prostate cancer diagnosed subject having a high versus low Gleason score. For example at least one constituent from Table 1 and/or Table 8 is measured in conjunction with PSA to distinguish a prostate cancer diagnosed subject having a Gleason score of 8-9 from a prostate cancer diagnosed subject having a Gleason score <8, wherein the at least one constituent is selected from the group consisting of C1QA, CCND2, COL6A2, and TIMP1. In another example, without limitation, at least 2 constituents from Table 1 and/or Table 8 are measured in conjunction with PSA to distinguish a prostate cancer diagnosed subject having a Gleason score of 8-9 from a prostate cancer diagnosed subject having a Gleason score <8, wherein the at least 2 constituents are CCND2 and COL6A2. As another example, without limitation, at least 3 constituents from Table 1 and/or Table 8 are measured in conjunction with PSA to distinguish a prostate cancer diagnosed subject having a Gleason score of 8-9 from a prostate cancer diagnosed subject having a Gleason score <8, wherein the at least 3 constituents are CCND2, COL6A2 and CDKN2A.

In a further example, at least 2 constituents are measured in conjunction with PSA to distinguish between prostate cancer subjects having a Gleason score of 7 (4+3)) or higher (i.e., more aggressive form of cancer) from those having less a Gleason score of 7(3+4) or lower (i.e., less aggressive form of cancer). For example, any of the 2- or 3-gene models enumerated in Table 7A, Table 9 or Table 10 can measured in conjunction with PSA to distinguish between prostate cancer subjects having a Gleason score of 7 (4+3)) or higher (i.e., more aggressive form of cancer) from those having less a Gleason score of 7(3+4) or lower (i.e., less aggressive form of cancer) with at least 55% accuracy, preferably at least 75% accuracy. In a particular embodiment, CD4 and TP53 are measured in conjunction with PSA. As a yet another example, as least three constituents from Table 1 and/or Table 8 are measured in conjunction with PSA to distinguish between prostate cancer subjects having a Gleason score of 7 (4+3)) or higher (i.e., more aggressive form of cancer) from those having less a Gleason score of 7(3+4) or lower (i.e., less aggressive form of cancer). In particular embodiments, CASP9, and two constituents selected from the following combination of constituents are measured in conjunction with PSA: PLEK2 and RB1; SIAH2 and VEGF; RB1 and XK; IGF2BP2 and VEGF; NCOA4 and VEGF; VEGF and XK; SRF and XK; and IGF2BP2 and RB1. In other particular embodiments, CASP1, and two constituents selected from the following combination of constituents are measured in conjunction with PSA: CD44 and POV1; EP300 and MTF1; NFKB1 and POV1; and IGF2BP2 and SERPING1. In yet other particular embodiments, CDKN2A, and two constituents selected from the following combination of constituents are measured in conjunction with PSA: CTSD and VHL; and KAI1 and VHL; In still another embodiment, MTA1, POV1 and RB1 are measured in conjunction with PSA. As a further example, PSA is measured in conjunction with CD44, POV1 and RB1. In yet another example, PSA is measured in conjunction with G1P3, PLEK2 and VEGF. In still another example, PSA is measured in conjunction with C1QB, CASP1 and KAI1. In yet another example, PSA is measured in conjunction with CD4, TP53 and E2F1.

As even further examples, at least two constituents from Table 1 and/or Table 8 are measured in conjunction with PSA to distinguish between prostate cancer subjects having a Gleason score of 7 or higher (i.e., more aggressive form of cancer) from those having less a Gleason score of 6 or lower (i.e., less aggressive form of cancer). For example, PSA is measured in conjunction with CASP9 and SOCS3. In even further examples, at least three constituents from Table 1 and/or Table 8 are measured in conjunction with PSA to distinguish between prostate cancer subjects having a Gleason score of 7 or higher (i.e., more aggressive form of cancer) from those having less a Gleason score of 6 or lower (i.e., less aggressive form of cancer). For example, ELA2, and two constituents selected from the following combination of constituents are measured in conjunction with PSA: RB1 and SIAH2; RB1 and XK; and PLEK2 and RB1. As another example, PSA is measured in conjunction with CASP1, ELA2 and PLEK2. As yet another example, ANLN, and two constituents selected from the following combination of constituents are measured in conjunction with PSA: CASP1 and PLEK2; and PLEK2 and RB1.

In yet other examples, any of the 2- or 3-gene models enumerated in Tables 9 or 10 can be measured in conjunction with PSA to distinguish between prostate cancer subjects having a high versus a low Gleason score (e.g., Gleason score 7(4+3) or higher versus Gleason score of 7(3+4) or less, or Gleason score 7 or higher versus Gleason score 6 or less).

Additionally, it has been discovered that valuable and unexpected results may be achieved when the quantitative measurement of constituents is performed under repeatable conditions (within a degree of repeatability of measurement of better than twenty percent, preferably ten percent or better, more preferably five percent or better, and more preferably three percent or better). For the purposes of this description and the following claims, a degree of repeatability of measurement of better than twenty percent may be used as providing measurement conditions that are “substantially repeatable”. In particular, it is desirable that each time a measurement is obtained corresponding to the level of expression of a constituent in a particular sample, substantially the same measurement should result for substantially the same level of expression. In this manner, expression levels for a constituent in a Gene Expression Panel (Precision Profile™) may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar as defined herein. When both of these criteria are satisfied, then measurement of the expression level of one constituent may be meaningfully compared with measurement of the expression level of another constituent in a given sample and from sample to sample.

The evaluation or characterization of prostate cancer is defined to be diagnosing prostate cancer, assessing the presence or absence of prostate cancer, or assessing the risk of developing prostate cancer, and may also include assessing the prognosis of a subject with prostate cancer, assessing the recurrence of prostate cancer or assessing the presence or absence of a metastasis. Similarly, the evaluation or characterization of an agent for treatment of prostate cancer includes identifying agents suitable for the treatment of prostate cancer. The agents can be compounds known to treat prostate cancer or compounds that have not been shown to treat prostate cancer.

The agent to be evaluated or characterized for the treatment of prostate cancer may be an alkylating agent (e.g., Cisplatin, Carboplatin, Oxaliplatin, BBR3464, Chlorambucil, Chlormethine, Cyclophosphamides, Ifosmade, Melphalan, Carmustine, Fotemustine, Lomustine, Streptozocin, Busulfan, Dacarbazine, Mechlorethamine, Procarbazine, Temozolomide, ThioTPA, and Uramustine); an anti-metabolite (e.g., purine (azathioprine, mercaptopurine), pyrimidine (Capecitabine, Cytarabine, Fluorouracil, Gemcitabine), and folic acid (Methotrexate, Pemetrexed, Raltitrexed)); a vinca alkaloid (e.g., Vincristine, Vinblastine, Vinorelbine, Vindesine); a taxane (e.g., paclitaxel, docetaxel, BMS-247550); an anthracycline (e.g., Daunorubicin, Doxorubicin, Epirubicin, Idarubicin, Mitoxantrone, Valrubicin, Bleomycin, Hydroxyurea, and Mitomycin); a topoisomerase inhibitor (e.g., Topotecan, Irinotecan Etoposide, and Teniposide); a monoclonal antibody (e.g., Alemtuzumab, Bevacizumab, Cetuximab, Gemtuzumab, Panitumumab, Rituximab, and Trastuzumab); a photosensitizer (e.g., Aminolevulinic acid, Methyl aminolevulinate, Porfimer sodium, and Verteporfin); a tyrosine kinase inhibitor (e.g., Gleevec™); an epidermal growth factor receptor inhibitor (e.g., Iressa™, erlotinib (Tarceva™), gefitinib); an FPTase inhibitor (e.g., FTIs (R115777, SCH66336, L-778,123)); a KDR inhibitor (e.g., SU6668, PTK787); a proteosome inhibitor (e.g., PS341); a TS/DNA synthesis inhibitor (e.g., ZD9331, Raltirexed (ZD1694, Tomudex), ZD9331, 5-FU)); an S-adenosyl-methionine decarboxylase inhibitor (e.g., SAM468A); a DNA methylating agent (e.g., TMZ); a DNA binding agent (e.g., PZA); an agent which binds and inactivates O⁶-alkylguanine AGT (e.g., BG); a c-raf-1 antisense oligo-deoxynucleotide (e.g., ISIS-5132 (CGP-69846A)); tumor immunotherapy; a steroidal and/or non-steroidal anti-inflammatory agent (e.g., corticosteroids, COX-2 inhibitors); or other agents such as Alitretinoin, Altretamine, Amsacrine, Anagrelide, Arsenic trioxide, Asparaginase, Bexarotene, Bortezomib, Celecoxib, Dasatinib, Denileukin Diftitox, Estramustine, Hydroxycarbamide, Imatinib, Pentostatin, Masoprocol, Mitotane, Pegaspargase, and Tretinoin.

Prostate cancer and conditions related to prostate cancer is evaluated by determining the level of expression (e.g., a quantitative measure) of an effective number (e.g., one or more) of constituents of the Gene Expression Panels (Precision Profile™) disclosed herein (i.e., Tables 1 and 9, respectively). By an effective number is meant the number of constituents that need to be measured in order to discriminate between a subject having prostate cancer and a normal, healthy subject or otherwise healthy subject with BPH, or the number of constituents that need to be measured in order to discriminate between a subject having an aggressive form of prostate cancer (e.g., Gleason score of 7 (4+3), 8 or 9) and a subject having a less aggressive form of prostate cancer (e.g., Gleason score of 7 (3+4), 6 or lower). Preferably the constituents are selected as to 1) discriminate between a subject having prostate cancer and a normal subject or an otherwise healthy subject with BPH with at least 55%, accuracy, more preferably 60%, 65%, 70%, 75%, 80%, 85%, 90%, 95%, 97%, 98%, 99% or greater accuracy; or 2) discriminate between a subject having an aggressive form of prostate cancer (e.g., Gleason score of 7 (4+3), 8 or 9) and a subject having a less aggressive form of prostate cancer (e.g., Gleason score of 7 (3+4), 6 or lower), with at least 55% accuracy, more preferably 60%, 65%, 70%, 75%, 80%, 85%, 90%, 95%, 97%, 98%, 99% or greater accuracy.

The level of expression is determined by any means known in the art, such as for example quantitative PCR. The measurement is obtained under conditions that are substantially repeatable. Optionally, the qualitative measure of the constituent is compared to a reference or baseline level or value (e.g. a baseline profile set). In one embodiment, the reference or baseline level is a level of expression of one or more constituents in one or more subjects known not to be suffering from prostate cancer (e.g., normal, healthy individual(s), or otherwise healthy individuals with BPH). Alternatively, the reference or baseline level is derived from the level of expression of one or more constituents in one or more subjects known to be suffering from prostate cancer. Optionally, the baseline level is derived from the same subject from which the first measure is derived. For example, the baseline is taken from a subject prior to receiving treatment or surgery for prostate cancer, or at different time periods during a course of treatment. Such methods allow for the evaluation of a particular treatment for a selected individual. Comparison can be performed on test (e.g., patient) and reference samples (e.g., baseline) measured concurrently or at temporally distinct times. An example of the latter is the use of compiled expression information, e.g., a gene expression database, which assembles information about expression levels of cancer associated genes.

A reference or baseline level or value as used herein can be used interchangeably and is meant to be relative to a number or value derived from population studies, including without limitation, such subjects having similar age range, subjects in the same or similar ethnic group, sex, or, in female subjects, pre-menopausal or post-menopausal subjects, or relative to the starting sample of a subject undergoing treatment for prostate cancer. Such reference values can be derived from statistical analyses and/or risk prediction data of populations obtained from mathematical algorithms and computed indices of prostate cancer. Reference indices can also be constructed and used using algorithms and other methods of statistical and structural classification.

In one embodiment of the present invention, the reference or baseline value is the amount of expression of a cancer associated gene in a control sample derived from one or more subjects who are both asymptomatic and lack traditional laboratory risk factors for prostate cancer. In another embodiment of the invention, the reference or baseline value is the amount of expression of a cancer associated gene in a control sample derived from one or more subjects with BPH. In yet another embodiment of the present invention, the reference or baseline value is the level of cancer associated genes in a control sample derived from one or more subjects who are not at risk or at low risk for developing prostate cancer.

In a further embodiment, such subjects are monitored and/or periodically retested for a diagnostically relevant period of time (“longitudinal studies”) following such test to verify continued absence from prostate cancer (disease or event free survival). Such period of time may be one year, two years, two to five years, five years, five to ten years, ten years, or ten or more years from the initial testing date for determination of the reference or baseline value. Furthermore, retrospective measurement of cancer associated genes in properly banked historical subject samples may be used in establishing these reference or baseline values, thus shortening the study time required, presuming the subjects have been appropriately followed during the intervening period through the intended horizon of the product claim.

A reference or baseline value can also comprise the amounts of cancer associated genes derived from subjects who show an improvement in cancer status as a result of treatments and/or therapies for the cancer being treated and/or evaluated.

In another embodiment, the reference or baseline value is an index value or a baseline value. An index value or baseline value is a composite sample of an effective amount of cancer associated genes from one or more subjects who do not have cancer (i.e., normal, healthy subjects and/or otherwise healthy subjects with BPH).

For example, where the reference or baseline level is comprised of the amounts of cancer associated genes derived from one or more subjects who have not been diagnosed with prostate cancer, are not known to be suffering from prostate cancer, or are diagnosed with BPH, change (e.g., increase or decrease) in the expression level of a cancer associated gene in the patient-derived sample as compared to the expression level of such gene in the reference or baseline level indicates that the subject is suffering from or is at risk of developing prostate cancer. In contrast, when the methods are applied prophylacticly, a similar level of expression in the patient-derived sample of a prostate cancer associated gene compared to such gene in the baseline level indicates that the subject is not suffering from or is at risk of developing prostate cancer.

Where the reference or baseline level is comprised of the amounts of cancer associated genes derived from one or more subjects who have been diagnosed with prostate cancer, or are known to be suffering from prostate cancer, a similarity in the expression pattern in the patient-derived sample of a prostate cancer associated gene compared to the prostate cancer baseline level indicates that the subject is suffering from or is at risk of developing prostate cancer.

Expression of a prostate cancer associated gene or constituent also allows for the course of treatment of prostate cancer to be monitored. In this method, a biological sample is provided from a subject undergoing treatment, e.g., if desired, biological samples are obtained from the subject at various time points before, during, or after treatment. Expression of a prostate cancer associated gene is then determined and compared to a reference or baseline profile. The baseline profile may be taken or derived from one or more individuals who have been exposed to the treatment. Alternatively, the baseline level may be taken or derived from one or more individuals who have not been exposed to the treatment. For example, samples may be collected from subjects who have received initial treatment for prostate cancer and subsequent treatment for prostate cancer to monitor the progress of the treatment.

Differences in the genetic makeup of individuals can result in differences in their relative abilities to metabolize various drugs. Accordingly, the Precision Profile™ for Prostate Cancer Detection (Table 1) disclosed herein, allows for a putative therapeutic or prophylactic to be tested from a selected subject in order to determine if the agent is suitable for treating or preventing prostate cancer in the subject. Additionally, other genes known to be associated with toxicity may be used. By suitable for treatment is meant determining whether the agent will be efficacious, not efficacious, or toxic for a particular individual. By toxic it is meant that the manifestations of one or more adverse effects of a drug when administered therapeutically. For example, a drug is toxic when it disrupts one or more normal physiological pathways.

To identify a therapeutic that is appropriate for a specific subject, a test sample from the subject is exposed to a candidate therapeutic agent, and the expression of one or more prostate cancer associated genes is determined. A subject sample is incubated in the presence of a candidate agent and the pattern of prostate cancer gene expression in the test sample is measured and compared to a baseline profile, e.g., a prostate cancer baseline profile or a non-prostate cancer baseline profile or an index value. The test agent can be any compound or composition. For example, the test agent is a compound known to be useful in the treatment of prostate cancer. Alternatively, the test agent is a compound that has not previously been used to treat prostate cancer.

If the reference sample, e.g., baseline is from a subject that does not have prostate cancer (i.e., a normal, healthy subject or otherwise healthy subject with BPH), a similarity in the pattern of expression of prostate cancer associated genes in the test sample compared to the reference sample indicates that the treatment is efficacious. Whereas a change in the pattern of expression of prostate cancer associated genes in the test sample compared to the reference sample indicates a less favorable clinical outcome or prognosis. By “efficacious” is meant that the treatment leads to a decrease of a sign or symptom of prostate cancer in the subject or a change in the pattern of expression of a prostate cancer associated gene such that the gene expression pattern has an increase in similarity to that of a reference or baseline pattern. Assessment of prostate cancer is made using standard clinical protocols. Efficacy is determined in association with any known method for diagnosing or treating prostate cancer.

A Gene Expression Panel (Precision Profile™) is selected in a manner so that quantitative measurement of RNA constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel (Precision Profile™) and (ii) a baseline quantity.

Additional embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; (d) to monitor a biological condition of a subject; (e) for measurement of therapeutic efficacy of natural or synthetic compositions or stimuli that may be formulated individually or in combinations or mixtures for a range of targeted biological conditions; (f) for predictions of toxicological effects and dose effectiveness of a composition or mixture of compositions for an individual or for a population or set of individuals or for a population of cells; (g) for determination of how two or more different agents administered in a single treatment might interact so as to detect any of synergistic, additive, negative, neutral of toxic activity (h) for performing pre-clinical and clinical trials by providing new criteria for pre-selecting subjects according to informative profile data sets for revealing disease status and conducting preliminary dosage studies for these patients prior to conducting Phase 1 or 2 trials.

Gene expression profiling and the use of index characterization for a particular condition or agent or both may be used to reduce the cost of Phase 3 clinical trials and may be used beyond Phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; managing the health care of a patient; and quality control for different batches of an agent or a mixture of agents.

### The Subject

The methods disclosed herein may be applied to cells of humans, mammals or other organisms without the need for undue experimentation by one of ordinary skill in the art because all cells transcribe RNA and it is known in the art how to extract RNA from all types of cells.

A subject can include those who have not been previously diagnosed as having prostate cancer or a condition related to prostate cancer. Alternatively, a subject can also include those who have already been diagnosed as having prostate cancer or a condition related to prostate cancer. Diagnosis of prostate cancer is made, for example, from any one or combination of the following procedures: a medical history, physical examination, e.g., digital rectal examination, blood tests, e.g., a PSA test, and screening tests and tissue sampling procedures e.g., cytoscopy and transrectal ultrasonography, and biopsy, in conjunction with Gleason score.

Optionally, the subject has been previously treated with a surgical procedure for removing prostate cancer or a condition related to prostate cancer, including but not limited to any one or combination of the following treatments: prostatectomy (including radical retropubic and radical perineal prostatectomy), transurethral resection, orchiectomy, and cryosurgery. Optionally, the subject has previously been treated with radiation therapy including but not limited to external beam radiation therapy and brachytherapy). Optionally, the subject has been treated with hormonal therapy, including but not limited to orchiectomy, anti-androgen therapy (e.g., flutamide, bicalutamide, nilutamide, cyproterone acetate, ketoconazole and aminoglutethimide), and GnRH agonists (e.g., leuprolide, goserelin, triptorelin, and buserelin). Optionally, the subject has previously been treated with chemotherapy for palliative care (e.g., docetaxel with a corticosteroid such as prednisone). Optionally, the subject has previously been treated with any one or combination of such radiation therapy, hormonal therapy, and chemotherapy, as previously described, alone, in combination, or in succession with a surgical procedure for removing prostate cancer as previously described. Optionally, the subject may be treated with any of the agents previously described; alone, or in combination with a surgical procedure for removing prostate cancer and/or radiation therapy as previously described.

A subject can also include those who are suffering from, or at risk of developing prostate cancer or a condition related to prostate cancer, such as those who exhibit known risk factors for prostate cancer or a condition related to prostate cancer. Known risk factors for prostate cancer include, but are not limited to: age (increased risk above age 50), race (higher prevalence among African American men), nationality (higher prevalence in North America and northwestern Europe), family history, and diet (increased risk with a high animal fat diet).

### Selecting Constituents of a Gene Expression Panel

### Precision Profile™

The general approach to selecting constituents of a Gene Expression Panel (Precision Profile™) has been described in PCT application publication number WO 01/25473, incorporated herein in its entirety. A wide range of Gene Expression Panels (Precision Profiles™) have been designed and experimentally validated, each panel providing a quantitative measure of biological condition that is derived from a sample of blood or other tissue. For each panel, experiments have verified that a Gene Expression Profile using the panel's constituents is informative of a biological condition. (It has also been demonstrated that in being informative of biological condition, the Gene Expression Profile is used, among other things, to measure the effectiveness of therapy, as well as to provide a target for therapeutic intervention).

The Precision Profile™ for Prostate Cancer Detection (Table 1) and the Prostate Cancer Clinically Tested Precision Profile™ (Table 8), include relevant genes associated with cancer and inflammation, which may be selected for a given Precision Profile™, such as the Precision Profiles™ demonstrated herein to be useful in the evaluation of prostate cancer and conditions related to prostate cancer.

### Inflammation and Cancer

Evidence has shown that cancer in adults arises frequently in the setting of chronic inflammation. Epidemiological and experimental studies provide strong support for the concept that inflammation facilitates malignant growth. Inflammatory components have been shown to 1) induce DNA damage, which contributes to genetic instability (e.g., cell mutation) and transformed cell proliferation (Balkwill and Mantovani, Lancet 357:539-545 (2001)); 2) promote angiogenesis, thereby enhancing tumor growth and invasiveness (Coussens L. M. and Z. Werb, Nature 429:860-867 (2002)); and 3) impair myelopoiesis and haemopoiesis, which cause immune dysfunction and inhibit immune surveillance (Kusmartsev and Gabrilovic, Cancer Immunol. Immunother. 51:293-298 (2002); Serafini et al., Cancer Immunol. Immunther. 53:64-72 (2004)).

Studies suggest that inflammation promotes malignancy via proinflammatory cytokines, including but not limited to IL-113, which enhance immune suppression through the induction of myeloid suppressor cells, and that these cells down regulate immune surveillance and allow the outgrowth and proliferation of malignant cells by inhibiting the activation and/or function of tumor-specific lymphocytes. (Bunt et al., J. Immunol. 176: 284-290 (2006). Such studies are consistent with findings that myeloid suppressor cells are found in many cancer patients, including lung and breast cancer, and that chronic inflammation in some of these malignancies may enhance malignant growth (Coussens L. M. and Z. Werb, 2002).

Additionally, many cancers express an extensive repertoire of chemokines and chemokine receptors, and may be characterized by dis-regulated production of chemokines and abnormal chemokine receptor signaling and expression. Tumor-associated chemokines are thought to play several roles in the biology of primary and metastatic cancer such as: control of leukocyte infiltration into the tumor, manipulation of the tumor immune response, regulation of angiogenesis, autocrine or paracrine growth and survival factors, and control of the movement of the cancer cells. Thus, these activities likely contribute to growth within/outside the tumor microenvironment and to stimulate anti-tumor host responses.

As tumors progress, it is common to observe immune deficits not only within cells in the tumor microenvironment but also frequently in the systemic circulation. Whole blood contains representative populations of all the mature cells of the immune system as well as secretory proteins associated with cellular communications. The earliest observable changes of cellular immune activity are altered levels of gene expression within the various immune cell types. Immune responses are now understood to be a rich, highly complex tapestry of cell-cell signaling events driven by associated pathways and cascades—all involving modified activities of gene transcription. This highly interrelated system of cell response is immediately activated upon any immune challenge, including the events surrounding host response to prostate cancer and treatment. Modified gene expression precedes the release of cytokines and other immunologically important signaling elements.

As such, inflammation genes, such as a subset of the genes listed in the Precision Profile™ for Prostate Cancer Detection (Table 1) are useful for distinguishing between subjects suffering from prostate cancer and normal subjects, in addition to the other gene panels, i.e., Precision Profiles™, described herein.

Several gene expression profiles have been derived from the Gene Expression Panels (Precision Profiles™) described herein and experimentally validated as described herein, as being capable of discrimination between prostate cancer subjects and normal, healthy subjects (or otherwise healthy subjects with BPH) with a surprisingly high degree of sensitivity and specificity. As described herein, several of the genes (i.e., constituents) of the Precision Profile™ for Prostate Cancer Detection are differentially expressed in fractionated blood. Without intending to be bound by theory, such differential expression may reflect a modulation of specific immune cells found in the blood. Examples genes that are differentially expressed in fractionated blood include RP51077B9.4, CD97, CDKN2A, SP1, S100A6 and IQGAP1. Surprisingly, several of the most statistically significant gene expression profiles (i.e., gene models) described herein comprise one or more of these six genes.

### Design of Assays

Typically, a sample is run through a panel in replicates of three for each target gene (assay); that is, a sample is divided into aliquots and for each aliquot the concentrations of each constituent in a Gene Expression Panel (Precision Profile™) is measured. From over thousands of constituent assays, with each assay conducted in triplicate, an average coefficient of variation was found (standard deviation/average)*100, of less than 2 percent among the normalized ΔCt measurements for each assay (where normalized quantitation of the target mRNA is determined by the difference in threshold cycles between the internal control (e.g., an endogenous marker such as 18S rRNA, or an exogenous marker) and the gene of interest. This is a measure called “intra-assay variability”. Assays have also been conducted on different occasions using the same sample material. This is a measure of “inter-assay variability”. Preferably, the average coefficient of variation of intra-assay variability or inter-assay variability is less than 20%, more preferably less than 10%, more preferably less than 5%, more preferably less than 4%, more preferably less than 3%, more preferably less than 2%, and even more preferably less than 1%.

It has been determined that it is valuable to use the quadruplicate or triplicate test results to identify and eliminate data points that are statistical “outliers”; such data points are those that differ by a percentage greater, for example, than 3% of the average of all three or four values. Moreover, if more than one data point in a set of three or four is excluded by this procedure, then all data for the relevant constituent is discarded.

### Measurement of Gene Expression for a Constituent in the Panel

For measuring the amount of a particular RNA in a sample, methods known to one of ordinary skill in the art were used to extract and quantify transcribed RNA from a sample with respect to a constituent of a Gene Expression Panel (Precision Profile™). (See detailed protocols below. Also see PCT application publication number WO 98/24935 herein incorporated by reference for RNA analysis protocols). Briefly, RNA is extracted from a sample such as any tissue, body fluid, cell (e.g., circulating tumor cell) or culture medium in which a population of cells of a subject might be growing. For example, cells may be lysed and RNA eluted in a suitable solution in which to conduct a DNAse reaction. Subsequent to RNA extraction, first strand synthesis may be performed using a reverse transcriptase. Gene amplification, more specifically quantitative PCR assays, can then be conducted and the gene of interest calibrated against an internal marker such as 18S rRNA (Hirayama et al., Blood 92, 1998: 46-52). Any other endogenous marker can be used, such as 28S-25S rRNA and 5S rRNA. Samples are measured in multiple replicates, for example, 3 replicates. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, quantitation of the reporter signal for an internal marker generated by the exponential increase of amplified product may also be used. Amplification of the target template may be accomplished by isothermic gene amplification strategies or by gene amplification by thermal cycling such as PCR.

It is desirable to obtain a definable and reproducible correlation between the amplified target or reporter signal, i.e., internal marker, and the concentration of starting templates. It has been discovered that this objective can be achieved by careful attention to, for example, consistent primer-template ratios and a strict adherence to a narrow permissible level of experimental amplification efficiencies (for example 80.0 to 100%+/−5% relative efficiency, typically 90.0 to 100%+/−5% relative efficiency, more typically 95.0 to 100%+/−2%, and most typically 98 to 100%+/−1% relative efficiency). In determining gene expression levels with regard to a single Gene Expression Profile, it is necessary that all constituents of the panels, including endogenous controls, maintain similar amplification efficiencies, as defined herein, to permit accurate and precise relative measurements for each constituent. Amplification efficiencies are regarded as being “substantially similar”, for the purposes of this description and the following claims, if they differ by no more than approximately 10%, preferably by less than approximately 5%, more preferably by less than approximately 3%, and more preferably by less than approximately 1%. Measurement conditions are regarded as being “substantially repeatable, for the purposes of this description and the following claims, if they differ by no more than approximately +/−10% coefficient of variation (CV), preferably by less than approximately +/−5% CV, more preferably +/−2% CV. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, tests are run to assure that these conditions are satisfied. For example, the design of all primer-probe sets are done in house, experimentation is performed to determine which set gives the best performance. Even though primer-probe design can be enhanced using computer techniques known in the art, and notwithstanding common practice, it has been found that experimental validation is still useful. Moreover, in the course of experimental validation, the selected primer-probe combination is associated with a set of features:

The reverse primer should be complementary to the coding DNA strand. In one embodiment, the primer should be located across an intron-exon junction, with not more than four bases of the three-prime end of the reverse primer complementary to the proximal exon. (If more than four bases are complementary, then it would tend to competitively amplify genomic DNA.)

In an embodiment of the invention, the primer probe set should amplify cDNA of less than 110 bases in length and should not amplify, or generate fluorescent signal from, genomic DNA or transcripts or cDNA from related but biologically irrelevant loci.

A suitable target of the selected primer probe is first strand cDNA, which in one embodiment may be prepared from whole blood as follows:

(a) Use of Whole Blood for Ex Vivo Assessment of a Biological Condition

Human blood is obtained by venipuncture and prepared for assay. Cells are lysed and nucleic acids, e.g., RNA, are extracted by various standard means.

Nucleic acids, RNA and or DNA, are purified from cells, tissues or fluids of the test population of cells. RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), e.g., using a filter-based RNA isolation system from Ambion (RNAqueous™, Phenol-free Total RNA Isolation Kit, Catalog #1912, version 9908; Austin, Tex.) or the PAXgene™ Blood RNA System (from Pre-Analytix).

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples (see, for example, RT PCR, Chapter 15 in RNA Methodologies, A Laboratory Guide for Isolation and Characterization, 2nd edition, 1998, Robert E. Farrell, Jr., Ed., Academic Press; or Chapter 22 pp. 143-151, RNA Isolation and Characterization Protocols, Methods in Molecular Biology, Volume 86, 1998, R. Rapley and D. L. Manning Eds., Human Press, or Chapter 14 Statistical refinement of primer design parameters; or Chapter 5, pp. 55-72, PCR Applications: protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7900 obtained from Applied Biosystems, Foster City, Calif.; see Nucleic acid detection methods, pp. 1-24, in Molecular Methods for Virus Detection, D. L. Wiedbrauk and D. H., Farkas, Eds., 1995, Academic Press). Amplified nucleic acids are detected using fluorescent-tagged detection oligonucleotide probes (see, for example, Taqman™ PCR Reagent Kit, Protocol, part number 402823, Revision A, 1996, Applied Biosystems, Foster City Calif.) that are identified and synthesized from publicly known databases as described for the amplification primers.

For example, without limitation, amplified cDNA is detected and quantified using detection systems such as the ABI Prism® 7900 Sequence Detection System (Applied Biosystems (Foster City, Calif.)), the Cepheid SmartCycler® and Cepheid GeneXpert® Systems, the Fluidigm BioMark™ System, and the Roche LightCycler® 480 Real-Time PCR System. Amounts of specific RNAs contained in the test sample can be related to the relative quantity of fluorescence observed (see for example, Advances in Quantitative PCR Technology: 5′ Nuclease Assays, Y. S. Lie and C. J. Petropolus, Current Opinion in Biotechnology, 1998, 9:43-48, or Rapid Thermal Cycling and PCR Kinetics, pp. 211-229, chapter 14 in PCR applications: protocols for functional genomics, M. A. Innis, D. H. Gelfand and J. J. Sninsky, Eds., 1999, Academic Press). Examples of the procedure used with several of the above-mentioned detection systems are described below. In some embodiments, these procedures can be used for both whole blood RNA and RNA extracted from cultured cells (e.g., without limitation, CTCs, and CECs). In some embodiments, any tissue, body fluid, or cell(s) (e.g., circulating tumor cells (CTCs) or circulating endothelial cells (CECs)) may be used for ex vivo assessment of predicted survivability and/or survival time affected by an agent. Methods herein may also be applied using proteins where sensitive quantitative techniques, such as an Enzyme Linked ImmunoSorbent Assay (ELISA) or mass spectroscopy, are available and well-known in the art for measuring the amount of a protein constituent (see WO 98/24935 herein incorporated by reference).

An example of a procedure for the synthesis of first strand cDNA for use in PCR amplification is as follows:

Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent).

Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| 1 reaction (mL) | 11X, e.g. 10 samples (μL) | |
|---|---|---|
| 10X RT Buffer | 10.0 | 110.0 |
| 25 mM MgC1₂ | 22.0 | 242.0 |
| dNTPs | 20.0 | 220.0 |
| Random Hexamers | 5.0 | 55.0 |
| RNAse Inhibitor | 2.0 | 22.0 |
| Reverse Transcriptase | 2.5 | 27.5 |
| Water | 18.5 | 203.5 |
| Total: | 80.0 | 880.0 |
| | | (80 μL per sample) |

4. Bring each RNA sample to a total volume of 20 μL in a 1.5 mL microcentrifuge tube (for example, remove 10 μL RNA and dilute to 20 μL with RNase/DNase free water, for whole blood RNA use 20 μL total RNA) and add 80 μL RT reaction mix from step 5, 2, 3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and β-actin.

Following the synthesis of first strand cDNA, one particular embodiment of the approach for amplification of first strand cDNA by PCR, followed by detection and quantification of constituents of a Gene Expression Panel (Precision Profile™) is performed using the ABI Prism® 7900 Sequence Detection System as follows:

Materials

1. 20× Primer/Probe Mix for each gene of interest.

2. 20× Primer/Probe Mix for 18S endogenous control.

3. 2× Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism® 7700 or 7900 Sequence Detector.

Methods

1. Make stocks of each Primer/Probe mix containing the Primer/Probe for the gene of interest, Primer/Probe for 18S endogenous control, and 2×PCR Master Mix as follows. Make sufficient excess to allow for pipetting error e.g., approximately 10% excess. The following example illustrates a typical set up for one gene with quadruplicate samples testing two conditions (2 plates).

| | 1X (1 well) (μL) |
|---|---|
| 2X Master Mix | 7.5 |
| 20X 18S Primer/Probe Mix | 0.75 |
| 20X Gene of interest Primer/Probe Mix | 0.75 |
| Total | 9.0 |

2. Make stocks of cDNA targets by diluting 95 μL of cDNA into 20004 of water. The amount of cDNA is adjusted to give Ct values between 10 and 18, typically between 12 and 16.

3. Pipette 9 μL of Primer/Probe mix into the appropriate wells of an Applied Biosystems 384-Well Optical Reaction Plate.

4. Pipette 10 μL of cDNA stock solution into each well of the Applied Biosystems 384-Well Optical Reaction Plate.

5. Seal the plate with Applied Biosystems Optical Caps, or optical-clear film.

6. Analyze the plate on the ABI Prism® 7900 Sequence Detector.

In another embodiment of the invention, the use of the primer probe with the first strand cDNA as described above to permit measurement of constituents of a Gene Expression Panel (Precision Profile™) is performed using a QPCR assay on Cepheid SmartCycler® and GeneXpert® Instruments as follows:
1. To run a QPCR assay in duplicate on the Cepheid SmartCycler® instrument containing three target genes and one reference gene, the following procedure should be followed.

A. With 20× Primer/Probe Stocks.

Materials
1. SmartMix™-HM lyophilized Master Mix.
2. Molecular grade water.
3. 20× Primer/Probe Mix for the 18S endogenous control gene. The endogenous control gene will be dual labeled with VIC-MGB or equivalent.
4. 20× Primer/Probe Mix for each for target gene one, dual labeled with FAM-BHQ1 or equivalent.
5. 20× Primer/Probe Mix for each for target gene two, dual labeled with Texas Red-BHQ2 or equivalent.
6. 20× Primer/Probe Mix for each for target gene three, dual labeled with Alexa 647-BHQ3 or equivalent.
7. Tris buffer, pH 9.0
8. cDNA transcribed from RNA extracted from sample.
9. SmartCycler® 25 μL tube.
10. Cepheid SmartCycler® instrument.

Methods
1. For each cDNA sample to be investigated, add the following to a sterile 650 μL tube.

| SmartMix ™-HM lyophilized Master Mix | 1 bead | |
|---|---|---|
| 20X 18S Primer/Probe Mix | 2.5 | μL |
| 20X Target Gene 1 Primer/Probe Mix | 2.5 | μL |
| 20X Target Gene 2 Primer/Probe Mix | 2.5 | μL |
| 20X Target Gene 3 Primer/Probe Mix | 2.5 | μL |
| Tris Buffer, pH 9.0 | 2.5 | μL |
| Sterile Water | 34.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18 S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

B. With Lyophilized SmartBeads™.

Materials
1. SmartMix™-HM lyophilized Master Mix.
2. Molecular grade water.
3. SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent and one dual labeled with Alexa 647-BHQ3 or equivalent.
4. Tris buffer, pH 9.0
5. cDNA transcribed from RNA extracted from sample.
6. SmartCycler® 25 μL tube.
7. Cepheid SmartCycler® instrument.

Methods
1. For each cDNA sample to be investigated, add the following to a sterile 650 μL tube.

| SmartMix ™-HM lyophilized Master Mix | 1 bead | |
|---|---|---|
| SmartBead ™ containing four primer/probe sets | 1 bead | |
| Tris Buffer, pH 9.0 | 2.5 | μL |
| Sterile Water | 44.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18 S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.
II. To run a QPCR assay on the Cepheid GeneXpert® instrument containing three target genes and one reference gene, the following procedure should be followed. Note that to do duplicates, two self contained cartridges need to be loaded and run on the GeneXpert® instrument.

Materials
1. Cepheid GeneXpert® self contained cartridge preloaded with a lyophilized SmartMix™-HM master mix bead and a lyophilized SmartBead™ containing four primer/probe sets.
2. Molecular grade water, containing Tris buffer, pH 9.0.
3. Extraction and purification reagents.
4. Clinical sample (whole blood, RNA, etc.)
5. Cepheid GeneXpert® instrument.

Methods
1. Remove appropriate GeneXpert® self contained cartridge from packaging.
2. Fill appropriate chamber of self contained cartridge with molecular grade water with Tris buffer, pH 9.0.
3. Fill appropriate chambers of self contained cartridge with extraction and purification reagents.
4. Load aliquot of clinical sample into appropriate chamber of self contained cartridge.
5. Seal cartridge and load into GeneXpert® instrument.
6. Run the appropriate extraction and amplification protocol on the GeneXpert® and analyze the resultant data.

In yet another embodiment of the invention, the use of the primer probe with the first strand cDNA as described above to permit measurement of constituents of a Gene Expression Panel (Precision Profile™) is performed using a QPCR assay on the Roche LightCycler® 480 Real-Time PCR System as follows:

Materials
1. 20× Primer/Probe stock for the 18S endogenous control gene. The endogenous control gene may be dual labeled with either VIC-MGB or VIC-TAMRA.
2. 20× Primer/Probe stock for each target gene, dual labeled with either FAM-TAMRA or FAM-BHQ1.
3. 2× LightCycler® 490 Probes Master (master mix).
4. 1×cDNA sample stocks transcribed from RNA extracted from samples.
5. 1×TE buffer, pH 8.0.
6. LightCycler® 480 384-well plates.
7. Source MDx 24 gene Precision Profile™ 96-well intermediate plates.
8. RNase/DNase free 96-well plate.
9. 1.5 mL microcentrifuge tubes.
10. Beckman/Coulter Biomek® 3000 Laboratory Automation Workstation.
11. Velocity11 Bravo™ Liquid Handling Platform.
12. LightCycler® 480 Real-Time PCR System.

Methods
1. Remove a Source MDx 24 gene Precision Profile™ 96-well intermediate plate from the freezer, thaw and spin in a plate centrifuge.
2. Dilute four (4) 1×cDNA sample stocks in separate 1.5 mL microcentrifuge tubes with the total final volume for each of 540 μL.
3. Transfer the 4 diluted cDNA samples to an empty RNase/DNase free 96-well plate using the Biomek® 3000 Laboratory Automation Workstation.
4. Transfer the cDNA samples from the cDNA plate created in step 3 to the thawed and centrifuged Source MDx 24 gene Precision Profile™ 96-well intermediate plate using Biomek® 3000 Laboratory Automation Workstation. Seal the plate with a foil seal and spin in a plate centrifuge.
5. Transfer the contents of the cDNA-loaded Source MDx 24 gene Precision Profile™ 96-well intermediate plate to a new LightCycler® 480 384-well plate using the Bravo™ Liquid Handling Platform. Seal the 384-well plate with a LightCycler® 480 optical sealing foil and spin in a plate centrifuge for 1 minute at 2000 rpm.
6. Place the sealed in a dark 4° C. refrigerator for a minimum of 4 minutes.
7. Load the plate into the LightCycler® 480 Real-Time PCR System and start the LightCycler® 480 software. Chose the appropriate run parameters and start the run.
8. At the conclusion of the run, analyze the data and export the resulting CP values to the database.

In some instances, target gene FAM measurements may be beyond the detection limit of the particular platform instrument used to detect and quantify constituents of a Gene Expression Panel (Precision Profile™). To address the issue of “undetermined” gene expression measures as lack of expression for a particular gene, the detection limit may be reset and the “undetermined” constituents may be “flagged”. For example without limitation, the ABI Prism® 7900HT Sequence Detection System reports target gene FAM measurements that are beyond the detection limit of the instrument (>40 cycles) as “undetermined”. Detection Limit Reset is performed when at least 1 of 3 target gene FAM C_{T} replicates are not detected after 40 cycles and are designated as “undetermined”. “Undetermined” target gene FAM C_{T} replicates are re-set to 40 and flagged. C_{T} normalization (ΔC_{T}) and relative expression calculations that have used re-set FAM C_{T} values are also flagged.

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition, e.g., prostate cancer. The concept of a biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, and/or a particular agent.

The choice of a baseline profile data set for creating a calibrated profile data set is related to the biological condition to be evaluated, monitored, or predicted, as well as, the intended use of the calibrated panel, e.g., as to monitor drug development, quality control or other uses. It may be desirable to access baseline profile data sets from the same subject for whom a first profile data set is obtained or from different subject at varying times, exposures to stimuli, drugs or complex compounds; or may be derived from like or dissimilar populations or sets of subjects. The baseline profile data set may be normal, healthy baseline. Alternatively, the baseline profile data set may be derived from otherwise healthy subjects with BPH.

The profile data set may arise from the same subject for which the first data set is obtained, where the sample is taken at a separate or similar time, a different or similar site or in a different or similar biological condition. For example, a sample may be taken before stimulation or after stimulation with an exogenous compound or substance, such as before or after therapeutic treatment. Alternatively the sample is taken before or include before or after a surgical procedure for prostate cancer. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library along with or separate from the baseline profile data base and optionally the first profile data set al. though the first profile data set would normally become incorporated into a baseline profile data set under suitable classification criteria. The remarkable consistency of Gene Expression Profiles associated with a given biological condition makes it valuable to store profile data, which can be used, among other things for normative reference purposes. The normative reference can serve to indicate the degree to which a subject conforms to a given biological condition (healthy or diseased) and, alternatively or in addition, to provide a target for clinical intervention.

### Calibrated Data

Given the repeatability achieved in measurement of gene expression, described above in connection with “Gene Expression Panels” (Precision Profiles™) and “gene amplification”, it was concluded that where differences occur in measurement under such conditions, the differences are attributable to differences in biological condition. Thus, it has been found that calibrated profile data sets are highly reproducible in samples taken from the same individual under the same conditions. Similarly, it has been found that calibrated profile data sets are reproducible in samples that are repeatedly tested. Also found have been repeated instances wherein calibrated profile data sets obtained when samples from a subject are exposed ex vivo to a compound are comparable to calibrated profile data from a sample that has been exposed to a sample in vivo.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within 20%, and typically within 10%. In accordance with embodiments of the invention, a pattern of increasing, decreasing and no change in relative gene expression from each of a plurality of gene loci examined in the Gene Expression Panel (Precision Profile™) may be used to prepare a calibrated profile set that is informative with regards to a biological condition, biological efficacy of an agent treatment conditions or for comparison to populations or sets of subjects or samples, or for comparison to populations of cells. Patterns of this nature may be used to identify likely candidates for a drug trial, used alone or in combination with other clinical indicators to be diagnostic or prognostic with respect to a biological condition or may be used to guide the development of a pharmaceutical or nutraceutical through manufacture, testing and marketing.

The numerical data obtained from quantitative gene expression and numerical data from calibrated gene expression relative to a baseline profile data set may be stored in databases or digital storage mediums and may be retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites.

The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the prostate cancer or condition related to prostate cancer to be evaluated, with the calibrated profile data set being a comparison between the first profile data set and the baseline profile data set, thereby providing evaluation of prostate cancer or a condition related to prostate cancer of the subject.

In yet other embodiments, the function is a mathematical function and is other than a simple difference, including a second function of the ratio of the corresponding member of first profile data set to the corresponding member of the baseline profile data set, or a logarithmic function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

In other embodiments, a clinical indicator may be used to assess the prostate cancer or condition related to prostate cancer of the relevant set of subjects by interpreting the calibrated profile data set in the context of at least one other clinical indicator, wherein the at least one other clinical indicator is selected from the group consisting of blood chemistry, (e.g., PSA levels) X-ray or other radiological or metabolic imaging technique, molecular markers in the blood, other chemical assays, and physical findings.

### Index Construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel (Precision Profile™) giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of a biological condition.

An index may be constructed using an index function that maps values in a Gene Expression Profile into a single value that is pertinent to the biological condition at hand. The values in a Gene Expression Profile are the amounts of each constituent of the Gene Expression Panel (Precision Profile™). These constituent amounts form a profile data set, and the index function generates a single value—the index—from the members of the profile data set.

The index function may conveniently be constructed as a linear sum of terms, each term being what is referred to herein as a “contribution function” of a member of the profile data set. For example, the contribution function may be a constant times a power of a member of the profile data set. So the index function would have the form

I=ΣCiMi^{P(i)},

where I is the index, Mi is the value of the member i of the profile data set, Ci is a constant, and P(i) is a power to which Mi is raised, the sum being formed for all integral values of i up to the number of members in the data set. We thus have a linear polynomial expression. The role of the coefficient Ci for a particular gene expression specifies whether a higher ΔCt value for this gene either increases (a positive Ci) or decreases (a lower value) the likelihood of prostate cancer, the ΔCt values of all other genes in the expression being held constant.

The values Ci and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Mass., called Latent Gold®. Alternatively, other simpler modeling techniques may be employed in a manner known in the art. The index function for prostate cancer may be constructed, for example, in a manner that a greater degree of prostate cancer (as determined by the profile data set for the Precision Profile™ listed in Table 1 described herein correlates with a large value of the index function.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

As an example, the index can be constructed, in relation to a normative Gene Expression Profile for a population or set of healthy subjects, in such a way that a reading of approximately 1 characterizes normative Gene Expression Profiles of healthy subjects. Let us further assume that the biological condition that is the subject of the index is prostate cancer; a reading of 1 in this example thus corresponds to a Gene Expression Profile that matches the norm for healthy subjects (i.e., normal, healthy subjects or otherwise healthy subjects with BPH). A substantially higher reading then may identify a subject experiencing prostate cancer, or a condition related to prostate cancer. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since it was determined that Gene Expression Profile values (and accordingly constructed indices based on them) tend to be normally distributed, the 0-centered index constructed in this manner is highly informative. It therefore facilitates use of the index in diagnosis of disease and setting objectives for treatment.

Still another embodiment is a method of providing an index pertinent to prostate cancer or a condition related to prostate cancer of a subject based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the presumptive signs of prostate cancer, the panel including at least one constituent of any of the genes listed in the Precision Profile™ for Prostate Cancer Detection (Table 1). In deriving the profile data set, such measure for each constituent is achieved under measurement conditions that are substantially repeatable, at least one measure from the profile data set is applied to an index function that provides a mapping from at least one measure of the profile data set into one measure of the presumptive signs of prostate cancer, so as to produce an index pertinent to the prostate cancer or condition related to prostate cancer of the subject.

As another embodiment of the invention, an index function I of the form

I=C₀+ΣCᵢM₁ᵢ^{P1(i)}M₂ᵢ^{P2(i)},

can be employed, where M₁ and M₂ are values of the member i of the profile data set, Cᵢ is a constant determined without reference to the profile data set, and P1 and P2 are powers to which M₁ and M₂ are raised. The role of P1(i) and P2(i) is to specify the specific functional form of the quadratic expression, whether in fact the equation is linear, quadratic, contains cross-product terms, or is constant. For example, when P1=P2=0, the index function is simply the sum of constants; when P1=1 and P2=0, the index function is a linear expression; when P1=P2=1, the index function is a quadratic expression.

The constant C₀ serves to calibrate this expression to the biological population of interest that is characterized by having prostate cancer. In this embodiment, when the index value equals 0, the odds are 50:50 of the subject having prostate cancer vs a normal subject or otherwise healthy subject with BPH. More generally, the predicted odds of the subject having prostate cancer is [exp(Iᵢ)], and therefore the predicted probability of having prostate cancer is [exp(Iᵢ)]/[1+exp((Iᵢ)]. Thus, when the index exceeds 0, the predicted probability that a subject has prostate cancer is higher than 0.5, and when it falls below 0, the predicted probability is less than 0.5.

The value of C₀ may be adjusted to reflect the prior probability of being in this population based on known exogenous risk factors for the subject. In an embodiment where C₀ is adjusted as a function of the subject's risk factors, where the subject has prior probability pᵢ of having prostate cancer based on such risk factors, the adjustment is made by increasing (decreasing) the unadjusted C₀ value by adding to C₀ the natural logarithm of the following ratio: the prior odds of having prostate cancer taking into account the risk factors/the overall prior odds of having prostate cancer without taking into account the risk factors.

### Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having prostate cancer is based on whether the subjects have an “effective amount” or a “significant alteration” in the levels of a cancer associated gene. By “effective amount” or “significant alteration”, it is meant that the measurement of an appropriate number of cancer associated gene (which may be one or more) is different than the predetermined cut-off point (or threshold value) for that cancer associated gene and therefore indicates that the subject has prostate cancer for which the cancer associated gene(s) is a determinant.

The difference in the level of cancer associated gene(s) between normal and abnormal is preferably statistically significant. As noted below, and without any limitation of the invention, achieving statistical significance, and thus the preferred analytical and clinical accuracy, generally but not always requires that combinations of several cancer associated gene(s) be used together in panels and combined with mathematical algorithms in order to achieve a statistically significant cancer associated gene index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining an effective amount or a significant alteration of cancer associated gene(s), which thereby indicates the presence of a prostate cancer in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, desirably at least 0.775, more desirably at least 0.800, preferably at least 0.825, more preferably at least 0.850, and most preferably at least 0.875.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing prostate cancer, and the bottom quartile comprising the group of subjects having the lowest relative risk for developing prostate cancer. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease. Often such lower diagnostic accuracy tests must be combined with additional parameters in order to derive meaningful clinical thresholds for therapeutic intervention, as is done with the aforementioned global risk assessment indices.

A health economic utility function is yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those at risk for having a bone fracture) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the cancer associated gene(s) of the invention allows for one of skill in the art to use the cancer associated gene(s) to identify, diagnose, or prognose subjects with a pre-determined level of predictability and performance.

Results from the cancer associated gene(s) indices thus derived can then be validated through their calibration with actual results, that is, by comparing the predicted versus observed rate of disease in a given population, and the best predictive cancer associated gene(s) selected for and optimized through mathematical models of increased complexity. Many such formula may be used; beyond the simple non-linear transformations, such as logistic regression, of particular interest in this use of the present invention are structural and syntactic classification algorithms, and methods of risk index construction, utilizing pattern recognition features, including established techniques such as the Kth-Nearest Neighbor, Boosting, Decision Trees, Neural Networks, Bayesian Networks, Support Vector Machines, and Hidden Markov Models, as well as other formula described herein.

Furthermore, the application of such techniques to panels of multiple cancer associated gene(s) is provided, as is the use of such combination to create single numerical “risk indices” or “risk scores” encompassing information from multiple cancer associated gene(s) inputs. Individual B cancer associated gene(s) may also be included or excluded in the panel of cancer associated gene(s) used in the calculation of the cancer associated gene(s) indices so derived above, based on various measures of relative performance and calibration in validation, and employing through repetitive training methods such as forward, reverse, and stepwise selection, as well as with genetic algorithm approaches, with or without the use of constraints on the complexity of the resulting cancer associated gene(s) indices.

The above measurements of diagnostic accuracy for cancer associated gene(s) are only a few of the possible measurements of the clinical performance of the invention. It should be noted that the appropriateness of one measurement of clinical accuracy or another will vary based upon the clinical application, the population tested, and the clinical consequences of any potential misclassification of subjects. Other important aspects of the clinical and overall performance of the invention include the selection of cancer associated gene(s) so as to reduce overall cancer associated gene(s) variability (whether due to method (analytical) or biological (pre-analytical variability, for example, as in diurnal variation), or to the integration and analysis of results (post-analytical variability) into indices and cut-off ranges), to assess analyte stability or sample integrity, or to allow the use of differing sample matrices amongst blood, cells, serum, plasma, urine, etc.

### Kits

The invention also includes a prostate cancer detection reagents, i.e., nucleic acids and or proteins that specifically identify one or more prostate cancer or condition related to prostate cancer nucleic acids (e.g., any gene listed in Table 1 and Table 8, oncogenes, tumor suppression genes, tumor progression genes, angiogenesis genes and lymphogenesis genes; sometimes referred to herein as prostate cancer associated genes or prostate cancer associated constituents) by having homologous nucleic acid sequences, such as oligonucleotide sequences, complementary to a portion of the prostate cancer associated gene nucleic acids or antibodies to proteins encoded by the prostate cancer associated gene nucleic acids packaged together in the form of a kit. The oligonucleotides can be fragments of the prostate cancer associated genes. For example the oligonucleotides can be 200, 150, 100, 50, 25, 10 or less nucleotides in length. In another embodiment, the detection reagent is one or more antibodies that specifically identify one or more prostate cancer detection proteins.

The kit may contain in separate containers a nucleic acid or antibody (either already bound to a solid matrix or packaged separately with reagents for binding them to the matrix), control formulations (positive and/or negative), and/or a detectable label. Instructions (i.e., written, tape, VCR, CD-ROM, etc.) for carrying out the assay may be included in the kit. The assay may for example be in the form of PCR, a Northern hybridization or a sandwich ELISA, as known in the art.

For example, the kit may comprise one or more antibodies or antibody fragments which specifically bind to a protein equivalent of a constituent of the Precision Profile™ for Prostate Cancer (Table 1) or protein equivalent of a constituent of the Prostate Cancer Clinically Tested Precision Profile™ (Table 8). The antibodies may be conjugated to a solid support suitable for a diagnostic assay (e.g., beads, plates, slides or wells formed from materials such as latex or polystyrene) in accordance with known techniques, such as precipitation. Antibodies as described herein may likewise be conjugated to detectable groups such as radiolabels (e.g., 35 S, 125 I, 131 I), enzyme labels (e.g., horseradish peroxidase, alkaline phosphatase), and fluorescent labels (e.g., fluorescein) in accordance with known techniques. Alternatively the kit comprises (a) an antibody conjugated to a solid support and (b) a second antibody of the invention conjugated to a detectable group, or (a) an antibody, and (b) a specific binding partner for the antibody conjugated to a detectable group.

In another embodiment, prostate cancer associated gene detection reagents can be immobilized on a solid matrix such as a porous strip to form at least one prostate cancer associated gene detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of prostate cancer associated genes present in the sample. The detection sites may be configured in any suitably detectable shape and are typically in the shape of a bar or dot spanning the width of a test strip.

Alternatively, prostate cancer associated genes can be labeled (e.g., with one or more fluorescent dyes) and immobilized on lyophilized beads to form at least one prostate cancer gene detection site. The beads may also contain sites for negative and/or positive controls. Upon addition of the test sample, the number of sites displaying a detectable signal provides a quantitative indication of the amount of prostate cancer associated genes present in the sample.

Alternatively, the kit contains a nucleic acid substrate array comprising one or more nucleic acid sequences. The nucleic acids on the array specifically identify one or more nucleic acid sequences represented by prostate cancer associated genes (see Table 1). In various embodiments, the expression of 1, 2, 3, 4, 5, 6, 7, 8, 9, 10, 15, 20, 25, 40 or 50 or more of the sequences represented by prostate cancer associated genes (see Table 1) can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

The skilled artisan can routinely make antibodies, nucleic acid probes, i.e., oligonucleotides, aptamers, siRNAs, antisense oligonucleotides, against any of the prostate cancer detection genes a listed in Tables 1 and 8.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Example 1

### Patient Population

Screening for prostate cancer with PSA testing is limited by a high number of false positives, particularly in the setting of benign prostatic hypertrophy (BPH). The goal of the studies described herein was to develop whole blood RNA transcript-based diagnostic tests that improve the diagnosis of untreated, localized prostate cancer over the use of the PSA test alone.

Several multi-gene models (i.e., Precision Profiles™) having improved discrimination between prostate cancer subjects and normal, healthy, or otherwise healthy subjects with BPH, over the use of PSA alone are described herein. Multi-gene models (i.e., Precision Profiles™) with improved discrimination between prostate cancer subjects having different grades of cancer (i.e., non-aggressive prostate cancer versus aggressive prostate cancer, based on Gleason score) are also described herein. These multi-gene models were identified using RNA samples isolated from a “Training Set” of subjects, and validated using RNA samples isolated from a “Test Set” of subjects.

RNA was isolated from whole blood that was collected in PaxGene™ Blood RNA Tubes from a total of 204 prostate cancer subjects and 2 control groups consisting of age-matched, medically defined normal subjects (N=170) and otherwise healthy subjects with BPH(N=110), for a total of 484 subject samples. Blood RNA tubes were manually processed to total RNA. RNA quality and quantity was assessed on the Agilent Bioanalyzer 2100. RNA was converted to cDNA in a random hexamer primed reaction with reverse transcriptase. cDNA was quality checked and used as the template in a quantitative PCR assay optimized for precision and calibration. The subject samples were divided into a Training set and Test set as follows:

### Training Set:

A total of 76 untreated, localized prostate cancer subjects, 76 age-matched, medically defined normal, healthy subjects, and 30 age-matched BPH subjects (Nₜₒₜₐₗ=182) were selected to identify a preliminary biomarker panel. The 174 inflammation and cancer-related genes listed in the Precision Profile™ for Prostate Cancer Detection (Table 1) were assayed against RNA samples isolated from the training set. The resulting gene models identified using the gene expression analysis from these subject samples are described in Examples 3-5 below.

### Test Set:

A total of 128 untreated, localized prostate cancer subject, 94 medically defined age-matched normal subjects and 80 age-matched BPH subjects (Nₜₒₜₐₗ=302) were selected for validating the biomarker panel identified using the Training set. Twenty-one genes (selected from the training set) were assayed against RNA sample isolated from the test set. The resulting gene models identified using gene expression analysis based on these subject samples are described in Example 6 below.

### Age-Matching/Age-Adjusted PSA Cut-Offs:

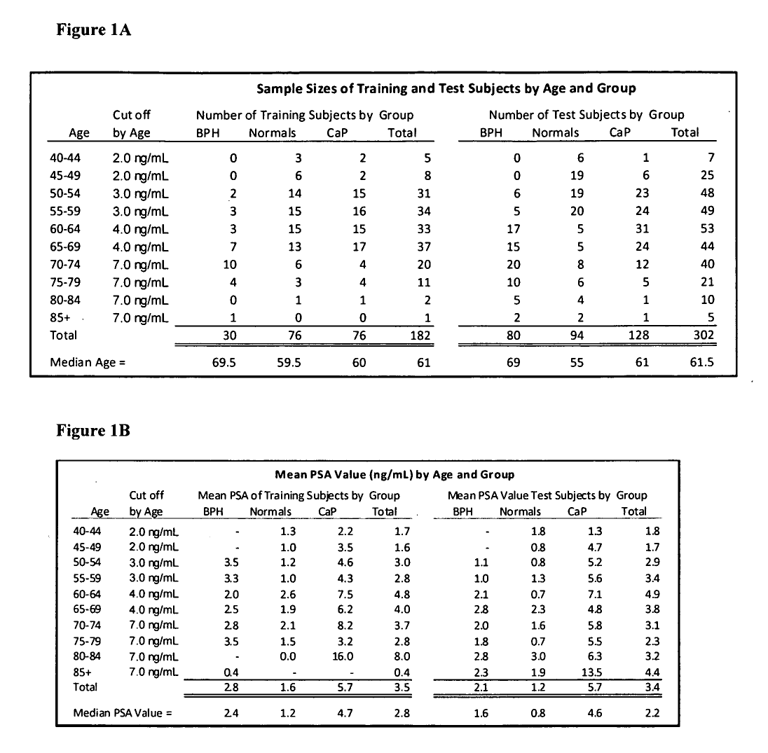

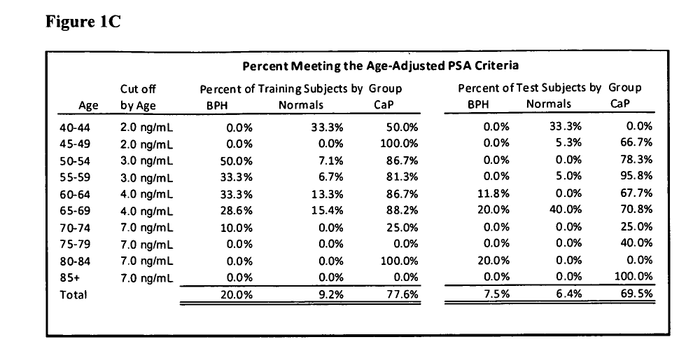

The prostate cancer subjects and normal subjects (with and without BPH) were age matched (i.e., selected to be similar in age to each other) within 5 years in both the Training and Test datasets, as reflected in column 1 of FIG. 1A below. In some examples, PSA levels of the subjects were also age-adjusted (represented by dummy (dichotomous) variable coded 1 for all subjects (normal, BPH or CaP) if their PSA level fell above a given cut-off dependent on their age, as shown in FIG. 1. The PSA cut-off levels applied to each given age range are shown in Column 2 of FIG. 1A. The mean PSA value by age and group (CaP, normal, BPH) is shown in FIG. 1B and the percent meeting the age-adjusted PSA criteria is shown in FIG. 1C.

Examples 3-6 below describe multi-gene logistic regregression models capable of distinguishing between prostate cancer subjects normal, healthy subjects or otherwise healthy subjects with BPH.

### Example 2

### Enumeration and Classification Methodology Based on Logistic Regression Models

### Introduction

The following methods were used to generate gene models capable of distinguishing between subjects diagnosed with prostate cancer and normal subjects, with at least 75% classification accuracy, as described in Examples 3-6 below.

Given measurements on G genes from samples of N₁ subjects belonging to group 1 and N₂ members of group 2, the purpose was to identify models containing g<G genes which discriminate between the 2 groups. The groups might be such that one consists of reference subjects (e.g., healthy, normal subjects) while the other group might have a specific disease, or subjects in group 1 may have disease A while those in group 2 may have disease B.

Specifically, parameters from a linear logistic regression model were estimated to predict a subject's probability of belonging to group 1 given his (her) measurements on the g genes in the model. After all the models were estimated (all G 1-gene models were estimated, as well as all

$$( \begin{matrix}G \\ 2\end{matrix} ) = {G}^{*} ( G \text{-} 1 ) / 2 \; 2 \text{-} gene models ,$$

and all (G 3)=G*(G−1)*(G−2)/6 3-gene models based on G genes (number of combinations taken 3 at a time from G)), they were evaluated using a 2-dimensional screening process. The first dimension employed a statistical screen (significance of incremental p-values) that eliminated models that were likely to overfit the data and thus may not validate when applied to new subjects. The second dimension employed a clinical screen to eliminate models for which the expected misclassification rate was higher than an acceptable level. As a threshold analysis, the gene models showing less than 75% discrimination between N₁ subjects belonging to group 1 and N₂ members of group 2 (i.e., misclassification of 25% or more of subjects in either of the 2 sample groups), and genes with incremental p-values that were not statistically significant, were eliminated.

### Methodological, Statistical and Computing Tools Used

The Latent GOLD program (Vermunt and Magidson, 2005) was used to estimate the logistic regression models. For efficiency in processing the models, the LG-Syntax™ Module available with version 4.5 of the program (Vermunt and Magidson, 2007) was used in batch mode, and all g-gene models associated with a particular dataset were submitted in a single run to be estimated. That is, all 1-gene models were submitted in a single run, all 2-gene models were submitted in a second run, etc.

### The Data

The data consists of ΔC_{T} values for each sample subject in each of the 2 groups (e.g., prostate cancer subject vs. reference (e.g., healthy, normal subjects or otherwise healthy subjects with BPH) on each of G(k) genes obtained from a particular class k of genes (e.g., the 174 inflammation and prostate cancer specific genes shown in Table 1).

### Analysis Steps

The steps in a given analysis of the G(k) genes measured on N₁ subjects in group 1 and N₂ subjects in group 2 are as follows:
1) Eliminate low expressing genes: In some instances, target gene FAM measurements were beyond the detection limit (i.e., very high ΔC_{T} values which indicate low expression) of the particular platform instrument used to detect and quantify constituents of a Gene Expression Panel (Precision Profile™). To address the issue of “undetermined” gene expression measures as lack of expression for a particular gene, the detection limit was reset and the “undetermined” constituents were “flagged”, as previously described. C_{T} normalization (ΔC_{T}) and relative expression calculations that have used re-set FAM C_{T} values were also flagged. In some instances, these low expressing genes (i.e., re-set FAM C_{T} values) were eliminated from the analysis in step 1 if 50% or more ΔC_{T} values from either of the 2 groups were flagged. Although such genes were eliminated from the statistical analyses described herein, one skilled in the art would recognize that such genes may be relevant in a disease state.
2) Estimate logistic regression (logit) models predicting P(i)=the probability of being in group 1 for each subject i=1, 2, . . . , N₁+N₂. Since there are only 2 groups, the probability of being in group 2 equals 1−P(i). The maximum likelihood (ML) algorithm implemented in Latent GOLD 4.0 (Vermunt and Magidson, 2005) was used to estimate the model parameters. All 1-gene models were estimated first, followed by all 2-gene models and in cases where the sample sizes N₁ and N₂ were sufficiently large, all 3-gene models were estimated.
3) Screen out models that fail to meet the statistical or clinical criteria: Regarding the statistical criteria, models were retained if the incremental p-values for the parameter estimates for each gene (i.e., for each predictor in the model) fell below the cut-off point alpha=0.05. Regarding the clinical criteria, models were retained if the percentage of cases within each group (e.g., disease group, and reference group (e.g., healthy, normal subjects) that was correctly predicted to be in that group was at least 75%. For technical details, see the section “Application of the Statistical and Clinical Criteria to Screen Models”.
4) Each model yielded an index that could be used to rank the sample subjects. Such an index value could also be computed for new cases not included in the sample. See the section “Computing Model-based Indices for each Subject” for details on how this index was calculated.
5) A cut-off value somewhere between the lowest and highest index value was selected and based on this cut-off, subjects with indices above the cut-off were classified (predicted to be) in the disease group, those below the cut-off were classified into the reference group (i.e., normal, healthy subjects). Based on such classifications, the percent of each group that is correctly classified was determined. See the section labeled “Classifying Subjects into Groups” for details on how the cut-off was chosen.
6) Among all models that survived the screening criteria (Step 3), an entropy-based R² statistic was used to rank the models from high to low, i.e., the models with the highest percent classification rate to the lowest percent classification rate. The top 5 such models are then evaluated with respect to the percent correctly classified and the one having the highest percentages was selected as the single “best” model.

While there are several possible R² statistics that might be used for this purpose, it was determined that the one based on entropy was most sensitive to the extent to which a model yields clear separation between the 2 groups. Such sensitivity provides a model which can be used as a tool by a practitioner (e.g., primary care physician, oncologist, etc.) to ascertain the necessity of future screening or treatment options. For more detail on this issue, see the section labeled “Using R²Statistics to Rank Models” below.

### Computing Model-Based Indices for Each Subject

The model parameter estimates were used to compute a numeric value (logit, odds or probability) for each diseased and reference subject (e.g., healthy, normal subject) in the sample. For illustrative purposes only, in an example of a 2-gene logit model for prostate cancer containing the genes ALOX5 and S100A6, the following parameter estimates listed in Table A were obtained:

TABLE A

| | | |
|---|---|---|
| Prostate Cancer | alpha(1) | 18.37 |
| Normals | alpha(2) | −18.37 |
| Predictors | | |
| ALOX5 | beta(1) | −4.81 |
| S100A6 | beta(2) | 2.79 |

For a given subject with particular ΔC_{T} values observed for these genes, the predicted logit associated with prostate cancer vs. reference (i.e., normals) was computed as:

LOGIT(ALOX5,S100A6)=[alpha(1)−alpha(2)]+beta(1)*ALOX5+beta(2)*S100A6.

The predicted odds of having prostate cancer would be:

ODDS(ALOX5,S100A6)=exp[LOGIT(ALOX5,S100A6)]

and the predicted probability of belonging to the prostate cancer group is:

P(ALOX5,S100A6)=ODDS(ALOX5,S100A6)/[1+ODDS(ALOX5,S100A6)]

Note that the ML estimates for the alpha parameters were based on the relative proportion of the group sample sizes. Prior to computing the predicted probabilities, the alpha estimates may be adjusted to take into account the relative proportion in the population to which the model will be applied (e.g., the incidence of prostate cancer in the population of adult men in the U.S.)

Classifying Subjects into Groups

The “modal classification rule” was used to predict into which group a given case belongs. This rule classifies a case into the group for which the model yields the highest predicted probability. Using the same prostate cancer example previously described (for illustrative purposes only), use of the modal classification rule would classify any subject having P>0.5 into the prostate cancer group, the others into the reference group (e.g., healthy, normal subjects). The percentage of all N₁ prostate cancer subjects that were correctly classified were computed as the number of such subjects having P≧0.5 divided by N₁. Similarly, the percentage of all N₂ reference (e.g., normal healthy) subjects that were correctly classified were computed as the number of such subjects having P 0.5 divided by N₂. Alternatively, a cut-off point P₀ could be used instead of the modal classification rule so that any subject i having P(i)>P₀ is assigned to the prostate cancer group, and otherwise to the Reference group (e.g., normal, healthy group).

### Application of the Statistical and Clinical Criteria to Screen Models

### Clinical Screening Criteria

In order to determine whether a model met the clinical 75% correct classification criteria, the following approach was used:
A. All sample subjects were ranked from high to low by their predicted probability P (e.g., see Table B).
B. Taking P₀(i)=P(i) for each subject, one at a time, the percentage of group 1 and group 2 that would be correctly classified, P₁(i) and P₂(i) was computed.
C. The information in the resulting table was scanned and any models for which none of the potential cut-off probabilities met the clinical criteria (i.e., no cut-offs P₀(i) exist such that both P₁(i)>0.75 and P₂(i)>0.75) were eliminated. Hence, models that did not meet the clinical criteria were eliminated.

The example shown in Table B has many cut-offs that meet this criteria. For example, the cut-off P₀=0.4 yields correct classification rates of 92% for the reference group (i.e., normal, healthy subjects), and 93% for Prostate Cancer subjects.

### Statistical Screening Criteria

In order to determine whether a model met the statistical criteria, the following approach was used to compute the incremental p-value for each gene g=1, 2, . . . , G as follows:
i. Let LSQ(0) denote the overall model L-squared output by Latent GOLD for an unrestricted model.
ii. Let LSQ(g) denote the overall model L-squared output by Latent GOLD for the restricted version of the model where the effect of gene g is restricted to 0.
iii. With 1 degree of freedom, use a ‘components of chi-square’ table to determine the p-value associated with the LR difference statistic LSQ(g)−LSQ(0).
   Note that this approach required estimating g restricted models as well as 1 unrestricted model.

### Discrimination Plots

For a 2-gene model, a discrimination plot consisted of plotting the ΔC_{T} values for each subject in a scatterplot where the values associated with one of the genes served as the vertical axis, the other serving as the horizontal axis. Two different symbols were used for the points to denote whether the subject belongs to group 1 or 2.

A line was appended to a discrimination graph to illustrate how well the 2-gene model discriminated between the 2 groups. The slope of the line was determined by computing the ratio of the ML parameter estimate associated with the gene plotted along the horizontal axis divided by the corresponding estimate associated with the gene plotted along the vertical axis. The intercept of the line was determined as a function of the cut-off point.

For a 3-gene model, a 2-dimensional slice defined as a linear combination of 2 of the genes was plotted along one of the axes, the remaining gene being plotted along the other axis. The particular linear combination was determined based on the parameter estimates. For example, if a 3^{rd} gene were added to the 2-gene model consisting of ALOX5 and S100A6 and the parameter estimates for ALOX5 and S100A6 were beta(1) and beta(2) respectively, the linear combination beta(1)*ALOX5+beta(2)*S100A6 could be used. This approach can be readily extended to the situation with 4 or more genes in the model by taking additional linear combinations. For example, with 4 genes one might use beta(1)*ALOX5+beta(2)*S100A6 along one axis and beta(3)*gene3+beta(4)*gene4 along the other, or beta(1)*ALOX5+beta(2)*S100A6+beta(3)*gene3 along one axis and gene4 along the other axis. When producing such plots with 3 or more genes, genes with parameter estimates having the same sign were chosen for combination.

### Using R²Statistics to Rank Models

The R² in traditional OLS (ordinary least squares) linear regression of a continuous dependent variable can be interpreted in several different ways, such as 1) proportion of variance accounted for, 2) the squared correlation between the observed and predicted values, and 3) a transformation of the F-statistic. When the dependent variable is not continuous but categorical (in our models the dependent variable is dichotomous—membership in the diseased group or reference group), this standard R² defined in terms of variance (see definition 1 above) is only one of several possible measures. The term ‘pseudo R²’ has been coined for the generalization of the standard variance-based R² for use with categorical dependent variables, as well as other settings where the usual assumptions that justify OLS do not apply.

The general definition of the (pseudo) R² for an estimated model is the reduction of errors compared to the errors of a baseline model. For the purpose of the present invention, the estimated model is a logistic regression model for predicting group membership based on 1 or more continuous predictors (ΔC_{T} measurements of different genes). The baseline model is the regression model that contains no predictors; that is, a model where the regression coefficients are restricted to 0. More precisely, the pseudo R² is defined as:

R²=[Error(baseline)−Error(model)]/Error(baseline)

Regardless how error is defined, if prediction is perfect, Error(model)=0 which yields R²=1. Similarly, if all of the regression coefficients do in fact turn out to equal 0, the model is equivalent to the baseline, and thus R²=0. In general, this pseudo R² falls somewhere between 0 and 1.

When Error is defined in terms of variance, the pseudo R² becomes the standard R². When the dependent variable is dichotomous group membership, scores of 1 and 0, −1 and +1, or any other 2 numbers for the 2 categories yields the same value for R². For example, if the dichotomous dependent variable takes on the scores of 1 and 0, the variance is defined as P*(1−P) where P is the probability of being in 1 group and 1−P the probability of being in the other.

A common alternative in the case of a dichotomous dependent variable, is to define error in terms of entropy. In this situation, entropy can be defined as P*ln(P)*(1−P)*ln(1−P) (for further discussion of the variance and the entropy based R², see Magidson, Jay, “Qualitative Variance, Entropy and Correlation Ratios for Nominal Dependent Variables,” Social Science Research 10 (June), pp. 177-194).

The R² statistic was used in the enumeration methods described herein to identify the “best” gene-model. R² can be calculated in different ways depending upon how the error variation and total observed variation are defined. For example, four different R² measures output by Latent GOLD are based on:

a) Standard variance and mean squared error (MSE)
b) Entropy and minus mean log-likelihood (−MLL)
c) Absolute variation and mean absolute error (MAE)
d) Prediction errors and the proportion of errors under modal assignment (PPE)

Each of these 4 measures equal 0 when the predictors provide zero discrimination between the groups, and equal 1 if the model is able to classify each subject into their actual group with 0 error. For each measure, Latent GOLD defines the total variation as the error of the baseline (intercept-only) model which restricts the effects of all predictors to 0. Then for each, R² is defined as the proportional reduction of errors in the estimated model compared to the baseline model. For the 2-gene prostate cancer example used to illustrate the enumeration methodology described herein, the baseline model classifies all cases as being in the diseased group since this group has a larger sample size, resulting in 50 misclassifications (all 50 normal subjects are misclassified) for a prediction error of 50/107=0.467. In contrast, there are only 10 prediction errors (= 10/107=0.093) based on the 2-gene model using the modal assignment rule, thus yielding a prediction error R² of 1−0.093/0.467=0.8. As shown in Exhibit 1, 4 normal and 6 cancer subjects would be misclassified using the modal assignment rule. Note that the modal rule utilizes P₀=0.5 as the cut-off. If P₀=0.4 were used instead, there would be only 8 misclassified subjects.

To reduce the likelihood of obtaining models that capitalize on chance variations in the observed samples the models may be limited to contain only M genes as predictors in the model. (Although a model may meet the significance criteria, it may overfit data and thus would not be expected to validate when applied to a new sample of subjects.) For example, for M=2, all models would be estimated which contain:

$$A . 1 \text{-} gene - G such models$$

$$B . 2 \text{-} gene models - ( \begin{matrix}G \\ 2\end{matrix} ) = {G}^{*} ( G \text{-} 1 ) / 2 such models$$

$$C . 3 \text{-} gene models - ( G 3 ) = {{G}^{*} ( G \text{-} 1 )}^{*} ( G \text{-} 2 ) / 6 such models$$

TABLE B
ΔC_{T} Values and Model Predicted Probability
of Prostate Cancer for Each Subject

| ALOX5 | S100A6 | P | Group |
|---|---|---|---|
| 13.92 | 16.13 | 1.0000 | Cancer |
| 13.90 | 15.77 | 1.0000 | Cancer |
| 13.75 | 15.17 | 1.0000 | Cancer |
| 13.62 | 14.51 | 1.0000 | Cancer |
| 15.33 | 17.16 | 1.0000 | Cancer |
| 13.86 | 14.61 | 1.0000 | Cancer |
| 14.14 | 15.09 | 1.0000 | Cancer |
| 13.49 | 13.60 | 0.9999 | Cancer |
| 15.24 | 16.61 | 0.9999 | Cancer |
| 14.03 | 14.45 | 0.9999 | Cancer |
| 14.98 | 16.05 | 0.9999 | Cancer |
| 13.95 | 14.25 | 0.9999 | Cancer |
| 14.09 | 14.13 | 0.9998 | Cancer |
| 15.01 | 15.69 | 0.9997 | Cancer |
| 14.13 | 14.15 | 0.9997 | Cancer |
| 14.37 | 14.43 | 0.9996 | Cancer |
| 14.14 | 13.88 | 0.9994 | Cancer |
| 14.33 | 14.17 | 0.9993 | Cancer |
| 14.97 | 15.06 | 0.9988 | Cancer |
| 14.59 | 14.30 | 0.9984 | Cancer |
| 14.45 | 13.93 | 0.9978 | Cancer |
| 14.40 | 13.77 | 0.9972 | Cancer |
| 14.72 | 14.31 | 0.9971 | Cancer |
| 14.81 | 14.38 | 0.9963 | Cancer |
| 14.54 | 13.91 | 0.9963 | Cancer |
| 14.88 | 14.48 | 0.9962 | Cancer |
| 14.85 | 14.42 | 0.9959 | Cancer |
| 15.40 | 15.30 | 0.9951 | Cancer |
| 15.58 | 15.60 | 0.9951 | Cancer |
| 14.82 | 14.28 | 0.9950 | Cancer |
| 14.78 | 14.06 | 0.9924 | Cancer |
| 14.68 | 13.88 | 0.9922 | Cancer |
| 14.54 | 13.64 | 0.9922 | Cancer |
| 15.86 | 15.91 | 0.9920 | Cancer |
| 15.71 | 15.60 | 0.9908 | Cancer |
| 16.24 | 16.36 | 0.9858 | Cancer |
| 16.09 | 15.94 | 0.9774 | Cancer |
| 15.26 | 14.41 | 0.9705 | Cancer |
| 14.93 | 13.81 | 0.9693 | Cancer |
| 15.44 | 14.67 | 0.9670 | Cancer |
| 15.69 | 15.08 | 0.9663 | Cancer |
| 15.40 | 14.54 | 0.9615 | Cancer |
| 15.80 | 15.21 | 0.9586 | Cancer |
| 15.98 | 15.43 | 0.9485 | Cancer |
| 15.20 | 14.08 | 0.9461 | Normal |
| 15.03 | 13.62 | 0.9196 | Cancer |
| 15.20 | 13.91 | 0.9184 | Cancer |
| 15.04 | 13.54 | 0.8972 | Cancer |
| 15.30 | 13.92 | 0.8774 | Cancer |
| 15.80 | 14.68 | 0.8404 | Cancer |
| 15.61 | 14.23 | 0.7939 | Normal |
| 15.89 | 14.64 | 0.7577 | Normal |
| 15.44 | 13.66 | 0.6445 | Cancer |
| 16.52 | 15.38 | 0.5343 | Cancer |
| 15.54 | 13.67 | 0.5255 | Normal |
| 15.28 | 13.11 | 0.4537 | Cancer |
| 15.96 | 14.23 | 0.4207 | Cancer |
| 15.96 | 14.20 | 0.3928 | Normal |
| 16.25 | 14.69 | 0.3887 | Cancer |
| 16.04 | 14.32 | 0.3874 | Cancer |
| 16.26 | 14.71 | 0.3863 | Normal |
| 15.97 | 14.18 | 0.3710 | Cancer |
| 15.93 | 14.06 | 0.3407 | Normal |
| 16.23 | 14.41 | 0.2378 | Cancer |
| 16.02 | 13.91 | 0.1743 | Normal |
| 15.99 | 13.78 | 0.1501 | Normal |
| 16.74 | 15.05 | 0.1389 | Normal |
| 16.66 | 14.90 | 0.1349 | Normal |
| 16.91 | 15.20 | 0.0994 | Normal |
| 16.47 | 14.31 | 0.0721 | Normal |
| 16.63 | 14.57 | 0.0672 | Normal |
| 16.25 | 13.90 | 0.0663 | Normal |
| 16.82 | 14.84 | 0.0596 | Normal |
| 16.75 | 14.73 | 0.0587 | Normal |
| 16.69 | 14.54 | 0.0474 | Normal |
| 17.13 | 15.25 | 0.0416 | Normal |
| 16.87 | 14.72 | 0.0329 | Normal |
| 16.35 | 13.76 | 0.0285 | Normal |
| 16.41 | 13.83 | 0.0255 | Normal |
| 16.68 | 14.20 | 0.0205 | Normal |
| 16.58 | 13.97 | 0.0169 | Normal |
| 16.66 | 14.09 | 0.0167 | Normal |
| 16.92 | 14.49 | 0.0140 | Normal |
| 16.93 | 14.51 | 0.0139 | Normal |
| 17.27 | 15.04 | 0.0123 | Normal |
| 16.45 | 13.60 | 0.0116 | Normal |
| 17.52 | 15.44 | 0.0110 | Normal |
| 17.12 | 14.46 | 0.0051 | Normal |
| 17.13 | 14.46 | 0.0048 | Normal |
| 16.78 | 13.86 | 0.0047 | Normal |
| 17.10 | 14.36 | 0.0041 | Normal |
| 16.75 | 13.69 | 0.0034 | Normal |
| 17.27 | 14.49 | 0.0027 | Normal |
| 17.07 | 14.08 | 0.0022 | Normal |
| 17.16 | 14.08 | 0.0014 | Normal |
| 17.50 | 14.41 | 0.0007 | Normal |
| 17.50 | 14.18 | 0.0004 | Normal |
| 17.45 | 14.02 | 0.0003 | Normal |
| 17.53 | 13.90 | 0.0001 | Normal |
| 18.21 | 15.06 | 0.0001 | Normal |
| 17.99 | 14.63 | 0.0001 | Normal |
| 17.73 | 14.05 | 0.0001 | Normal |
| 17.97 | 14.40 | 0.0001 | Normal |
| 17.98 | 14.35 | 0.0001 | Normal |
| 18.47 | 15.16 | 0.0001 | Normal |
| 18.28 | 14.59 | 0.0000 | Normal |
| 18.37 | 14.71 | 0.0000 | Normal |

### Example 3

### Discrimination of Prostate Cancer Subjects from Healthy, Normal Subjects (Excluding BPH Subjects) Using RNA Transcript-Based Gene Expression: Training Dataset

The cDNA derived from patient blood samples, as described in Example 1, was quality checked and used as the template in a quantitative PCR assay optimized for precision and calibration. Custom primers and probes were prepared for the targeted 174 genes shown in the Precision Profile™ for Prostate Cancer Detection (shown in Table 1), selected to be informative relative to biological state of inflammation and prostate cancer. Individual target genes were multiplexed with 18s rRNA endogenous control. Assays were configured in a 384-well plate formatted for triplicate measures and run on the ABI Prism® 7900HT Sequence Detection System. Gene expression profiles for the 174 prostate cancer specific genes were analyzed using the RNA samples obtained from the Training Dataset (i.e., 76 prostate cancer, 76 medically defined age-matched normals, and 30 age-matched BPH), described in Example 1.

Logistic regression models yielding the best discrimination between subjects diagnosed with prostate cancer and normal subjects (excluding subjects with BPH) were generated using the enumeration and classification methodology described in Example 2. Data files were “filtered-by-rule” to ensure all replicate values met predefined metrics. Normalized gene expression values (delta C_{T} values) for each amplified target gene were calculated (target gene CT−endogenous control CT). Logistic regression methodology was used to obtain all possible 1-, 2- and 3-gene models. Top qualifying 3-gene models were used to develop higher order models (4-6 gene) through stepwise regression technique. Several thousand logistic regression models were identified as capable of distinguishing between subjects diagnosed with prostate cancer and normal subjects (excluding subjects BPH) with at least 75% accuracy. For example, a total of 11,105 3-gene models capable of distinguishing between subjects diagnosed with prostate cancer and normal subjects (excluding BPH) were identified. No additional predictors which discriminate between prostate cancer and normal subjects (e.g., PSA or age) were used in conjunction with these gene-models. As used in this Example, sensitivity refers to the percentage of prostate cancer subjects correctly classified by the gene models described herein, whereas specificity refers to the percentage of normal subjects (without BPH) correctly classified.

The 11,105 gene models capable of distinguishing between subjects diagnosed with prostate cancer (CaP) and normal subjects (excluding BPH) are shown in Table 2A. As shown in Table 2A, the 3-gene models are identified in the first three columns (respectively) on the left side of Table 2A, ranked by their entropy R² value (shown in column 4, ranked from high to low). The number of subjects correctly classified or misclassified by each 3-gene model for each patient group (i.e., CaP vs. Normal (excluding BPH) is shown in columns 5-8. The percent normal subjects and percent prostate cancer subjects correctly classified by the corresponding gene model is shown in columns 9 and 10.

For example, the “best” 3-gene logistic regression model capable of distinguishing between prostate cancer subjects and normal, healthy subjects (defined as the model with the highest entropy R² value, as described in Example 2) based on the 174 genes included in the Precision Profile™ for Prostate Cancer Detection is CD97, CDK2 and SP1, capable of classifying normal subjects with 81.6% accuracy (81.6% specificity), and prostate cancer subjects with 81.6% accuracy (81.6% sensitivity). Each of the 76 normal RNA samples and the 76 prostate cancer RNA samples were analyzed for this 3-gene model, no values were excluded. This 3-gene model correctly classifies 62 of the normal subjects as being in the normal patient population, and misclassifies 14 of the normal subjects as being in the prostate cancer patient population. This 3-gene model correctly classifies 62 of the prostate cancer subjects as being in the prostate cancer patient population and misclassifies 14 of the prostate cancer subjects as being in the normal patient population.

A ranking of the top genes for which gene expression profiles were obtained, from most to least significant, is shown in Table 2B. Table 2B summarizes the mean expression levels of the genes listed in the Precision Profile™ for Prostate Cancer Detection (Table 1) measured in the RNA samples obtained from the prostate cancer subjects in the Training Dataset, as well as the results of significance tests (likelihood ratio p-values) for the difference in the mean expression levels between the normal and prostate cancer subjects.

### Example 4

### Discrimination of Prostate Cancer Subjects from Healthy, Normal Subjects Excluding BPH Using RNA Transcript-Based Gene Expression and PSA Values: Training Dataset

The PSA test is currently used as a predictor for identifying subjects with prostate cancer. However, such test is unreliable and results in a high incidence of false positives, especially in the setting of BPH, resulting in additional costly and unnecessary testing.

PSA values were available for the 76 untreated, localized prostate cancer subjects and 76 age-matched normal subjects from the Training Dataset described in Example 1. The prostate cancer subjects and age-matched normal subjects had a median age of 60 years.

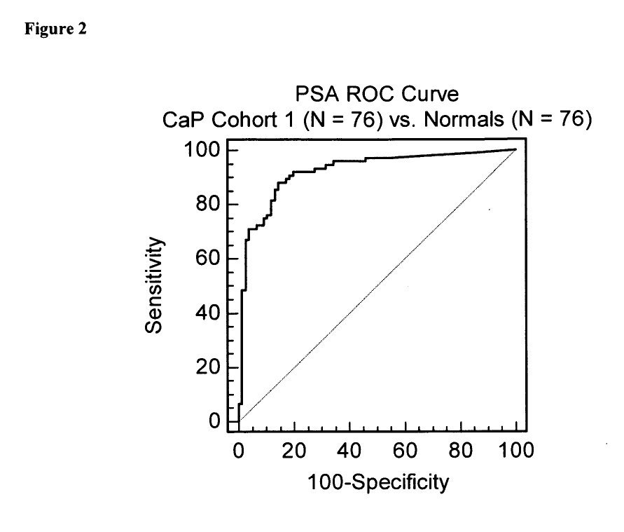

These PSA values were used as the sole predictor to discriminate the prostate cancer subjects from the age-matched normal subjects. As shown in the ROC curve in FIG. 2, PSA alone had a specificity of 94.7%, but sensitivity of only 71.1% for diagnosis of prostate cancer, using a cut-off of 4 ng/ml. When age-adjusted PSA was used as the sole predictor, age-adjusted PSA alone had a specificity of 90.8% but a sensitivity of only 77.6% for diagnosis of prostate cancer. As used in this Example, sensitivity refers to the percentage of prostate cancer subjects correctly classified by the gene models described herein, whereas specificity refers to the percentage of normal subjects (without BPH) correctly classified.

Stepwise methodology was used to determine whether transcript based gene expression combined with PSA levels could improve the sensitivity (i.e., percentage of prostate cancer subjects correctly classified) and specificity (i.e., percentage of normal, healthy subjects (without BPH) correctly classified) over the use of PSA testing alone. Both gene expression data and PSA were available for the 76 untreated, localized prostate cancer subjects and the 76 age-matched normal subjects from the Training Dataset described in Example 1. All possible 1-, 2- and 3-gene logit models were estimated based on the 174 target genes assayed (Table 1) and PSA using the methodology described in Example 2.

Thirty one 2-gene models were found to be statistically significant and were rank ordered (high to low) according to entropy R². Models meeting a minimum 75% correct classification criteria and predictor p-value criteria of <0.05 were retained. The top five 2-gene models were selected for validation (shown in Table 4).

In addition to the 1- and 2-gene models, all possible 3-gene logit models were also estimated from all 174 genes, resulting in an enumeration of 862,924 additional models. Models meeting a minimum entropy R² of 0.6 were retained yielding a total of 3,533 models which displayed a specificity and sensitivity for diagnosis of prostate cancer of over 88%. These 3,533 models are shown in Table 3. Note that the variable plnPSA used in these logit models was a logarithmic transformation of PSA in which PSA values less than 1 were recorded to 1 prior to taking the natural logarithm.

As shown in Table 3, the 3-gene models are identified in the first three columns (respectively) on the left side of Table 3, ranked by their entropy R² value (shown in column 4, ranked from high to low). The number of subjects correctly classified or misclassified by each 3-gene model for each patient group (i.e., CaP vs. Normal (excluding BPH) is shown in columns 5-8. The percent normal subjects and percent prostate cancer subjects correctly classified by the corresponding gene model is shown in columns 9 and 10.

For example, the “best” 3-gene logistic regression model capable of distinguishing between prostate cancer subject and normal healthy subjects when combined with PSA values (defined as the model with the highest entropy R² value, as described in Example 2) based on the 174 genes included in the Precision Profile™ for Prostate Cancer Detection is CD97, RP51077B9.4 and SP1, capable of classifying normal subjects with 89.5% accuracy, and prostate cancer subjects with 89.5% accuracy. Each of the 76 normal RNA samples and the 76 prostate cancer RNA samples were analyzed for this 3-gene model, no values were excluded. This 3-gene model correctly classifies 68 of the normal subjects as being in the normal patient population, and misclassifies 8 of the normal subjects as being in the prostate cancer patient population. This 3-gene model correctly classifies 68 of the prostate cancer subjects as being in the prostate cancer patient population and misclassifies 8 of the prostate cancer subjects as being in the normal patient population.

The top five 3-gene models based on entropy R², were used to generate higher order (>3-gene) models. Higher order models (4- and 6-gene) were developed by starting with the top five 3-gene models (which included PSA) and applying Stepwise Regression technique resulting in four 6-gene models. Two of four 6-gene models cross validated successfully (based on K-fold cross validation with K=10) and the remaining two 6-gene models were reduced to two 4-gene models in order to meet the cross-validation criteria. This yielded an additional two 4-gene models and two 6-gene models for validation (shown in Table 4).

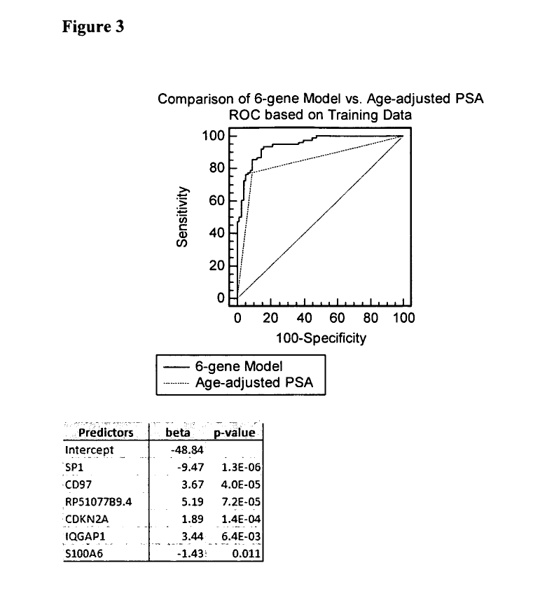

For example, the 3-gene logit model (CD97, RP51077B9.4 and SP1) was used to develop a 6-gene model, RP51077B9.4, CD97, CDKN2A, SP1, S100A6 and IQGAP1, based on the Stepwise regression technique. This 6-gene model significantly improved prediction of prostate cancer compared with age-adjusted PSA. This 6-gene model was capable of distinguishing between prostate cancer subjects and normal, healthy subjects (without BPH) with 97.4% sensitivity and 96.1% specificity. A ROC curve for this 6-gene model compared to age-adjusted PSA criteria is shown in FIG. 3. As shown in FIG. 3, there is improved area under the ROC curve for the 6-gene model (AUC=0.946) as compared to age-adjusted PSA criteria alone (AUC=0.842). The AUC difference 0.104 is statistically significant (p-value=0.005).

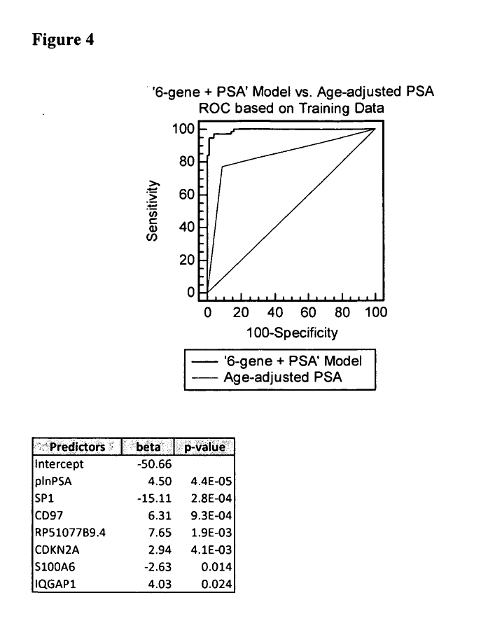

Transcript based gene expression levels of the 6-gene model, combined with PSA values of the 76 prostate cancer subjects and 76 age-matched normal subjects from the Training Dataset, gave even higher specificity (96.1%) and a much improved sensitivity (97.4%) for prostate cancer diagnosis (criterion: Prob (CaP)>0.5) over the use of PSA alone (94.7% specificity, 71.1% sensitivity). A ROC curve for the 6-gene model+PSA model compared to age-adjusted PSA alone is shown in FIG. 4. Improved area under the ROC curve further supports the improved discrimination of prostate cancer versus age-matched normal subjects when combining PSA with gene expression as compared to PSA alone. As shown in FIG. 4, the area under the ROC curve is 0.842 for age-adjusted PSA alone compared to 0.994 for PSA+6 genes. This improvement is statistically significant (p-value=2.0E-06).

The 6-Gene Model+PSA retains its superiority over age-adjusted PSA alone when BPH Subjects were included with the normal subjects without BPH. The 6-gene+PSA model yielded a sensitivity of 97.4% and specificity of 91.5% for discriminating between prostate cancer subjects and normal subjects (with and without BPH; CaP (N=76) vs. Normals (N=76), BPH(N=30)). In contrast, age-adjusted PSA alone yielded a sensitivity of only 77.6% and a specificity of only 87.7% when BPH subjects were included with normal subjects without BPH.

The 6-gene model, RP51077B9.4, CD97, CDKN2A, SP1, S100A6 and IQGAP1, did not over-fit based on K-fold cross-validation. The following analysis was done to test for over-fitting: a) data were randomly split into K=10 equal sized sub samples; b) target model was re-estimated 10 times, each time omitting 1 sub sample; c) re-estimated model was applied to omitted sub sample; d) results were accumulated across all sub samples; e) validation log likelihood (Validation LL) was calculated (standard LL always increases when an additional gene is included in the model, and should decrease if the additional gene is extraneous).

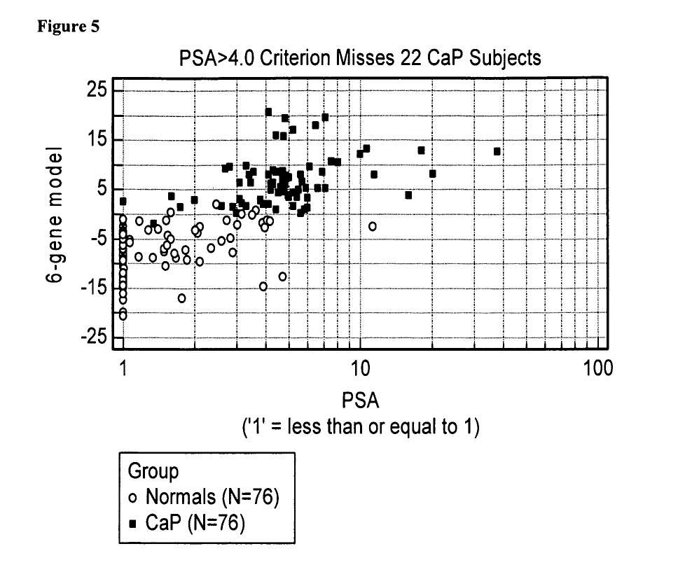

The subjects in the Training Dataset with PSA values between 2 ng/ml and 4 ng/ml included a large number of both prostate cancer subject and normal subjects. As shown in FIG. 5, when using PSA alone to discriminate between prostate cancer subjects and normal subjects with PSA values between 2 ng/ml and 4 ng/ml, 22 prostate cancer subjects are misclassified based on a cut-off of 4.0 ng/ml. However, 17 prostate cancer subjects and 17 age-matched normal subjects have PSA between 2 ng/ml and 4 ng/ml. Thus, reducing the cut-off below 4 ng/ml results in many false positive diagnoses. In contrast, when using the transcript based gene expression levels of the 6-gene model, RP51077B9.4, CD97, CDKN2A, SP1, S100A6 and IQGAP1, combined with PSA values, only 2 of the 76 prostate cancer subjects and 3 of the 76 normal subjects are misclassified based on a cut-off of 0.5 (cut-off shown by arrow on Y-axis, FIG. 5). Additionally, the 65 subjects with the highest model scores are all prostate cancer subjects, while the 65 subjects with the lowest model scores are all normal subjects. Thus, the use of the 6-gene model combined with PSA provides excellent discrimination between prostate cancer and age-matched normal subjects.

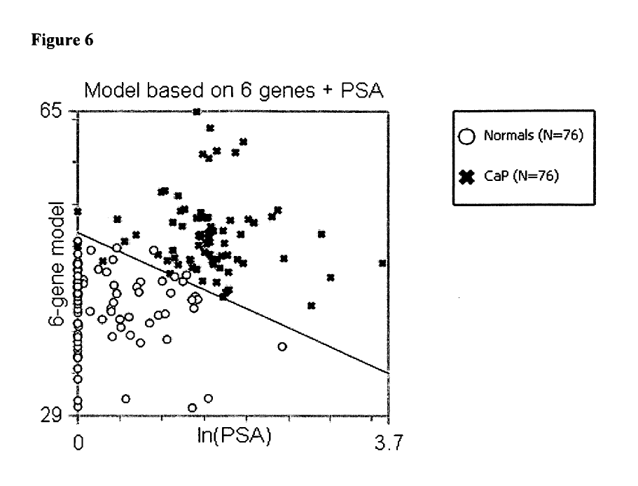

A discrimination plot of the 6-gene model, RP51077B9.4, CD97, CDKN2A, SP1, S100A6 and IQGAP1 combined with PSA is shown in FIG. 6. As shown in FIG. 6, the normal subjects are represented by circles, whereas prostate cancer subjects are represented by X's. The line appended to the discrimination graph in FIG. 6 illustrates how well the 6-gene model plus PSA discriminates between the 2 groups. Values above the line represent subjects predicted by the 6-gene plus PSA model to be in the prostate cancer population. Values below the line represent subjects predicted to be normal subject population. As shown in FIG. 6, only 3 normal subject (circles) and 2 prostate cancer subjects (X's) are classified in the wrong patient population.

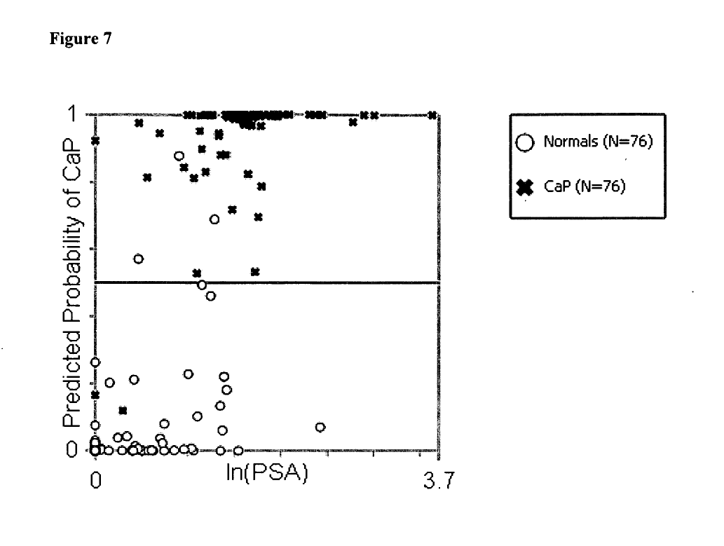

Individual subject predicted probability scores based on the 6-gene model, RP51077B9.4, CD97, CDKN2A, SP1, S100A6 and IQGAP1 combined with PSA also provides good separation of prostate cancer subjects from age-matched normal subjects. As shown in FIG. 7, many prostate cancer subjects have predicted probability of 0.8 or higher of having prostate cancer (above arrow shown on Y-axis, FIG. 7). Using a cut-off probability of 0.5 (probability (CaP) misclassifies only 2 prostate cancer subjects and only 3 normal subjects.

This 6-gene model was validated using RNA samples from the Test Dataset, as described in Example 6 below.

As stated above, in addition to this 6-gene model, five 2-gene models, two 4-gene models, and an additional 6-gene model, as shown in Table 4, all capable of distinguishing between prostate cancer subjects and normal subjects (without BPH) with over 75% sensitivity and specificity, will be validated using the Test data set population. Validation of these additional 2-gene, 4-gene and 6-gene models will be performed using strict tests as follows.

### Test 1—Strict Tests Based on Training Dataset Model Parameters and Cut-Offs:

For each of the models selected as described in Table 4, the model logit score will be computed using pre-specified coefficients (beta parameters) established in the training dataset. A pre-specified logit cut-point of 0 for all models will be applied to split the samples into two groups. Subjects with logit scores above the cut-point are predicted to be CaP patients and those whose scores fall below the cut-point are predicted to be healthy normal subjects. A 2×2 table of frequency counts (actual by predicted classification) will be constructed and a likelihood ratio chi-squared (L²) will be computed to test the null hypothesis that the model scores in each of the two groups are the same, with a 1-tailed p-value of less than 0.05 resulting in a successful validation (meaning test results deviate from independence with 95% confidence).

The five 2-gene models, two 4-gene models and two 6-gene models reflected in Table 4, all integrated with PSA, will be validated as required using the parameters established in the training set as specified in the Table 15 below:

Test 2a—Tests Based on Re-Estimated Parameters and Cut-Offs:

Repeat Test 1 using model coefficients (beta parameters) re-estimated on the test dataset. Validation is successful if the re-estimated beta parameters are in the same direction as the original model and the predictions based on the logit cut-point results in a p-value <0.05.

Test 2b—Tests Based on Re-Estimated Parameters Using a Likelihood Ratio (LR) Test:
The model is re-estimated on the test data and compared to a restricted model estimated with PSA only to obtain the likelihood ratio representing the incremental improvement of the genes in the model over the use of a model with PSA only. The p-value with degrees of freedom equal to G, where G is the number of genes in the model, will be computed.

### Test 3—Construction of ROC Curves and Area Under the Curves (AUC):

Using pre-specified model coefficients established in training dataset compute a model logit score. Construct comparative ROC curves using the model logit score vs. the age-adjusted PSA criterion. The model validates if the improvement in the area under the curve (AUC) associated with the logit model vs. age-adjusted PSA is significant (p<0.05).

The validation study of the five 2-gene models, two 4-gene models and two 6-gene models reflected in Table 4 is described in Example 12 below.

### Example 5

### Discrimination of Prostate Cancer Subjects from Healthy, Subjects with Benign Prostatic Hyperplasia (BPH) Using RNA Transcript-Based Gene Expression and Age-Adjusted PSA Values: Training Dataset

Example 4 describes several thousand 2-, 3- and 4-gene models and a 6-gene model which improve the specificity and sensitivity of prostate cancer screening when combined with PSA values, over the use of PSA testing alone. The data presented in this Example demonstrates that age-adjusted PSA values, when combined with transcript based gene expression, can also improve sensitivity (i.e., percentage of prostate cancer subjects correctly classified) and specificity (i.e., percentage of BPH subjects correctly classified) of prostate cancer screening over the use of age-adjusted PSA values alone.

The 76 prostate cancer subjects, 76 normal subjects, and 30 BPH subjects in the Training Dataset were age-matched as shown in FIG. 1, and PSA values were age-adjusted. Age-adjusted PSA criteria was represented by a dummy (dichotomous) variable coded 1 for all subjects (normal, BPH or CaP) if their PSA level fell above the given cut-off dependent on their age, as shown in FIG. 1. The prostate cancer cohort had a median age of 60 years, while the BPH cohort had a median age of 70 years.

Using age-adjusted PSA criteria as the sole predictor to screen for prostate cancer among the 76 untreated, localized prostate cancer subjects, 106 normal subjects (combined normal and BPH subjects) resulted in a specificity of 88.1% and sensitivity of only 77.6% for diagnosis of prostate cancer.

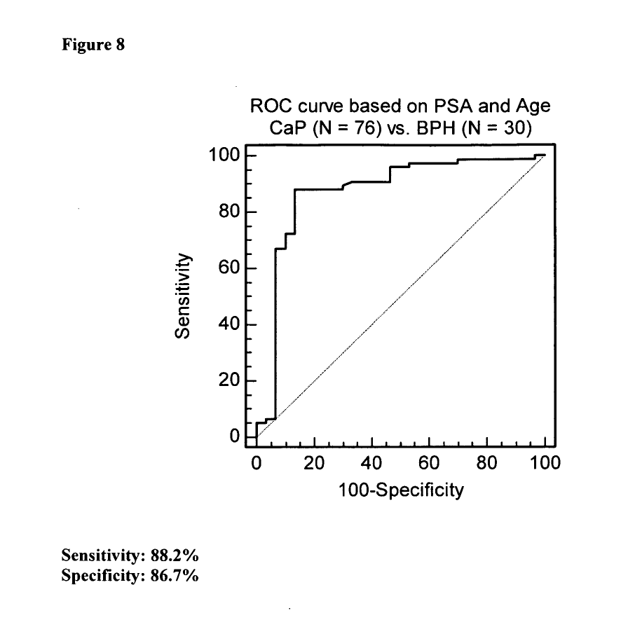

Using age-adjusted PSA criteria as the sole predictor to screen for prostate cancer among the 76 untreated, localized prostate cancer subjects and the 30 BPH subjects resulted in a specificity of 86.7% and sensitivity of 88.2%. A ROC curve demonstrating the ability of age and PSA to discriminate the prostate cancer patients from the BPH subjects of the Training Dataset is shown in FIG. 8.

Stepwise methodology was used to identify multi-gene models which combined with age-adjusted PSA levels could improve the sensitivity and specificity over the use of age-adjusted PSA values alone to discriminate between the prostate cancer subjects and BPH subjects. Both gene expression data and PSA values were available for the 76 untreated, localized prostate cancer subjects and 30 BPH subjects from the Training Dataset described in Example 1.

All possible 1 and 2-gene logit models were estimated based on 174 target genes assayed resulting in an enumeration of 15,225 models. 823 2-gene models were found to be statistically significant and were rank ordered (high to low) according to entropy R². Models meeting a minimum 75% correct classification criteria and predictor p-value criteria of <0.05 were retained. The top three 1-gene models and the top five 2-gene models were selected for validation (shown in Table 6).

All possible 3-gene logit models were also estimated based on the 174 target genes assayed (Table 1) and age-adjusted PSA values using the methodology described in Example 2, resulting in an enumeration of 5,597 3-gene models which discriminate between prostate cancer and BPH subjects with at least 75% correct classification and predictor p-value criteria of <0.05. The 5,597 3-gene models identified are shown in Table 5A.

As shown in Table 5A, the 3-gene models are identified in the first three columns (respectively) on the left side of Table 5A, ranked by their entropy R² value (shown in column 4, ranked from high to low). The number of subjects correctly classified or misclassified by each 3-gene model for each patient group (i.e., CaP vs. BPH) is shown in columns 5-8. The percent BPH subjects and percent prostate cancer subjects correctly classified by the corresponding gene model is shown in columns 9 and 10.

The “best” logistic regression model capable of distinguishing between prostate cancer subjects and BPH subjects when combined with age and PSA (defined as the model with the highest entropy R² value, as described in Example 2) based on the 174 genes included in the Precision Profile™ for Prostate Cancer Detection is MAP2K1, MYC and S100A6, capable of classifying BPH subjects with 90% accuracy, and prostate cancer subjects with 89.5% accuracy. Each of the 30 BPH RNA samples and the 76 prostate cancer RNA samples were analyzed for this 3-gene model, no values were excluded. This 3-gene model correctly classifies 27 of the BPH subjects as being in the BPH patient population, and misclassifies 3 of the BPH subjects as being in the prostate cancer patient population. This 3-gene model correctly classifies 68 of the prostate cancer subjects as being in the prostate cancer patient population and misclassifies 8 of the prostate cancer subjects as being in the BPH patient population.

A ranking of the top genes for which gene expression profiles were obtained, from most to least significant, is shown in Table 5B. Table 5B summarizes the mean expression levels of the genes listed in the Precision Profile™ for Prostate Cancer Detection (Table 1) measured in the RNA samples obtained from the prostate cancer subjects in the Training Dataset, as well as the results of significance tests (Wald p-values) for the difference in the mean expression levels between the BPH and prostate cancer subjects.

The top three 3-gene models based on entropy R2 were used to generate higher order (>3-gene) models. 4. Higher order models (5-gene) were developed by starting with the top three 3-gene models (which included PSA and age) and applying Stepwise Regression technique resulting in three 5-gene models selected for validation (shown in Table 6).

For example, the 3-gene logit model (MAP2K1, MYC and S100A6) was used to develop a 5-gene model, S100A6, MYC, MAP2K1, C1QA and RP51077B9.4, based on the Stepwise regression technique. Transcript based gene expression levels of the 5-gene model integrated with PSA and age gave higher specificity (93.3% of BPH subjects correctly classified) a much improved sensitivity (96.1% of prostate cancer subjects correctly classified) for prostate cancer diagnosis over the use of PSA and age alone (86.7% specificity, 88.2% sensitivity, as shown in FIG. 8).

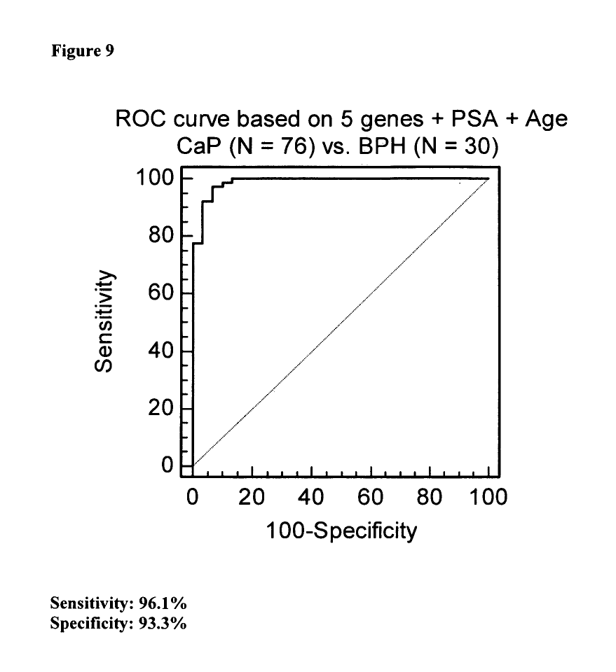

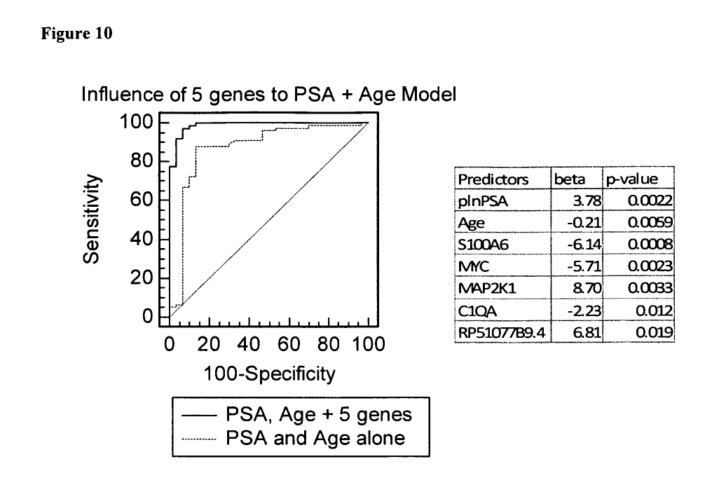

A ROC curve for the 5-gene model+PSA+Age is shown in FIG. 9. Improved area under the ROC curve further supports the improved discrimination of prostate cancer versus BPH subjects when combining PSA and age with gene expression as compared to age-adjusted PSA alone. As shown in FIG. 10, the area under the ROC curve is 0.871 for the model based on PSA and age alone, as compared to 0.989 when expression values for the 5-gene model are included with PSA and age. This improvement is statistically significant (p-value=0.0001).

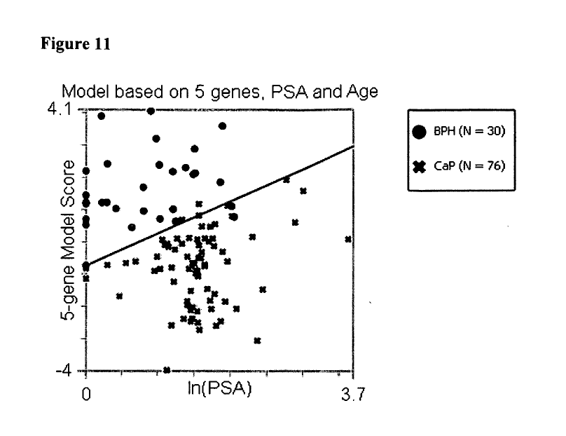

A discrimination plot of the 5-gene model, S100A6, MYC, MAP2K1, C1QA and RP51077B9.4, combined with PSA+age is shown in FIG. 11. As shown in FIG. 11, the BPH subjects are represented by circles, whereas prostate cancer subjects are represented by X's. The line appended to the discrimination graph in FIG. 11 illustrates how well the 5-gene model combined with PSA and age discriminates between the 2 groups. Values above the line represent subjects predicted by the 5-gene+PSA+age model to be in the BPH subject population. Values below the line represent subjects predicted to be prostate cancer population. As shown in FIG. 11, only 2 of the 30 BPH subject (circles) and 3 of the 76 prostate cancer subjects (X's) are classified in the wrong patient population. However, all 5 misclassifications are close to the discrimination line.

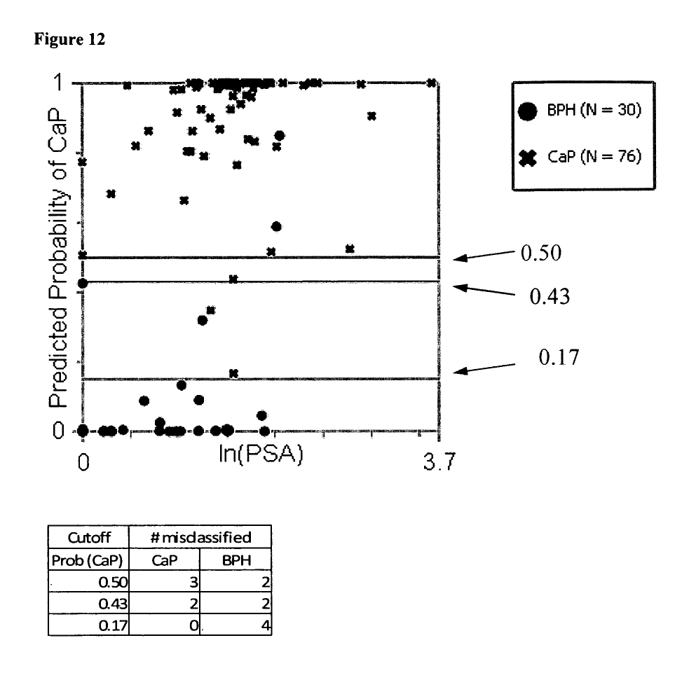

Individual subject predicted probability scores based on the 5-gene model, S100A6, MYC, MAP2K1, C1QA and RP51077B9.4 combined with PSA+age also provides good separation of prostate cancer subjects from BPH subjects, as shown in FIG. 12. The cut-off probability (probability (CaP)) can be modulated to alter sensitivity and specificity of the model. For example, a cut-off probability of 0.17 yields a sensitivity of 100% (all prostate cancer subjects above the cut-off line) which reduces the specificity to 87% (26 of 30 BPH subjects below the line).

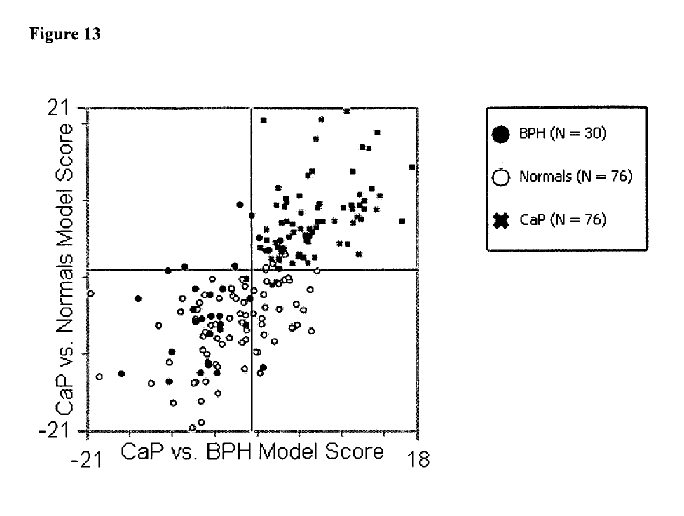

Two or more of the gene-models described herein can be used incrementally or iteratively to provide almost perfect discrimination of prostate cancer patients from non-prostate cancer patients (normals and BPH). For example, combining the 6-gene (RP51077B9.4, CD97, CDKN2A, SP1, S100A6 and IQGAP1)+PSA model which discriminates between prostate cancer and normal, healthy subjects (described in Example 4 and validated in Example 6) with the 5-gene (S100A6, MYC, MAP2K1, C1QA and RP51077B9.4)+PSA+age model which discriminates between prostate cancer and BPH subjects provides almost perfect discrimination. As shown in FIG. 13, prostate cancer subjects are almost exclusively in the upper right quadrant-above the cut-off on the prostate cancer versus normals model (cut off shown as the horizontal line intersecting the Y-axis) and above the cut-off on the prostate cancer versus BPH model (cut off shown as the vertical line intersecting the Y-axis).

This 5-gene model, in addition to the three 1-gene models, five 2-gene models, three 3-gene models, and three 5-gene models, as shown in Table 6, all capable of distinguishing between prostate cancer subjects and normal subjects with BPH with over 75% sensitivity and specificity, will be validated using the RNA samples from the Test Dataset.

Validation of the three 1-gene, five 2-gene, three 3-gene and three 5-gene models shown in Table 6 will be performed using strict tests as follows.

### Test 1—Strict Tests Based on Training Dataset Model Parameters and Cut-Offs:

For each of the models selected as described in section 2.3, the model logit score will be computed using pre-specified coefficients (beta parameters) established in the training dataset. A pre-specified logit cut-point of 0 for all models will be applied to split the samples into two groups. Subjects with logit scores above the cut-point are predicted to be CaP patients and those whose scores fall below the cut-point are predicted to be normal subjects presenting with BPH. A 2×2 table of frequency counts (actual by predicted classification) will be constructed and a likelihood ratio chi-squared (L²) will be computed to test the null hypothesis that the model scores in each of the two groups are the same, with a 1-tailed p-value of less than 0.05 resulting in a successful validation (meaning test results deviate from independence with 95% confidence). The following three 1-gene models, five 2-gene models, three 3-gene models and three 5-gene models, all integrated with PSA and age, will be validated as required using the parameters established in the training set as specified in Table 16 below:

Test 2a—Tests Based on Re-Estimated Parameters and Cut-Offs:

Repeat Test 1 using model coefficients (beta parameters) re-estimated on the test dataset. Validation is successful if the re-estimated beta parameters are in the same direction as the original model and the predictions based on the logit cut-point results in a p-value <0.05.

Test 2b—Tests Based on Re-Estimated Parameters Using a Likelihood Ratio (LR) Test
The model is re-estimated and compared to a model estimated with PSA only to obtain the likelihood ratio representing the incremental improvement of the genes in the model over the use of a model with PSA only. The p-value with degrees of freedom equal to G, where G is the number of genes in the model, will be computed.

### Test 3—Construction of ROC Curves and Area Under the Curves (AUC):

Using pre-specified model coefficients established in training dataset compute a model logit score. Construct comparative ROC curves using the model logit score vs. the age-adjusted PSA criterion. The model validates if the improvement in the area under the curve (AUC) associated with the 6-gene logit model vs. age-adjusted PSA is significant (p<0.05).

The validation study of the three 1-gene, five 2-gene, three 3-gene and three 5-gene models shown in Table 6 is described in Example 12 below.

### Example 6

### Discrimination of Prostate Cancer Subjects from Healthy, Normal Subjects (without BPH) Using RNA Transcript-Based Gene Expression: Validation Using Test Dataset

RNA samples from the Test Dataset were used to validate the 6-gene (RP51077B9.4, CD97, CDKN2A, SP1, S100A6 and IQGAP1) model's ability to discriminate between prostate cancer subjects and normal subjects (without BPH), identified using samples from the Training Dataset, as described in Example 4.

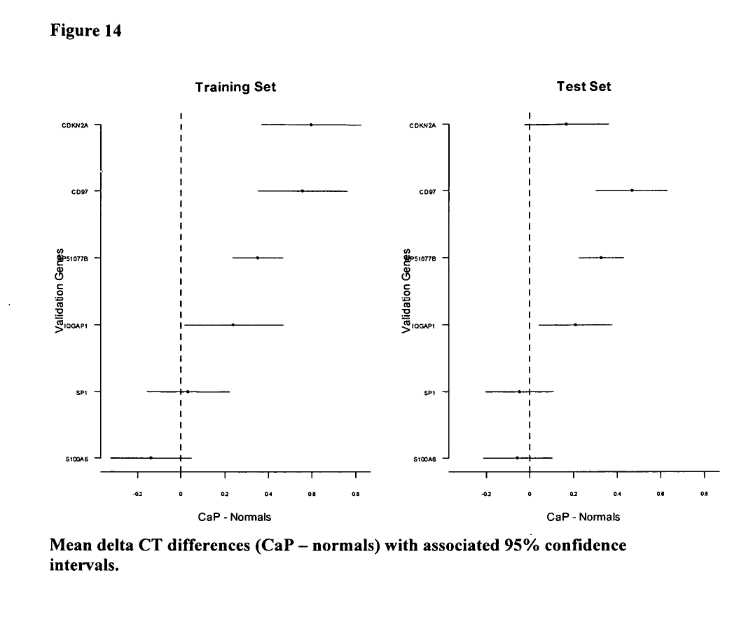

A comparison of differences in mean delta C_{T} values for prostate cancer patients versus normal subjects demonstrated high consistency between training and test sample measurements for the 6-gene (RP51077B9.4, CD97, CDKN2A, SP1, S100A6 and IQGAP1) model (see FIG. 14, mean delta C_{T} differences (CaP-normals) with associated 95% confidence intervals).

Validation of 6-Gene Logit Model Alone (i.e., not Combined with PSA)

Validation of the 6-gene logit model followed a pre-specified validation plan as follows:

### Test A:

a) Using pre-specified model coefficients (beta) established in TRAINING dataset (shown in Table C below) compute a model logit score.

TABLE C
pre-specified model coefficients based on Training Dataset

| Predictors | beta | p-value |
|---|---|---|
| Intercept | −48.84 | |
| SP1 | −9.47 | 1.3E−06 |
| CD97 | 3.67 | 4.0E−05 |
| RP5107789.4 | 5.19 | 7.2E−05 |
| CDKN2A | 1.39 | 1.4E−04 |
| IQGAP1 | 3.44 | 6.4E−03 |
| S100A6 | −1.43 | 0.011 |

b) Apply pre-specified cut-point established in the TRAINING dataset to yield 2 groups. Subjects with logit scores above 0 (predicted probability of CaP=0.5) are predicted to be CaP and those with scores below 0 are predicted to be healthy normal.
c) Form 2×2 table of frequency counts (actual by predicted classification). Compute likelihood ratio chi-squared (L²) and derive p-value with 1 degree of freedom. A validation p-value <0.05 constitutes a successful validation (meaning test results deviate from independence with 95% confidence).

### Test B:

a) Repeat Test A using model coefficients (beta parameters) re-estimated on the test dataset.
b) Validation is successful if the re-estimated beta parameters are in the same direction as the original model and the predictions based on the logit cut-point of 0 results in a p-value <0.05.

### Test C:

a) Using pre-specified model coefficients established in TRAINING dataset compute a model logit score.
b) Construct comparative ROC curves using the 6-gene model logit score vs. the age-adjusted PSA criterion. The model validates if the improvement in the area under the curve (AUC) associated with the 6-gene logit model vs. age-adjusted PSA is significant (p<0.05.).

### Test A Results:

Using the pre-specified model coefficients shown in Table C (5.19 RP51077B9.4, 3.67 CD97, 1.89 CDKN2A, −9.47 SP1, −1.43 S100A6, 3.44 IQGAP1) and a pre-specified cut point probability of 0.5, the 6-gene logit model demonstrated a sensitivity (% CaP subjects correctly classified) and specificity (% normal subjects (without BPH) correctly classified) of 85.9% and 83.0%, respectively with a validation p-value=1.3E-26.

### Test B Results:

The results from Test B are shown in Table D below.

TABLE D
6-gene Model—Parameter Estimates and p-values

| | Training | | Test | |
|---|---|---|---|---|
| Predictors | beta | p value | beta | p-value |
| Intercept | −48.84 | | −21.29 | |
| SP1 | −9.47 | 1.3E−06 | −6.50 | 4.7E−11 |
| CD97 | 3.67 | 4.0E−05 | 3.85 | 2.3E−06 |
| RP51077B9.4 | 5.19 | 7.2E−05 | 2.73 | 2.0E−04 |
| CDKN2A | 1.89 | 1.4E−04 | 0.53 | 0.09 |
| IQGAP1 | 3.44 | 6.4E−03 | 1.63 | 0.05 |
| S100A6 | −1.43 | 0.011 | −0.24 | 0.54 |

All coefficients estimated based on the test data have the same sign as the original model estimated on the training data.

### Test C Results:

Comparative test dataset ROC curves using the 6-gene model logit score vs. the age-adjusted PSA criterion were constructed. The area under the ROC curve (AUC) for the 6-gene logit model vs. the age-adjusted PSA was 0.898 vs. 0.816, respectively. This represents a statistically significant improvement with a validation p-value=0.014.

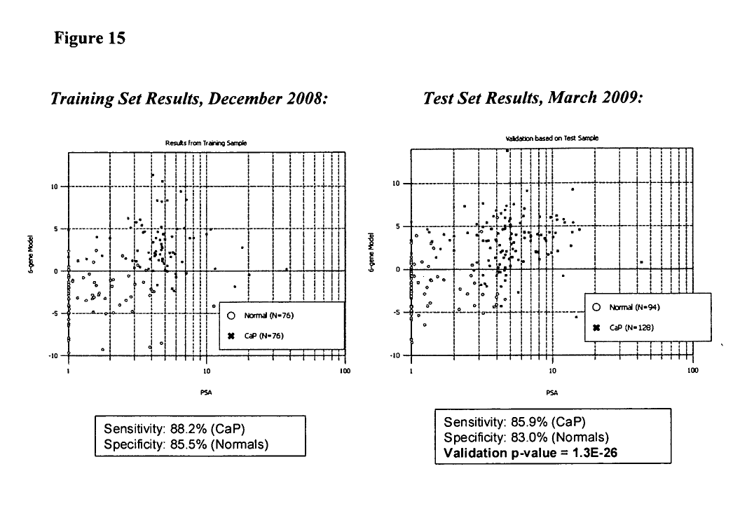

The Test Dataset confirms that the 6-gene logit model alone (i.e., not used in combination with PSA) is capable of discriminating prostate cancer patients from normal subjects (without BPH) with high statistical significance. A comparison of the Training Set results and the Test Set results is shown in FIG. 15. The results for the 6-gene model from the training sample yielded a sensitivity of 88.2% (CaP) and specificity of 85.5% (normals) while the test set results yielded a sensitivity of 85.9% (CaP) and specificity of 83% (normals). The test dataset exhibited a slight fall-off in sensitivity (88.2% to 85.9%) and specificity (85.5% to 83%) from the training dataset. In comparison, the age-adjusted PSA criteria only yielded a sensitivity of 69.5% and specificity of 93.6%.

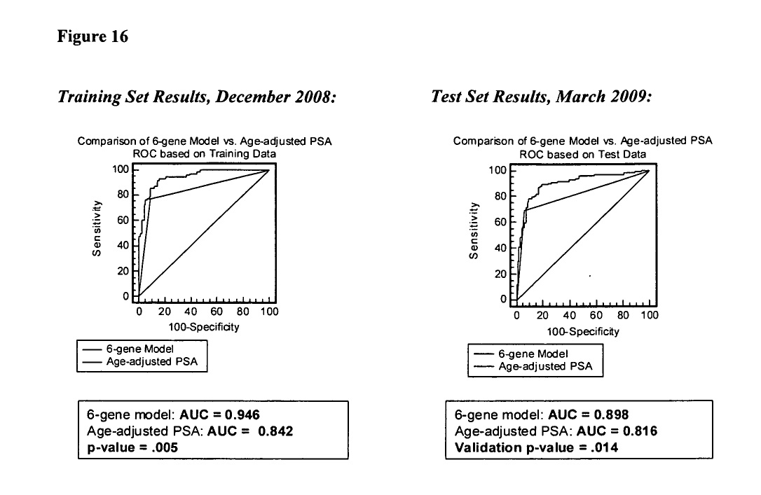

The Test Dataset further confirms that the area under the ROC curve (AUC) is significantly improved for the 6-gene model over the age-adjusted PSA criterion. As shown in FIG. 16, the AUC for the ROC curve for the 6-gene model in the results from the training set is 0.946 whereas the AUC for the curve for age-adjusted PSA criteria alone is 0.842 (p-value=0.005). The AUC for the ROC curve for the 6-gene model in the test set results is 0.898, whereas the AUC for the age-adjusted PSA alone is 0.816. Note that the AUC for the ROC curve is somewhat smaller on the test dataset for both the 6-gene model as well as the age-adjusted PSA criterion.

### Validation of 6-Gene+PSA Model:

Validation of the 6-gene logit model (RP51077B9.4, CD97, CDKN2A, SP1, S100A6 and IQGAP1)+PSA that discriminates prostate cancer patients from normal subjects (without BPH) followed a pre-specified plan as follows:

### Test A:

a) Using pre-specified model coefficients (beta) established in TRAINING dataset (shown in Table E below) compute a model logit score.

TABLE E
Pre-specified model coefficients established in Training Dataset

| Predictors | beta | p-value |
|---|---|---|
| Intercept | −50.66 | |
| pInPSA | 4.50 | 4.4E−05 |
| SP1 | −15.11 | 2.8E−04 |
| CD97 | 6.31 | 9.3E−04 |
| RP51077B9.4 | 7.65 | 1.9E−03 |
| CDKN2A | 2.94 | 4.1E−03 |
| S100A6 | −2.63 | 0.014 |
| IQGAP1 | 4.03 | 0.024 |

b) Apply pre-specified cut-point established in the TRAINING dataset to yield 2 groups. Subjects with logit scores above 0 (predicted probability of CaP=0.5) are predicted to be CaP and those with scores below 0 are predicted to be healthy normal.
c) Form 2×2 table of frequency counts (actual by predicted classification). Compute likelihood ratio chi-squared (L²) and derive p-value with 1 degree of freedom. A validation p-value <0.05 constitutes a successful validation (meaning test results deviate from independence with 95% confidence).

### Test B:

a) Repeat Test A using model coefficients (beta parameters) re-estimated on the test dataset.
b) Validation is successful if the re-estimated beta parameters are in the same direction as the original model and the predictions based on the logit cut-point of 0 results in a p-value <0.05.

### Test C:

a) Using pre-specified model coefficients established in TRAINING dataset compute a model logit score.
b) Construct comparative ROC curves using the 6-gene+PSA model logit score vs. the age-adjusted PSA criterion. The model validates if the improvement in the area under the curve (AUC) associated with the 6-gene logit model+PSA vs. age-adjusted PSA is significant (p<0.05.).

### Test A Results:

Using the pre-specified model coefficients shown in Table E (4.50 plnPSA, 7.65 RP51077B9.4, 6.31 CD97, 2.94 CDKN2A, −15.11 SP1, −2.63 S100A6, 4.03 IQGAP1) and a pre-specified cut point probability of 0.5, the 6-gene logit model+PSA demonstrated a sensitivity and specificity of 87.5% and 92.6%, respectively with a validation p-value=9.6E-37.

### Test B Results:

The Test B are shown in Table F below

TABLE F
′6-gene + PSA′ Model—Parameter Estimates and p-values

| | Training | | Test | |
|---|---|---|---|---|
| Predictors | beta | p-value | beta | p-value |
| Intercept | −50.66 | | −14.90 | |
| pInPSA | 4.50 | 4.4E−05 | 3.01 | 6.8E−10 |
| SP1 | −15.11 | 2.8E−04 | −5.64 | 2.3E−05 |
| CD97 | 6.31 | 9.3E−04 | 4.45 | 2.4E−04 |
| RP51077B9.4 | 7.65 | 1.9E−03 | 1.69 | 0.10 |
| CDKN2A | 2.94 | 4.1E−03 | 0.79 | 0.10 |
| 5100A6 | −2.63 | 0.014 | −0.19 | 0.71 |
| IQGAP1 | 4.03 | 0.02 | 0.24 | 0.84 |

Again, all coefficients estimated based on the test data have the same sign as the original model estimated on the training data.

### Test C Results:

Comparative test dataset ROC curves using the 6-gene+PSA model logit score vs. the age-adjusted PSA criterion were constructed. The area under the ROC curve (AUC) for the 6-gene+PSA logit model vs. the age-adjusted PSA was 0.962 and 0.816, respectively. This represents a statistically significant improvement with a validation p-value=1.5E-7.

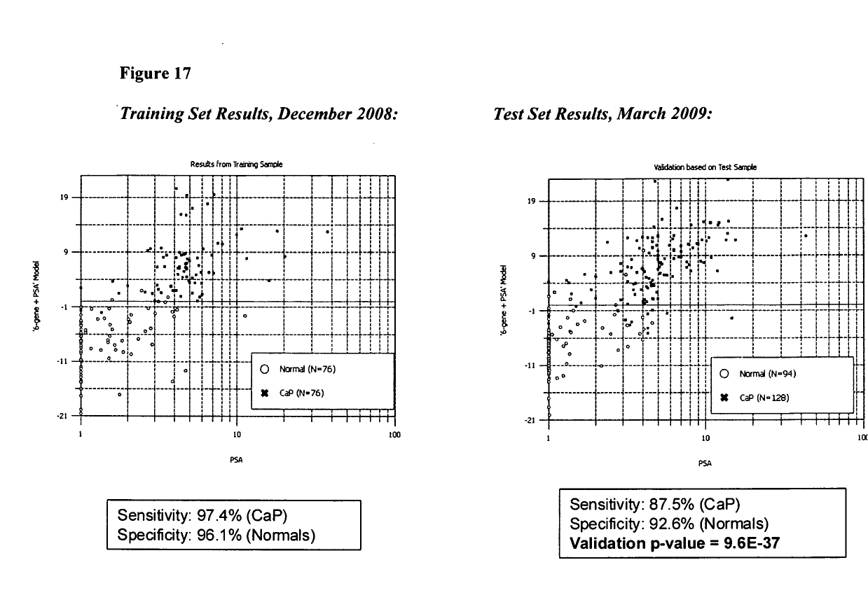

The Test Dataset confirms that the 6-gene logit model+PSA is capable of discriminating prostate cancer patients from normal subjects (without BPH) with high statistical significance. A comparison of the Training Set results and the Test Set results is shown in FIG. 17. The results for the 6-gene model+PSA from the training sample yielded a sensitivity of 97.4% (CaP) and specificity of 96.1% (normals) while the test set results yielded a sensitivity of 87.5% (CaP) and specificity of 92.6% (normals) (validation p-value=9.6E-37). The test dataset exhibited a slight fall-off in sensitivity (97.4% to 87.5%) and specificity (96.1% to 92.6%) from the training dataset. Inclusion of BPH subjects in the Training and independent validation sets reduced the sensitivity and specificity to 91.5% and 91.4% respectively. In comparison, the age-adjusted PSA criteria yielded a sensitivity of 69.5% and specificity of 93.6%.

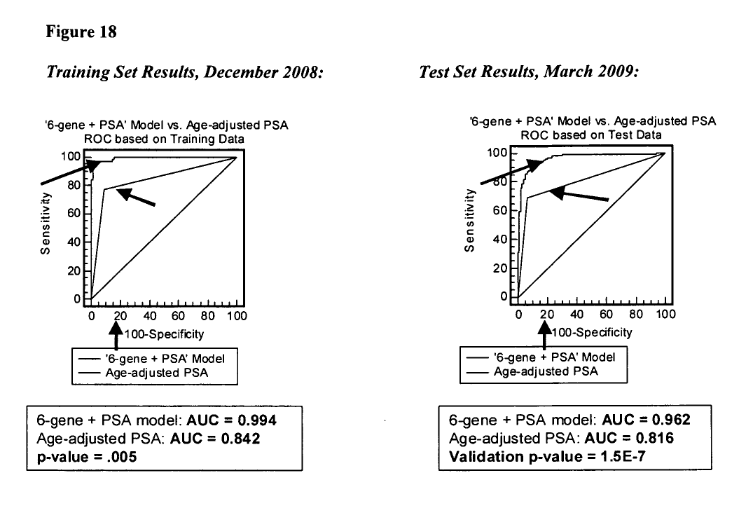

The Test Dataset further confirms that the area under the ROC curve (AUC) is significantly improved for the 6-gene model+PSA over the age-adjusted PSA criterion. As shown in FIG. 18, the AUC for the ROC curve for the 6-gene+PSA model in the results from the Training Set is 0.0.994 whereas the AUC for the curve for age-adjusted PSA criteria alone is 0.842 (p-value=0.005). The AUC for the ROC curve for the 6-gene+PSA model in the Test Set results is 0.962, whereas the AUC for the age-adjusted PSA alone is 0.816 (validation p-value=1.5E-7). Given a specificity range of 91-93%, the 6-gene+PSA model has higher sensitivity (97% training/87.5% test) compared to the age-adjusted PSA alone (<78% training/<70% test).

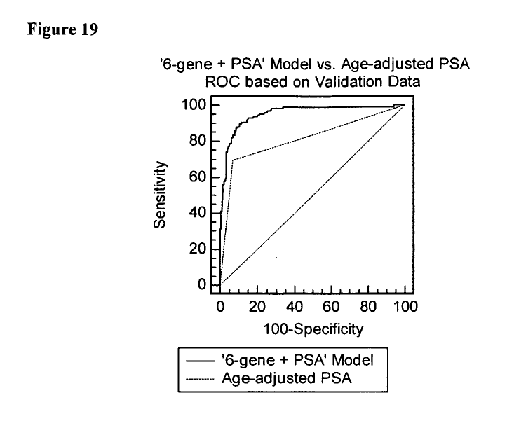

The 6-Gene Model+PSA retains its superiority over age-adjusted PSA alone when BPH Subjects are included with the normal subjects without BPH. As stated in Example 4, the Training set results of the 6-gene+PSA model yielded a sensitivity of 97.4% and specificity of 91.5% for discriminating between prostate cancer subjects and normal subjects (with and without BPH; CaP (N=76) vs. Normals (N=76), BPH(N=30)). These results were validated using the Test dataset, a sensitivity of 87.5% and specificity of 91.4% was yielded for the 6-gene+PSA model, as compared to a sensitivity of 69.5% and specificity of 93.1% for the age-adjusted PSA model alone. A ROC curve is shown in FIG. 19. The AUC for the 6-gene+PSA model is 0.953, the AUC for the 0.813. The AUC difference is statistically significant (p-value=9.0E-8).

Development of a 6-gene model in a Training set of samples that is further validated in an independent dataset strongly suggest that specific whole blood RNA transcript levels can assess abnormal gene expression levels associated with untreated, localized CaP. Validation of such a model with and without the inclusion of PSA supports its potential value as a diagnostic tool in the management of early stage prostate cancer with economic benefits to the healthcare system.

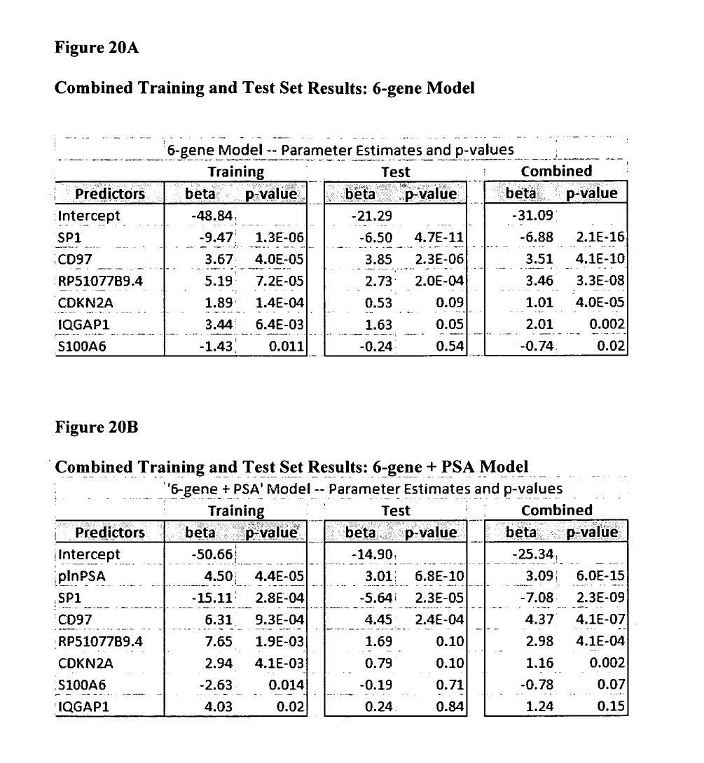

Re-estimation of model parameters based on the combined training and test datasets will be used to refine the 6-gene model (with and without PSA) for use in future multi-site validation studies (see FIG. 19; all coefficients estimated based on the test data have the same sign as the original model estimated on the training data). Using the combined training and test datasets with re-estimated parameters, improved sensitivity and specificity is observed when comparing the 6-gene model with PSA to the 6-gene model without PSA (see FIG. 20). The 6-gene+PSA model exhibited improvement in sensitivity (85.8% to 93.6%) and specificity (87.1% to 94.7%) when compared to the 6-gene model alone.

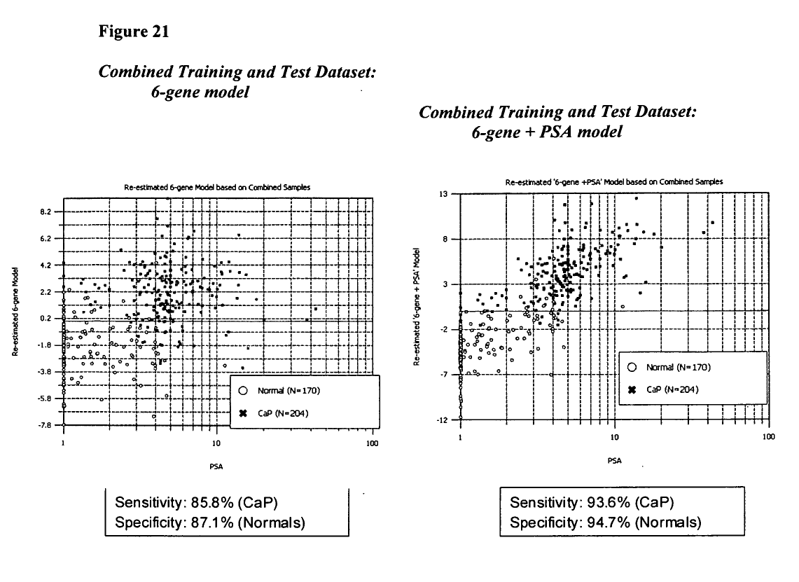

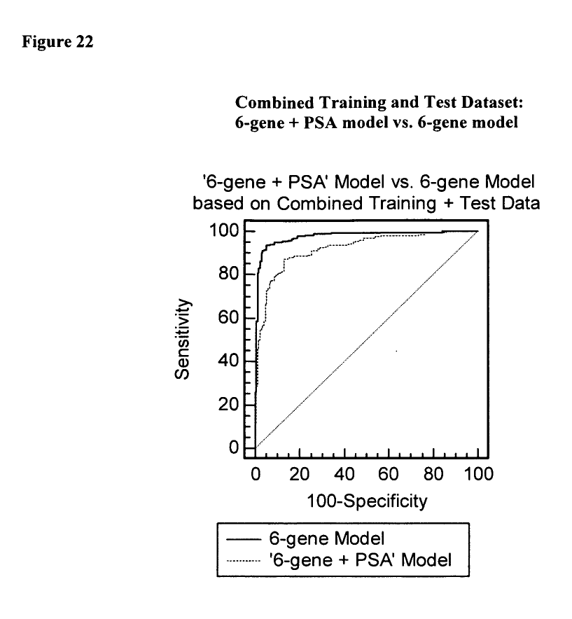

Using the combined Training and Test Datasets with re-estimated parameters, improved AUC for ROC curve is also observed when comparing the 6-Gene+PSA model (AUC=0.977) to the 6-Gene model without PSA (AUC=0.920), as shown in FIG. 21 (p-value=3.1E-7). However, when using the combined Training and Test datasets, the AUC for the ROC curve for the age-adjusted PSA criterion (AUC=0.825) does not provide statistically significant improvement over the global PSA>4 criterion (AUC=0.82) (p-value >0.05, see FIG. 22).

### Example 7

### RNA Transcript-Based Diagnostic Model for Predicting Prostate Cancer Patients with Gleason Scores of 8-9: Training Set

The Gleason grading or score of a prostate biopsy by a pathologist is used to help evaluate the prognosis of men with prostate cancer and guide treatment. A Gleason score is assigned to prostate cancer based upon microscopic appearance of prostate tissue biopsy. A pathologist reports a primary and secondary grade (1-5) which are then added to obtain a final Gleason score (2-10). A Gleason score of 7 or above generally results in treatment with scores of 8 and above considered aggressive prostate cancer. A Gleason score of 10 represents the worst prognosis. A Gleason score of 7 can be obtained by either a primary+secondary grade of (3+4) or (4+3), the former indicative of less aggressive tumors, and the latter with more aggressive tumors. Due to the limitations of the biopsy approximately 30% of men undergoing prostatectomy have an upgraded Gleason score when the cancerous tissue is analyzed by a pathologist after surgery.

The present example illustrates that a whole blood RNA transcript-based diagnostic test can predict prostate cancer patients with the most aggressive form of prostate cancer as represented by Gleason scores of 8-9.

Whole blood was collected in PaxGene™ Blood RNA Tubes from the 76 newly diagnosed, untreated localized prostate cancer (CaP) patients described in Example 1. Of the 76 CaP patients from which whole blood was collected, 9 patients had a Gleason score of 8-9, 22 patients had a Gleason score of 7, 44 patients had a Gleason score of 6, none of the patients had a Gleason score of 5 or lower, and 1 patient's Gleason score was undetermined. The 174 inflammation and cancer-related genes listed in the Precision Profile™ for Prostate Cancer Detection (Table 1) were assayed for each subject.

Ordinal Logit Methodology was used to obtain alternative models capable of discriminating between CaP subjects with Gleason scores of 8-9 (i.e., aggressive form of CaP) and Cap Subjects with Gleason scores of 7 or less (i.e., less aggressive form of CaP) based on gene expression and PSA values. All possible 2-gene logit models were estimated based on the 174 genes assayed, resulting in an enumeration of 14,196 2-gene models. Thirty four 2-gene models out of the 14,196 models identified were statistically significant and were rank ordered from high tot low according to their entropy R² value. The highest entropy R²²-gene models were used to develop 3-gene models based on Stepwise Regression technique. PSA values were also considered as the third gene for this Stepwise Regression analysis. The best 2-gene and 3-gene models did not over-fit based on K-fold cross-validation. The following analysis was done to test for over-fitting:

a) Data were randomly split into K=10 equal sized sub samples'

b) Target model was re-estimated 10 times, each time omitting 1 sub sample;

c) Re-estimated model was applied to the omitted sub sample;

d) Results were accumulated across all sub samples;

e) Validation log-likelihood (Validation LL): Standard LL always increases when an additional gene is included in the model; Validation LL should decrease is the additional gene is extraneous.

The best 2-gene model for predicting CaP subjects with Gleason scores of 8-9 using RNA transcript-based gene expression consisted of the 2 genes CCND2 and COL6A2, resulting in the Gleason score classifications shown in Table G below:

TABLE G
Gleason score classifications based on 2-gene
model CCND2 and COL6A2

| | | cutoff2 | | |
|---|---|---|---|---|
| | | 0 | 1 | Total |
| GleasonR | 6 | 39 | 5 | 44 |
| | | 88.6% | 11.4% | 100.0% |
| | 7 | 15 | 7 | 22 |
| | | 68.2% | 31.8% | 100.0% |
| | 8-9 | 2 | 7 | 9 |
| | | 22.2% | 77.8% | 100.0% |
| Total | | 56 | 19 | 75 |
| | | 74.7% | 25.3% | 100.0% |

When an alternative cutoff was used, the same 2-gene model resulted with the Gleason score classifications shown in Table H:

TABLE H
Gleason score classifications based on alternative cutoff
for 2-gene model CCND2 and COL6A2

| | | cutoff | | |
|---|---|---|---|---|
| | | 0 | 1 | Total |
| GleasonR | 6 | 31 | 13 | 44 |
| | | 70.5% | 29.5% | 100.0% |
| | 7 | 14 | 8 | 22 |
| | | 63.6% | 36.4% | 100.0% |
| | 8-9 | 0 | 9 | 9 |
| | | .0% | 100.0% | 100.0% |
| Total | | 45 | 30 | 75 |
| | | 60.0% | 40.0% | 100.0% |

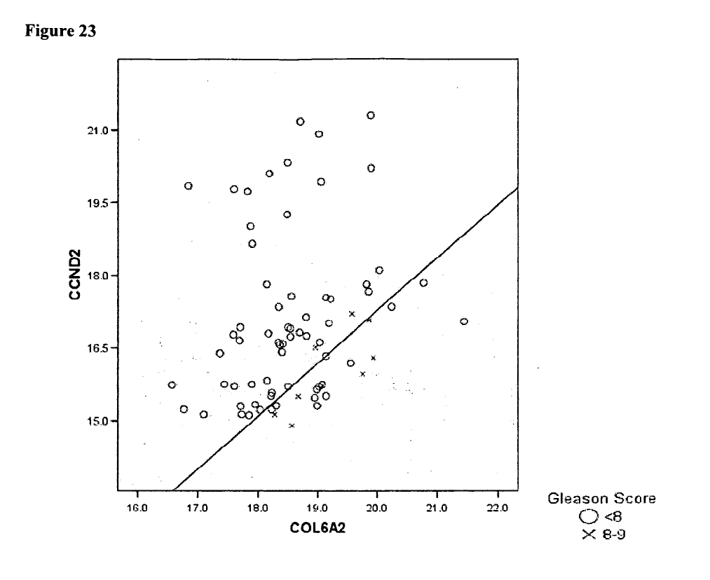

A discrimination plot based on the cutoff used in Table G is shown in FIG. 23. As depicted in FIG. 23, the 2-gene model CCND2 and COL6A2 is capable of correctly classifying 78.8% of CaP subjects having a Gleason score of 8-9, and correctly classifies 81.8% of CaP subjects having a Gleason score <8.

The inclusion of PSA values with the 2-gene model CCND2 and COL6A2 significantly improved the classification rate, resulting in the Gleason score classifications shown in Table I below:

TABLE I
Best 2-gene model including PSA values

| | | cutoff | | |
|---|---|---|---|---|
| | | 0 | 1 | Total |
| GleasonR | 6 | 37 | 7 | 44 |
| | | 84.1% | 15.9% | 100.0% |
| | 7 | 15 | 7 | 22 |
| | | 68.2% | 31.8% | 100.0% |
| | 8-9 | 0 | 9 | 9 |
| | | .0% | 100.0% | 100.0% |
| Total | | 52 | 23 | 75 |
| | | 69.3% | 30.7% | 100.0% |

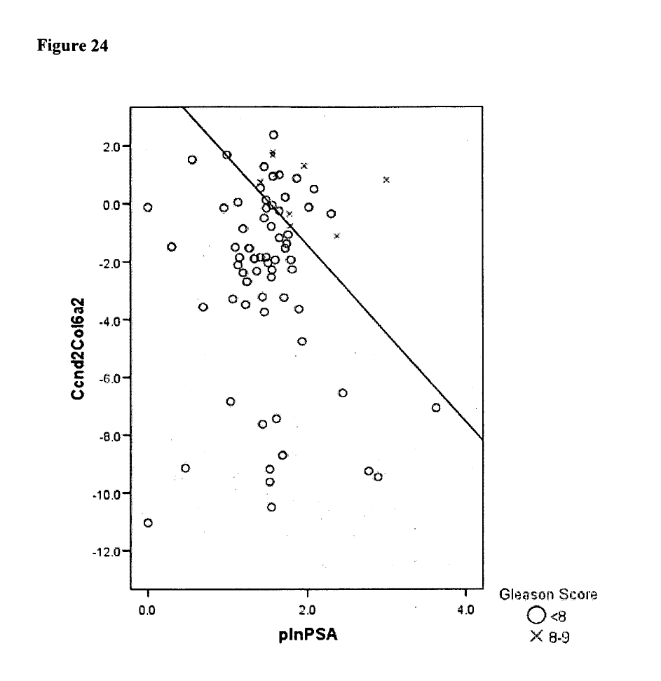

A discrimination plot based the 2-gene model CCND1 and COL6A2+PSA is shown in FIG. 24. As depicted in FIG. 24, this 2-gene model+PSA is capable of correctly classifying 100% of CaP subjects having a Gleason score of 8-9, and correctly classifies 78.8% of CaP subjects having a Gleason score <8.

The 2-gene model, CCND2 and COL6A2, plus PSA values, was developed on the 75 CaP subjects and applied to the healthy normal and BPH control subjects described in Example 1 per Table J below:

TABLE J
Results of Applying Model to Other Groups

| Group | N | positive | negative | % positive | % PSA >4 |
|---|---|---|---|---|---|
| Normals | 76 | 1 | 75 | 1% | 5% |
| BPH | 30 | 5 | 25 | 17% | 23% |

A model consisting of 3 genes (CCND2, COL6A2 and CDKN2A), identified as the best 3-gene model, resulted in the Gleason score classifications shown in Table K below:

TABLE K
Gleason score classifications based on 3-gene model
CCND2, COL6A2 and CDKN2A

| | | cutoff | | |
|---|---|---|---|---|
| | | 0 | 1 | Total |
| GleasonR | 6 | 38 | 6 | 44 |
| | | 86.4% | 13.6% | 100.0% |
| | 7 | 16 | 6 | 22 |
| | | 72.7% | 27.3% | 100.0% |
| | 8-9 | 0 | 9 | 9 |
| | | .0% | 100.0% | 100.0% |
| Total | | 54 | 21 | 75 |
| | | 72.0% | 28.0% | 100.0% |

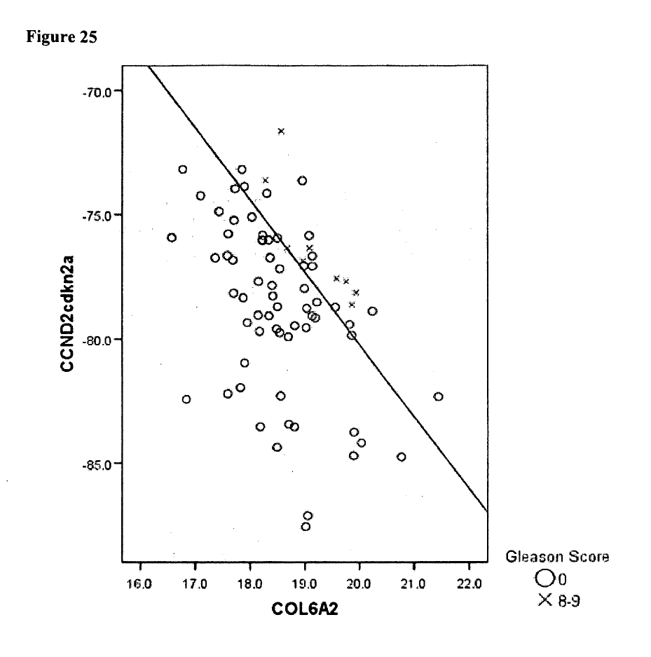

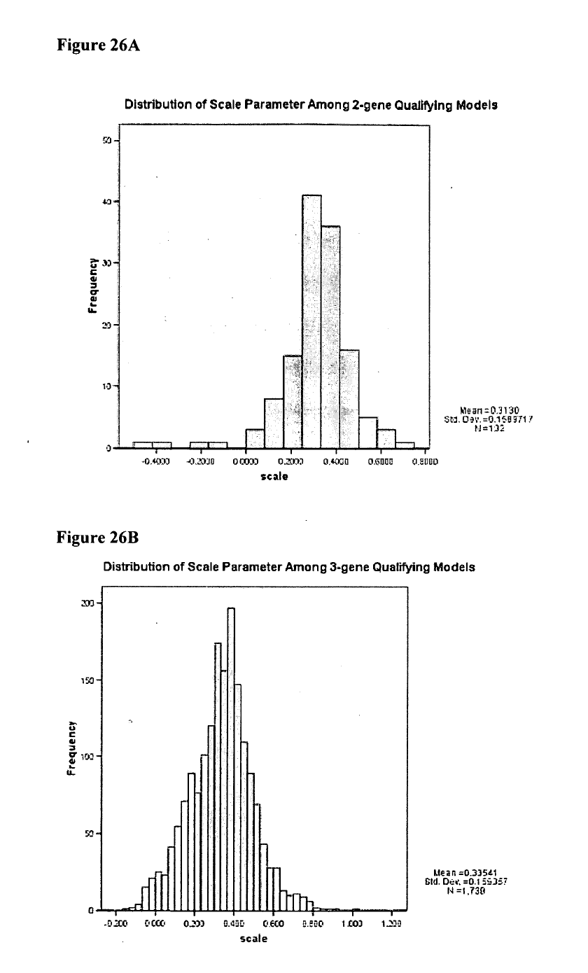

A discrimination plot based the 3-gene model CCND2, COL6A2 and CDKN2A is shown in FIG. 25. As depicted in FIG. 25, this 3-gene model is capable of correctly classifying 100% of CaP subjects having a Gleason score of 8-9, and correctly classifies 81.8% of CaP subjects having a Gleason score <8.

In addition to the 2-gene and 3-gene models described above, the top 1-gene logistic regression models capable of discriminating Gleason scores of <8 versus 8-9 were identified, as shown in Table L below:

TABLE L
Top 1-gene logistic regression models

| | Gleason 8-9 | Gleason <8 | Sum |
|---|---|---|---|
| Group Size | 12.0% | 88.0% | 100% |
| N= | 9 | 66 | 75 |

| | | | | Mean |
|---|---|---|---|---|
| Gene | Mean | Mean | LRp-value | Difference |
| C1QA | 19.3 | 20.0 | 0.033 | 0.7 |
| COND2 | 16.0 | 17.1 | 0.041 | 1.0 |
| COL6A2 | 19.2 | 18.5 | 0.042 | −0.7 |
| TIMP1 | 14.3 | 14.6 | 0.046 | 0.4 |
| SERPINE1 | 21.3 | 21.9 | 0.055 | 0.7 |
| plnPSA | 1.9 | 1.5 | 0.055 | −0.4 |
| SERPING1 | 18.7 | 19.4 | 0.057 | 0.7 |
| C1QB | 21.0 | 21.8 | 0.063 | 0.7 |

C1QA, CCND2, COL6A2 and TIMP1 are statistically significant 1-gene models. Without intending to be bound by any theory, it appears that the high Gleason scores are associated with a marked “pro-inflammatory/classically activated” monocyte pattern of gene expression with no evidence of changes in cellular/humoral immunity, based on these four statistically significant genes. It was hypothesized that enhanced pro-inflammatory gene expression may be more highly correlated with the “aggressiveness” of the cancer in localized CaP but not with immune system suppression as found in hormone-refractory CaP patients with high risk of death.

### Example 8

### RNA Transcript-Based Diagnostic Model for Predicting Prostate Cancer Patients with High Versus Low Gleason Scores: Training Set

As with the study described in Example 7 above, the goal of this study was to develop a whole blood RNA transcript-based diagnostic test that when used in conjunction with primary clinical measures, would serve to further stratify patients with lower Gleason scores as having more or less aggressive cancers, without the need for serial biopsies. Such a whole blood-based test is expected to be a more practical alternative to serial biopsies, particularly in a watching waiting strategy of treatment.

Gleason scores were available for the 76 untreated, localized prostate cancer subjects from the Training Dataset and the 128 untreated, localized prostate cancer subjects from the Test Dataset, described in Example 1. The percentage of cases for Gleason score classification amongst the subjects in both the Training and Test Datasets closely matched the incidence rates of approximately 10% Gleason scores of 8 or 9, 30% Gleason scores of 7 and 60% Gleason scores of 6, as shown in Table M below:

TABLE M

| | Training | | Test | | | | Median | |
|---|---|---|---|---|---|---|---|---|
| Gleason | Set | | Set | | Total | | PSA | |
| Score | N | % | N | % | N | % | Training | Test |
| 9 | 2 | 3% | 5 | 4% | 7 | 3% | 7.4 | 9.7 |
| 8 | 6 | 8% | 6 | 5% | 12 | 6% | 5.9 | 5.3 |
| 7 (4 + 3) | 6 | 8% | 10 | 8% | 16 | 8% | 5.7 | 4.4 |
| 7 (3 + 4) | 16 | 21% | 28 | 22% | 44 | 22% | 4.7 | 4.7 |
| 6 | 44 | 58% | 78 | 61% | 122 | 60% | 4.4 | 4.6 |
| 5 | 0 | 0% | 1 | 1% | 1 | 0% | — | — |
| Unknown | 2 | 3% | 0 | 0% | 2 | 1% | — | — |
| | 76 | 100% | 128 | 100% | 204 | 100% | 4.7 | 4.6 |

Special ordinal logit methodology was used to obtain models based on the gene expression of based on Precision Profile™ for Prostate Cancer Detection (Table 1) combined with PSA values to discriminate prostate cancer subjects with lower versus higher Gleason (“GL”) Scores.

The general agreement regarding treatment/no treatment for highest versus lowers Gleason score groups is as follows: a) GL6 (approximately 60%) should undergo “watchful waiting”; b) GL7(4+3), GL8 and GL9 (approximately 20%) should receive treatment. However, there is less agreement regarding GL(3+4) patients, regarding whether they should receive “watchful waiting” or be treated. A stereotype regression model (Anderson, J. Royal Statistical Society, Series B, 46:1-40 (1984); Magidson, Drug Information Journal, 30:143-170 (1996)) was used to examine whether the gene expressions for GL7(4+3) patients are more similar to the GL6 group or the GL7(4+3), GL8-9 group.

The dependent variable consisted of 3 Gleason score categories:

1) high scale score subgroups (GL7(4+3), GL8 and GL9-coded as ‘1’;

2) low scale score subgroup GL7(3+4)-coded as ‘s’, where ‘s’ denotes an unconstrained scale parameter estimated simultaneously with the predictor effects (betas); and

3) lowest scale score subgroup GL6-coded as ‘0’.

All possible 1-, 2- and 3-gene logit models that also included PSA as a predictor were estimated based on the 174 genes assayed (Table 1). Models were retained if they met the following qualifying criteria:

1) statistically significant betas for all predictors (including PSA);

2) sensitivity (higher GL) and specificity (lower GL) of at least 75%, where specificity is defined based upon:
a) GL6 combined with GL7(3+4)-Type 1 Model; or
b) GL6 alone-Type 2 Model.
   For all qualifying models, selection of models for validation was based upon the following criteria:

1) Wald p-values for each predictor ≦to 0.0094 or sensitivity and specificity both >90% and pvalues <0.05;

2) Scale coefficient s<0.4 (Type 1 Model) or s>0.6 (Type 2 Models);

3) Entropy R² (≧0.2); and

4) sensitivity and specificity (>75%).

The majority of the qualifying models suggested that patients with GL7(3+4) scores are most similar to patients with GL6 scores. Of the 132 qualifying 2-gene models, 100 had scale values less than 0.4 and only 3 had values greater than 0.6 (FIG. 26A). Of the 1,739 qualifying 3-gene models, 1,171 had scale values less than 0.4 and only 83 had values greater than 0.6 (FIG. 26B).

A listing of all 2- and 3-gene models of Type 1 (i.e., GL6-7(3+4) vs. GL7(4+3) or higher) having a specificity and sensitivity of at least 75% when combined with PSA values are shown Table 7A. A total of 1,984 2-gene and 3-gene models were identified. No 1-gene model met the 75% cut-off criteria. As used in this Example, specificity refers to the % of the low Gleason score group predicted correctly, and sensitivity refers to the % of the high Gleason score group predicted correctly.

As shown in Table 7A, the 2- and 3-gene models are identified in the first two and three columns (respectively) on the left side of Table 7A, ranked by their entropy R² value (shown in column 4, ranked from high to low). The number of subjects correctly classified or misclassified by each 3-gene model for each patient group (i.e., GL6-7(3+4) versus GLHigh) is shown in columns 5-8. The percent GL6-7(3+4) prostate cancer subjects and percent GLHigh prostate cancer subjects correctly classified by the corresponding gene model is shown in columns 9 and 10.

For example, the “best” logistic regression model (defined as the model with the highest entropy R² value, as described in Example 2) capable of distinguishing between prostate cancer subjects with GL6-7(3+4) and prostate cancer subjects with GLHigh (i.e., GL7(4+3) or higher) based on the 174 genes included in the Precision Profile™ for Prostate Cancer Detection, when combined with PSA, is shown in the first row of Table 7A, read left to right. The first row of Table 7A lists the 3-gene model, CASP9, RB1 and XK, capable of classifying GL6-7(3+4) prostate cancer subjects with 83.3% accuracy and GLHigh prostate cancer subjects with 85.7% accuracy. Each of the 60 GL6-7(3+4) and 14 GLHigh RNA samples were analyzed for this 3-gene model, no values were excluded. As shown in Table 7A, this 3-gene model correctly classifies 50 of the GL6-7(3+4) prostate cancer subjects as being in the GL6-7(3+4) patient population, and misclassifies 10 of the GL6-7(3+4) prostate cancer subjects as being in the GLHigh prostate cancer patient population. This 3-gene model correctly classifies 12 of the GLHigh prostate cancer subjects as being in the GLHigh prostate cancer patient population and misclassifies 2 of the GLHigh prostate cancer subjects as being in the GL6-7(3+4) prostate cancer population.

A ranking of the top genes for which gene expression profiles were obtained, from most to least significant, is shown in Table 7B. Table 7B summarizes the mean expression levels of the genes listed in the Precision Profile™ for Prostate Cancer Detection (Table 1) measured in the RNA samples obtained from the prostate cancer subjects in the Training Dataset, as well as the results of significance tests (Wald p-values) for the difference in the mean expression levels between the GL6-7(3+4) and GLHigh prostate cancer subjects.

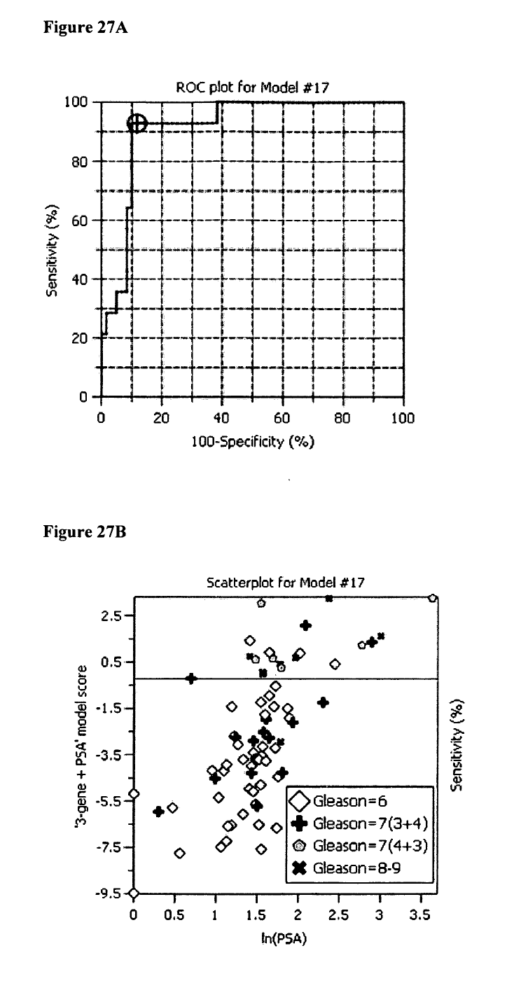

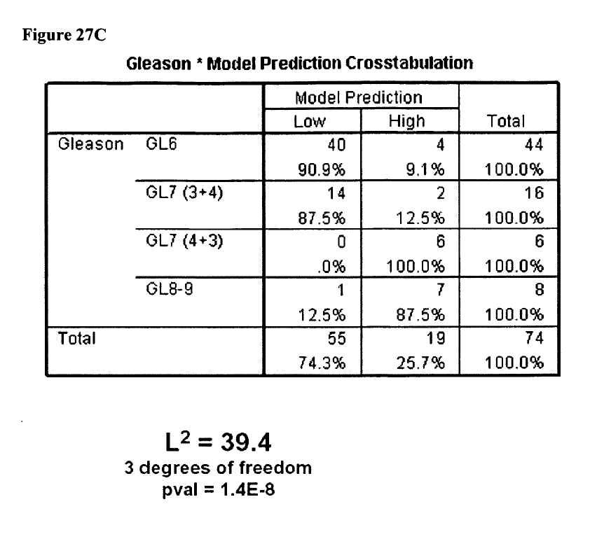

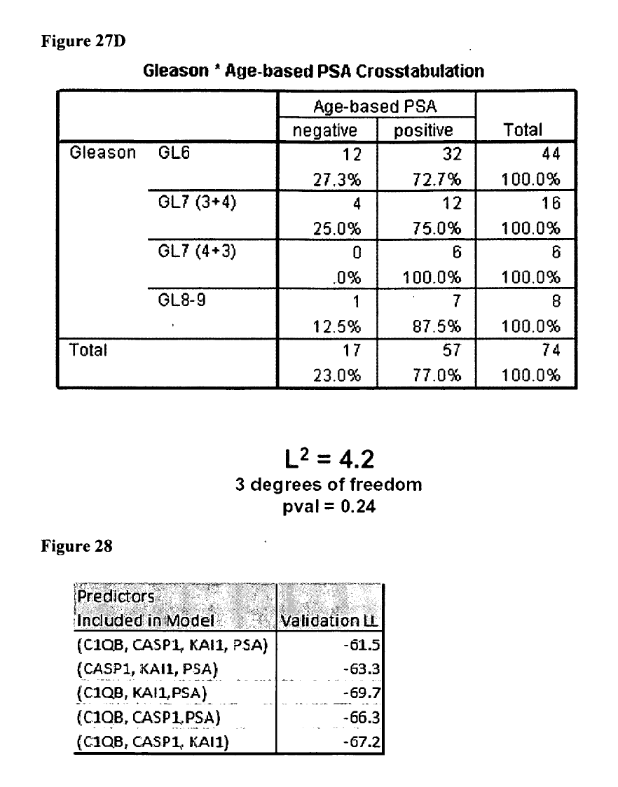

As another example, the 3-gene model C1QB, CASP1 and KAI1 combined with PSA yields a sensitivity of 92.9% (i.e., % GLHigh predicted correctly) and specificity of 90% (i.e., % GL6-7(3+4) predicted correctly). A ROC curve for this 3-gene+PSA model (C1QB, CASP1 and KAI1+PSA) is shown in FIG. 27A. The area under the ROC curve for the 3-gene+PSA model shown in FIG. 27A is 0.915 (p-val=0.0001). A discrimination plot for this 3-gene model is shown in FIG. 27B. The logit (Gleason high vs. low) for this 3-gene+PSA model equals 11.3+2.67*pLnPSA−1.56*C1QB+6.06*CASP1-3.83*KAI1. As shown in FIGS. 27C and 27D, this 3-gene+PSA model prediction of Gleason score group is highly significant (p-value=1.4E-8), whereas prediction of Gleason score groups based on age-adjusted PSA alone is not significant (p-value=0.24).

Furthermore, the 3-gene (C1QB, CASP1, and KAI1)+PSA model developed on the Training Dataset successfully cross-validated. This 3-gene+PSA model did not over fit according to K-fold cross-validation. The following analysis was done to test for overfitting:

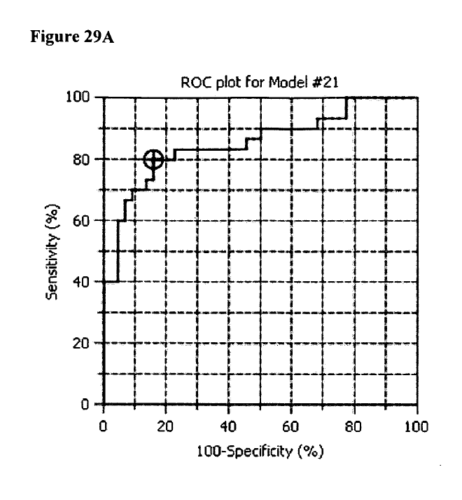

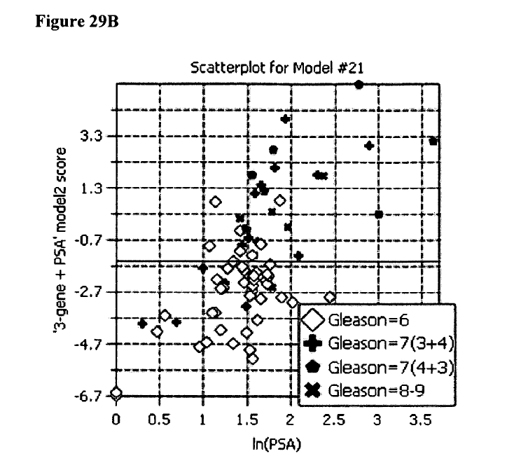

a) Data were randomly split into K=10 equal sized subsamples;
b) Target model was re-estimated 10 times, each time omitting 1 sub sample;
c) Re-estimated model was applied to omitted subsample;
d) Results were accumulated across all subsamples;
e) Validation log-likelihood (Validation LL) (standard LL always decreases when a predictor is excluded from a model; validation LL should increase if the excluded predictor is extraneous). As shown in FIG. 28, validation LL decreased from −61.5 for each of the four 3-predictor sub-models where 1 predictor is excluded. These results indicate a successful cross-validation (i.e., no extraneous predictors in the model).

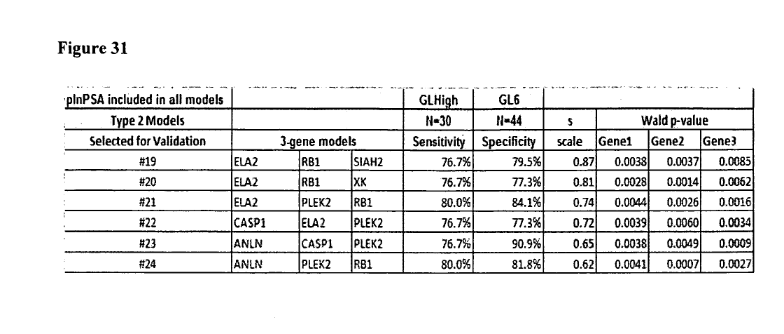

A listing of all 3-gene models of Type 2 (i.e., GL6 vs. GL7 or higher) having a specificity and sensitivity of at least 75% when combined with PSA values are shown FIG. 31. As used in this Example, specificity refers to the % of the low Gleason score group (GL6) predicted correctly, and sensitivity refers to the % of the high Gleason score group (GLHigh, i.e., GL7 or higher) predicted correctly.

For example, the 3-gene model, ELA2, PLEK2, RB1, plus PSA correctly classifies 84.1% of prostate cancer patients from the Training Dataset with higher Gleason scores (i.e., GL7, 8, 9) and 80% of prostate cancer patients from the Training Dataset with lower Gleason scores (i.e., GL6 or less). A ROC curve for this 3-gene (ELA1, PLEK2, RB1)+PSA model is shown in FIG. 29A. The logit (Gleason high vs. low) for this 3-gene+PSA model equals 71.36+2.14*pLnPSA−0.77*ELA2+1.12*PLEK2+3.38RB1. A scatter plot for this 3-gene+PSA model is shown in FIG. 29B.

### Future Validation Studies:

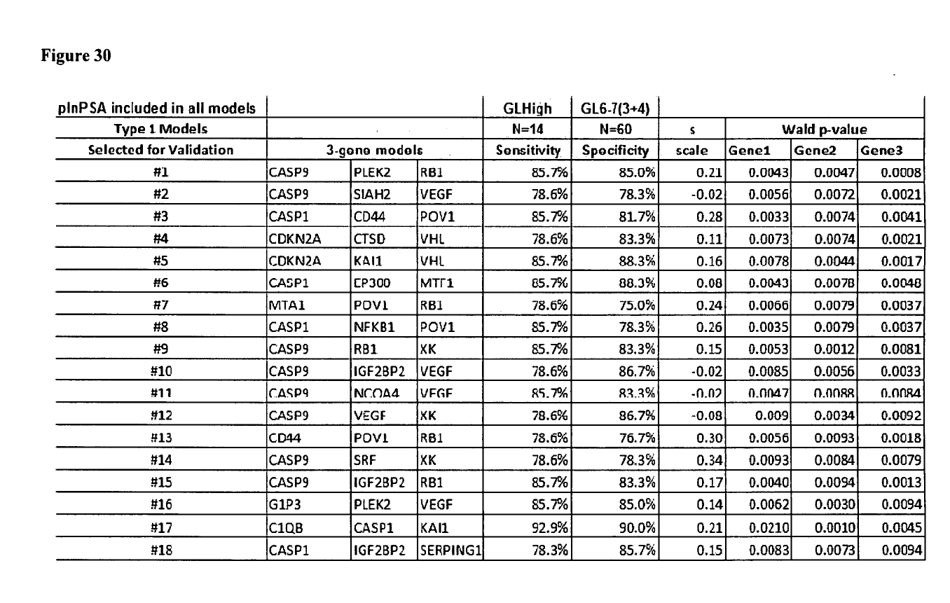

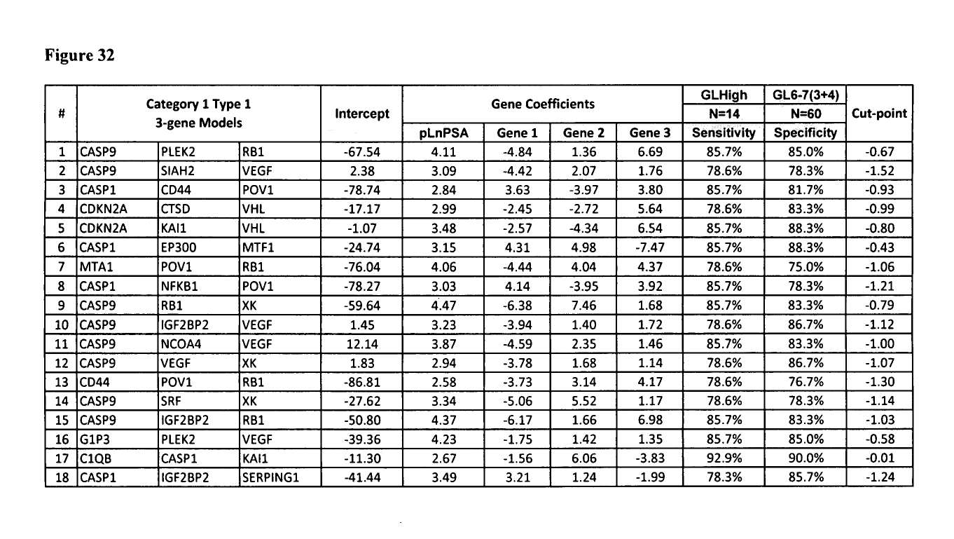

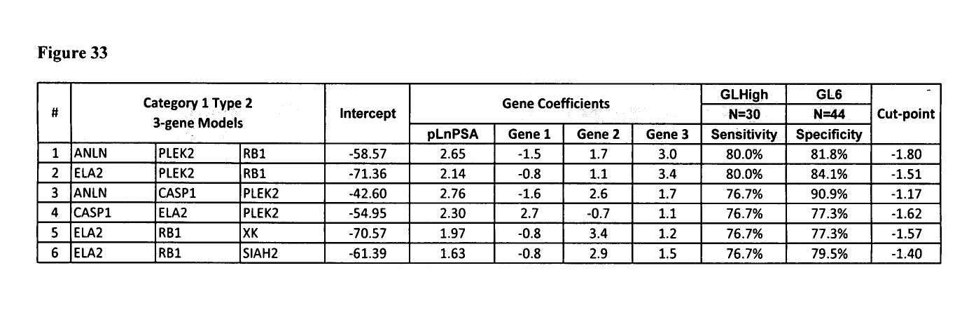

The top 18 3-gene+PSA Type 1 models that result when ranked by ‘s’ scale coefficient is shown FIG. 30. The top 6 3-gene+PSA Type 2 models is shown in FIG. 31. These Type 1 and Type 2 models will be validated using the subject samples from the Test Dataset, pre-specified gene coefficients and fixed cut-off points (FIGS. 32 and 33). A pre-specified plan will be followed:

### Test A:

a) Using pre-specified model coefficients (beta) established in TRAINING dataset (see FIGS. 30 and 31) compute a model logit score (log-odds of highest vs. lowest Gleason Score categories)
b) Apply pre-specified cut-point established in the TRAINING dataset to yield 2 groups. Subjects with logit scores above cut-off point are predicted to be CaP/High Gleason and those with scores below cut-off point are predicted to be CaP/Low Gleason.
c) Form 2×2 table of frequency counts (actual by predicted classification). Compute likelihood ratio chi-squared (L²) and derive p-value with 1 degree of freedom. A validation p-value <0.05 constitutes a successful validation (meaning test results deviate from independence with 95% confidence).

### Test B:

a) Repeat Test A using model coefficients (beta and scale parameters) re-estimated on the test dataset.
b) Validation is successful if the re-estimated beta parameters are in the same direction as the original model and the predictions based on the logit cut-point of 0 results in a p-value <0.05.

### Test C:

a) Using pre-specified model coefficients established in TRAINING dataset compute a model logit score.
b) Construct comparative ROC curves using the 2- or 3-gene model logit score vs. the age-adjusted PSA criterion. The model validates if the improvement in the area under the curve (AUC) associated with the 2- or 3-gene logit model vs. age-adjusted PSA is significant (p<0.05.).

### Example 9

### Incremental Use of Gene Models to Identify Highest Risk Prostate Cancer Subjects

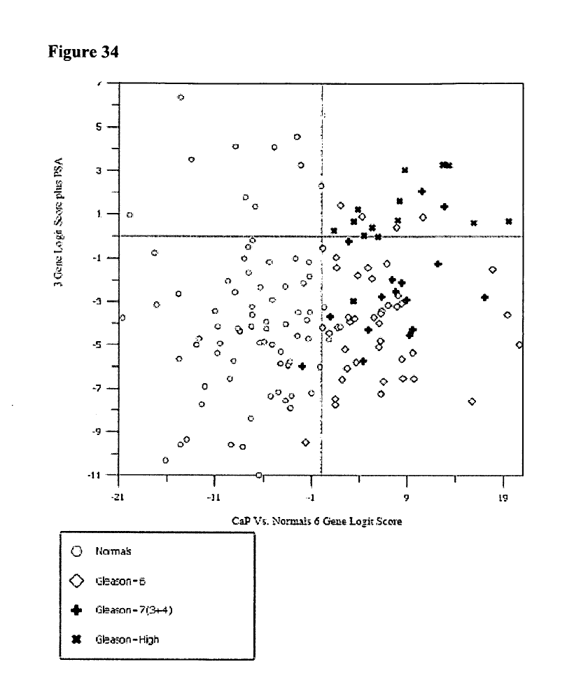

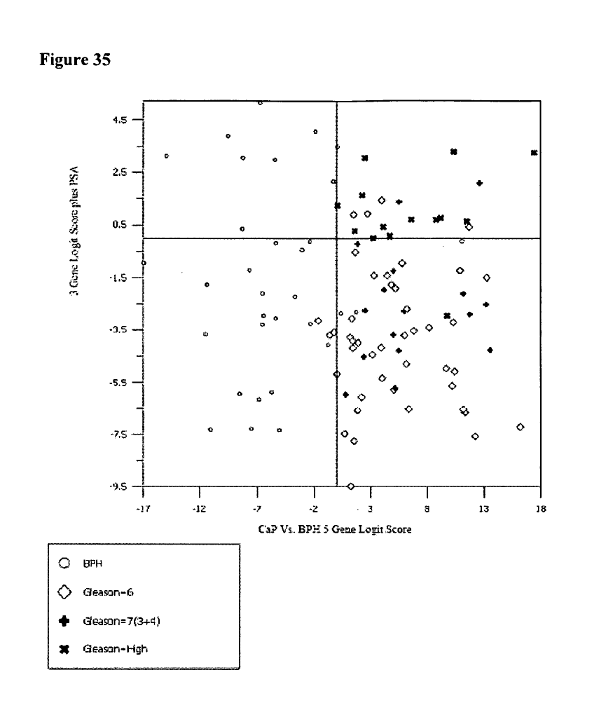

Two or more of the gene-models described herein can be used incrementally or iteratively to discriminate first prostate cancer patients from normal, healthy subjects, followed by further discrimination of prostate cancer patients into high and low Gleason score groups. The highest risk prostate cancer subjects can be identified using such methods. For example, the 6-gene model (RP51077B9.4, CD97, CDKN2A, SP1, S100A6, IQGAP1)+PSA model described in Examples 4 and 6, which discriminates prostate cancer subjects from normal, healthy subjects (without BPH), can be combined with the 3-gene (C1QB, CASP1, KAI1)+PSA model described in Example 7 which discriminates prostate cancer subjects with a low Gleason score (i.e., 6-7(3+4)) from prostate cancer subjects with a high Gleason score (i.e., 7(4+3) or higher), as shown in FIG. 34, to identify the highest risk prostate cancer subjects (upper right quadrant). As a further example, the 5-gene (S100A6, MYC, MAP2K1, C1QA, RP51077B9.4)+PSA+age model described in Example 6 which discriminates between prostate cancer subjects and BPH subjects can be combined with the 3-gene (C1QB, CASP1, KAI1)+PSA model described in Example 7 to identify the highest risk prostate cancer subjects, as shown in FIG. 35.

### Example 10

### RNA Transcript-Based Diagnostic Models for Predicting Prostate Cancer Patients with High vs. Low Gleason Scores: Preliminary Results of Extended Logit and Latent Class Modeling

Like the study described in Example 8, this study was designed to discriminate between localized prostate cancer (CaP) patients with Gleason scores of 6 and 7(3+4) vs. 7(4+3), 8 and 9 (Type 1 Models) and also between patients with 6 vs. 7, 8 and 9 (Type 2 Models).

Whole blood was collected in PaxGene™ Blood RNA Tubes from 198 CaP subjects and submitted to exploratory statistical analysis. A total of 216 inflammation, general cancer and prostate cancer related target genes (shown in the Prostate Cancer Clinically Tested Precision Profile™ in Table 8 below) were assayed for each subject and used as candidate predictors in 1-, 2- and 3-gene models. The Gleason score distribution among the 198 subjects is shown below in Table N.

TABLE N
Gleason Score distribution

| Gleason | | | Median |
|---|---|---|---|
| Score | N | % | PSA |
| 9 | 7 | 4% | 4.2 |
| 8 | 12 | 6% | 5.0 |
| 7 (4 + 3) | 17 | 9% | 5.0 |
| 7 (3 + 4) | 41 | 21% | 4.2 |
| 6 | 121 | 61% | 4.4 |
| Total | 198 | 100% | 4.7 |

As shown in Table N, the percentage of cases for Gleason score classification closely matches the incidence rates of approximately 10% Gleason scores of 8 or 9 (GL8 or GL9), 30% Gleason scores of 7 (GL7), and 60% Gleason scores of 6 (GL6). Generally, very few of the 60% GL6 patient population are believed to have aggressive growing tumor, but if the biopsy was not taken from the exact right location, it may miss a more extensive tumor. In addition, it is unclear as to what percentage of GL7 (3+4) patient population have less aggressive tumors and what percentage of patients have more aggressive tumors. Most of the GL7 (4+3), GL8 and GL9 patient population is believed to have an aggressive growing tumor, and all are treated.

The Gleason biopsy score is used to grade tumors in prostate cancer as to the expected biologic aggressive potential of the disease to spread to other organs. Since it is not a perfect indicator of such aggressive potential, it represents an example of an imperfect reference test.

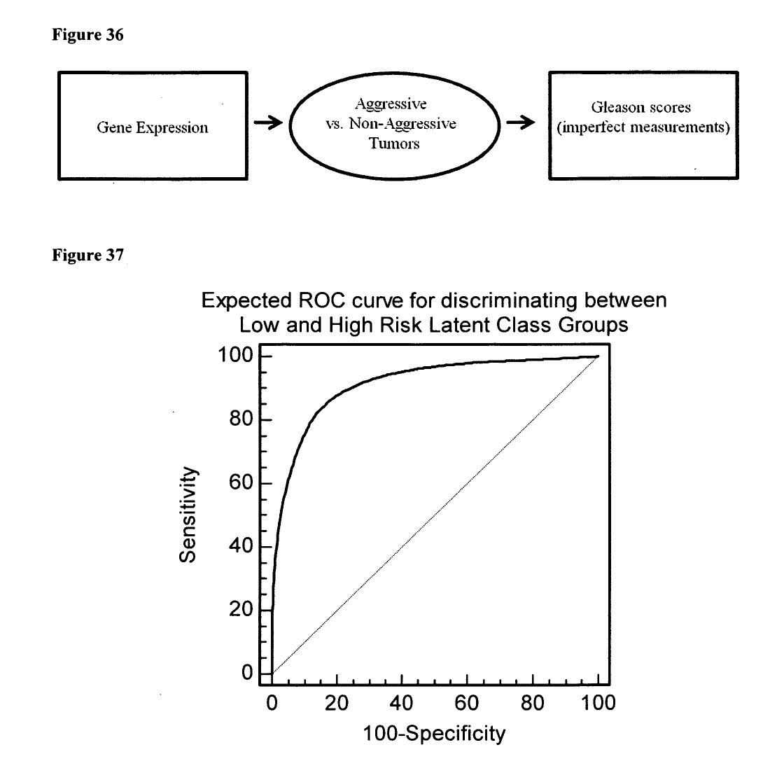

Latent class (LC) analysis is commonly used in the field of biometrics to estimate the magnitude of the error associated with imperfect reference tests where multiple measurements (i.e., multiple tests) are available. However, LC modeling is quite general. Thus a particular model was developed to account for the error in the Gleason score. The model developed in the present study differs from the more common applications because here the Gleason score is the only reference test. A particular kind of LC model was used here that employs a ‘supervised classification structure’ (See Vermunt and Magidson, Computational Statistics and Data Analysis, 41: 531-537 (2003)) which makes it appropriate for a single reference test. It is a basic type of LC model for classification which involves specifying a model for the conditional distribution of y given z, where a discrete hidden variable x serves as intervening variable. The assumed probably structure for P(y, z) is formally defined by the equation:

$$P ( y , z ) = P ( z ) P ( y \vert z ) = P ( z ) {\sum }_{x} P ( x \vert z ) P ( y \vert z , x ) ,$$

where P(z) is treated as fixed (See Vermunt and Magidson, 2003, page 532). The assumption of ‘local independence’ was added, which is depicted graphically in FIG. 36. The diagram shown in FIG. 36 has an arrow going from the gene expression to the latent variable called ‘aggressiveness’ to indicate that the gene expression is assumed to be capable of distinguishing between aggressive and non-aggressive cancers, and another arrow from ‘aggressiveness’ to the Gleason score to indicate that Gleason is an imperfect attempt to measure ‘aggressiveness’. The local independence assumption is represented by there being no direct arrow between the gene expression and the Gleason scores. Thus, the connection between gene expression and Gleason score is established through the intervening latent variable ‘aggressiveness’. That is, the effects of the gene expression (z variables) on the Gleason scores (y) go completely through the latent variable ‘aggressiveness’ (x).

The equation:

$$P ( y \vert z ) = {\sum }_{x} ⌊ {\prod }_{j} P ( {x}_{j} \vert z ) ⌋ P ( y \vert x ) ,$$

provides an additional equation which formalizes the local independence assumption mathematically and describes this special case of the more general model in equation (1)—“the effects of the z variables on the y go completely through x’. See Vermunt and Magidson, 2003, page 533).

Because Gleason score is an imperfect measure of ‘aggressiveness’, the AUC for the prediction of ‘aggressiveness’ by the gene expression (0.91) is reduced to 0.71 when the gene expression is used to predict Gleason score. The magnitude of the reduction is directly proportional to the amount of error in the Gleason score. It can be shown mathematically that under this hypothesized model structure, the more imperfect the measurement of ‘aggressiveness’ by the Gleason score, the greater the expected shrinkage in the AUC.

Since a gold standard is unavailable/unknown, it was hypothesized that two true latent states existed using a latent class model with 2 “true” latent subject classes: 1) Non-aggressive cancer group (low risk to be assigned to “watchful waiting”); and 2) Aggressive cancer group (higher risk group to receive “active treatment”). A two step approach was used to discriminate between lower vs. higher risk latent subject groups and two estimate the error in Gleason scores. In Step 1, extended logit models containing 2-3 genes were developed to predict 3 Gleason score categories as a function of gene expression and to determine whether GL7 (3+4) patients are more similar to the GL6 group or the GL7 (4+3) group, GL8-9 group (See Anderson, J. Royal Statistical Society, Series B, 46:1-40 (1984); Magidson, Drug Information Journal, 30:143-170 (1996)). In step 2, latent class models were developed based on 1 or more logit models, PSA and age (See Vermunt and Magidson (2008 (Latent GOLD Technical Guide, Belmont, Mass.: Statistical Innovations), and measurement error was estimated for each of the 3 Gleason categories to account for the fact that Gleason scores are imperfect measures of tumor aggressiveness (i.e., the gene expressions are predictive of the latent classes (subjects with aggressive vs. non-aggressive tumors) which in turn is measured (imperfectly) by the Gleason scores).

Expanding upon Step 1, extended logit regression methodology was used to obtain models that included gene expression to discriminate between CaP subjects with lower vs. higher Gleason (GL) scores. The observed dependent variable consisted of 3 Gleason score categories:

1) High scale score subgroups GL7 (4+3), GL8 and GL9-coded as ‘1’;
2) Low scale score subgroup GL7 (3+4)-coded as ‘s’, where s denotes an unconstrained scale parameter estimated simultaneously with the predictor effects (betas); and
3) Lowest scale score subgroup GL6-coded as ‘0’.

All possible 1-, 2- and 3-gene logit models that also included PSA as a predictor were estimated based on the 216 genes assayed. Models were retained if they met the following qualifying criteria:

1) statistically significant betas for all predictors;

2) sensitivity (higher GL) and specificity (lower GL) of at least 60%, where specificity is defined based upon:
a) GL6 combined with GL7(3+4)-Type 1 Model (scale parameter less than 0.5); or
b) GL6 alone-Type 2 Model (scale parameter >0.5).

A listing of the 2-gene qualifying models with significant p-values from Step 1 is shown in Table 9. The top 3-gene models from Step 1, ranked from highest to lowest based on Entropy R² values, is shown in Table 10. In Tables 9 and 10, only the 2- and 3-gene models for which all genes were statistically significant (p-val <0.05) and that met the correct classification criteria (>60%) based on either definition A or B (i.e., Type 1 or Type 2 model) were included.

For definition A (i.e., Type 2 model), GL7/3+4 are combined with the higher Gleason scores, so that the 60% criteria means that at least 60% of the subjects with GL6 biopsies (N=121) and at least 60% of subjects with higher Gleason scores (N=77) were correctly predicted by the model. For definition B (i.e., Type 1 models), GL7/3+4 are grouped with GL6, so that the 60% criteria means that at least 60% of the subjects with GL6 or GL7/3+4 biopsies (N=162) and at least 60% of subjects with higher Gleason scores (N=36) were correctly predicted by the model. The first column in Tables 9 and 10 indicate whether the correct classification rates, as shown in columns 5-10 for the 2-gene models in Table 9, and as shown in columns 6-11 for the 3-gene models shown in Table 10, are based on the A (i.e., Type 2) or B (i.e., Type 1) definition.

Models achieving at least 60% correct classification under both definitions A (i.e., Type 2) and B (i.e., Type 1) are shown underlined in Tables 9 and 10. For these models, the correct classification rates in the first set of columns are based on definition A (i.e., Type 2 model) and those associated with definition B (i.e, Type 1 model) are shown to the right in Columns. 18-24 in Table 9 and Columns 21-27 in Table 10. All low expressing genes are shown in bold, italicized font in both tables.

Three of the qualifying models were selected for inclusion in latent class models along with age and PSA (Step 2). Models were excluded if they contained a low expressing gene or had scale factor estimate significantly less than 0 or greater than 1. The following Type 1 and Type 2 models were selected from the Step 1 for inclusion in latent class model development in Step 2:

Type 1, 2-gene models (included a total of 28 models): CD4, TP53 (best 2-gene model);
Type 2, 2-gene models (included a total of 3 models): CASP9, SOCS3 (only model with scale parameter significantly >0);
Type 1, 3-gene models: CD4, TP53, E2F1 (best 3-gene model)

The results from latent class modeling based on the best 3-gene model (a Type 1 model, CD4, TP53 and E2F1) is shown in Table O below:

TABLE O
LC model based on CD4, TP53 and E2F1

| Covariates | Class1 | p-value |
|---|---|---|
| logit.tp53.cd4.e2f1 | −0.69 | 0.04 |

The results from the latent class modeling based on the Type 2 2-gene model selected for which the scale value differed significantly from 0 (CASP9 and SOCS3) is shown in Table P below:

TABLE P
LC model based on CASP9 and SOCS3

| Covariates | Class1 | p-value |
|---|---|---|
| logit.casp9.socs3 | −1.04 | 0.09 |

Results from LC models combining the best 3-gene model with the selected Type 2 2-gene model is shown below in Table Q:

TABLE Q
LC model based on combined Type 1
3-gene and Type 2 2-gene models

| Covariates | Class1 | p-value |
|---|---|---|
| logit.tp53.cd4.e2f1 | −0.93 | 0.03 |
| logit.casp9.socs3 | −1.60 | 0.03 |

Results from LC modeling combining the best 3-gene model (a Type 1 model) with the selected 2-gene Type 2 model plus age and PSA is shown in Table R and Table S below:

TABLE R
LC model based on combined Type 1
3-gene and Type 2 2-gene models and age

| Covariates | Class1 | p-value |
|---|---|---|
| logit.tp53.cd4.e2f1 | −0.85 | 0.01 |
| logit.casp9.socs3 | −1.81 | 0.02 |
| Age | −0.07 | 0.05 |

TABLE S
LC model based on combined Type 1
3-gene and Type 2 2-gene models and PSA

| Covariates | Class1 | p-value |
|---|---|---|
| logit.tp53.cd4.e2f1 | −0.76 | 0.00 |
| logit.casp9.socs3 | −1.57 | 0.02 |
| Age | −0.07 | 0.05 |
| plnPSA | −0.58 | 0.10 |

A comparison of results from LC modeling based on the 2-gene and 3-gene models is shown below in the Table T:

TABLE T

| | 2-gene LC | | 3-gene + Age LC | |
|---|---|---|---|---|
| | Genes | Beta | Genes | Beta |
| | (Constant) | −.48 | (Constant) | −11.56 |
| | SOCS3 | −2.82 | TP53 | 6.30 |
| | CASP9 | 3.16 | CD4 | −5.03 |
| | | | E2F1 | −1.70 |
| | Latent Class | | Latent Class | |
| | Low Risk | High Risk | Low Risk | High Risk |
| | 0.72 | 0.28 | 0.69 | 0.31 |
| | [.38,1.0] | [0.0,.62] | [0.42,.96] | [.04,.58] |
| Size | 99.4% | 0.6% | 99.5% | 0.5% |
| 95% Conf. Bounds | 81.1% | 18.9% | 77.3% | 22.7% |
| Gleason Scores | 39.5% | 60.5% | 37.1% | 62.9% |
| 6 | 0.92 | 0.08 | 0.85 | 0.15 |
| 7/3 + 4 | 0.44 | 0.56 | 0.80 | 0.20 |
| 7/4 + 3,8,9 | 0.38 | 0.62 | 0.05 | 0.95 |
| Sensitivity | 0.70 | | 0.73 | |
| (high risk) | | | | |
| Specificity | 0.69 | | 0.72 | |
| (low risk) | | | | |

Note the wide confidence interval regarding the size of the latent classes.

A comparison of the results from LC modeling based on combining the 2-gene and 3-gene models with and without age is shown below in Table U:

TABLE U

| | 3 + 2gene LC | | 3 + 2gene + Age LC | |
|---|---|---|---|---|
| | Genes | Beta | Genes | Beta |
| | (Constant) | −.48 | (Constant) | −11.56 |
| | TP53 | 6.89 | TP53 | 6.30 |
| | CD4 | −5.51 | CD4 | −5.03 |
| | E2F1 | −1.86 | E2F1 | −1.70 |
| | SOCS3 | −2.82 | SOCS3 | −3.19 |
| | CASP9 | 3.16 | CASP9 | 3.58 |
| | | | Age | .147 |
| | | Latent Class | | Latent Class |
| | Low Risk | High Risk | Low Risk | High Risk |
| Size | 0.85 | 0.15 | 0.83 | 0.17 |
| 95% Conf. Bounds | [0.75,.95] | [.05,.25] | [0.75,.91] | [.09,.25] |
| Gleason Scores | | | | |
| 6 | 99.4% | 0.6% | 99.5% | 0.5% |
| 7/3 + 4 | 81.1% | 18.9% | 77.3% | 22.7% |
| 7/4 + 3,8,9 | 39.5% | 60.5% | 37.1% | 62.9% |
| Sensitivity | 0.84 | | 0.86 | |
| (high risk) | | | | |
| Specificity | 0.83 | | 0.86 | |
| (low risk) | | | | |
| Area Under Curve | 0.91 | | 0.92 | |

As shown in Table U, the models predict the probability of being in each latent class. Additionally, the confidence interval is much tighter based on the combined models as compared to the confidence intervals shown in Table T. An expected ROC curve for the LC model consisting of combined 3-gene+2-gene models plus age is shown in FIG. 37 (AUC=0.92). As shown in FIG. 37, for each subject, the LC model provides a predicted probability of being in class 1 or class 2 based on the covariates in the model. To get the expected sensitivity/specificity, a subject with a probability of 0.8 of being in class 1 contributes 4 towards class 1 and 1 towards class 2.

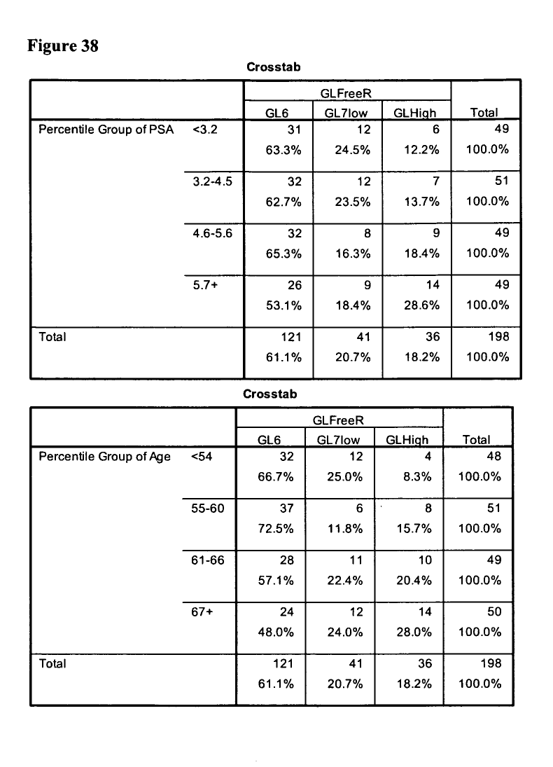

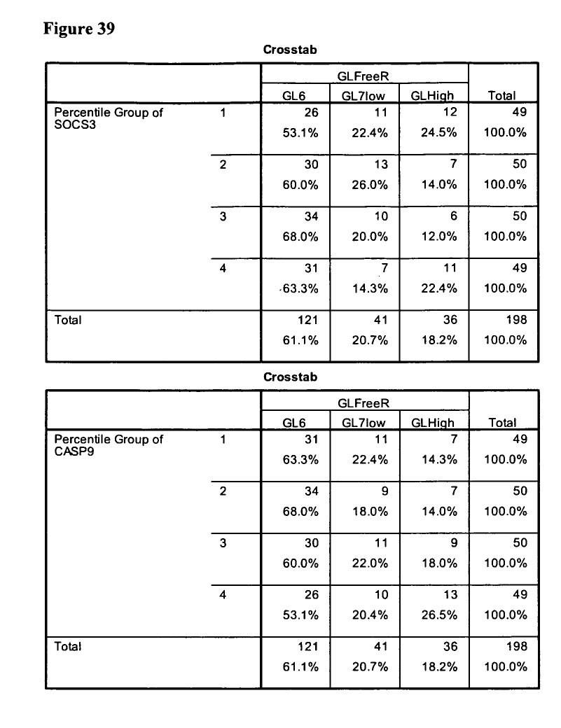

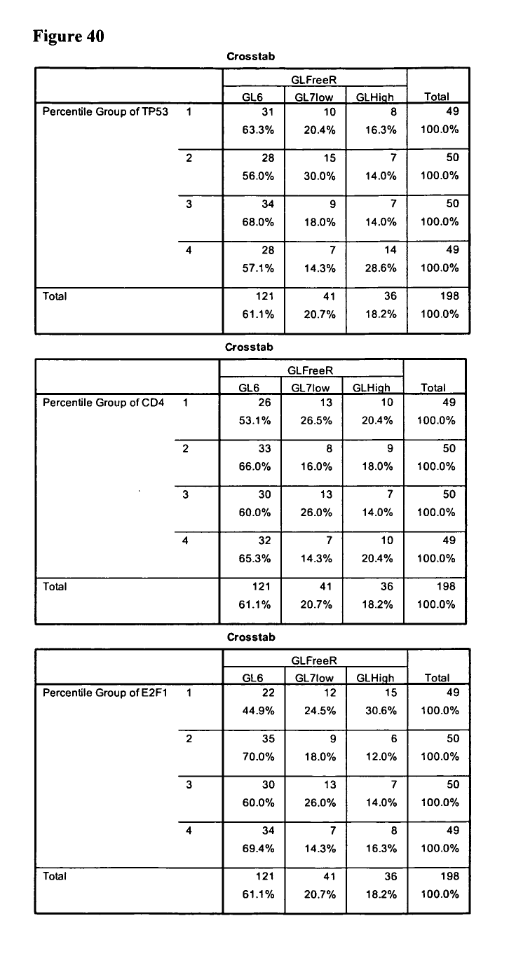

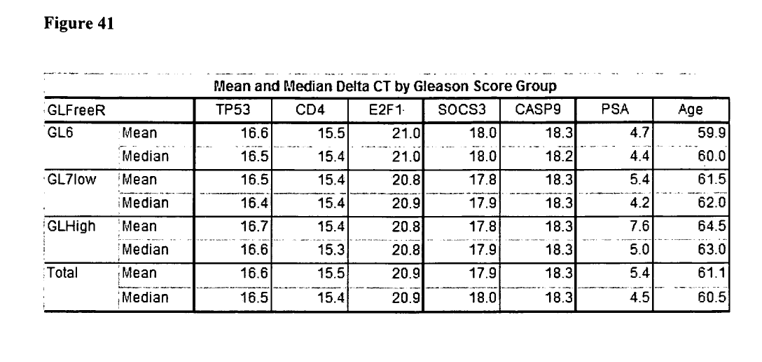

Additional Gleason statistics for PSA and age by Gleason Scores is shown in FIG. 38. Descriptive Gleason statistics of genes in the Type 2 model, CASP9 and SOCS3 is shown in FIG. 39. Descriptive Gleason statistics of genes in the Type 1 model, TP53, CD4 and E2F1, is shown in FIG. 40. Descriptive Gleason means and statistics for the genes in these Type 1 3-gene and Type 2 2-gene models is shown in FIG. 41.

### Example 11

### Cell Fractionation Study

The Cell Fractionation study described in this Example was designed to investigate the cellular origins of RNA transcript-based gene expression observed in whole blood collected from subjects that have been newly diagnosed with prostate cancer. In this study, whole blood samples from 14 individuals newly diagnosed with prostate cancer (Cohort 1) were collected in CPT tubes for purification of peripheral blood mononuclear cells (PBMC's). Four different cell types were subsequently enriched from the purified PBMC fraction (B cells, monocytes, NK cells, T cells), and levels of gene transcripts in nine samples/subject (8 cell fractions (enriched and depleted fractions) from four cell types: B cells, Monocytes, NK cells and T cells and the original PBMC fraction) were quantitatively analyzed using were quantitatively analyzed using proprietary optimized QPCR assays (Precision Profiles™). In addition, whole blood samples from 14 age and gender-matched medically defined Normal subjects (MDNO) were similarly collected in CPT tubes for purification of PBMC's and downstream analysis of enriched/depleted cell population fractions.

Eighteen target genes of interest (i.e., the Prostate Cancer (Cohort 1) Cell Fractionation Gene List shown in Table 11 below) were selected for analysis based upon previous in-house studies where statistically significant differences in mean levels of expression between cancer and normal subjects were observed in addition to relevant cell markers for specific cell populations. Normalized target gene expression values from PBMC samples were compared to those from enriched (and depleted) cell fractions to determine whether an increase in expression was observed in a specific cellular fraction(s). Expression levels of cell specific markers were also analyzed in parallel for each cellular fraction generated in the enrichment process, to determine the fold-enrichment of specific cell types. A comparison of the averaged relative expression values in enriched cell fractions from both normal and disease cohorts was performed to investigate potential differences in the levels of expression across cell types which may correlate to differences observed in whole blood.

### Methods

### Cell Enrichment and RNA Extraction:

Becton Dickinson IMag™ Cell Separation Reagents were used to magnetically enrich the four different cell types isolated from the PBMC fraction of whole blood following the manufacturers recommended protocol and Source MDx SOP 200-136.

### RNA Quality Assessment:

Integrity of purified RNA samples was visualized with electropherograms and gel-like images produced using the Bioanalyzer 2100 (Agilent Technologies) in combination with the RNA 6000 Nano or Pico LabChip.

cDNA First Strand Synthesis and QC:

First strand cDNA was synthesized from random hexamer-primed RNA templates using TaqMan® Reverse Transcription reagents. Quantitative PCR (QPCR) analysis of the 18S rRNA content of newly synthesized cDNA, using the ABI Prism® 7900 Sequence Detection System, served as a quality check of the first strand synthesis reaction.

### Quantitative PCR:

Target gene amplification was performed in a QPCR reaction using Applied Biosystem's TaqMan® 2× Universal Master Mix and Source MDx proprietary primer-probe sets. Individual target gene amplification was multiplexed with the 18S rRNA endogenous control and run in a 384-well format on the ABI Prism® 7900HT Sequence Detection System.

### QPCR Data Analysis:

QPCR Sequence Detection System data files generated, consist of triplicate target gene cycle threshold, or C_{T} values (FAM) and triplicate 18S rRNA endogenous control CT values (VIC). Normalized, delta C_{T} (ΔC_{T}) gene expression values for each amplified gene are calculated by taking the difference between C_{T} values of the target gene and its endogenous control. All replicate C_{T} values (target gene and endogenous control) are quality control checked to ensure that predefined criteria are met. An average delta C_{T} value is then calculated from gene specific FAM and VIC replicate sets. The difference in normalized gene expression values (ΔC_{T}) between samples is calculated to obtain a delta delta C_{T} (ΔΔC_{T}) value: ΔC_{T} (enriched sample) ΔC_{T} (PBMC control sample). The ΔΔC_{T} value is then used for the calculation of a relative expression value with the following equation: 2−(ΔΔC_{T}). Therefore, a difference of one C_{T}, as determined by the ΔΔC_{T} calculation, is equivalent to a two-fold difference in expression. Relative expression values were calculated for the enriched and depleted samples compared to the PBMC starting material to determine cell specific expression for the genes analyzed.

### Results

Gene expression Analysis of Fractionated Cell Samples from Prostate Cancer (Cht 1) Whole Blood Samples

A quantitative comparison of gene expression levels on a panel of eighteen target genes composed of the six prostate cancer early detection model genes, four cell marker genes and eight additional genes of interest (i.e., the Prostate Cancer (Cohort 1) Cell Fractionation gene Panel shown in Table 11) was analyzed.

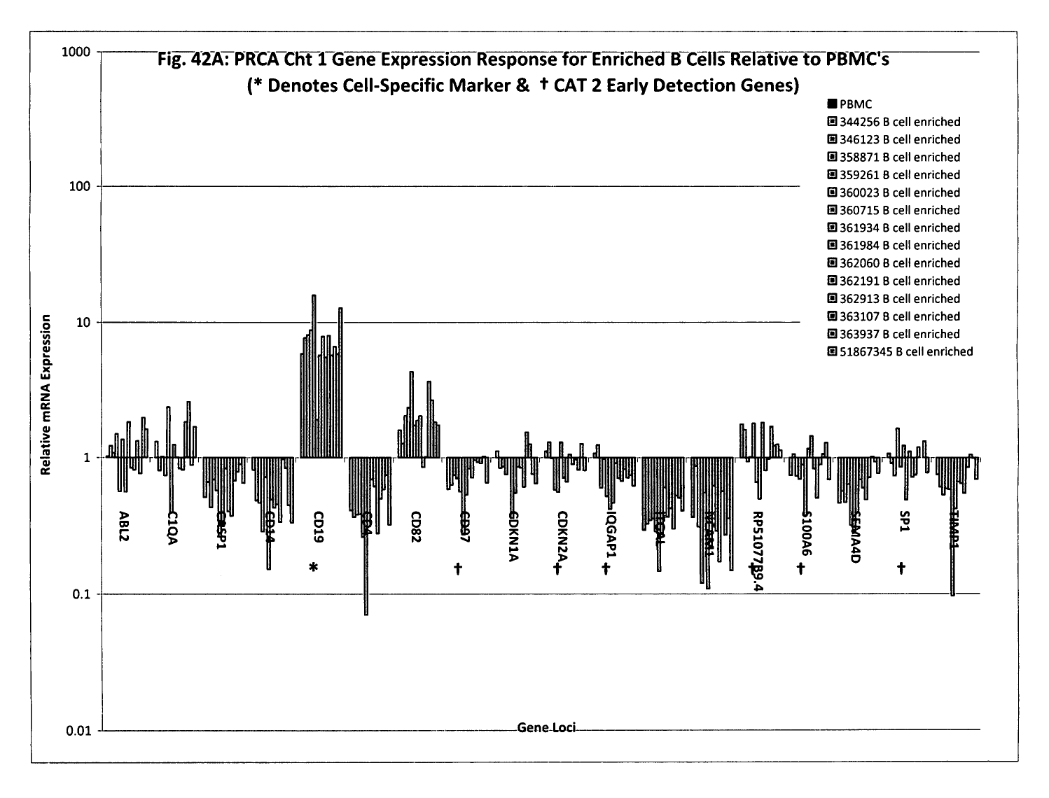

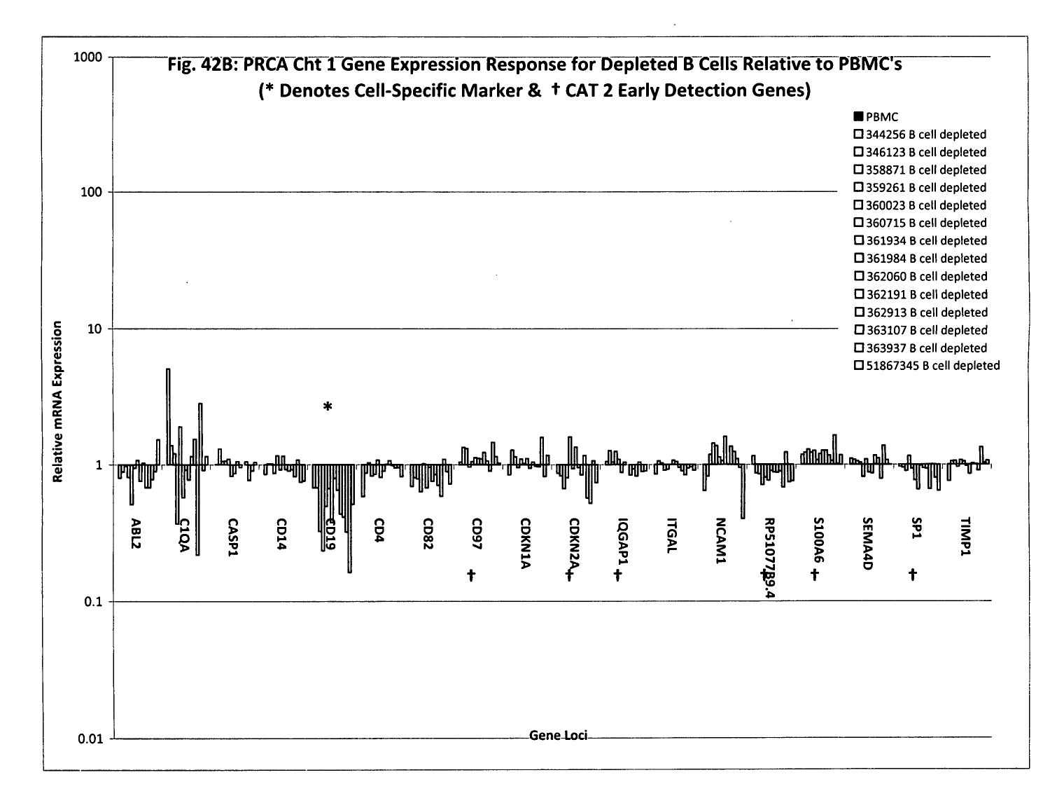

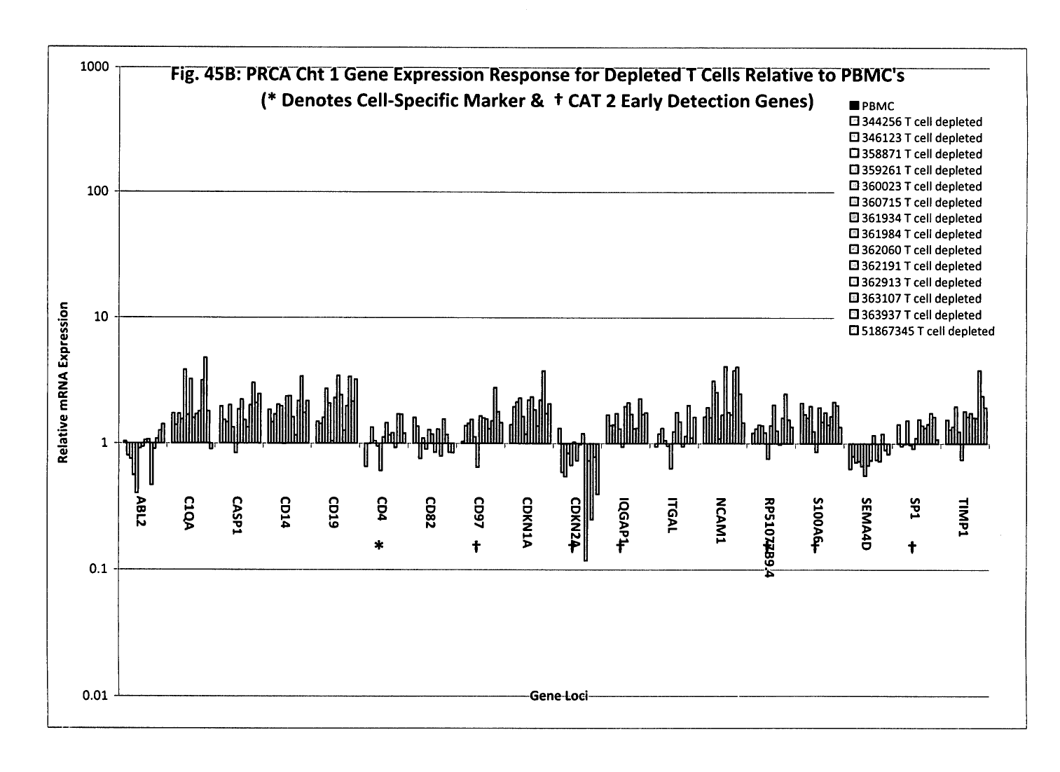

1. Expression levels of genes in enriched and depleted cell fractions were compared to those in the original PBMC fraction for all prostate cancer subjects. A graphical representation of the relative gene expression response for individual prostate cancer subjects in both enriched and depleted cell fractions is presented in FIGS. 42A&B through FIGS. 45A&B. Note: all “A” figures show the response in enriched fractions and “B” figures the depleted fractions.
Key observations included the following:

The gene expression profile was very similar between the 14 prostate cancer patient samples for the majority of genes in specific cell fractions, indicating a consistency in cell-specific gene expression across individuals.

The magnitude of the cell-specific response was slightly variable between individual subject samples.

Genes showing an induction in enriched cell fractions, had a corresponding decrease in expression in the depleted cell fraction for the same cell type.

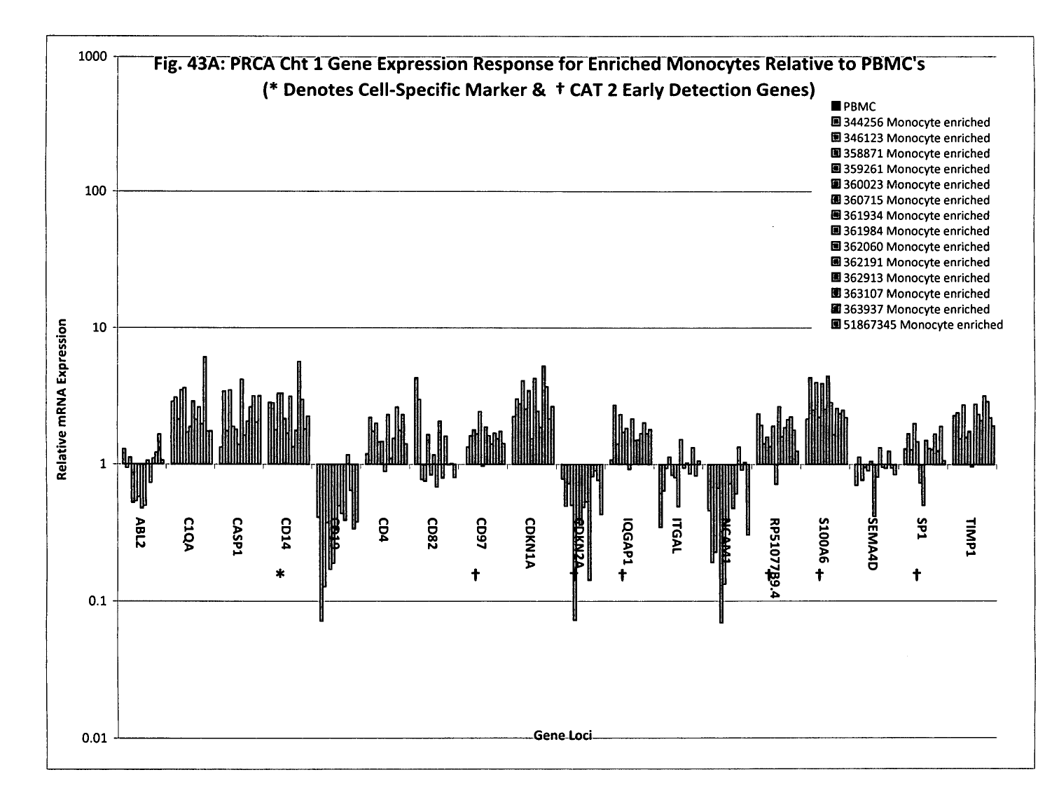

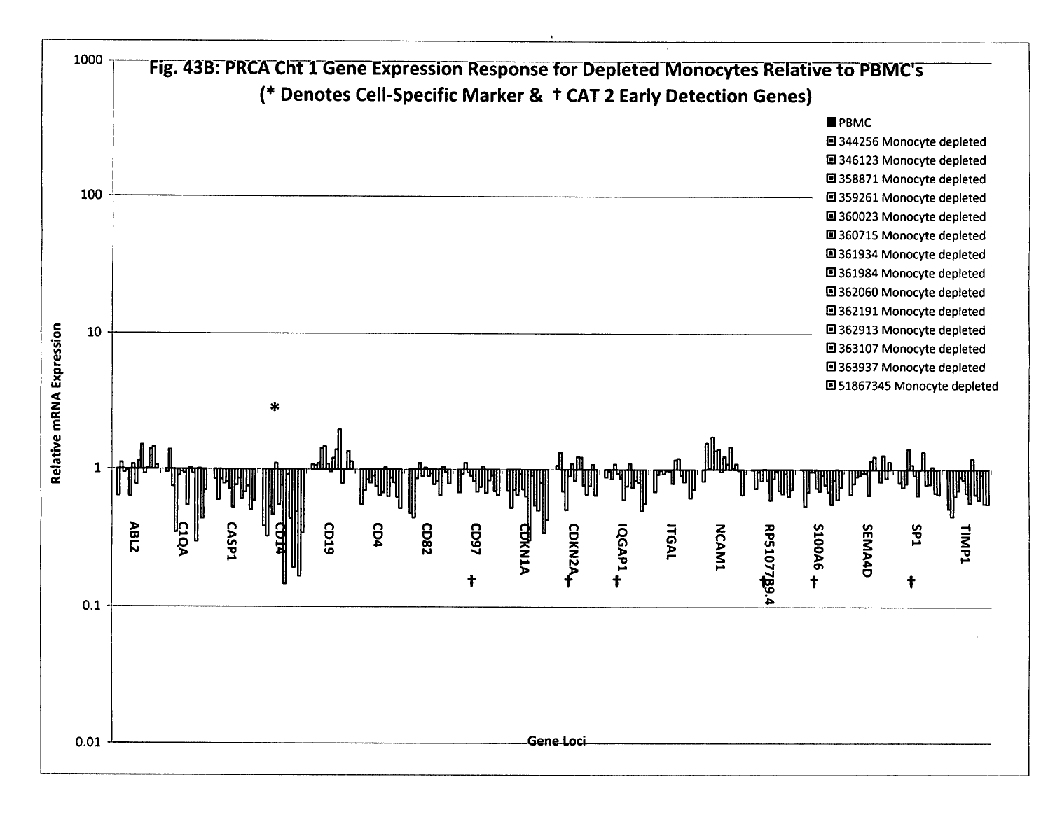

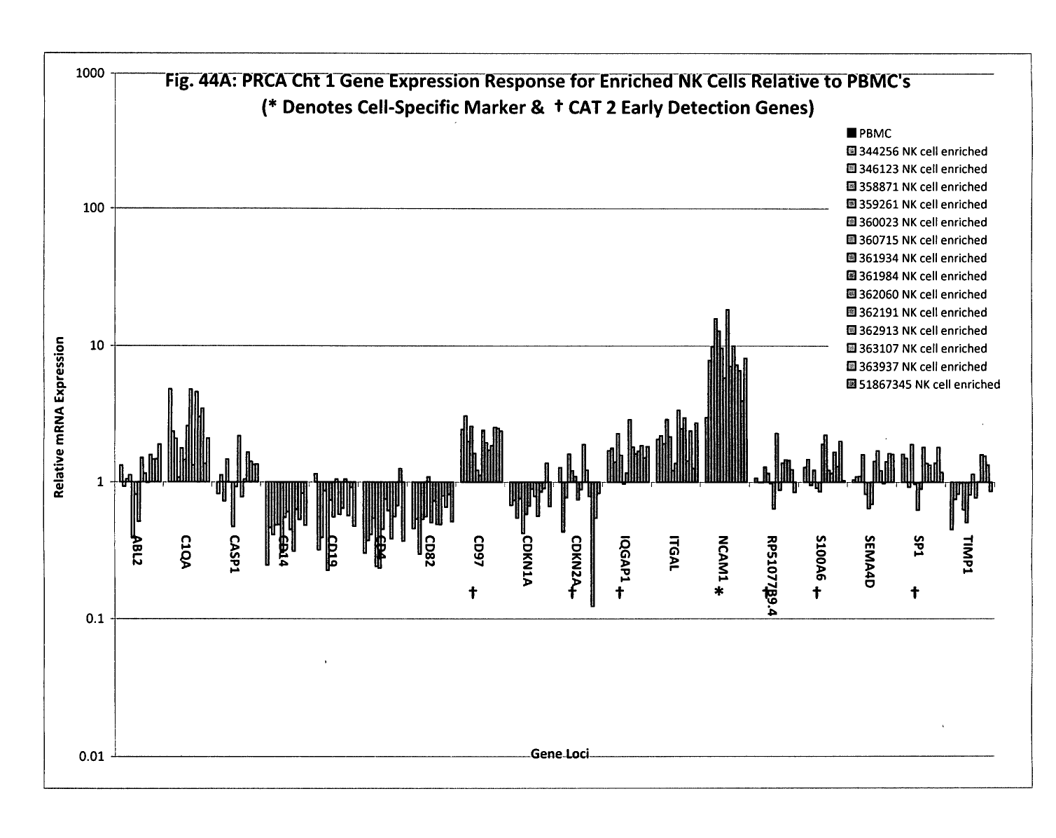

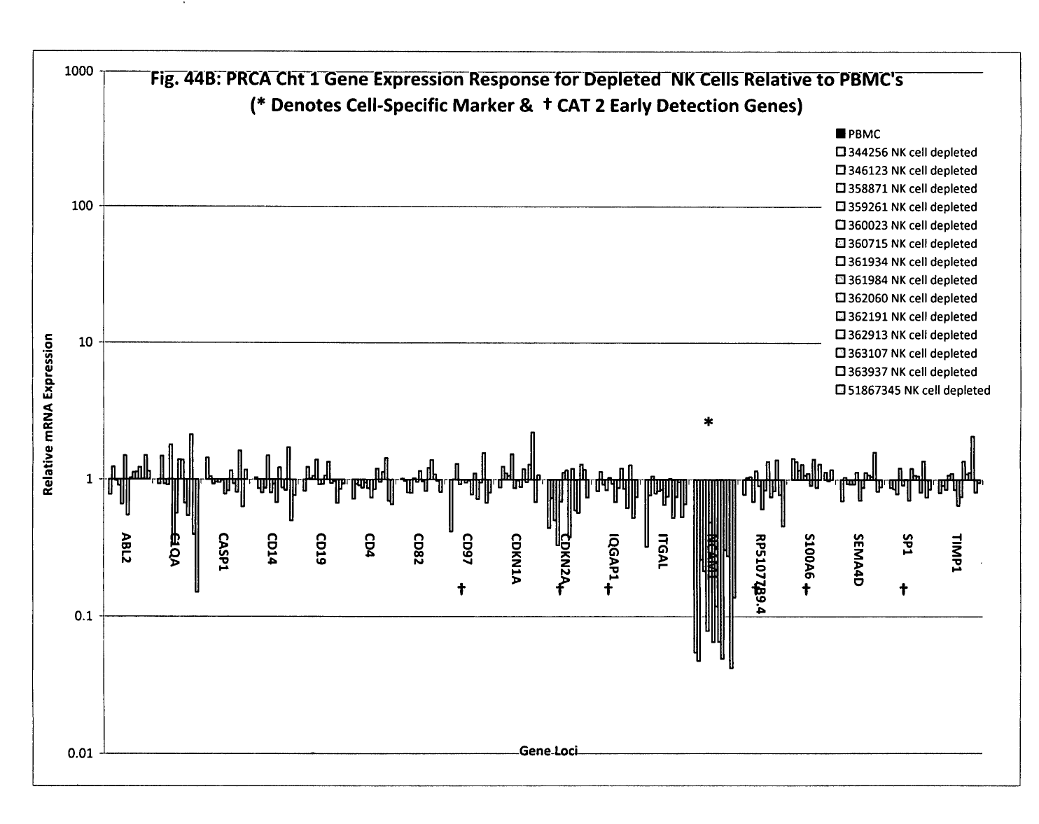

Five of the early detection model genes (CD97, IQGAP1, RP51077B9.4, S100A6 and SP1) had an increased expression in enriched monocytes (FIG. 43A) and corresponding slight decrease in expression in the depleted fraction (FIG. 43B).

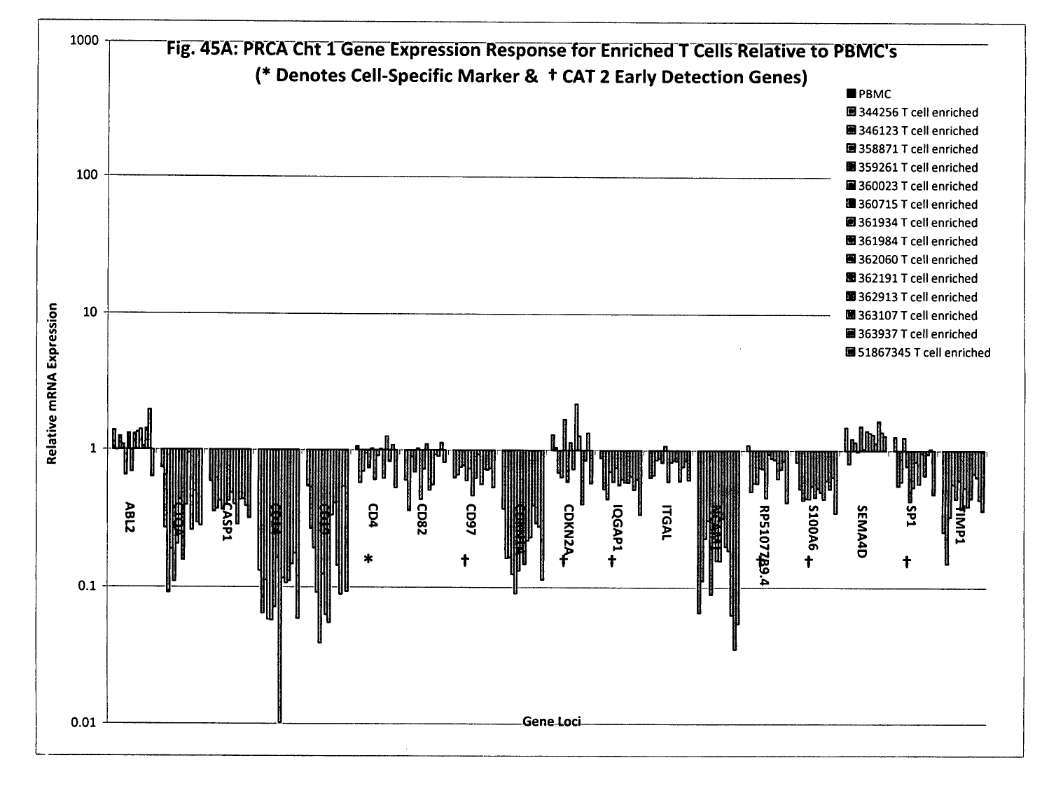

Three early detection model genes (CD97, IQGAP1 and SP1) had similar levels of increased in expression in NK cells as those observed in enriched monocytes (FIG. 44A). Two genes model genes, S100A6 and RP51077B9.4 also showed a slight increased expression in enriched NK cells, though of a much lesser magnitude than that observed in monocytes.

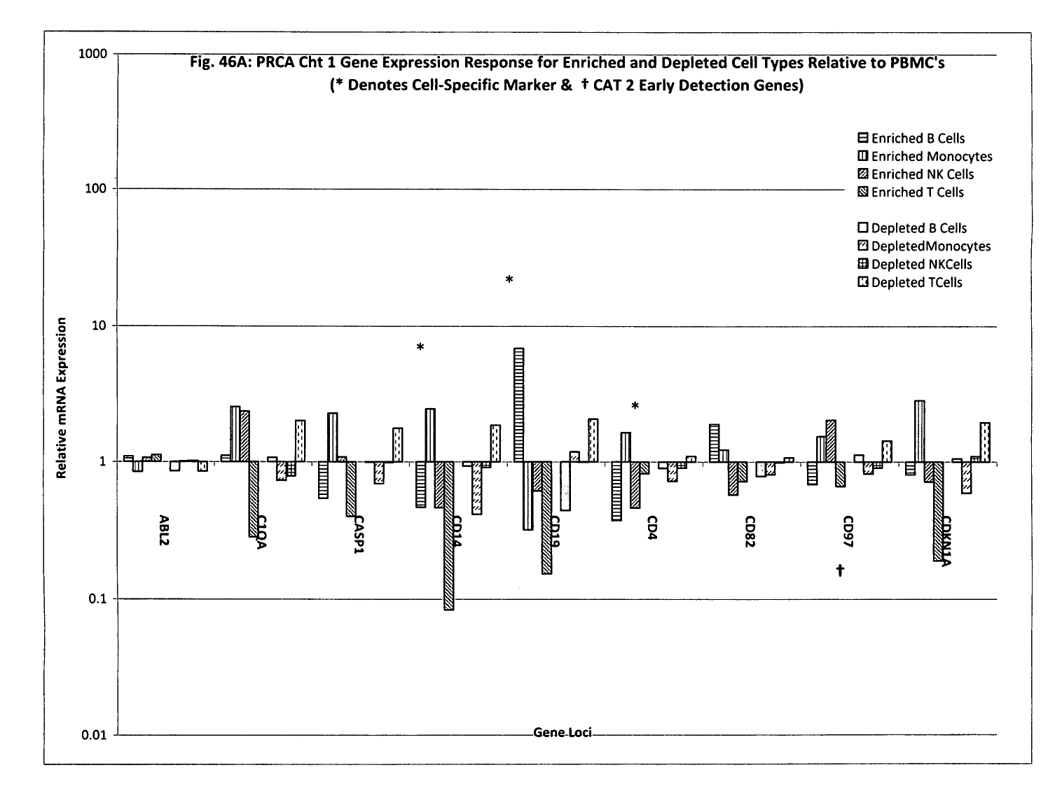

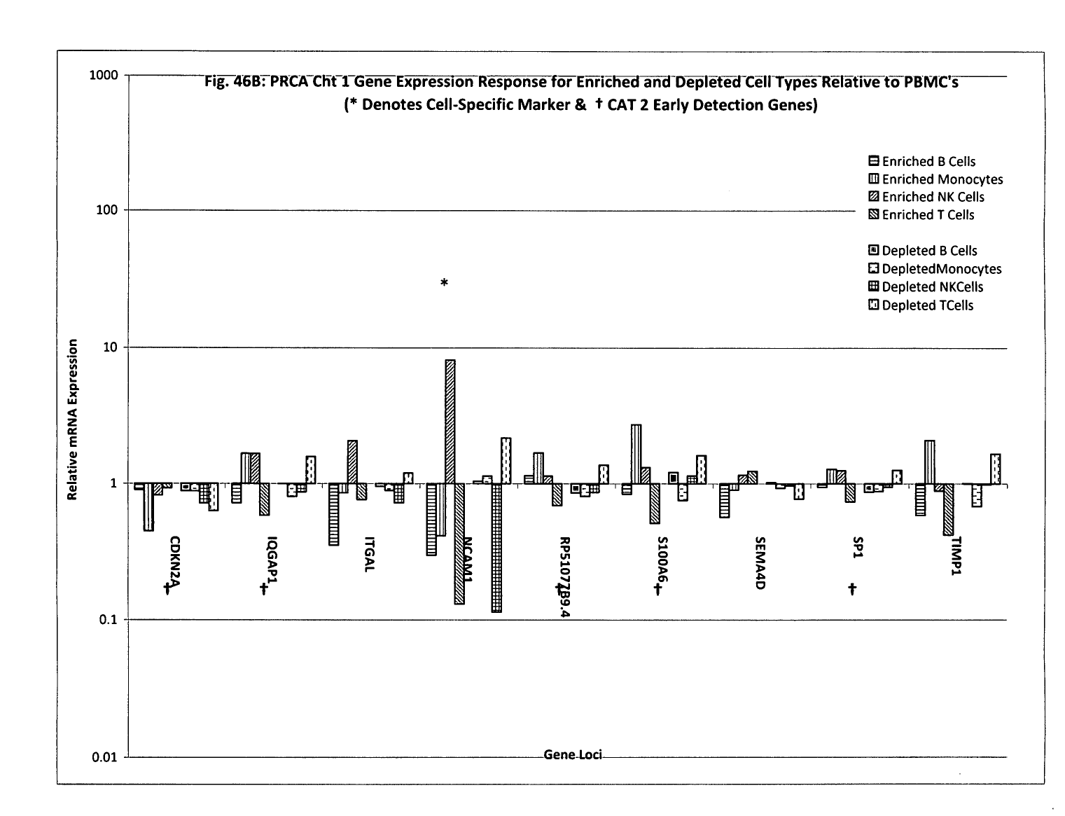

2. Averaged relative expression values, calculated for each of the 18 genes analyzed from all fourteen prostate cancer Cohort 1 patient samples in enriched and depleted cell fractions, are presented in Table 12. A graphical representation of the data is shown in FIGS. 46A & 46B.
Key observations included the following:

A differential pattern of expression across the four enriched cell types can be observed in a heat map of the averaged relative expression values for each of the 18 genes analyzed (Table 12), indicating that some genes are more highly expressed in specific cell types upon enrichment from PBMC's. Not unexpectedly, cell specific marker genes exhibited a greatly increased expression in their enriched, cell specific fraction and a concomitant decrease in expression is observed in enriched, non-specific cell fractions. For example, the B cell marker CD19 is induced 6.88-fold in enriched B cells and has a decreased expression in enriched monocytes, NK cells and T cells (0.32-fold, 0.62-fold and 0.08-fold, respectively).

Many genes other than cell-specific markers also exhibited an increased expression in only one enriched cell fraction, potentially indicating that these genes may be preferentially or more highly expressed, in one specific cell type. For example, the genes CASP1, CDKN1A and TIMP1 showed a 2.27, 2.84 and 2.09-fold increase in expression in enriched monocytes, respectively and a decrease in expression in the three other enriched cell types, possibly indicating that monocytes may be responsible for the majority of expression observed for these genes in whole blood (Table 12 and FIGS. 46A & 46B).

A few genes also exhibited an increased expression in multiple enriched fractions, indicating that expression in whole blood originates from multiple cell types. C1QA, CD97 and IQGAP1 are examples of such genes as all are induced in enriched monocytes and NK cells (Table 12 and FIGS. 46A & 46B).

The majority of genes analyzed exhibited an increased expression in enriched monocytes (C1QA, CASP1, CD4, CD82, CD97, CDKN1A, IQGAP1, RP51077B9.4, S100A6, SP1, TIMP1, and CD14), while fewer genes exhibited increased expression in enriched B cells (ABL2, C1QA, CD82, RP51077B9.4 and CD19), NK cells (C1QA, CD97, IQGAP1, ITGAL, S100A6, SEMA4D and NCAM1) and T cells (ABL2, CDKN2A and SEMA4D) (Table 12 and FIGS. 46A & 46B).

Gene Expression Analysis of Fractionated Cell Samples from Medically Defined Normal (MDNO) Whole Blood Samples

A quantitative comparison of gene expression levels between fractionated cell samples was also conducted from fourteen medically defined normal (MDNO) subjects using the same panel of 18 target genes.

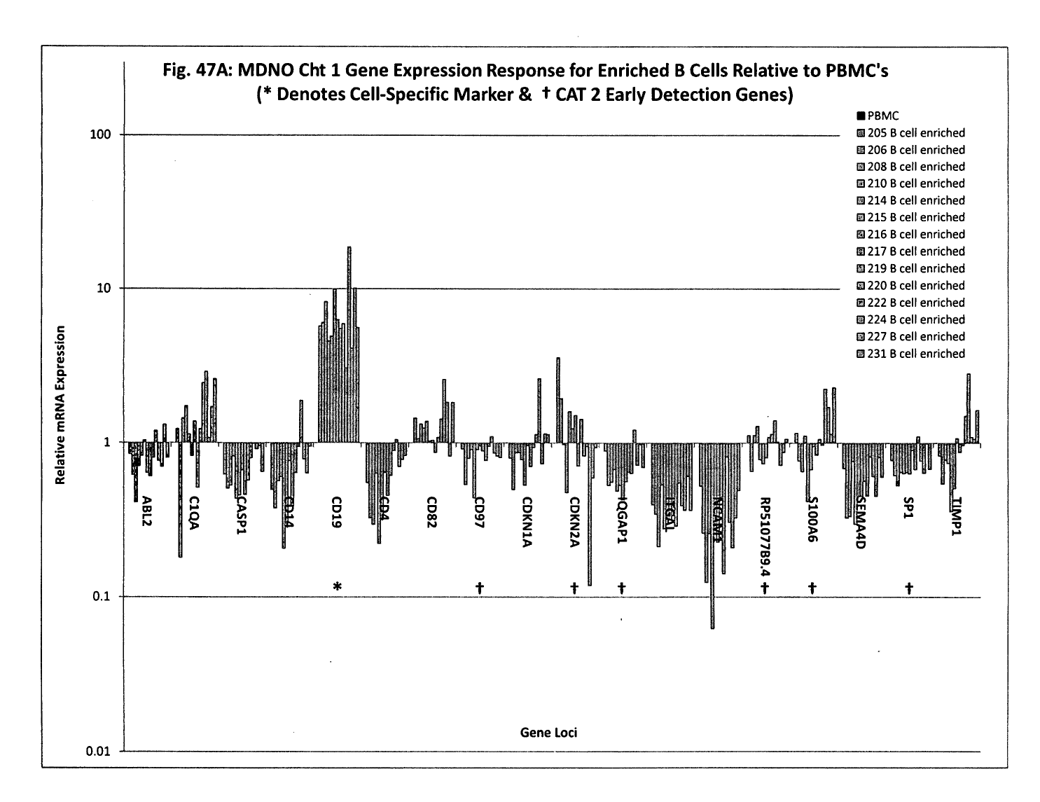

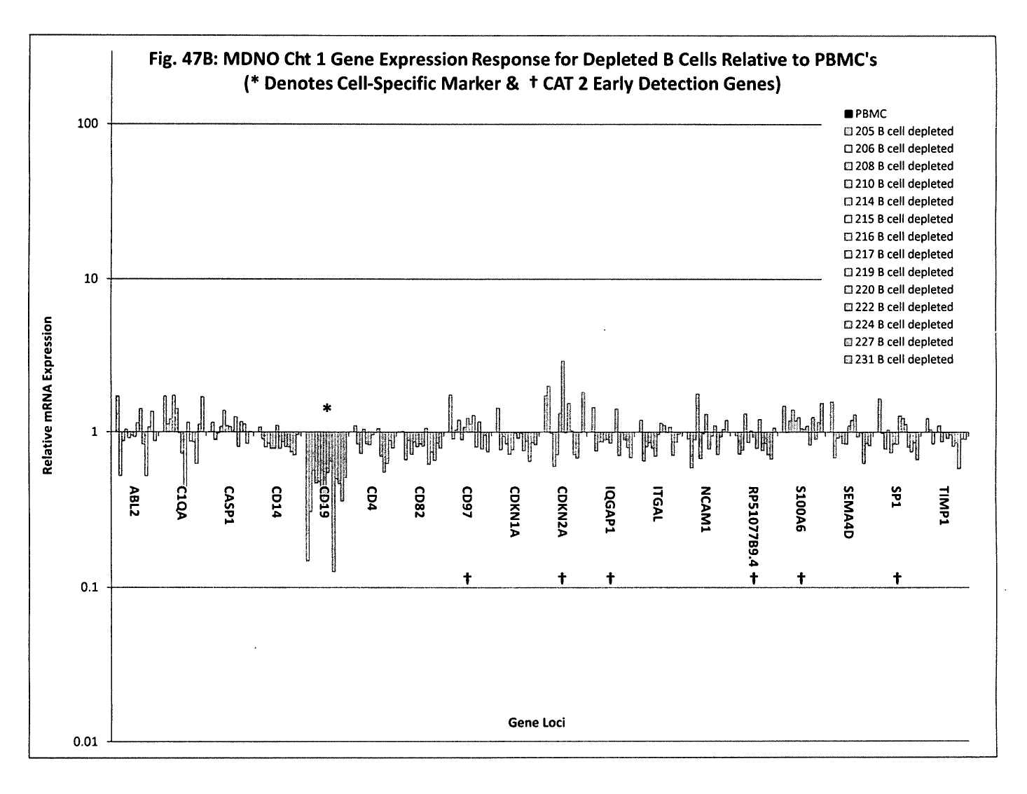

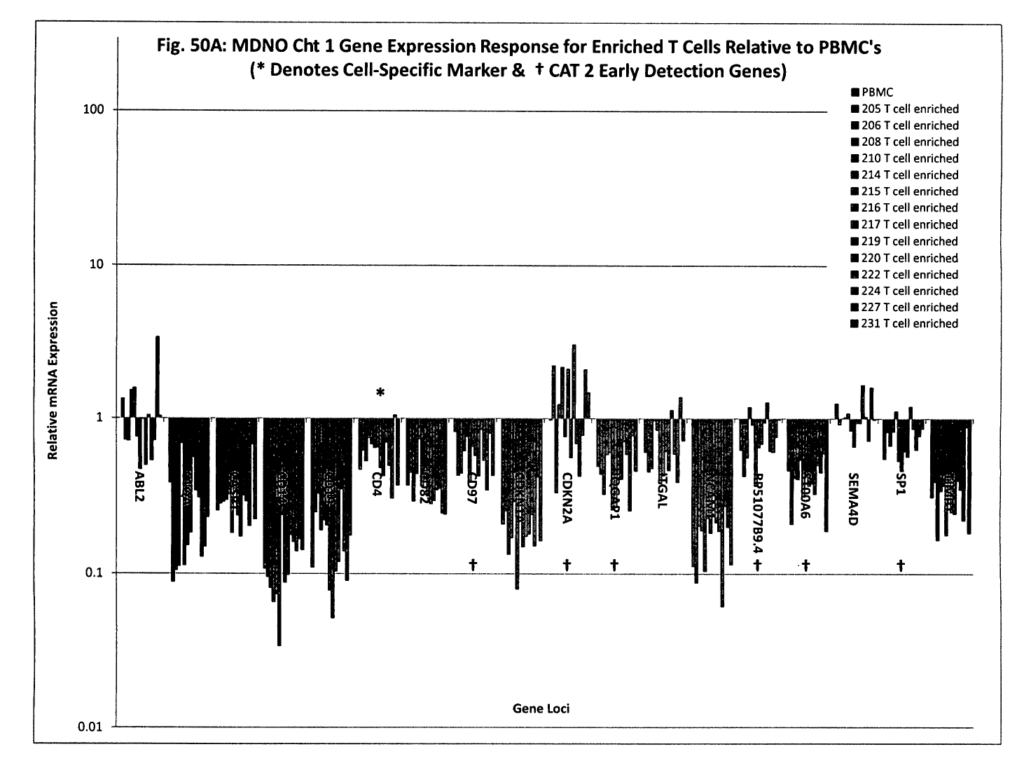

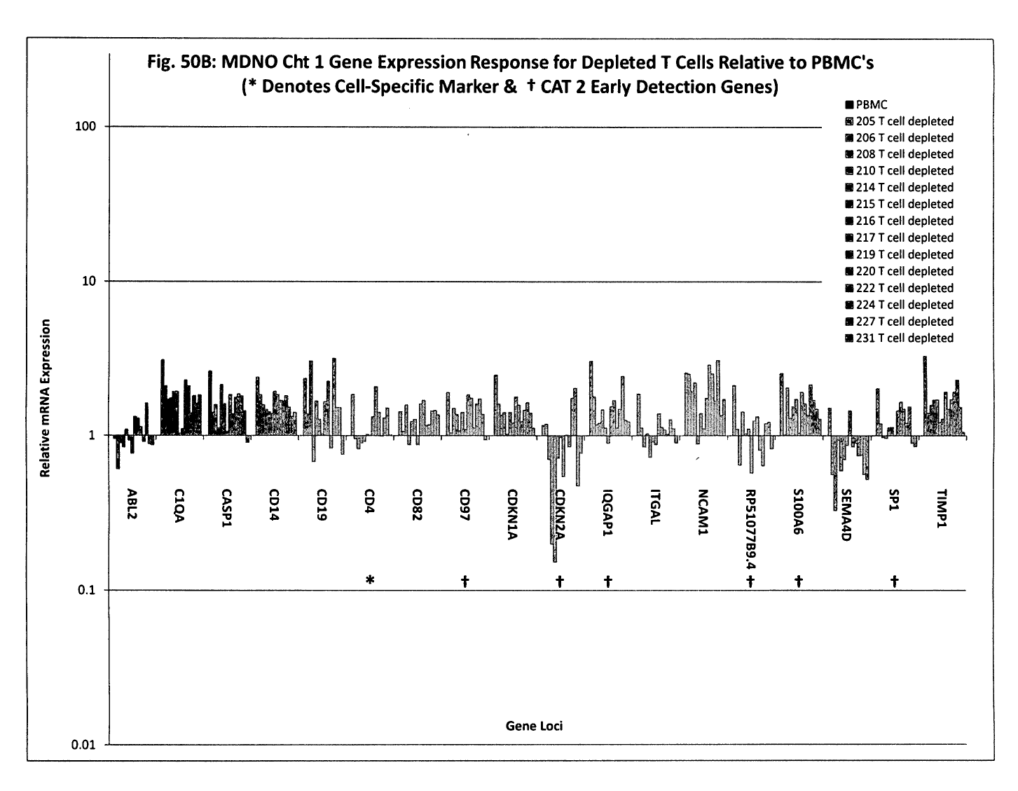

1. Expression levels of genes in enriched and depleted cell fractions were compared to expression in the original PBMC fraction for all medically defined Normal subjects. A graphical representation of the relative gene expression response for individual MDNO subjects in both enriched and depleted cells is presented in FIGS. 47A & 47B through FIGS. 50A & 50B. Note: all “A” figures show the response in enriched fractions and “B” figures the depleted fractions. Many of the same findings as in the PRCA Cohort 1 patient sample analysis were observed. Key observations included:

The gene expression profile was very similar between the 14 MDNO patient samples for the majority of genes in specific cell fractions, indicating a consistency in cell-specific gene expression across individuals.

The magnitude of response was slightly variable between individual subject samples.

Genes showing an induction in enriched cell fractions, had a corresponding decrease in expression in the depleted cell fraction for the same cell type.

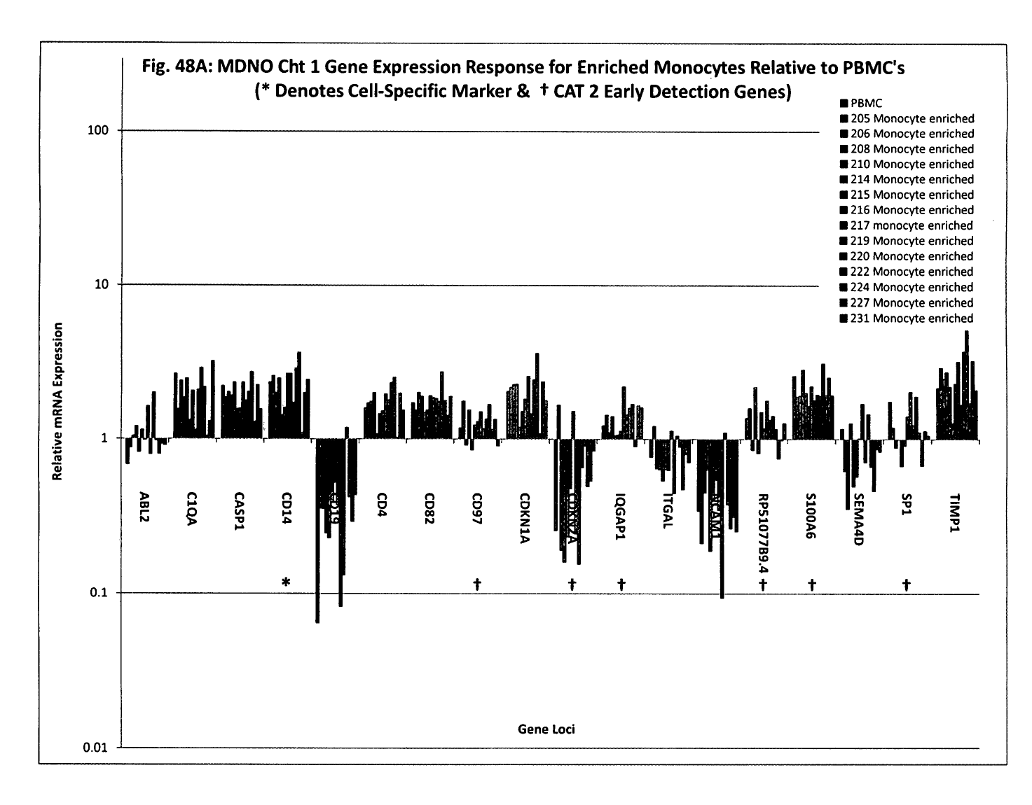

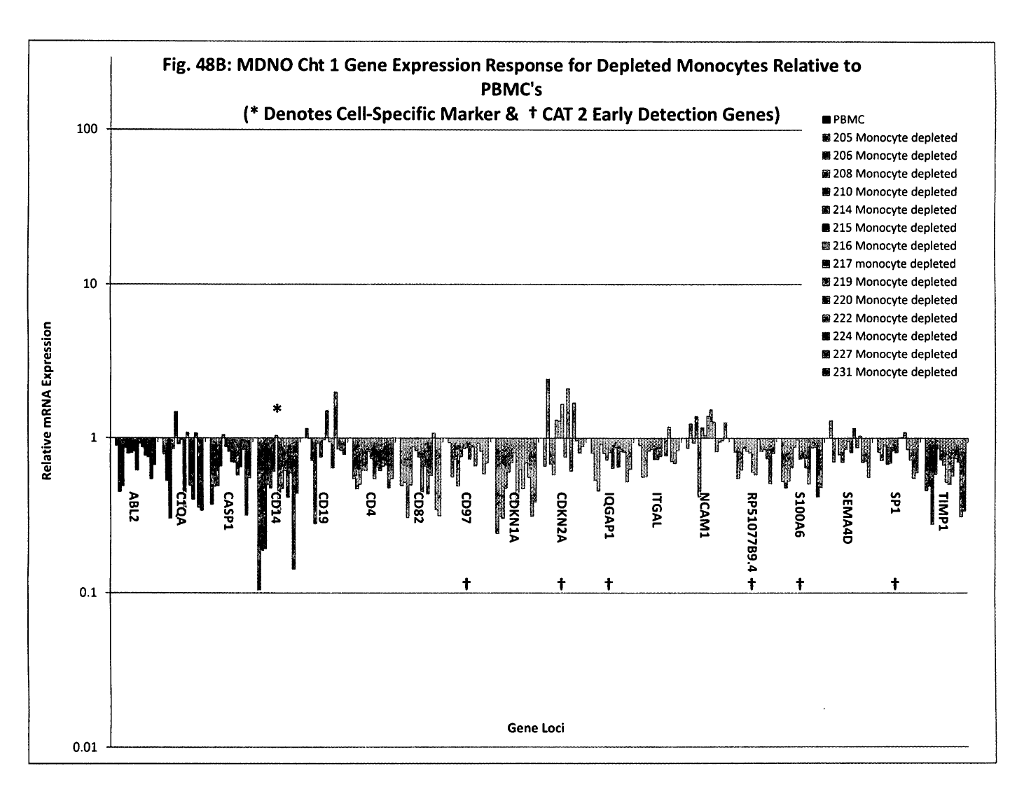

Five of the early detection model genes (CD97, IQGAP1 and RP51077B9.4, S100A6 and SP1) had an increased expression in enriched monocytes (FIG. 48A) and corresponding decreased expression in the depleted fraction (FIG. 48B).

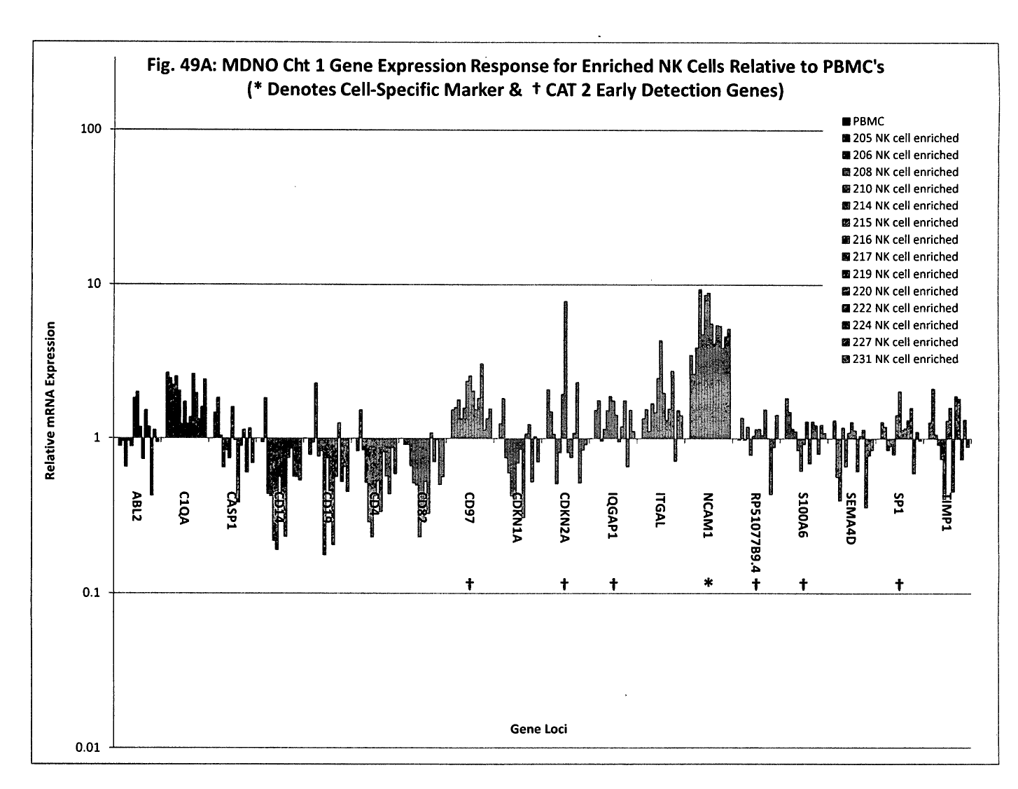

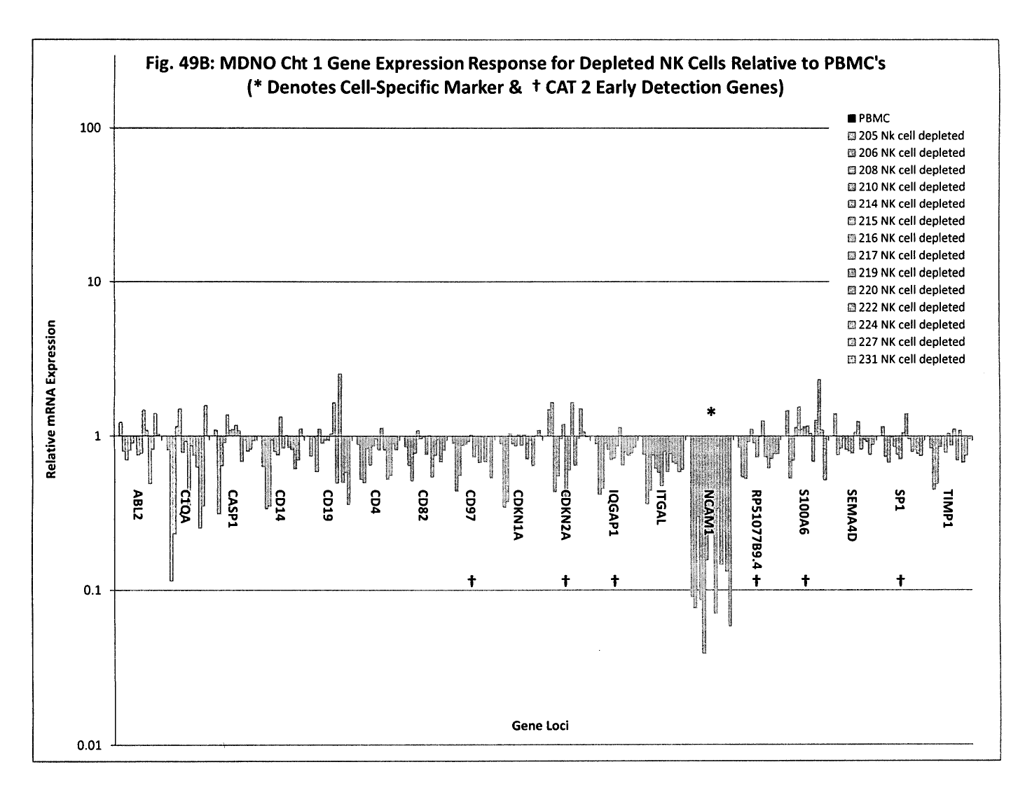

Three early detection model genes (CD97, IQGAP1 and SP1) had similar levels of increased in expression in NK cells as those observed in enriched monocytes (FIG. 49A).

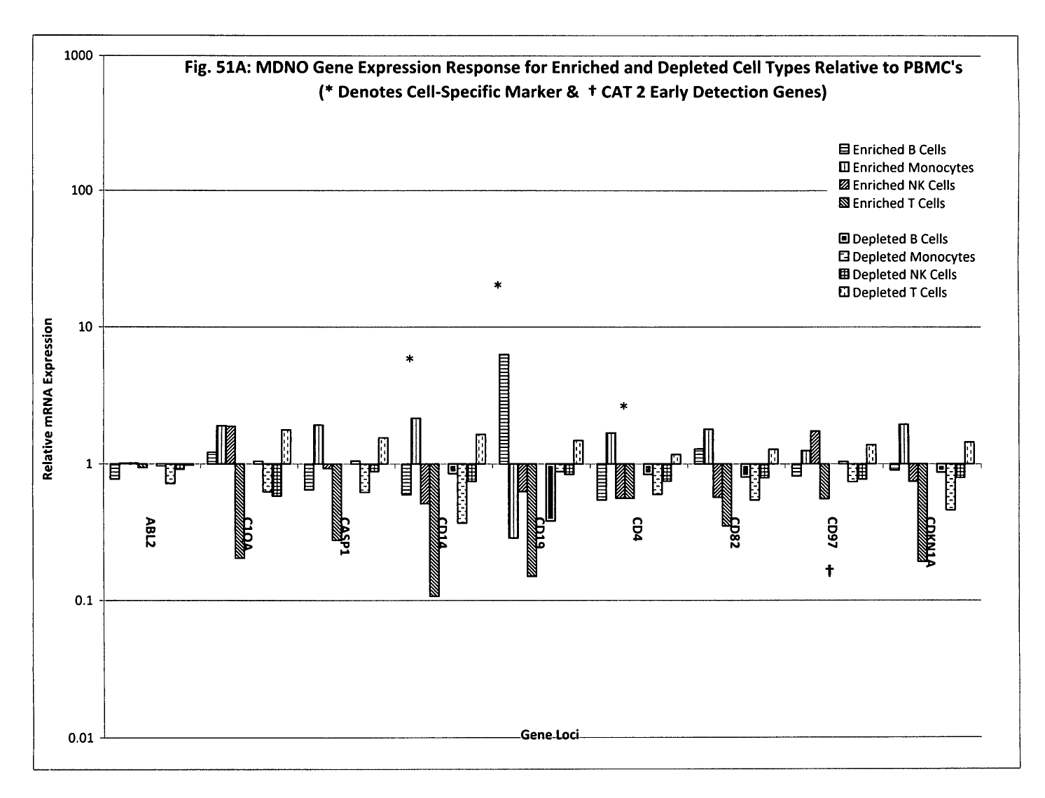

2. Averaged relative expression values, calculated for each of the 18 genes analyzed from all fourteen medically defined normal (MDNO) patient samples in enriched and depleted cell fractions, are presented in Table 13. A graphical representation of the data is shown in FIGS. 51A & 51B. Key Observations included:

A differential pattern of expression across the four enriched cell types can be observed in a heat map of the averaged relative expression values for each of the 18 genes analyzed (Table 13).

Many genes other than cell-specific markers also exhibited an increased expression in only one enriched cell fraction, potentially indicating that these genes may be preferentially expressed in one specific cell type. For example, the genes CASP1 and CDKN1A showed a 1.93 and 1.96-fold increase in expression in enriched monocytes, respectively and a decrease in expression in the three other enriched cell types, possibly indicating that monocytes may be responsible for the majority of expression observed for these genes in whole blood (Table 13 and FIG. 51A).

A few genes also exhibited an increased expression in multiple enriched fractions, indicating that expression in whole blood originates from multiple cell types. C1QA, CD97 and IQGAP1 are again examples of such genes as both are induced in enriched monocytes and NK cells (Table 13 and FIGS. 51A & 51B).

The majority of genes analyzed exhibited an increased expression in enriched monocytes (C1QA, CASP1, CD4, CD82, CD97, CDKN1A, IQGAP1, RP51077B9.4, S100A6, SP1, TIMP1, and CD14), while fewer genes exhibited increased expression in enriched B cells (C1QA, CD82 and CD19), NK cells (C1QA, CD97, CDKN2A, IQGAP1, ITGAL, and NCAM1) and T cells (CDKN2A) (Table 13 and FIGS. 51A & 51B).

### Comparative Gene Expression Analysis Between PRCA (Cohort 1) and MDNO Subjects

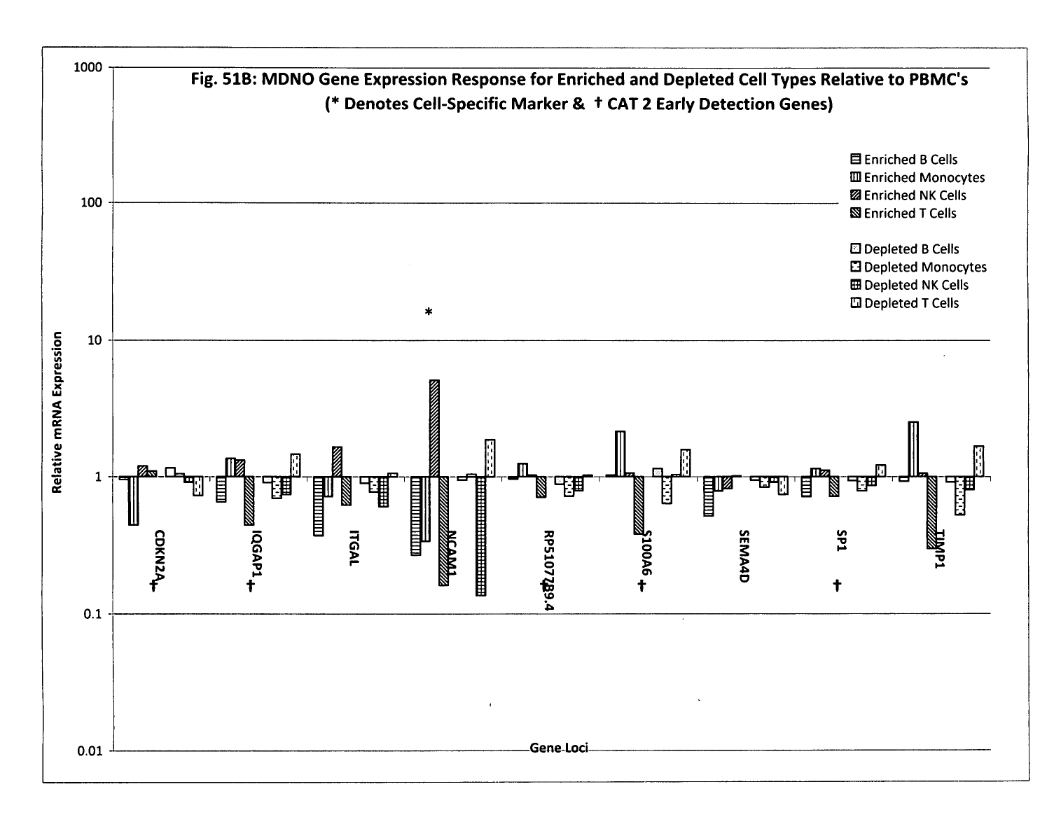

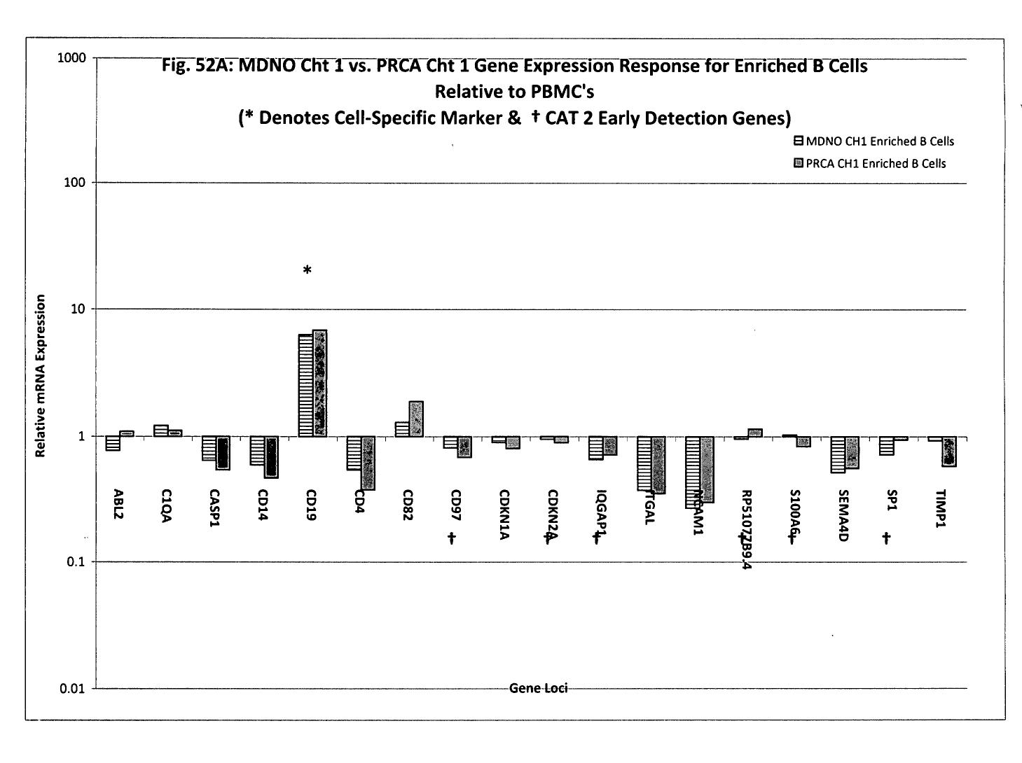

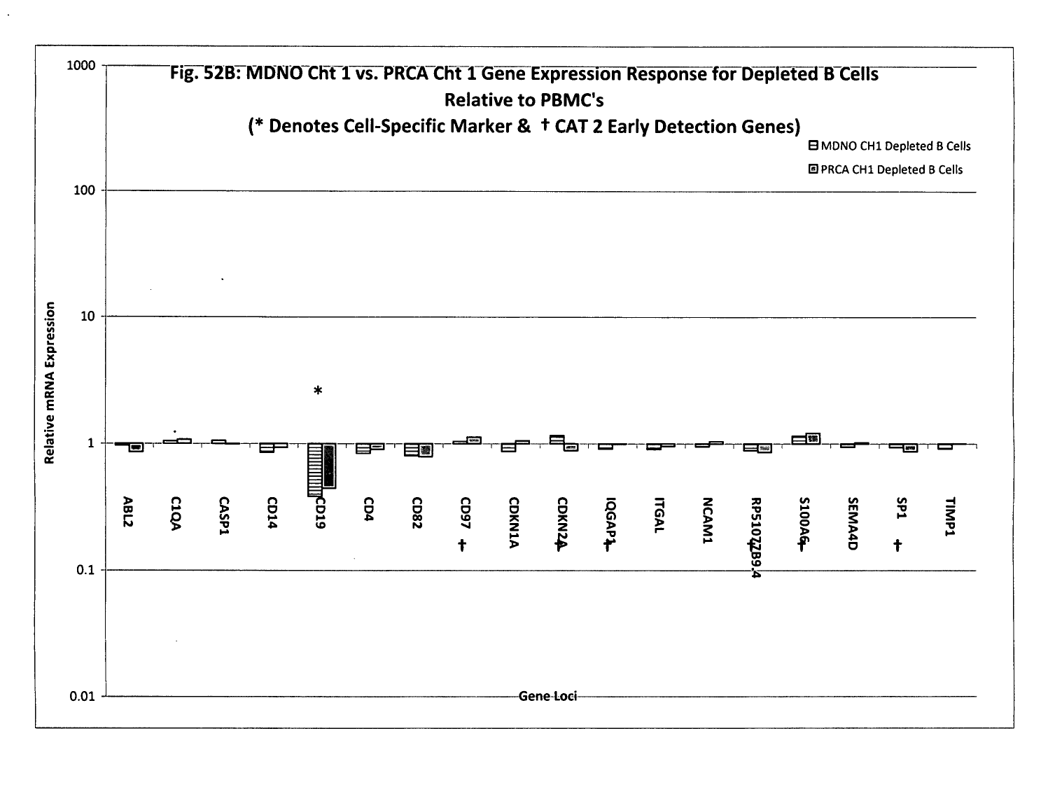

A quantitative comparison of the gene expression levels (relative to respective PBMC's) was made between the 14 prostate cancer (PRCA) and the 14 medically defined normal (MDNO) subjects in all fractionated cell samples. The averaged relative expression values for enriched and depleted cell fractions from both patient cohorts (prostate cancer and normals) are presented in Table 3. The graphical representation of this data is shown in FIGS. 52A and 52B through 55A and 55B. As previously noted, all “A” figures show the response in enriched fractions and all “B” figures show the depleted fractions.

1. A comparison of the gene expression profiles between disease and normal subjects revealed a strong similarity in expression pattern for all enriched cell types, though a small number of genes do exhibit slight differences in the magnitude of expression in certain enriched fractions between the two subject cohorts: prostate cancer (PRCA) and medically defined normals (MDNO). Genes having potentially different magnitudes of expression in enriched fractions included the following:

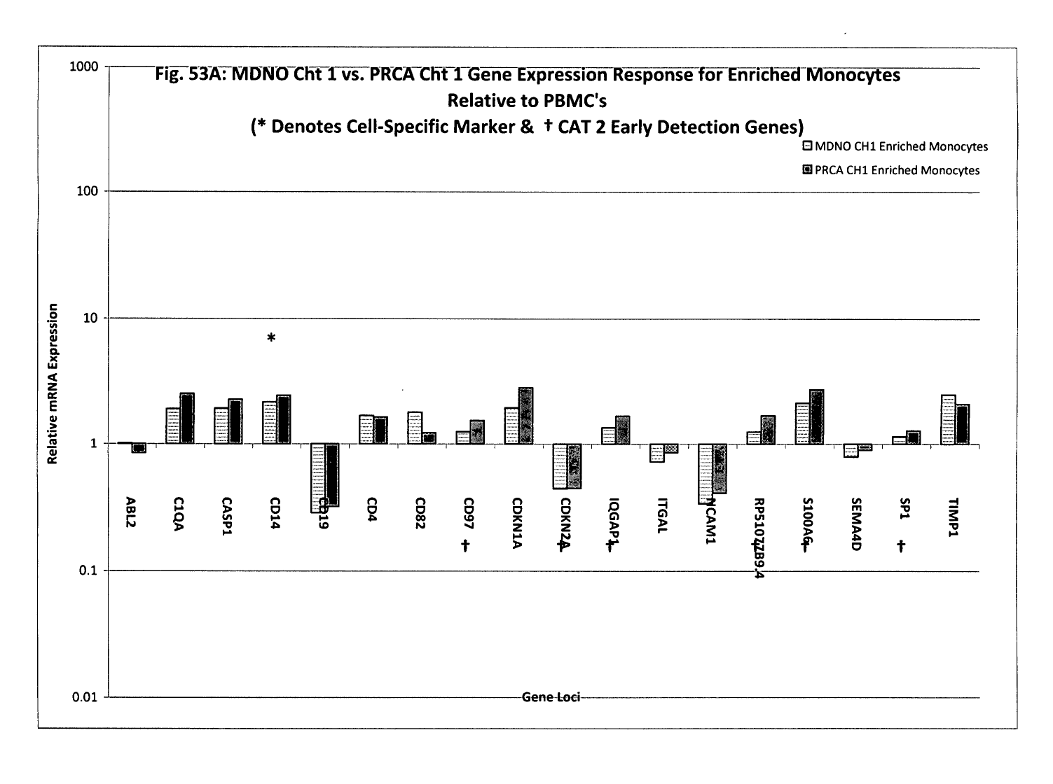

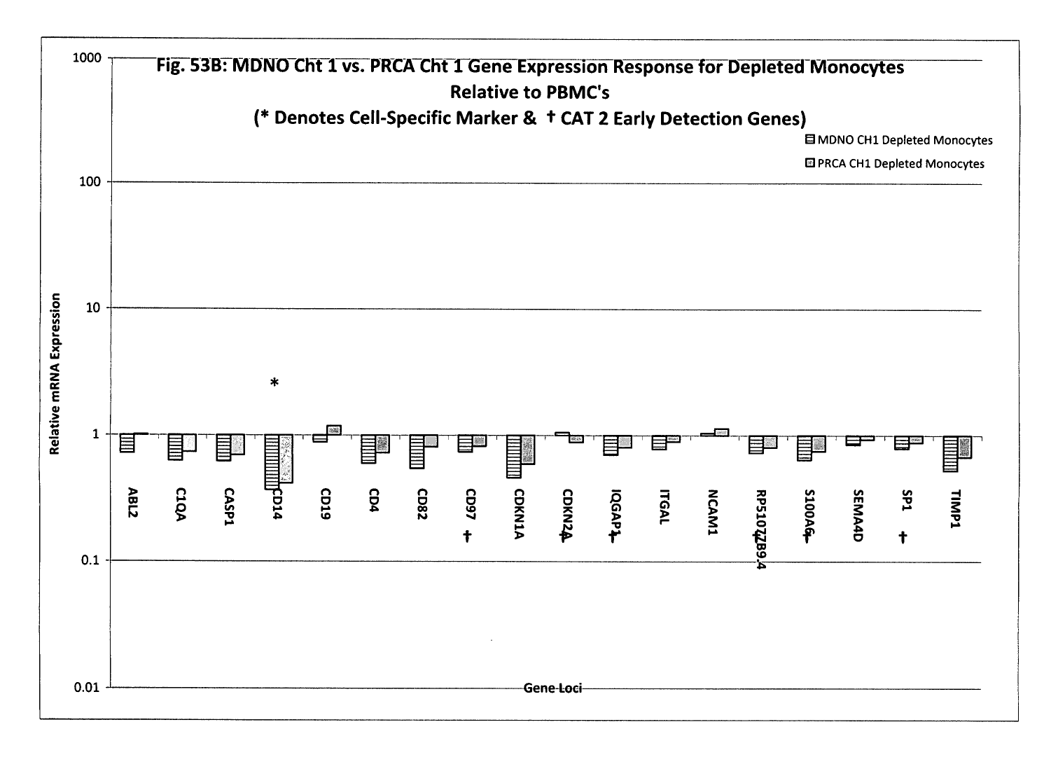

S100A6 has an average 2.74-fold increased expression in enriched monocytes from prostate cancer patients compared to a 2.13-fold increase in expression in enriched monocytes from normal subjects (Table 14 and FIG. 53A).

CDKN1A had an average 2.84-fold increased expression in enriched monocytes from prostate cancer patients compared to a 1.96-fold increase in expression in enriched monocytes from normal subjects (Table 14 and FIG. 53A).

C1QA had an average 2.53-fold increased expression in enriched monocytes from prostate cancer patients compared to a 1.91-fold increase in expression in enriched monocytes from normal subjects (Table 14 and FIG. 53A).

TIMP1 had an average 2.09-fold increased expression in enriched monocytes from prostate cancer patients compared to a 2.49-fold increase in expression in enriched monocytes from normal subjects (Table 14 and FIG. 53A).

CD82 had an average 1.23-fold increased expression in enriched monocytes from prostate cancer patients compared to a 1.80-fold increase in expression in enriched monocytes from normal subjects (Table 14 and FIG. 53A).

CD82 had an average 1.89-fold increased expression in enriched B cells from prostate cancer patients compared to a 1.29-fold increase in expression in enriched B cells from normal subjects (Table 14 and FIG. 52A). This profile is the opposite of that observed in monocytes, in which the PRCA cohort exhibited a smaller increase in the magnitude of expression compared with the MDNO cohort.

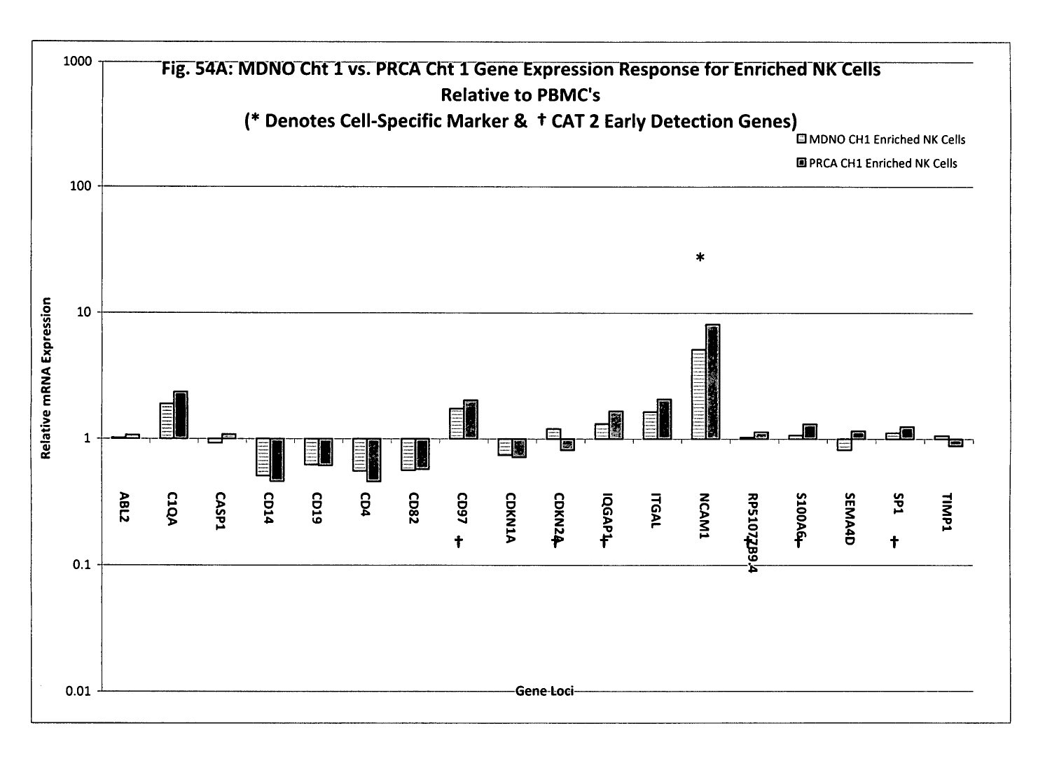

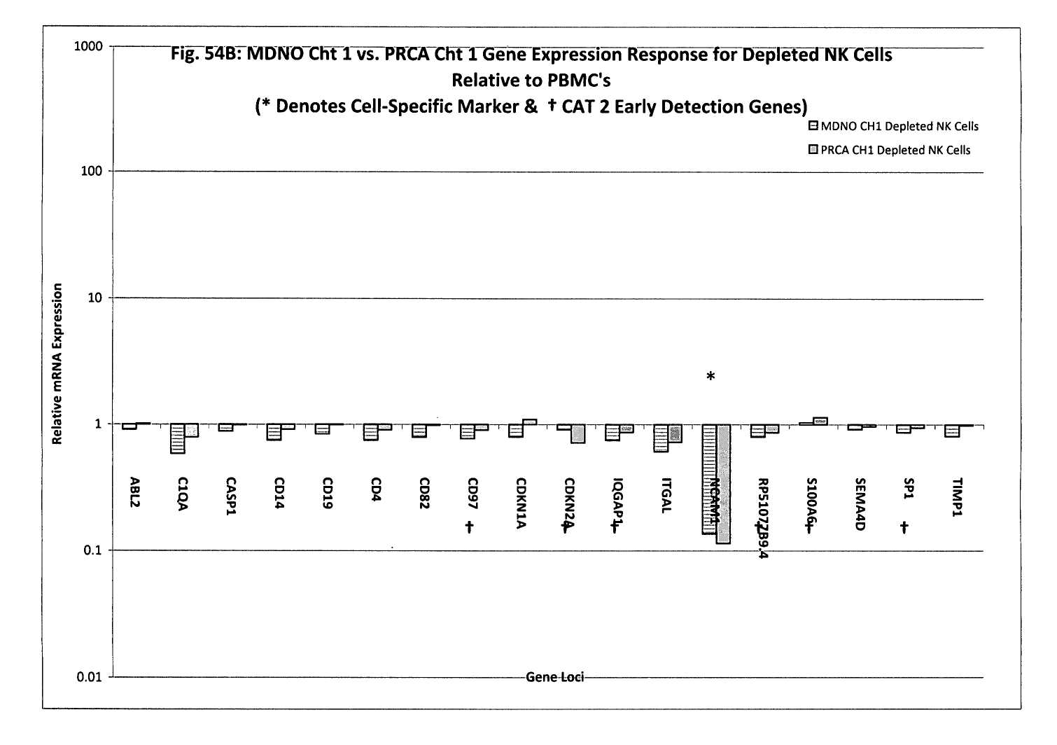

CDKN2A had an average 0.82-fold decreased expression in enriched NK cells from prostate cancer patients compared to a 1.20-fold increase in expression in enriched NK cells from normal subjects (Table 14 and FIG. 54A).

RP51077B9.4 had an average 1.69-fold increased expression in enriched monocytes from prostate cancer patients compared to a 1.25-fold increase in expression in enriched monocytes from normal subjects (Table 14 and FIG. 53A)

The differences in the magnitude of expression observed for the genes listed above indicate a difference in the number of RNA transcripts present in the enriched cell fraction, relative to the PBMC starting material, in prostate cancer compared to Normal subjects. These differences may in be part responsible for the differential expression observed in whole blood. One important caveat for the analysis of the cell fractionation data is the verification that the fold-enrichment for all cell fractions in prostate and normal samples is extremely similar.

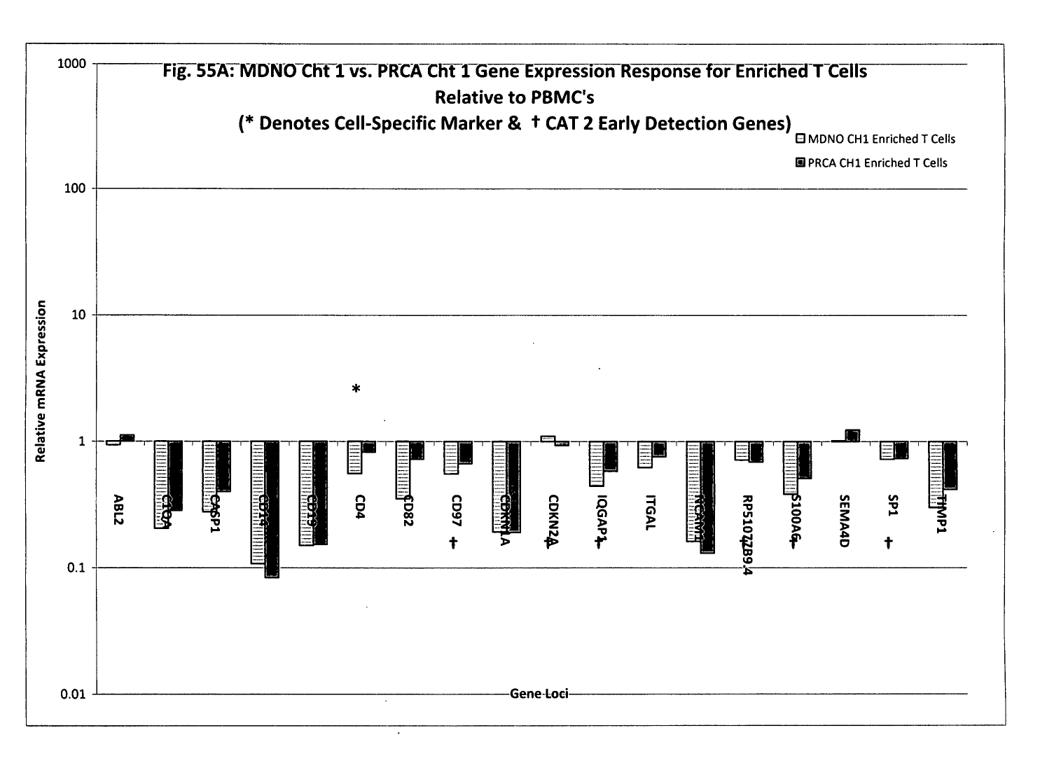

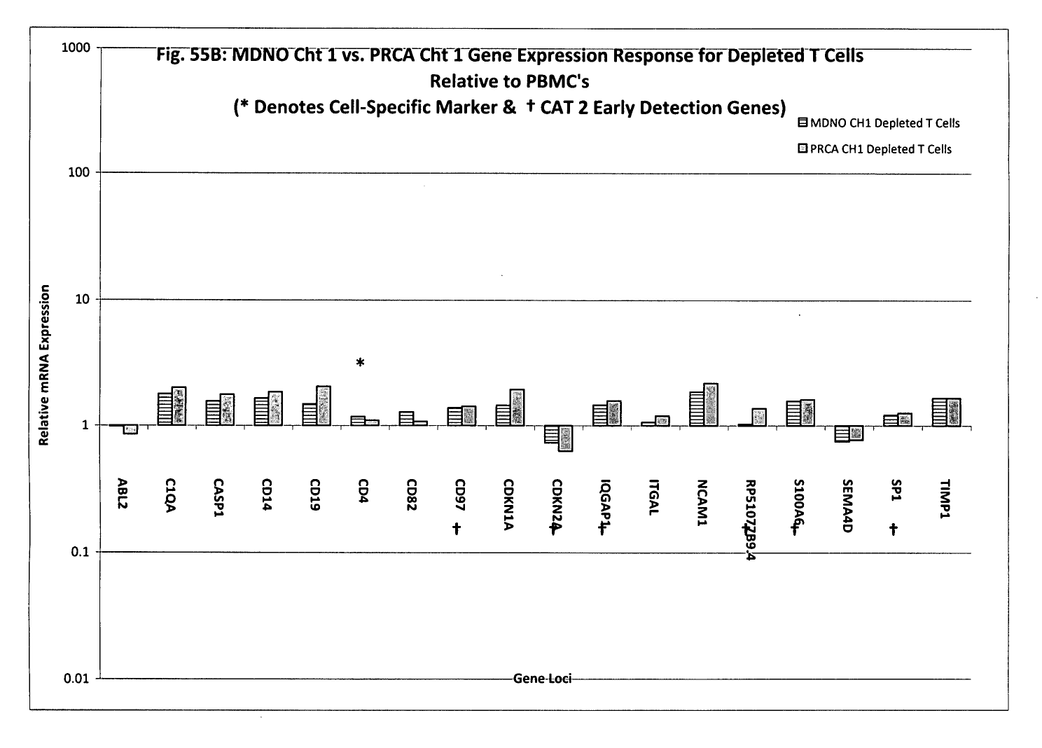

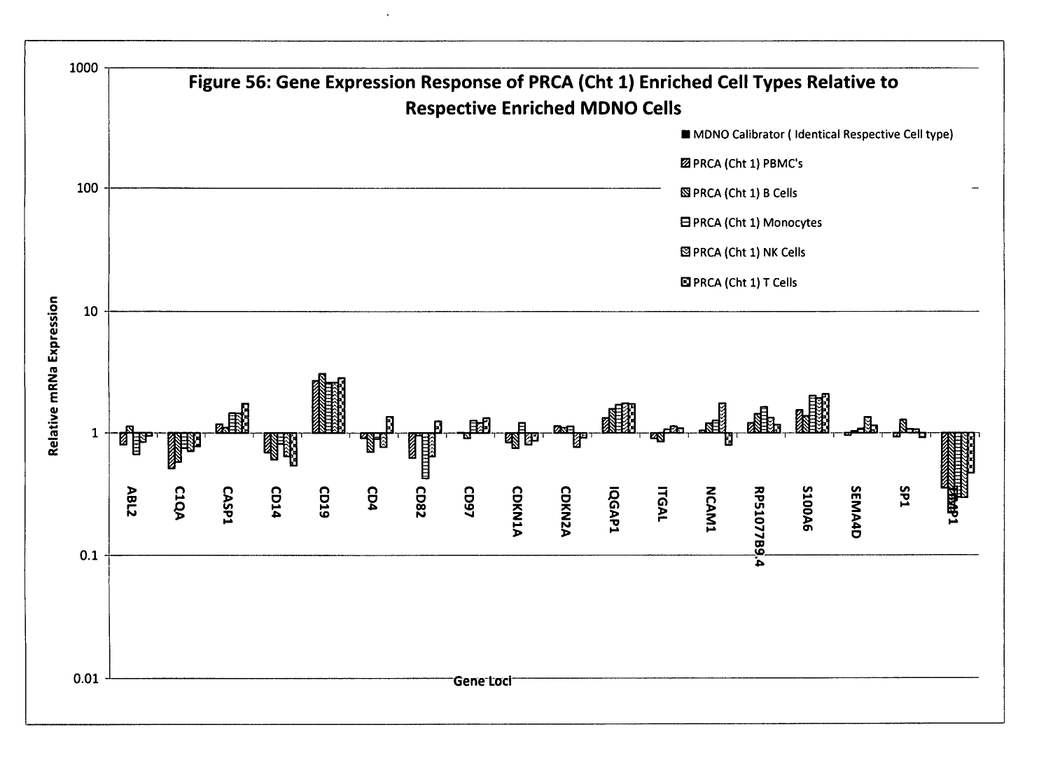

It is also of interest to note gene expression differences that may be present in both the starting PBMC's and enriched cellular fractions between subject cohorts themselves (rather than as a relative comparison to respective cohort PBMC's). The averaged gene expression response of prostate cancer subjects relative to MDNO subjects for all cell types, is presented in FIG. 56. From this analysis, multiple genes show a significant difference in the magnitude of expression between subject cohorts for many of the enriched cell types including the PBMC starting material. Two of these genes, CD82 and TIMP1 had already been identified as potentially having a differential expression (decrease) between the prostate cancer subject cohort compared with the MDNO cohort (FIG. 56). In contrast, an increased expression was observed for IQGAP1, RP51077B9.4 and S100A6 in the prostate cancer cohort. Interestingly, the B cell marker, CD19, had an increased expression in PRCA subject samples relative to the MDNO cohort samples.

In summary, the six prostate cancer early detection model genes have been shown to be preferentially expressed in three different enriched cell fractions in both prostate cancer and normal subjects. CD97, IQGAP1 and SP1 show an increased expression in enriched monocytes and NK cells. RP51077B9.4 and S100A6 have a significantly increased expression in enriched monocytes and CDKN2A shows a slight increase in expression in enriched T cells (and a corresponding decreased expression in the depleted T cell fraction). A slight enrichment of CDKN2A expression in NK cells was observed in the normal patient cohort, though interestingly not in the prostate cancer cohort. Though the genes are expressed in the same enriched cell type in the two different patient cohorts, the magnitude of expression is somewhat different for some of the early detection model genes.

### Example 12

### Discrimination of Prostate Cancer Subjects from Healthy, Normal Subjects (without BPH) and Discrimination of Prostate Cancer Subjects from Healthy, Subjects with Benign Prostatic Hyperplasia (BPH) Using RNA Transcript-Based Gene Expression: Validation Using Test Dataset

Validation of the 2-, 4- and 6-gene models shown in Table 4, which are capable of discriminating prostate cancer patients (CaP) from healthy normal subjects (referred to in this Example as Category 2 models) and the 1-, 2-, 3-, and 5-gene models, which are capable of discriminating CaP patients from subjects presenting with benign prostate hyperplasia (referred to in this Example as Category 3 models) is described herein.

All 9 Category 2 models described in Table 4, and 9 of the 14 Category 3 models described in Table 6, successfully validated according to the tests and procedures specified in the validation plan. These results further strengthen the inclusion of these models in a multi-site validation effort

### Validation Results

### Category 2 (CaP vs Normals) Model Validation Tests and Specifications

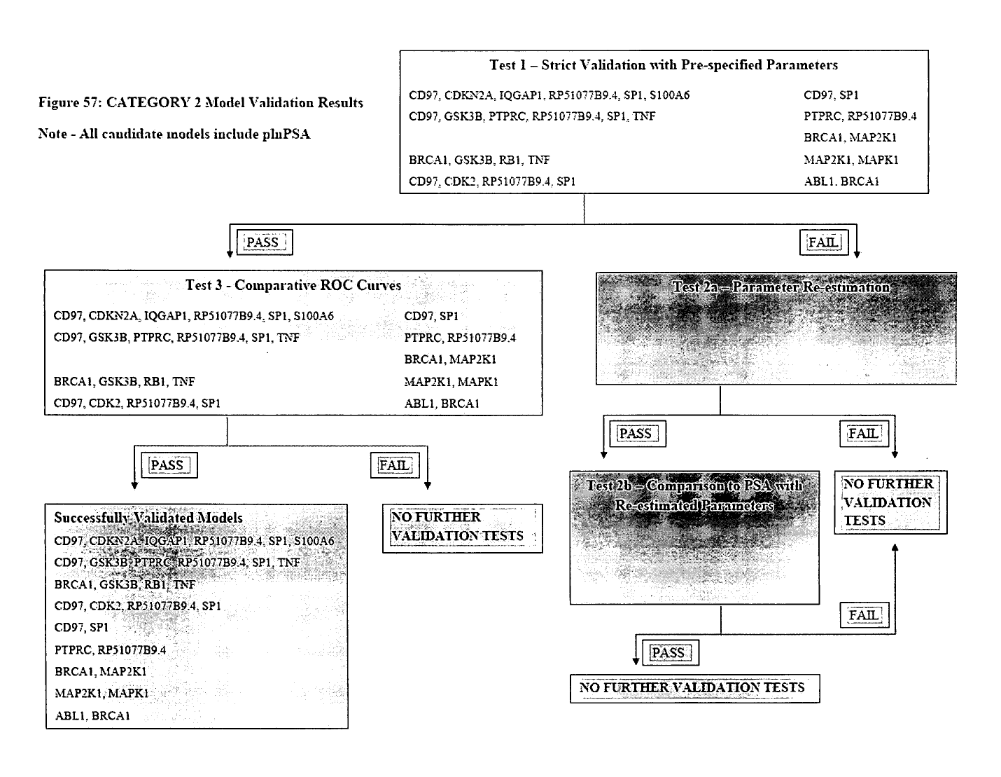

The five 2-gene models, two 4-gene models and two 6-gene models, all integrated with PSA, described in Table 4, were submitted for validation using the parameters established in the training set, as shown in Table 15. The following Tests (1-3) were performed as necessary on the test dataset as a formal validation of all candidate Category 2 models. The progression of testing and results (pass/fail) for all models is summarized and illustrated in the flowchart provided in FIG. 57 with further supporting details and results that follow.

### Test 1—Strict Tests Based on Training Dataset Model Parameters and Cut-Offs:

For each of the candidate models, the model logit score was computed using pre-specified coefficients (beta parameters) established in the training dataset (referred to in Table 15 below). A pre-specified logit cut-point of 0 for all models was applied to split the samples into two groups. Subjects with logit scores above the cut-point were predicted to be CaP patients and those whose scores fell below the cut-point were predicted to be healthy normal subjects as shown in Tables Va and Vb. The age-adjusted PSA criterion misclassified 39 of the 128 CaP patients and 6 of the 94 normal subjects in this test dataset, with comparable figures for the gene expression models provided in Table Va. Based on the total number misclassified and overall sensitivity and specificity, all gene expression models outperformed the age-adjusted PSA criterion.

TABLE Va
CaP vs Normals Frequency Counts by Model

| CATEGORY 2 Models (including plnPSA) | | | | | | Number Misclassified | | |
|---|---|---|---|---|---|---|---|---|
| Gene 1 | Gene 2 | Gene 3 | Gene 4 | Gene 5 | Gene 6 | CaP (N = 128) | Normals (N = 94) | Total |
| ABL1 | BRCA1 | | | | | 19 | 8 | 27 |
| MAP2K1 | MAPK1 | | | | | 24 | 12 | 36 |
| BRCA1 | MAP2K1 | | | | | 25 | 9 | 34 |
| PTPRC | RP51077B9.4 | | | | | 20 | 6 | 26 |
| CD97 | SP1 | | | | | 11 | 8 | 19 |
| CD97 | CDK2 | RP51077B9.4 | SP1 | | | 15 | 5 | 20 |
| BRCA1 | GSK3B | RB1 | TNF | | | 18 | 9 | 27 |
| CD97 | GSK3B | PTPRC | RP51077B9.4 | SP1 | TNF | 13 | 9 | 22 |
| CD97 | CDKN2A | IQGAP1 | RP51077B9.4 | SP1 | S100A6 | 17 | 6 | 23 |
| Age-adjusted PSA | | | | | | 39 | 6 | 45 |

TABLE Vb
Sensitivity and Specificity by Model

| CATEGORY 2 Models (including plnPSA) | | | | | | Classification | |
|---|---|---|---|---|---|---|---|
| Gene 1 | Gene 2 | Gene 3 | Gene 4 | Gene 5 | Gene 6 | Sensitivity | Specificity |
| ABL1 | BRCA1 | | | | | 85.2% | 91.5% |
| MAP2K1 | MAPK1 | | | | | 81.3% | 87.2% |
| BRCA1 | MAP2K1 | | | | | 80.5% | 90.4% |
| PTPRC | RP51077B9.4 | | | | | 84.4% | 93.6% |
| CD97 | SP1 | | | | | 91.4% | 91.5% |
| CD97 | CDK2 | RP51077B9.4 | SP1 | | | 88.3% | 94.7% |
| BRCA1 | GSK3B | RB1 | TNF | | | 85.9% | 90.4% |
| CD97 | GSK3B | PTPRC | RP51077B9.4 | SP1 | TNF | 89.8% | 90.4% |
| CD97 | CDKN2A | IQGAP1 | RP51077B9.4 | SP1 | S100A6 | 86.7% | 93.6% |
| Age-adjusted PSA | | | | | | 69.5% | 93.6% |

A 2×2 table of frequency counts (actual by predicted classification) was constructed and a likelihood ratio chi-squared (L²) was computed to test the null hypothesis that the model scores in each of the two groups are the same (with a 1-tailed p-value of less than 0.05 resulting in a successful validation) as shown in Table Vc. All candidate gene models demonstrated statistically significant model scores in each of the two groups and all candidate gene models resulted in lower validation p-values than the age-adjusted PSA criterion (p-value=8.7E-24).

TABLE Vc
Model Score Significance

| CATEGORY 2 Models (including plnPSA) | | | | | | Chi-Square Test |
|---|---|---|---|---|---|---|
| Gene 1 | Gene 2 | Gene 3 | Gene 4 | Gene 5 | Gene 6 | p-value |
| ABL1 | BRCA1 | | | | | 2.3E−33 |
| MAP2K1 | MAPK1 | | | | | 3.6E−26 |
| BRCA1 | MAP2K1 | | | | | 2.3E−28 |
| PTPRC | RP51077B9.4 | | | | | 6.3E−35 |
| CD97 | SP1 | | | | | 7.8E−40 |
| CD97 | CDK2 | RP51077B9.4 | SP1 | | | 5.1E−40 |
| BRCA1 | GSK3B | RB1 | TNF | | | 5.0E−33 |
| CD97 | GSK3B | PTPRC | RP51077B9.4 | SP1 | TNF | 6.0E−37 |
| CD97 | CDKN2A | IQGAP1 | RP51077B9.4 | SP1 | S100A6 | 3.7E−37 |
| Age-adjusted PSA | | | | | | 8.7E−24 |

Test 2a—Tests Based on Re-Estimated Parameters and Cut-Offs:

The repeat of Test 1 using model coefficients (beta parameters) re-estimated on the test dataset was not performed since all candidate models passed validation under the strict specifications of Test 1.

Test 2b—Tests Based on Re-Estimated Parameters Using a Likelihood Ratio (LR) Test:

Similarly, model re-estimation on the test dataset with a comparison to a restricted model estimated with PSA only was not performed since all candidate models passed validation under the strict specifications of Test 1.

### Test 3—Construction of ROC Curves and Area Under the Curves (AUC):

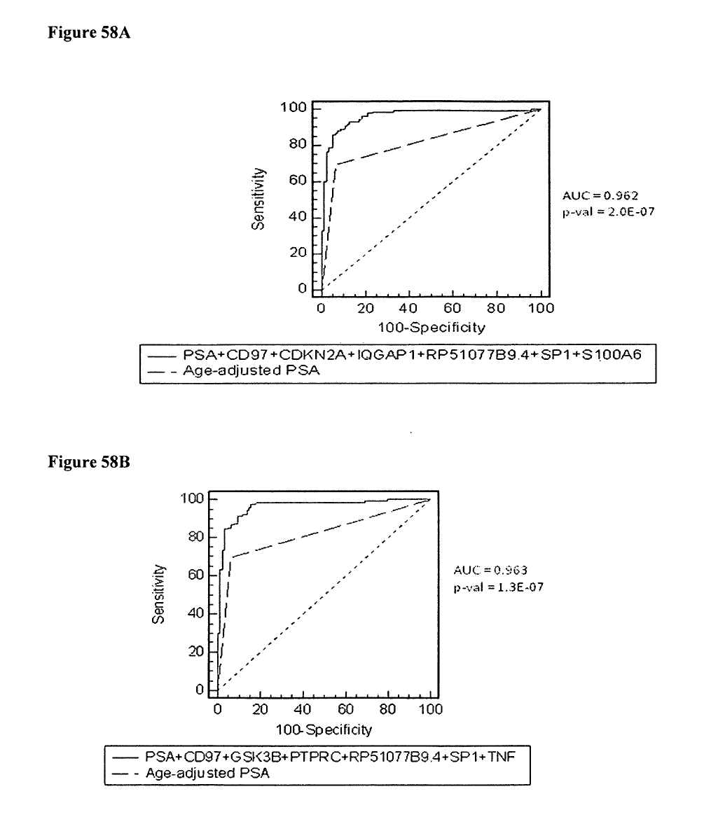

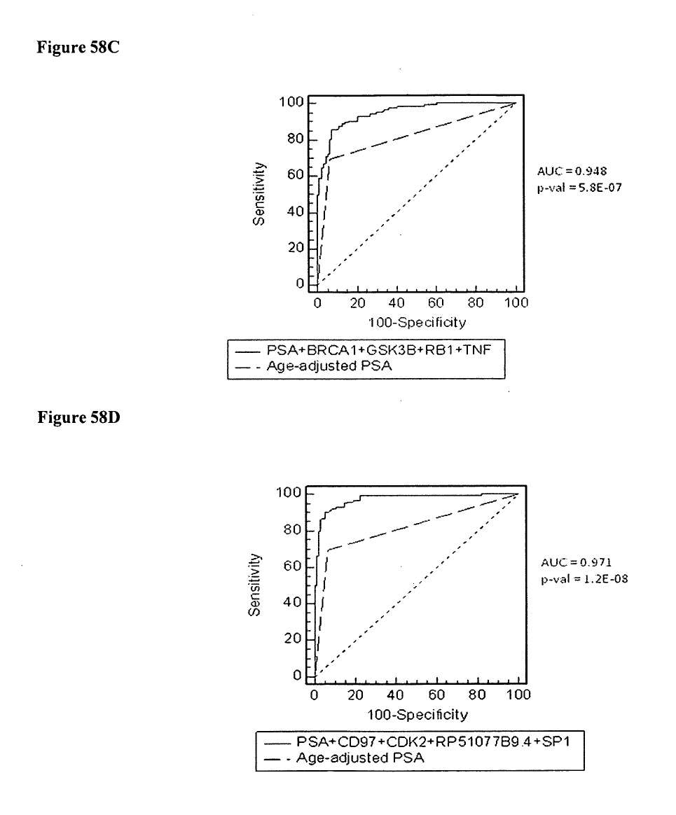

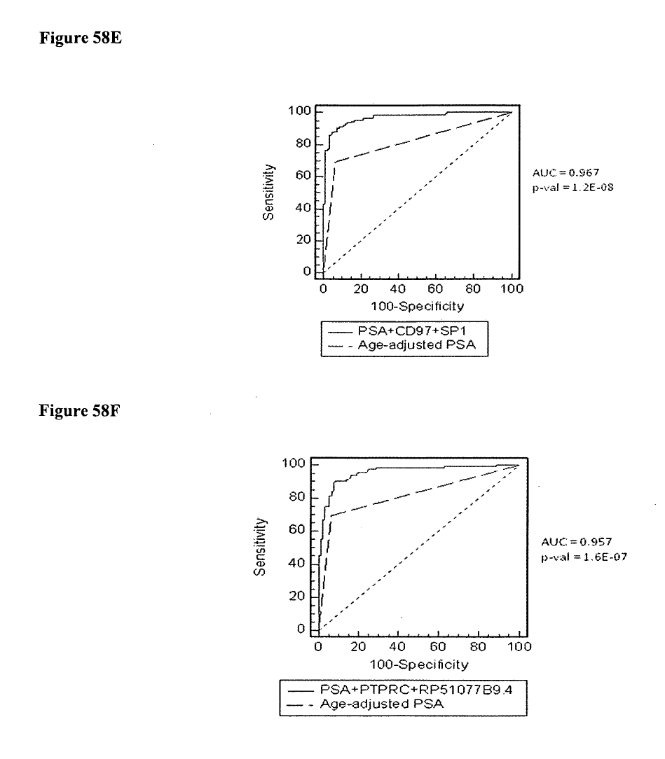

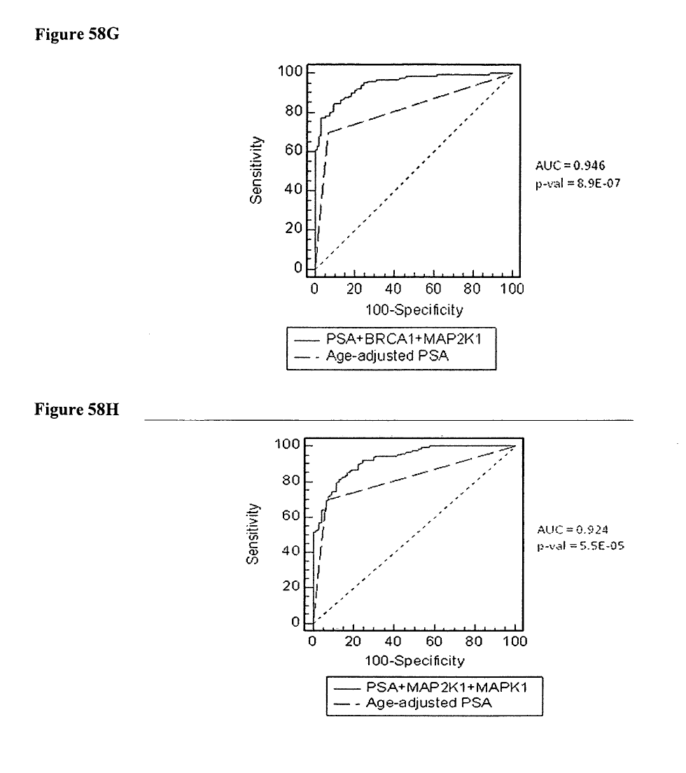

Using pre-specified coefficients established in the training dataset for each model, a model logit score was computed as in Test 1. Comparative ROC curves were constructed using the model logit score vs. the age-adjusted PSA criterion. Model validation was demonstrated by the significant improvement (p-value <0.05) in the area under the curve (AUC) associated with the logit model vs. age-adjusted PSA as shown in Table Vd below. Individual comparative ROC curves for all candidate models corresponding to Table Vd are provided in FIGS. 58A-58I.

TABLE Vd
Improvement in Area Under the ROC Curve -
Model vs Age-adjusted PSA

| Model + PSA | AUC | p-value* |
|---|---|---|
| CD97 + CDKN2A + IQGAP1 + RP51077B9.4 + | 0.962 | 2.0E−07 |
| SP1 + S100A6 | | |
| CD97 + GSK3B + PTPRC + RP51077B9.4 + SP1 + TNF | 0.963 | 1.3E−07 |
| BRCA1 + GSK3B + RB1 + TNF | 0.948 | 5.8E−07 |
| CD97 + CDK2 + RP51077B9.4 + SP1 | 0.971 | 1.2E−08 |
| CD97 + SP1 | 0.967 | 1.2E−08 |
| PTPRC + RP51077B9.4 | 0.957 | 1.6E−07 |
| BRCA1 + MAP2K1 | 0.946 | 8.9E−07 |
| MAP2K1 + MAPK1 | 0.924 | 5.5E−05 |
| ABL1 + BRCA1 | 0.946 | 6.4E−07 |
| *Test of improvement over AUC for Age-adjusted PSA | 0.816 | |

### Category 3 (CaP Versus BPH) Model Validation Tests and Specifications

The three 1-gene models, five 2-gene models, three 3-gene models and three 5-gene models, described in Table 6, all integrated with PSA and age, were submitted for validation using the parameters established in the training set as specified in Table 16 below.

The following Tests (1-3) were performed as necessary on the test dataset as a formal validation of all candidate Category 3 models. The progression of testing (pass/fail) and results for all models is summarized and illustrated in the flowchart provided in FIG. 59 with further supporting details and results that follow.

### Test 1—Strict Tests Based on Training Dataset Model Parameters and Cut-Offs:

For each of the models, the model logit score was computed using pre-specified coefficients (beta parameters) established in the training dataset (referred to in Table 16 below). A pre-specified logit cut-point of 0 for all models was applied to split the samples into two groups. Subjects with logit scores above the cut-point were predicted to be CaP patients and those whose scores fell below the cut-point were predicted to be normal subjects presenting with BPH as shown in Tables Wa and Wb. The age-adjusted PSA criterion misclassified 39 of the 128 CaP patients and 6 of the 80 BPH subjects in this test dataset, with comparable figures for the gene expression models provided in Table Wa. Based on the total number misclassified, all but 2 of the models (highlighted in Table Wa below) outperformed the age-adjusted PSA criterion. All gene expression models outperformed the age-adjusted PSA criterion in overall sensitivity and specificity as shown in Table Wb.

TABLE Wa
CaP vs BPH Frequency Counts by Model

| CATEGORY 3 Models (including plnPSA) | | | | | Number Misclassified | | |
|---|---|---|---|---|---|---|---|
| Gene 1 | Gene 2 | Gene 3 | Gene 4 | Gene 5 | CaP (N = 128) | BPH (N = 80) | Total |
| IL18 | | | | | 19 | 9 | 28 |
| RP51077B9.4 | | | | | 32 | 14 | 46 |
| S100A6 | | | | | 26 | 10 | 36 |
| CD97 | S100A6 | | | | 30 | 15 | 45 |
| IL18 | RP51077B9.4 | | | | 18 | 14 | 32 |
| MAP2K1 | S100A6 | | | | 26 | 16 | 42 |
| RP51077B9.4 | S100A6 | | | | 17 | 16 | 33 |
| RP51077B9.4 | SP1 | | | | 26 | 9 | 35 |
| MAP2K1 | MYC | S100A6 | | | 11 | 15 | 26 |
| MAP2K1 | S100A6 | TP53 | | | 21 | 20 | 41 |
| MAP2K1 | S100A6 | SMAD3 | | | 16 | 19 | 35 |
| MAP2K1 | MYC | S100A6 | RP51077B9.4 | C1QA | 19 | 13 | 32 |
| MAP2K1 | SMAD3 | S100A6 | CCNE1 | TP53 | 20 | 17 | 37 |
| MAP2K1 | TP53 | S100A6 | CCNE1 | ST14 | 24 | 18 | 42 |
| Age-adjusted PSA | | | | | 39 | 6 | 45 |

TABLE Wb
Sensitivity and Specificity by Model

| CATEGORY 3 Models (including plnPSA) | | | | | Classification | |
|---|---|---|---|---|---|---|
| Gene 1 | Gene 2 | Gene 3 | Gene 4 | Gene 5 | Sensitivity | Specificity |
| IL18 | | | | | 85.2% | 88.8% |
| RP51077B9.4 | | | | | 75.0% | 82.5% |
| S100A6 | | | | | 79.7% | 87.5% |
| CD97 | S100A6 | | | | 76.6% | 81.3% |
| IL18 | RP51077B9.4 | | | | 85.9% | 82.5% |
| MAP2K1 | S100A6 | | | | 79.7% | 80.0% |
| RP51077B9.4 | S100A6 | | | | 86.7% | 80.0% |
| RP51077B9.4 | SP1 | | | | 79.7% | 88.8% |
| MAP2K1 | MYC | S100A6 | | | 91.4% | 81.3% |
| MAP2K1 | S100A6 | TP53 | | | 83.6% | 75.0% |
| MAP2K1 | S100A6 | SMAD3 | | | 87.5% | 76.3% |
| MAP2K1 | MYC | S100A6 | RP51077B9.4 | C1QA | 85.2% | 83.8% |
| MAP2K1 | SMAD3 | S100A6 | CCNE1 | TP53 | 84.4% | 78.8% |
| MAP2K1 | TP53 | S100A6 | CCNE1 | ST14 | 81.3% | 77.5% |
| Age-adjusted PSA | | | | | 69.5% | 92.5% |

A 2×2 table of frequency counts (actual by predicted classification) was constructed and a likelihood ratio chi-squared (L²) was computed to test the null hypothesis that the model scores in each of the two groups are the same (with a 1-tailed p-value of less than 0.05 resulting in a successful validation) as shown in Table We. All candidate gene models demonstrated statistically significant model scores in each of the two groups and most of the gene expression models (with exceptions highlighted in Table We) resulted in lower validation p-values than that for the PSA age-adjusted criterion (p-value=1.2E-19).

TABLE Wc
Model Score Significance

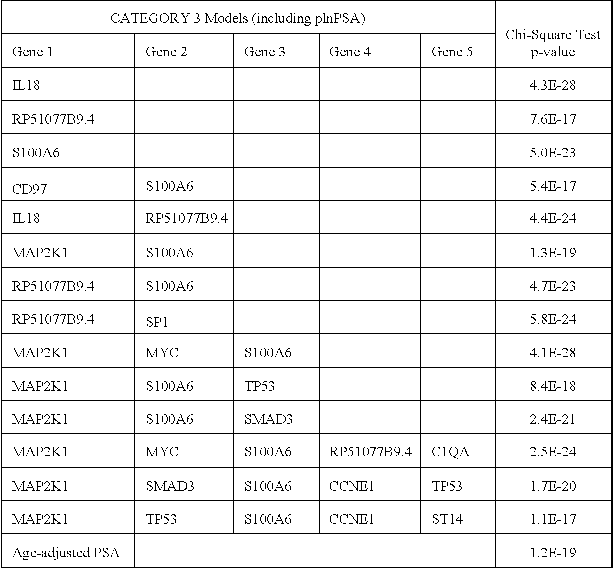

Test 2a—Tests Based on Re-Estimated Parameters and Cut-Offs:

The repeat of Test 1 using model coefficients (beta parameters) re-estimated on the test dataset was not performed since all candidate models passed validation under the strict specifications of Test 1.

Test 2b—Tests Based on Re-Estimated Parameters Using a Likelihood Ratio (LR) Test:

Similarly, model re-estimation on the test dataset with a comparison to a restricted model estimated with PSA only was not performed since all candidate models passed validation under the strict specifications of Test 1.

### Test 3—Construction of ROC Curves and Area Under the Curves (AUC):

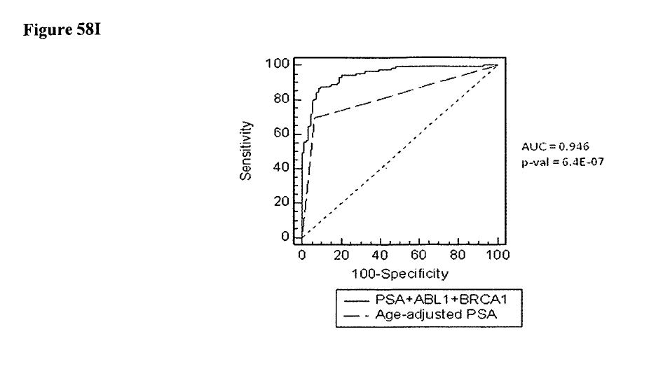

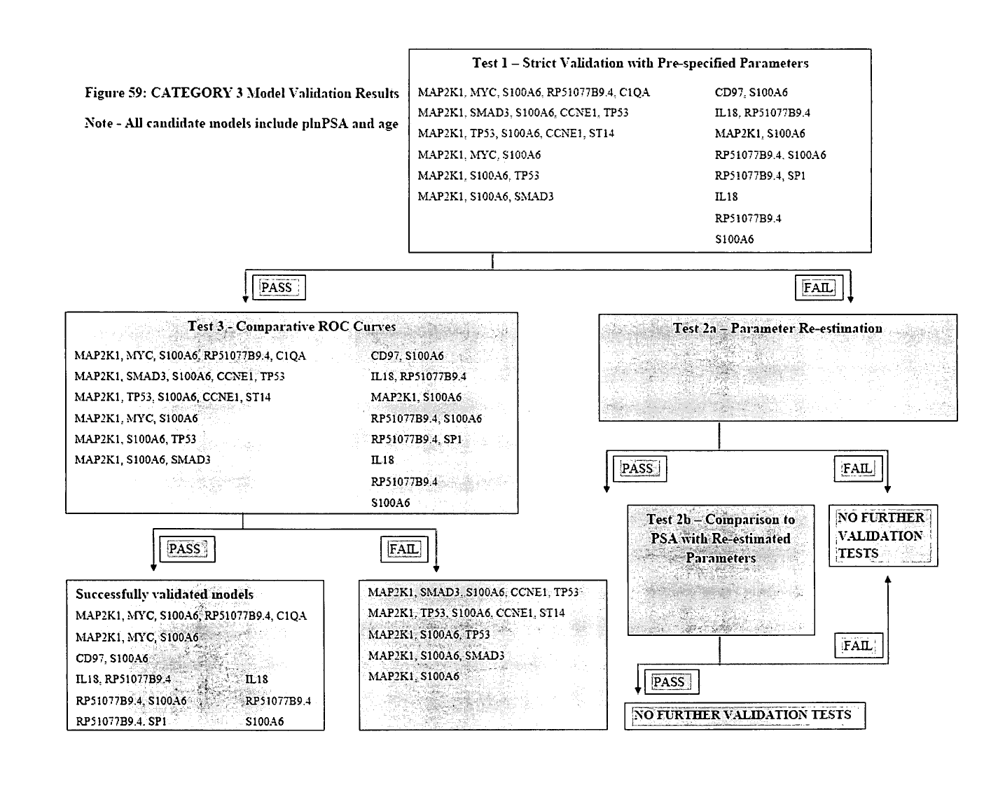

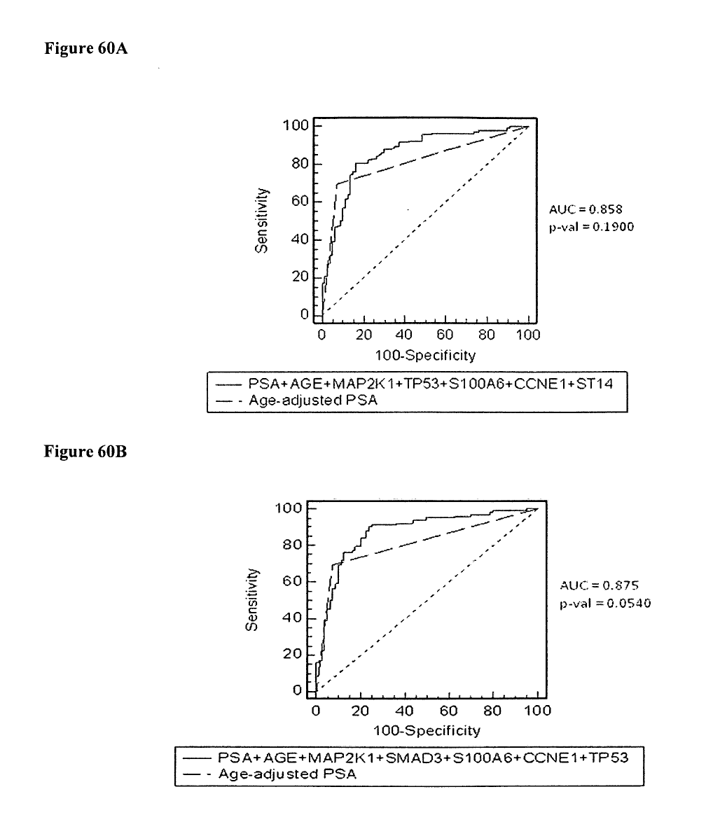

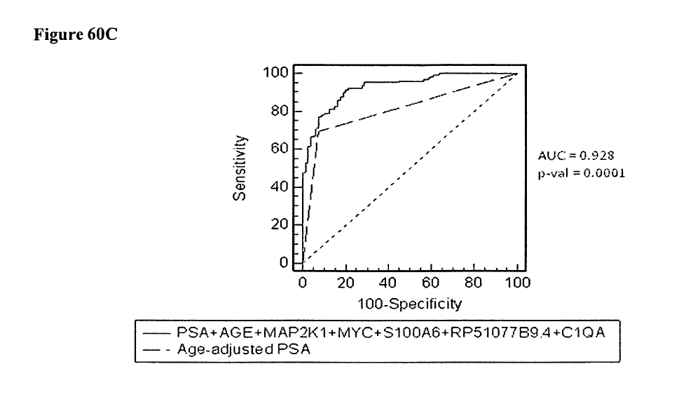

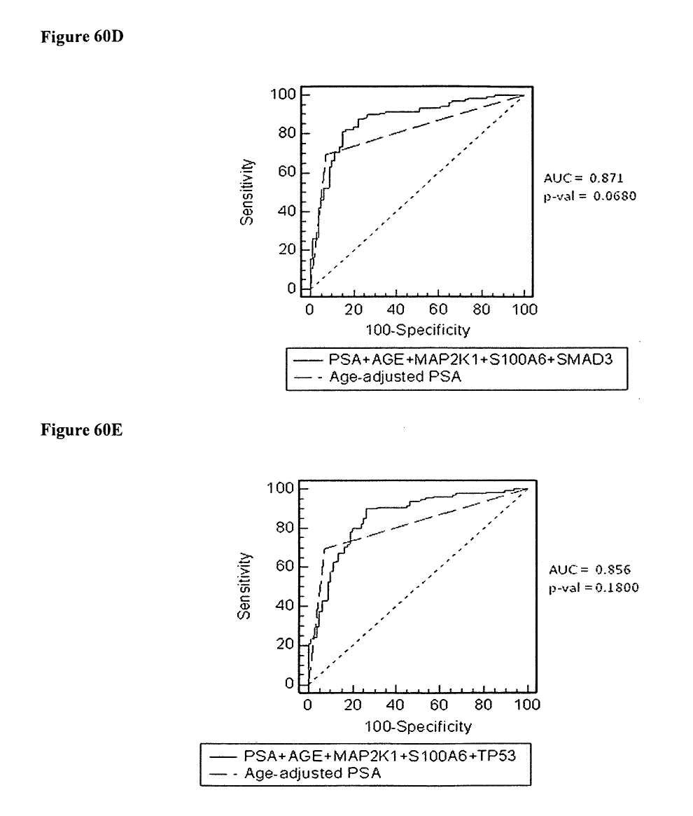

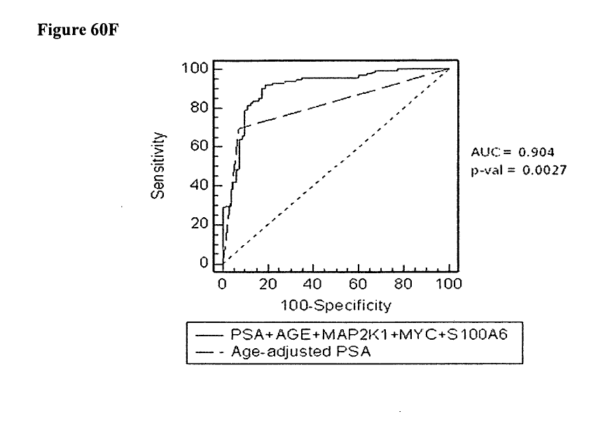

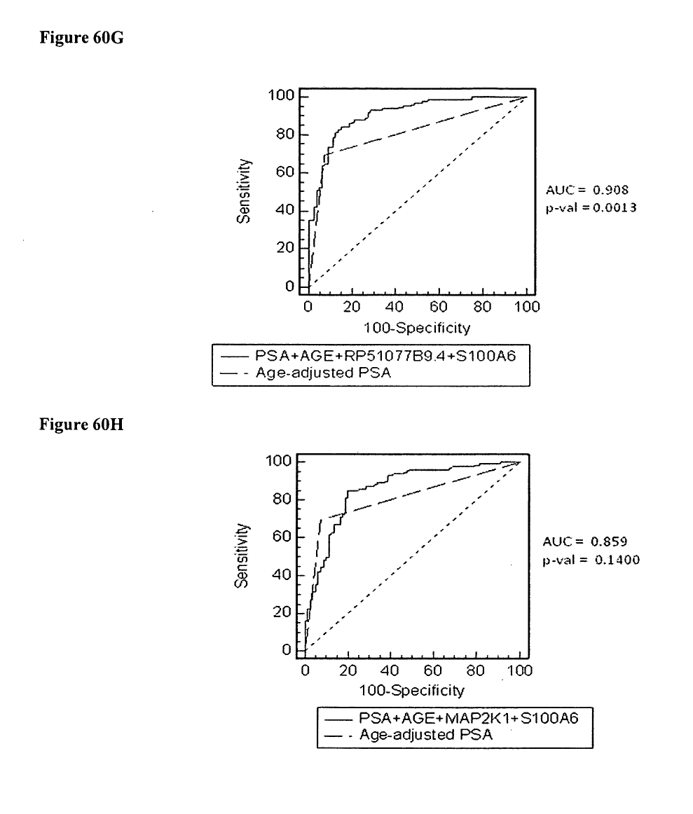

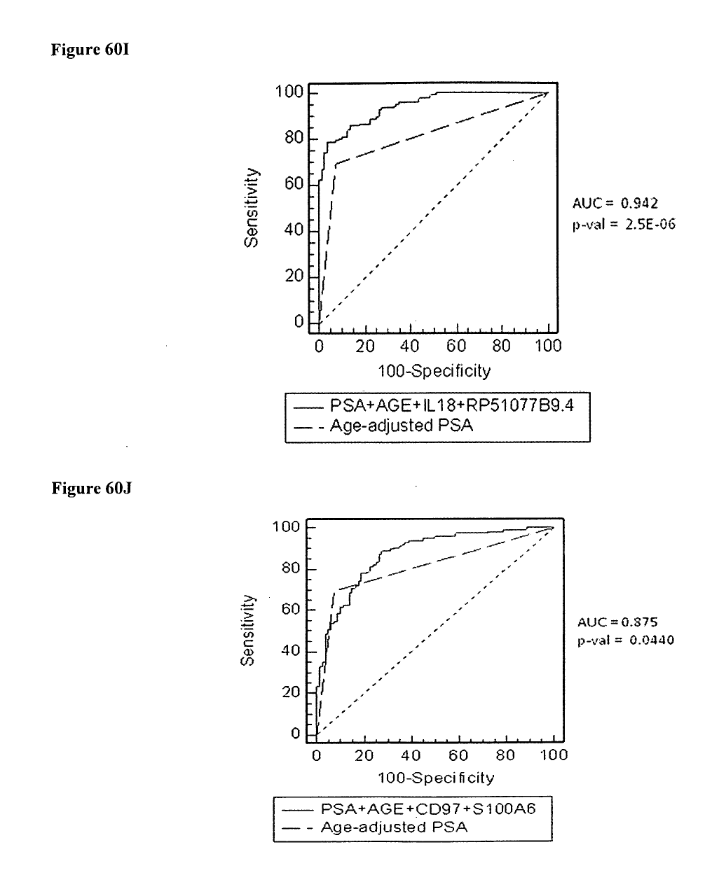

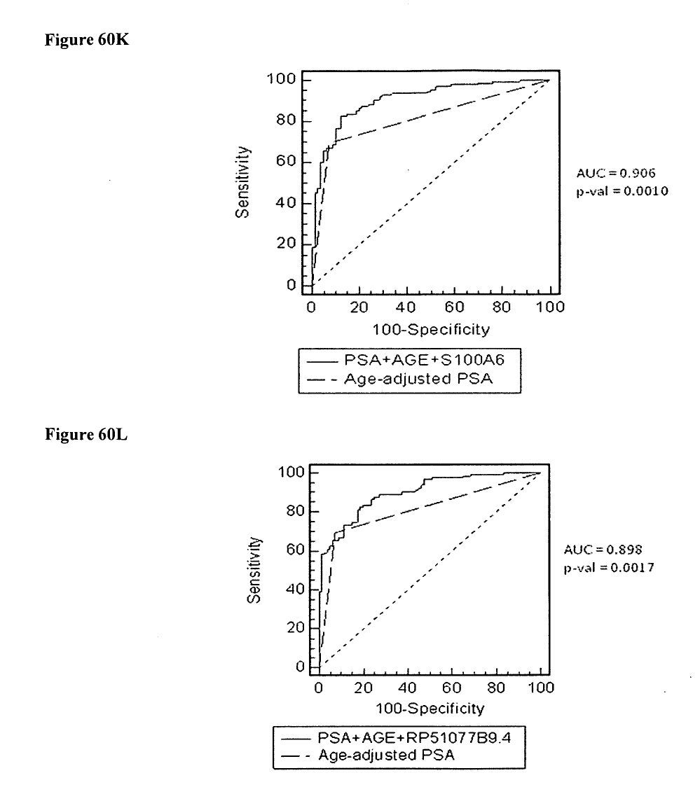

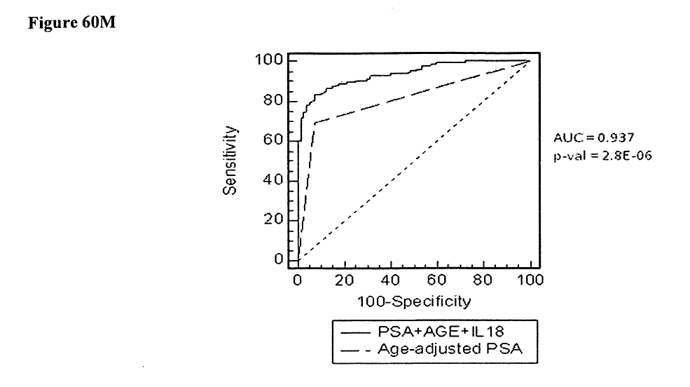

Using pre-specified coefficients established in the training dataset for each model, a model logit score was computed as in Test 1. Comparative ROC curves were constructed using the model logit score vs. the age-adjusted PSA criterion. Nine of fourteen models validated by demonstrating significant improvement (p-value <0.05) in the area under the curve (AUC) associated with the logit model vs. age-adjusted PSA as shown in Table Wd below (exceptions highlighted). Individual comparative ROC curves for all candidate models corresponding to Table Wd are provided in FIGS. 60A-60M.

TABLE Wd
Improvement in Area Under the ROC Curve— Model vs Age-adjusted PSA

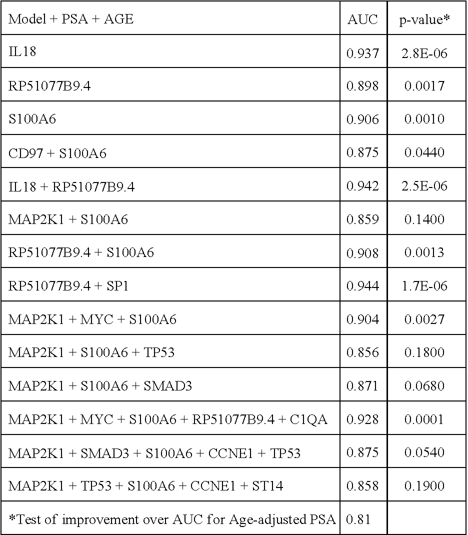

### Example 13

### Discrimination of Prostate Cancer Subjects from Healthy, Normal Subjects (Excluding BPH) Using RNA Transcript-Based Gene Expression (w/o PSA Values): Combined Training and Test Datasets

The Training Dataset and Test Datasets described in Example 1 were combined and stepwise methodology was used to enumerate 1- and 2-gene models capable of discriminating prostate cancer subjects from normal, healthy subjects (without BPH) without coincidental measurement of PSA values, based on the 22 genes that were included in the Category 2 and Category 3 models validated in Example 12 above. Separate training and validation sets were not performed since all 22 genes had already validated in one or more of the Category 2 or Category 3 models as described in Example 12. A listing of the 1- and 2-gene models based on the 22 validated genes described in Example 12 is shown in Table 17A.

Stepwise logistic regression was then used to further identify all possible 8-gene models capable of discriminating prostate cancer subjects from normal, healthy subjects (without BPH) without coincidental measurement of PSA values. Enumeration of possible 8-gene models was Approximately 9,000 8-gene models with over 75% correct classification and about 1,000 8-gene models with over 85% correct classification were identified. A subset of these 8-gene models is shown in Table 17B.

As shown in Table 17B, the 8-gene models are identified in the first eight columns (respectively) on the left side of Table 17B, ranked by their entropy R² value (shown in column 9, ranked from high to low). The number of subjects correctly classified or misclassified by each 8-gene model for each patient group (i.e., CaP vs. Normal (excluding BPH) is shown in columns 10-13. The percent normal subjects and percent prostate cancer subjects correctly classified by the corresponding gene model is shown in columns 14 and 15. The incremental p-values for each of the 8-genes is shown in columns 16-23, and the gene coefficients are shown in columns 24-31.

For example, the “best” 8-gene logistic regression model capable of distinguishing between prostate cancer subjects and normal, healthy subjects (defined as the model with the highest entropy R² value, as described in Example 2) based on the 22 genes analyzed is BRCA1, CD97, CDK2, IQGAP1, PTPRC, RP51077B9.4, SP1 and TNF, capable of classifying normal subjects with 87.7% accuracy (87.7% specificity), and prostate cancer subjects with 88.7% accuracy (88.7% sensitivity). This 8-gene model correctly classifies 149 of the normal subjects as being in the normal patient population, and misclassifies 21 of the normal subjects as being in the prostate cancer patient population (i.e., 87.7% correct classification). This 8-gene model correctly classifies 181 of the prostate cancer subjects as being in the prostate cancer patient population and misclassifies 23 of the prostate cancer subjects as being in the normal patient population (i.e., 88.7% correct classification).

As a further example, the 8-gene model ABL1, BRCA1, CD97, IL18, IQGAP1, RP51077B9.4, SP1 and TNF, shown in Table 17B, is capable of classifying normal subjects with 90% accuracy (90% specificity), and prostate cancer subjects with 89.2% accuracy (89.2% sensitivity). This 8-gene model correctly classifies 153 of the normal subjects as being in the normal patient population, and misclassifies 17 of the normal subjects as being in the prostate cancer patient population (i.e., 87.7% correct classification). This 8-gene model correctly classifies 182 of the prostate cancer subjects as being in the prostate cancer patient population and misclassifies 22 of the prostate cancer subjects as being in the normal patient population (i.e., 89.2% correct classification).

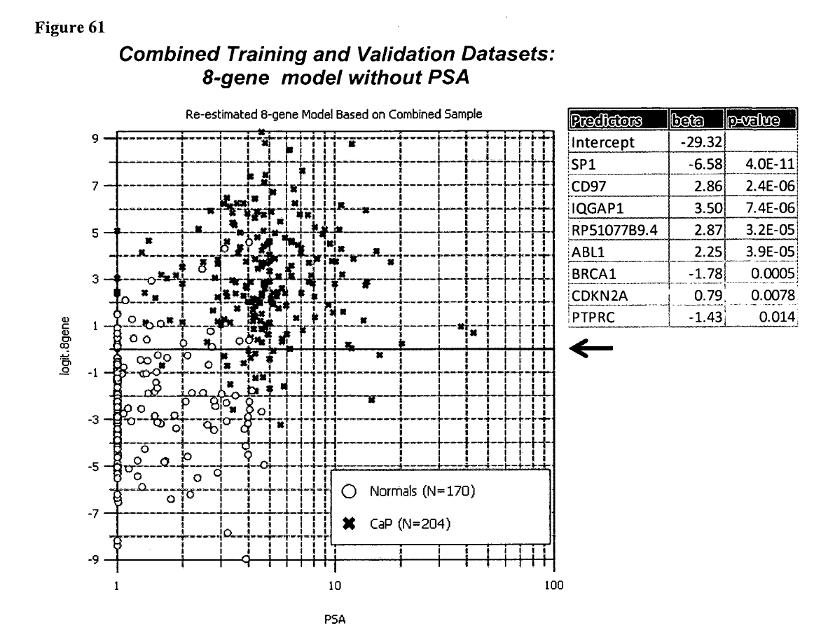

An example of an 8-gene model, SP1, CD97, IQGAP1, RP51077B9.4, ABL1, BRCA1, CDKN2A and PTPRC, capable of discriminating between prostate cancer subjects and normal, healthy subjects, is shown in FIG. 61. As shown in the 8-gene model shown in FIG. 61, 87.7% of the CaP subjects are correctly predicted by the model (above the arrow indicated line) while 87.6% of the Normal subjects are correctly predicted by the model (below the arrow indicated line).

A ranking of the top genes for which gene expression profiles were obtained, from most to least significant, is shown in Table 17C. Table 17C summarizes the mean expression levels of the 22 genes measured in the RNA samples obtained from the prostate cancer subjects in the Training and Test Datasets, as well as the results of significance tests (Wald p-values) for the difference in the mean expression levels between the normal and prostate cancer subjects.

The data described herein supports that Gene Expression Profiles with sufficient precision and calibration as described herein (1) can determine subsets of individuals with a known biological condition, particularly individuals with prostate cancer or individuals with conditions related to prostate cancer and individuals with aggressive vs. non-aggressive forms of prostate cancer; (2) may be used to monitor the response of patients to therapy; (3) may be used to assess the efficacy and safety of therapy; and (4) may be used to guide the medical management of a patient by adjusting therapy to bring one or more relevant Gene Expression Profiles closer to a target set of values, which may be normative values or other desired or achievable values.

Gene Expression Profiles are used for characterization and monitoring of treatment efficacy of individuals with prostate cancer, or individuals with conditions related to prostate cancer, and for characterizing and monitoring of individuals with aggressive vs. non-aggressive forms of prostate cancer. Use of the algorithmic and statistical approaches discussed above to achieve such identification and to discriminate in such fashion is within the scope of various embodiments herein.

The references listed below are hereby incorporated herein by reference.

### REFERENCES

Magidson, J. GOLDMineR User's Guide (1998). Belmont, Mass.: Statistical Innovations Inc.
Vermunt and Magidson (2005). Latent GOLD 4.0 Technical Guide, Belmont Mass.: Statistical Innovations.
Vermunt and Magidson (2007). LG-Syntax™ User's Guide: Manual for Latent GOLD® 4.5 Syntax Module, Belmont Mass.: Statistical Innovations.
Vermunt J. K. and J. Magidson. Latent Class Cluster Analysis in (2002) J. A. Hagenaars and A. L. McCutcheon (eds.), Applied Latent Class Analysis, 89-106. Cambridge: Cambridge University Press.
Magidson, J. “Maximum Likelihood Assessment of Clinical Trials Based on an Ordered Categorical Response.” (1996) Drug Information Journal, Maple Glen, Pa.: Drug Information Association, Vol. 30, No. 1, pp 143-170.

Lengthy table referenced here
US20120301887A1-20121129-T00001
Please refer to the end of the specification for access instructions.

Lengthy table referenced here
US20120301887A1-20121129-T00002
Please refer to the end of the specification for access instructions.

Lengthy table referenced here
US20120301887A1-20121129-T00003
Please refer to the end of the specification for access instructions.

Lengthy table referenced here
US20120301887A1-20121129-T00004
Please refer to the end of the specification for access instructions.

Lengthy table referenced here
US20120301887A1-20121129-T00005
Please refer to the end of the specification for access instructions.

Lengthy table referenced here
US20120301887A1-20121129-T00006
Please refer to the end of the specification for access instructions.

Lengthy table referenced here
US20120301887A1-20121129-T00007
Please refer to the end of the specification for access instructions.

Lengthy table referenced here
US20120301887A1-20121129-T00008
Please refer to the end of the specification for access instructions.

Lengthy table referenced here
US20120301887A1-20121129-T00009
Please refer to the end of the specification for access instructions.

Lengthy table referenced here
US20120301887A1-20121129-T00010
Please refer to the end of the specification for access instructions.

Lengthy table referenced here
US20120301887A1-20121129-T00011
Please refer to the end of the specification for access instructions.

Lengthy table referenced here
US20120301887A1-20121129-T00012
Please refer to the end of the specification for access instructions.

Lengthy table referenced here
US20120301887A1-20121129-T00013
Please refer to the end of the specification for access instructions.

Lengthy table referenced here
US20120301887A1-20121129-T00014
Please refer to the end of the specification for access instructions.

Lengthy table referenced here
US20120301887A1-20121129-T00015
Please refer to the end of the specification for access instructions.

Lengthy table referenced here
US20120301887A1-20121129-T00016
Please refer to the end of the specification for access instructions.

Lengthy table referenced here
US20120301887A1-20121129-T00017
Please refer to the end of the specification for access instructions.

Lengthy table referenced here
US20120301887A1-20121129-T00018
Please refer to the end of the specification for access instructions.

Lengthy table referenced here
US20120301887A1-20121129-T00019
Please refer to the end of the specification for access instructions.

Lengthy table referenced here
US20120301887A1-20121129-T00020
Please refer to the end of the specification for access instructions.

Lengthy table referenced here
US20120301887A1-20121129-T00021
Please refer to the end of the specification for access instructions.

Lengthy table referenced here
US20120301887A1-20121129-T00022
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20120301887A1). An electronic copy of the table will also be available from the USPTO upon request and payment of the fee set forth in 37 CFR 1.19(b)(3).

1-7. (canceled)

8. A method for evaluating the presence of aggressive prostate cancer in a subject based on a sample from the subject, the sample providing a source of RNAs, comprising:
a) determining a quantitative measure of the amount of at least one constituent of Table 1 or Table 8, and
b) predicting the likelihood of the subject having aggressive prostate cancer, wherein if the constituent has a positive coefficient, the amount of the constituent negatively correlates with an increased likelihood of the subject having aggressive prostate cancer, and if the constituent has a negative coefficient, the amount of the constituent positively correlates with an increased likelihood of the subject having aggressive prostate cancer.

9. The method of claim 8, wherein the at least one constituent is SPARC and wherein the amount of SPARC positively correlates with an increased likelihood of the subject having aggressive prostate cancer.

10-13. (canceled)

14. The method of claim 8, wherein the subject is predicted to have an aggressive prostate cancer if the subject is predicted to have a prostate tumor with a Gleason score of 7 or higher.

15-16. (canceled)

17. The method of claim 8, wherein the sample is selected from blood, a blood fraction, a body fluid, a cell, and a tissue.

18-24. (canceled)

25. The method of claim 8, wherein the sample is a biopsy sample.

26. The method of claim 8, wherein the amount of the at least one constituent is normalized relative to the amount of one or more reference genes.

27. The method of claim 8, wherein the amount of the at least one constituent is determined by quantitative reverse transcription polymerase chain reaction (RT-PCR).

28. The method of claim 14, wherein the subject is predicted to have an aggressive prostate cancer if the subject is predicted to have a prostate tumor with a Gleason score of 7(4+3) or higher.

29. The method of claim 14, wherein the subject is predicted to have an aggressive prostate cancer if the subject is predicted to have a prostate tumor with a Gleason score of 8 or higher.
